(19) United States
(12) Patent Application Publication
(10) Pub. No.: US 2009/0118332 A1
(43) Pub. Date: May 7, 2009

(54) Therapeutic Agents - 551

(75) Inventors: Roger John Butlin, Macclesfield (GB); Peter William Rodney Caulkett, Macclesfield (GB); Andrew Leach, Macclesfield (GB); Nicholas John Newcombe, Macclesfield (GB); Charles John O'Donnell, Macclesfield (GB); James Matthew Wood, Macclesfield (GB)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 11/961,843

(22) Filed: Dec. 20, 2007

Related U.S. Application Data
(60) Provisional application No. 60/871,198, filed on Dec. 21, 2006.
(60) Provisional application No. 60/910,268, filed on Apr. 5, 2007.

(51) Int. Cl. A61K 31/451; C07D 211/60; A61P 3/04; A61P 35/00; A61P 31/00

(52) U.S. Cl. 514/330; 546/226

(57) ABSTRACT

A compound of formula I

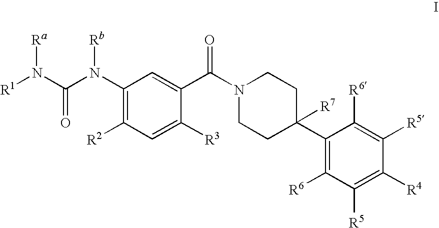

or a pharmaceutically acceptable salt thereof, processes for preparing such compounds, their use as Fatty Acid Synthase inhibitors, methods for their therapeutic use, particularly in the treatment of obesity, diabetes mellitus, cancer and infection and pharmaceutical compositions containing them.

This application claims the benefit under 35 U.S.C. § 119(e) of Application No. 60/871,198 (US sort No. 1) filed on 21 Dec. 2006, and Application No. 60/910,268 (US sort No. 2) filed on 5 Apr. 2007.

### FIELD OF INVENTION

The present invention relates to ureas, particularly to substituted N-[3-[4-(4-cyanophenyl)piperidine-1-carbonyl]phenyl]-N′-(substituted alkyl)ureas, to processes for preparing such compounds, to their use as Fatty Acid Synthase inhibitors, to methods for their therapeutic use, particularly in the treatment of obesity and diabetes mellitus, and to pharmaceutical compositions containing them.

### BACKGROUND OF THE INVENTION

Obesity and diabetes are reaching epidemic proportions in the USA, EU, Japan and developing countries. Obesity is the major driver of the co-morbidities of the metabolic syndrome, particularly type 2 diabetes. Since no effective pharmacotherapies for obesity are available to date and current diabetes therapies do not stop the progression of the disease, there is a huge unmet medical need.

Fatty Acid Synthase (FAS) is a critical enzyme for endogenous lipogenesis and plays an important role in the modulation of key intermediates of lipid and carbohydrate cellular metabolism. FAS is highly expressed in the tissues with high metabolic activity (for example liver, adipose tissue and brain) and there are good reasons to believe that a FAS inhibitor would cause beneficial metabolic effects in peripheral tissues. In addition, inhibition of FAS in the hypothalamus may result in reduced food intake. The non-specific irreversible FAS inhibitors cerulenin and C-75 have been reported in the literature to decrease brain levels of orexigenic neuropeptides and to decrease food intake.

Therefore there is a need for an effective FAS inhibitor to treat obesity and diabetes.

### DESCRIPTION OF THE INVENTION

The present invention provides a compound of formula I

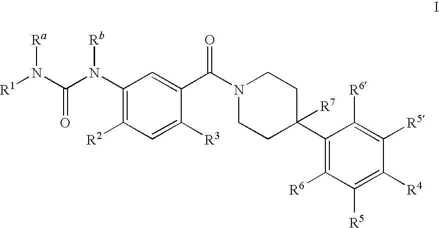

or a pharmaceutically acceptable salt thereof, in which
R¹ represents 1) a C₁₋₆alkyl group optionally substituted by one or two groups selected from A-Y below and/or by one to five groups selected from X below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) a group CONR^{e}R^{f} in which R^{e} and R^{f} are as defined below; vii) C₁₋₆alkanoyl; viii) benzoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulfamoyl; xvi) N—C₁₋₆alkylsulfamoyl; xvii) N,N-diC₁₋₆alkylsulfamoyl; xviii) benzyl or benzyloxy; xix) nitro; xx) heteroaryl; xxi) heteroaryloxy; xxii) phenyl xxiii) phenoxy xxiv) phenylsulfamoyl; xxv) heteroarylsulfamoyl; xxvi) a carbon linked saturated or partially unsaturated 4 to 10 membered heterocyclic group as defined in c) below; xxvii) phenylsulfonyl; xxviii) heteroarylsulfonyl;
xxix) a group of formula NR^{c}R^{d} in which R^{c} and R^{d} independently represent:

### a) H;

b) C₁₋₆alkanoyl optionally substituted by carboxy or a C₁₋₆alkoxycarbonyl group;
c) a carbon linked saturated or partially unsaturated 4 to 10 membered heterocyclic group containing one or more N, S or O, wherein the S may be in its oxidised form of SO or SO₂, which is optionally fused to a benz ring and any ring is optionally substituted by one or more of the following: hydroxy, halo, oxo, carboxy, a C₁₋₆ alkoxycarbonyl group, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 10 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; halo; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl or nitro;
k) heteroaryl optionally substituted by one or more carboxy; fluoro; hydroxy; a C₁₋₆alkyl group (which is optionally substituted by one or more of the following: a C₁₋₆alkoxy group, hydroxy or a group of formula NR^{e}R^{f} in which R^{e} and R^{f} are as defined above); a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group optionally substituted by phenyl;
n) heteroarylcarbonyl;
o) sulfamoyl optionally substituted by one or two independently selected C₁₋₆alkyl groups or the terminal nitrogen is included in a 5 or 6 membered saturated or partially unsaturated heterocyclic ring optionally containing an additional N, S or O, wherein the S may be in its oxidised form of SO or SO₂;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 10 membered heterocyclic group containing one or more N, S or O, wherein the S may be in its oxidised form of SO or SO₂, which is optionally fused to a benz ring or a heteroaryl ring and is optionally substituted on any ring by one or more of the following: hydroxy; halo; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; a C₁₋₆alkyl optionally substituted by one or more hydroxy or C₁₋₆alkoxy; or a C₁₋₆ alkoxycarbonyl group;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) C₁₋₆alkylthio;
R) ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
S) phenoxy;
T) hydroxy;

### U) oxo

V) carboxy;
W) cyano;
X) sulfamoyl optionally substituted by one or two independently selected C₁₋₆alkyl groups or the nitrogen is included in a 4 or 7 membered saturated or partially unsaturated heterocyclic ring optionally containing an additional N, S or O, wherein the S may be in its oxidised form of SO or SO₂;
Y) sulfamoylamino optionally substituted by one or two independently selected C₁₋₆alkyl groups or the terminal nitrogen is included in a 4 or 7 membered saturated or partially unsaturated heterocyclic ring optionally containing an additional N, S or O, wherein the S may be in its oxidised form of SO or SO₂;
Z) fluoro or chloro;
or R¹ represents
2) a C₃₋₁₀cycloalkyl group optionally substituted by one or two groups selected from A to Y above and/or by one to five groups selected from Z above;
3) a C₂₋₆alkynyl group optionally substituted by one or two groups selected from A to Y above and/or by one to five groups selected from Z above;
4) a carbon linked saturated or partially unsaturated 4 to 10 membered heterocyclic group containing one or more N, S or O, wherein the S may be in its oxidised form of SO or SO₂, which is optionally fused to a benz ring or a heteroaryl ring and any ring is optionally substituted by one or two groups A to Y as defined above and/or by one to five groups selected from Z above;
5) a C₂₋₆alkenyl group optionally substituted by one or two groups selected from A to Y above and/or by one to five groups selected from Z above;
wherein any alkyl chain mentioned in any of the definitions from A to Y above or in any of the definitions i to xxix above is optionally substituted by 1) one or two groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; a C₁₋₄alkanoyloxy group or a C₁₋₄alkyl optionally substituted by one or more hydroxy, C₁₋₃alkoxy or a group —NR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 10 membered heterocyclic group in the list of optional substituents from A to Y above or in any of the definitions i to xxix above, for which specific substitution has not been previously mentioned, is optionally substituted by one, two or three groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R^{a} represents H; or a C₁₋₄alkyl group, a C₃₋₆cycloalkyl group or a C₃₋₆cycloalkylC₁₋₄alkyl group each of which groups is optionally substituted by one or more carboxy; fluoro; hydroxy; a C₁₋₃alkoxy group optionally substituted on C2 or C3 by carboxy; a group of formula NR^{e}R^{f} in which R^{e} and R^{f} are as defined above; or a group CONR^{e}R^{f} in which R^{e} and R^{f} are as defined above;
or R¹ and R^{a} together with the nitrogen atom to which they are attached represent a saturated or partially unsaturated 4 to 10 membered heterocyclic ring optionally containing an additional sulphur including oxidised as SO or SO₂, oxygen or nitrogen which is optionally fused to a benz ring and wherein any ring is optionally substituted by one or two of the groups A to Y above and/or by from 1 to 5 groups Z;
R^{b} represents H;
R² represents H, halo, cyano, a C₁₋₃alkyl group which is optionally substituted by one or more of the following: halo; a C₁₋₃alkoxy group; or by a group C₁₋₃alkylS(O)ᵤ— which is optionally substituted by one or more fluoro and u is 0, 1 or 2; or R² represents a C₁₋₃alkoxy group optionally substituted by one or more halo or R² represents a C₁₋₆alkylS(O)ₐ(O)_{b}— group wherein the C₁₋₆alkyl is optionally substituted by one or more fluoro and a is 0, 1 or 2 and b is 0 except when a is 2 then b may also be 1;
R³ represents H, halo, cyano, a C₁₋₃alkyl group which is optionally substituted by one or more of the following: halo; C₁₋₃alkoxy group; or by a group C₁₋₃alkylS(O)ₜ— which is optionally substituted by one or more fluoro and t is 0, 1 or 2; or R² represents a C₁₋₃alkoxy group optionally substituted by one or more halo or R² represents a C₁₋₆alkylS(O)_{c}(O)_{d}— group wherein the C₁₋₆alkyl is optionally substituted by one or more fluoro and c is 0, 1 or 2 and d is 0 except when c is 2 then d may also be 1;
R⁴ represents i) H
ii) a C₁₋₃alkyl group optionally substituted by cyano, hydroxy, a C₁₋₃alkoxy group or optionally substituted by one or more halo
iii) a C₁₋₃alkoxy group optionally substituted by one or more halo or optionally substituted by cyano, hydroxy, a C₁₋₃alkoxy group, an amino group of formula NR^{u}R^{v} in which R^{u} and R^{v} independently represent H, a C₁₋₃alkylsulphonyl group, an aminoC₁₋₃alkylsulphonyl group in which the amino is optionally substituted by one or more C₁₋₃alkyl groups, a C₁₋₃alkanoyl group, a C₁₋₃alkoxycarbonyl group or a C₁₋₃alkyl group optionally substituted by hydroxy or R^{u} and R^{v} together with the nitrogen atom to which they are attached represent azetidinyl, pyrrolidinyl, piperidinyl, piperazinyl or morpholinyl each of which is optionally substituted by one or more of the following: oxo, C₁₋₃alkyl or hydroxy;
iv) halo v) nitro vi) cyano
vii) a C₁₋₆alkylS(O)_{y}(O)_{z}— optionally substituted by one or more fluoro wherein y is 0, 1 or 2 and z is 0 except when y is 2 when z is 0 or 1
viii) a group -L-R^{g} in which L represents a bond, a C₃₋₆cycloalkylene group, a C₃₋₆cycloalkylidene group, a C₁₋₆alkylene group or a C₁₋₆alkoxyC₁₋₆alkylene group wherein each group is optionally substituted by one or more of the following: carboxy, hydroxy, a C₁₋₃alkyl group optionally substituted by hydroxy; and R^{g} represents carboxy or a group NR^{u}R^{v} in which R^{u} and R^{v} are as defined above and additionally R^{v} represents cyano or R^{g} represents a group CO₂R^{w} in which R^{w} is a C₁₋₃alkyl group; or R^{g} represents a group CONR^{x}R^{y} in which R^{x} and R^{y} independently represent H, a C₁₋₃alkylsulphonyl group, a C₁₋₃alkyl group or a C₃₋₆cycloalkyl group wherein the alkyl and cycloalkyl groups are optionally substituted by one or more hydroxy, carboxy or NR^{u}R^{v} in which R^{u} and R^{v} are as previously defined, or R^{x} and R^{y} together with the nitrogen atom to which they are attached represent azetidinyl; pyrrolidinyl, piperidinyl or morpholinyl; or R^{g} represents tetrazolyl, thiazolidin-2,4-dion-5-yl or R^{g} represents ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
ix) a group -L₁-N(R^{h})SO₂-L₂-Rⁱ in which L₁ and L₂ independently represent a bond or a C₁₋₆alkylene optionally substituted by one or more C₁₋₃alkyl groups, R^{h} is H or C₁₋₃alkyl and Rⁱ represents cyano or a group NR^{u}R^{v} in which R^{u} and R^{v} are as previously defined, or Rⁱ represents a group CO—R^{j} in which R^{j} represents hydroxy, C₁₋₃alkoxy or a group NR^{u}R^{v} in which R^{u} and R^{v} are as previously defined;
x) phenyl(O)_{f}— wherein f is 0 or 1 optionally substituted by one or more halo, C₁₋₃alkyl optionally substituted by one or more halo or C₁₋₃alkoxy optionally substituted by one or more halo;
xi) phenylthio optionally substituted by one or more halo, C₁₋₃alkyl optionally substituted by one or more halo or C₁₋₃alkoxy optionally substituted by one or more halo;
xii) monocyclic heteroaryl(O)_{g}— wherein g is 0 or 1 optionally substituted by one or more halo, C₁₋₃alkyl optionally substituted by one or more halo or C₁₋₃alkoxy optionally substituted by one or more halo;
xiii) a nitrogen containing 5 or 6 membered heteroarylCO— wherein the heteroaryl is linked through nitrogen to the carbonyl group optionally substituted by one or more halo, C₁₋₃alkyl optionally substituted by one or more halo or C₁₋₃alkoxy optionally substituted by one or more halo;
xiv) a C₂₋₆alkynyl group optionally substituted by one or more C₁₋₃alkyl, hydroxy, C₁₋₃alkoxy, C₁₋₃alkoxyC₁₋₃alkoxy, or a group —NR^{u}R^{v} as defined above;
xv) a group -L₃-S(O)ₑC₁₋₆alkyl in which L₃ is a C₁₋₆alkylene optionally substituted by one or more of the following: hydroxy or a C₁₋₃alkyl group, and e is 0, 1 or 2;
xvi) a group SO₂NR^{o}R^{p} in which R^{o} and R^{p} independently represent H; a C₁₋₆alkyl group optionally substituted by one or more of the following: hydroxy, C₁₋₆alkoxy or a group —NR^{u}R^{v} in which R^{k} and R^{l} are as defined above, or R^{o} and R^{p} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₃alkoxy group; carboxy; a C₁₋₃alkylsulfonyl group; C₁₋₃alkanoyl; benzoyl; hydroxy; oxo; carboxy; or by a C₁₋₃alkyl group optionally substituted by one or more of the following: hydroxy, C₁₋₃alkoxy or carboxy; or
xvii) —C(NH₂)═N—OH
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and

### R⁷ is H or OH.

In another aspect the present invention provides a compound of formula I

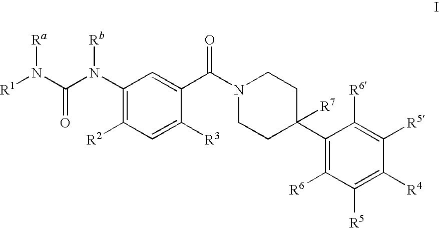

or a pharmaceutically acceptable salt thereof, in which
R¹ represents 1) a C₁₋₆alkyl group optionally substituted by one or two groups selected from A-W below and/or by one to five groups selected from X below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) a group CONR^{e}R^{f} in which R^{e} and R^{f} are as defined below; vii) C₁₋₆alkanoyl; viii) benzoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl or benzyloxy; xix) nitro; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl;
xxix) a group of formula NR^{c}R^{d} in which R^{c} and R^{d} independently represent:

### a) H;

b) C₁₋₆alkanoyl optionally substituted by carboxy or a C₁₋₆alkoxycarbonyl group;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, halo, oxo, carboxy, a C₁₋₆ alkoxycarbonyl group, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; halo; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl or nitro;
k) heteroaryl optionally substituted by one or more carboxy; fluoro; hydroxy; a C₁₋₆alkyl group (which is optionally substituted by one or more of the following: a C₁₋₆alkoxy group, hydroxy or a group of formula NR^{e}R^{f} in which R^{e} and R^{f} are as defined above); a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group optionally substituted by phenyl;
n) heteroarylcarbonyl;
o) sulfamoyl optionally substituted by one or two independently selected C₁₋₆alkyl groups or the terminal nitrogen is included in a 5 or 6 membered saturated or partially unsaturated heterocyclic ring optionally containing an additional N, S or O, wherein the S may be in its oxidised form of SO or SO₂;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring or a heteroaryl ring and is optionally substituted on any ring by one or more of the following: hydroxy; halo; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; a C₁₋₆alkyl optionally substituted by one or more hydroxy or C₁₋₆alkoxy; or a C₁₋₆ alkoxycarbonyl group;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) C₁₋₆alkylthio;
R) ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
S) phenoxy;
T) hydroxy;
U) oxo;
V) carboxy;
W) cyano;
X) fluoro;
or R¹ represents
2) a C₃₋₁₀ cycloalkyl group optionally substituted by one or two groups selected from A to X above;
3) a C₂₋₆alkynyl group optionally substituted by one or two groups selected from A to X above;
4) a carbon linked saturated or partially unsaturated 4 to 10 membered heterocyclic group containing one or more N, S or O, wherein the S may be in its oxidised form of SO or SO₂, which is optionally fused to a benz ring or a heteroaryl ring and any ring is optionally substituted by one or two groups A to X as defined above;
5) a C₂₋₆alkenyl group optionally substituted by one or two groups selected from A to X above;
wherein any alkyl chain mentioned in any of the definitions from A to R above or in any of the definitions i to xxix above is optionally substituted by 1) one or two groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; a C₁₋₄alkanoyloxy group or a C₁₋₄alkyl optionally substituted by one or more hydroxy, C₁₋₃alkoxy or a group —NR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 10 membered heterocyclic group in the list of optional substituents from A to P above or in any of the definitions i to xxix above, for which specific substitution has not been previously mentioned, is optionally substituted by one, two or three groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R^{a} represents H; or a C₁₋₄alkyl group, a C₃₋₆cycloalkyl group or a C₃₋₆cycloalkylC₁₋₄alkyl group each of which groups is optionally substituted by one or more carboxy; fluoro; hydroxy; a C₁₋₃alkoxy group optionally substituted on C2 or C3 by carboxy; a group of formula NR^{e}R^{f} in which R^{e} and R^{f} are as defined above; or a group CONR^{e}R^{f} in which R^{e} and R^{f} are as defined above;
or R¹ and R^{a} together with the nitrogen atom to which they are attached represent a saturated or partially unsaturated 4 to 8 membered heterocyclic ring optionally containing an additional sulphur including oxidised as SO or SO₂, oxygen or nitrogen which is optionally fused to a benz ring and wherein any ring is optionally substituted by one or two of the groups A to W above and/or by from 1 to 5 groups X;
R^{b} represents H,
R² represents H, halo, cyano, a C₁₋₃alkyl group optionally substituted by one or more halo, or a C₁₋₃alkoxy group optionally substituted by one or more halo;
R³ represents H, halo, cyano, a C₁₋₃alkyl group optionally substituted by one or more halo, or a C₁₋₃alkoxy group optionally substituted by one or more halo;
R⁴ represents i) H, ii) a C₁₋₃alkyl group optionally substituted by one or more halo iii) a C₁₋₃alkoxy group optionally substituted by one or more halo iv) halo, v) nitro, vi) cyano, vii) a C₁₋₆alkylS(O)_{y}(O)_{z}— wherein y is 0, 1 or 2 and z is 0 except when y is 2 when z is 0 or 1 viii) a group CH₂NR^{u}R^{v} in which R^{u} and R^{v} independently represent H; a C₁₋₃alkylsulphonyl group, a C₁₋₃alkanoyl group or a C₁₋₃alkyl group or R^{u} and R^{v} together with the nitrogen atom to which they are attached represent azetidinyl, pyrrolidinyl, piperidinyl or morpholinyl; ix) a group CO₂R^{w} in which R^{w} is a C₁₋₃alkyl group; or x) a group CONR^{x}R^{y} in which R^{x} and R^{y} independently represent H; or a C₁₋₃alkyl group or R^{x} and R^{y} together with the nitrogen atom to which they are attached represent azetidinyl; pyrrolidinyl, piperidinyl or morpholinyl;
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and

### R⁷ is H or OH.

In a further aspect the present invention provides a compound of formula I

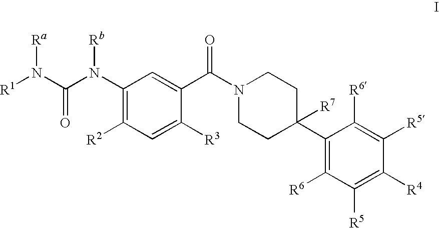

or a pharmaceutically acceptable salt thereof, in which
R¹ represents 1) a C₁₋₆alkyl group optionally substituted by one or two groups selected from A-S below and/or by one to five groups selected from T below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; yl) carbamoyl; vii) N—C₁₋₆alkylcarbamoyl; viii) N,N-diC₁₋₆alkylcarbamoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl xix) benzyloxy; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl; xxix) a group of formula NR^{c}R^{d} in which R^{c} and R^{d} independently represent:

### a) H;

b) C₁₋₆alkanoyl;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl;
k) heteroaryl optionally substituted by one or more carboxy; fluoro; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; or a C₁₋₆alkyl optionally substituted by one or more hydroxy or C₁₋₆alkoxy;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) hydroxy;
R) oxo;
S) carboxy;
T) fluoro
or R¹ represents
2) a C₃₋₇cycloalkyl group optionally substituted by one or two groups selected from A to T above;
3) a C₂₋₆alkynyl group optionally substituted by one or two groups selected from A to T above;
4) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by a group A to T as defined above;
5) a C₂₋₆alkenyl group optionally substituted by one or two groups selected from A to T above;
wherein any alkyl chain mentioned in any of the definitions from A to P above or in any of the definitions i to xxix above is optionally substituted by 1) one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 8 membered heterocyclic group in the list of optional substituents from A to P above or in any of the definitions i to xxix above, for which specific substitution has not been previously mentioned, is optionally substituted by one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R^{a} represents H; or a C₁₋₄alkyl group, a C₃₋₆cycloalkyl group or a C₃₋₆cycloalkylC₁₋₄alkyl group each of which groups is optionally substituted by one or more carboxy; fluoro; hydroxy; a C₁₋₃alkoxy group optionally substituted on C2 or C3 by carboxy; a group of formula NR^{e}R^{f} in which R^{e} and R^{f} are as defined above; or a group CONR^{e}R^{f} in which R^{e} and R^{f} are as defined above;
or R¹ and R^{a} together with the nitrogen atom to which they are attached represent a saturated or partially unsaturated 4 to 8 membered heterocyclic ring optionally containing an additional sulphur including oxidised as SO or SO₂, oxygen or nitrogen which is optionally fused to a benz ring and wherein any ring is optionally substituted by one or two of the groups above and/or by from 1 to 5 groups T;
R^{b} represents H,
R² represents H, halo, cyano, a C₁₋₃alkyl group optionally substituted by one or more halo, or a C₁₋₃alkoxy group optionally substituted by one or more halo;
R³ represents H, halo, cyano, a C₁₋₃alkyl group optionally substituted by one or more halo, or a C₁₋₃alkoxy group optionally substituted by one or more halo;
R⁴ represents i) H, ii) a C₁₋₃alkyl group optionally substituted by one or more halo iii) a C₁₋₃alkoxy group optionally substituted by one or more halo iv) halo, v) nitro, vi) cyano, vii) a C₁₋₆alkylS(O)_{y}(O)_{z}— wherein y is 0, 1 or 2 and z is 0 except when y is 2 when z is 0 or 1 viii) a group CH₂NR^{u}R^{v} in which R^{u} and R^{v} independently represent H; a C₁₋₃alkylsulphonyl group, a C₁₋₃alkanoyl group or a C₁₋₃alkyl group or R^{u} and R^{v} together with the nitrogen atom to which they are attached represent azetidinyl, pyrrolidinyl, piperidinyl or morpholinyl; ix) a group CO₂R^{w} in which R^{w} is a C₁₋₃alkyl group; or x) a group CONR^{x}R^{y} in which R^{x} and R^{y} independently represent H; or a C₁₋₃alkyl group or R^{x} and R^{y} together with the nitrogen atom to which they are attached represent azetidinyl; pyrrolidinyl, piperidinyl or morpholinyl;
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and

### R⁷ is H or OH.

In a further aspect the present invention provides a compound of formula II

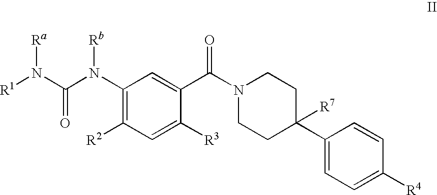

or a pharmaceutically acceptable salt thereof, in which
R¹ represents 1) a C₁₋₆alkyl group optionally substituted by one or two groups selected from A-S below and/or by one to five groups selected from T below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) carbamoyl; vii) N—C₁₋₆alkylcarbamoyl; viii) N,N-diC₁₋₆alkylcarbamoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl xix) benzyloxy; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl;
xxix) a group of formula NR^{c}R^{d} in which R^{c} and R^{d} independently represent:

### a) H;

b) C₁₋₆alkanoyl;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl;
k) heteroaryl optionally substituted by one or more carboxy; fluoro; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; or a C₁₋₆alkyl optionally substituted by one or more hydroxy or C₁₋₆alkoxy;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) hydroxy;
R) oxo;
S) carboxy;
T) fluoro
or R¹ represents
2) a C₃₋₇cycloalkyl group optionally substituted by one or two groups selected from A to T above;
3) a C₂₋₆alkynyl group optionally substituted by one or two groups selected from A to T above;
4) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by a group A to T as defined above;
5) a C₂₋₆alkenyl group optionally substituted by one or two groups selected from A to T above;
wherein any alkyl chain mentioned in any of the definitions from A to P above or in any of the definitions i to xxix above is optionally substituted by 1) one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro; and further wherein any cycloalkyl, phenyl, heteroaryl ring or carbon linked saturated or partially saturated 4 to 8 membered heterocyclic group in the list of optional substituents from A to P above or in any of the definitions i to xxix above, for which specific substitution has not been previously mentioned, is optionally substituted by one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R^{a} represents H; or a C₁₋₄alkyl group, a C₃₋₆cycloalkyl group or a C₃₋₆cycloalkylC₁₋₄alkyl group each of which groups is optionally substituted by one or more carboxy; fluoro; hydroxy; a C₁₋₃alkoxy group optionally substituted on C2 or C3 by carboxy; a group of formula NR^{e}R^{f} in which R^{e} and R^{f} are as defined above; or a group CONR^{e}R^{f} in which R^{e} and R^{f} are as defined above;
or R¹ and R^{a} together with the nitrogen atom to which they are attached represent a saturated or partially unsaturated 4 to 8 membered heterocyclic ring optionally containing an additional sulphur including oxidised as SO or SO₂, oxygen or nitrogen which is optionally fused to a benz ring and wherein any ring is optionally substituted by one or two of the groups A to S above and/or by from 1 to 5 groups T;
R^{b} represents H,
R² represents H, halo, cyano, a C₁₋₃alkyl group optionally substituted by one or more halo, or a C₁₋₃alkoxy group optionally substituted by one or more halo;
R³ represents H, halo, cyano, a C₁₋₃alkyl group optionally substituted by one or more halo, or a C₁₋₃alkoxy group optionally substituted by one or more halo;
R⁴ represents cyano, halo or a C₁₋₄alkylsulphonyl group: and
R⁷ represents H or hydroxy.

In a further aspect the present invention provides a compound of formula IIA

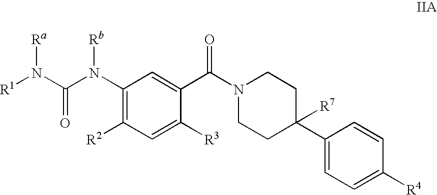

or a pharmaceutically acceptable salt thereof in which
R¹ represents 1) a C₁₋₆alkyl group optionally substituted by one or more of the following:
a) phenyl optionally substituted by one or more of the following: halo; a C₁₋₄alkoxy group or cyano; b) pyridyl c) a carbon linked saturated 5 or 6 membered heterocyclic group containing one N or O; d) a C₁₋₆alkoxycarbonyl group or e) a C₂₋₄alkynyl group or 2) a C₃₋₇cycloalkyl group
R^{a} represents H; or a C₁₋₄alkyl group;
or R¹ and R^{a} together with the nitrogen atom to which they are attached represent morpholinyl, pyrrolidinyl or piperidinyl;
R^{b} represents H,
R² represents H, halo, trifluoromethoxy, a C₁₋₃alkyl group; a C₁₋₃alkoxy group; cyano;
or when R¹ is other than phenyl then R^{b} together with the nitrogen to which is attached plus the carbon on the phenyl ring to which the nitrogen is attached and R² together with the carbon to which it is attached together represent a pyrrolidine ring fused to phenyl;
R³ represents H, halo, trifluoromethoxy, a C₁₋₃alkyl group; a C₁₋₃alkoxy group; cyano;
R⁴ represents bromo, cyano or a C₁₋₂alkylsulphonyl group: and
R⁷ represents H or hydroxy.
Further sub-definitions of the meaning of R¹, R^{a}, R², R^{b}, R³, R⁴, and R⁷, in compounds of formula I, II and IIA now follow. It will be understood that any combination of these sub-definitions may be used instead of the original definitions where appropriate in any of the compound groups, claims or embodiments defined hereinbefore or hereinafter.
In one group of compounds of formula IIA, R⁷ represents H and R¹, R^{a}, R², R^{b}, R³, R⁴ are as described above.

In a second group of compounds of formula IIA, R⁷ represents H, R¹ represents 1) a C₁₋₆alkyl group optionally substituted by one or more of the following: a) phenyl optionally substituted by one or more of the following: halo; a C₁₋₄alkoxy group or cyano; b) pyridyl c) oxan-4-yl d) a C₁₋₄alkoxycarbonyl group or e) a C₂₋₄alkynyl group 2) a C₃₋₇cycloalkyl group and R^{a} represents H or R¹ and R^{a} together with the nitrogen atom to which they are attached represent morpholino or pyrrolidino, and R², R^{b}, R³, R⁴ are as described above provided that one of R² and R³ is other than H.

In a third group of compounds of formula IIA, R² is methyl and R³ is H.

In a fourth group of compounds of formula IIA, R² and R³ are both methyl.

In a fifth group of compounds of formula IIA, R⁴ is cyano or methylsulphonyl.

In a sixth group of compounds of formula IIA, R^{a} is H.

In a seventh group of compounds of formula IIA, R³ is methyl and R² is H.

In an eighth group of compounds of formula IIA, R¹ represents pyrrolidinyl or piperidinyl optionally substituted on nitrogen by a C₁₋₃alkylsulphonyl group.

In a ninth group of compounds of formula IIA, R¹ represents a C₂₋₄alkylene chain terminally substituted by one of the following: a C₁₋₃alkylsulphonyl group or a group —NR¹⁰R¹¹ in which R¹⁰ represents H and R¹¹ represents H, a C₁₋₃alkylsulphonyl group or a sulphamoyl group which is optionally terminally substituted by one or two independently selected C₁₋₃alkyl groups.

In a tenth group of compounds of formula IIA, R⁴ is cyano.

It will be understood that each of these ten groups also apply to formula I and to formula II.

“Pharmaceutically acceptable salt”, where such salts are possible, includes both pharmaceutically acceptable acid and base addition salts. A suitable pharmaceutically acceptable salt of a compound of formula I is, for example, an acid-addition salt of a compound of formula I which is sufficiently basic, for example an acid-addition salt with an inorganic or organic acid such as hydrochloric, hydrobromic, sulphuric, trifluoroacetic, citric or maleic acid; or, for example a base-addition salt of a compound of formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates including solvates of the free compounds or solvates of a salt of the compound. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions that will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

The term “C₃₋₁₀cycloalkyl group which may be monocyclic, bicyclic or tricyclic and optionally may be bridged” includes cyclopropyl, cyclobutyl, cyclopentyl, cyclohexyl, cycloheptyl, cyclooctyl, cyclodecyl, bicyclo(2.2.1)heptyl, bicyclo(2.2.2)octyl, perhydroindanyl and adamantyl.

The term “heteroaryl” includes an aromatic 5- or 6-membered monocyclic ring or unless specified otherwise, an 8-, 9- or 10-membered bicyclic ring, with up to five ring heteroatoms selected from oxygen, nitrogen and sulfur, which may, unless otherwise specified be carbon or nitrogen linked. In one embodiment heteroaryl is an aromatic 5- or 6-membered monocyclic ring with up to five ring heteroatoms selected from oxygen, nitrogen and sulfur, which may, unless otherwise specified be carbon or nitrogen linked and includes pyrrolyl, thienyl, furyl, pyrazolyl, imidazolyl, oxazolyl, isoxazolyl, thiazolyl, isothiazolyl, thiadiazolyl, 1,3,4-oxadiazolyl, 1,2,4-oxadiazolyl, triazolyl, furazanyl, tetrazolyl, pyridyl, pyrimidyl, pyrazinyl, pyridazinyl, 1,3,5-triazinyl and imidazothiazolyl. Other heteroaryls include quinolyl, isoquinolyl, benzothienyl, benzofuranyl, benzofurazanyl, benzoxazolyl, benzimidazolyl, indolyl, benzthiazolyl, indazolyl, cinnolinyl, quinazolinyl, quinoxalinyl, phthalazinyl, 1,5-naphthyridinyl, 1,6-naphthyridinyl, 1,7-naphthyridinyl, 1,8-naphthyridinyl, pyrrolopyridinyl, pyrrolopyrazinyl, pyrazolopyridinyl or imidazopyridinyl.

The term “heteroaryl including N-oxides” includes heteroaryls as described immediately above and in addition N-oxides of such heteroaryls where such N-oxides are known to those skilled in the art to exist and are known to be stable at ambient conditions for example pyridine-N-oxides.

The term “a carbon linked saturated or partially saturated 4 to 10 (or 4 to 8) membered heterocyclic group containing one or more N, S or O, wherein the S may be in its oxidised form of SO or SO₂, which is optionally fused to a benz ring or a heteroaryl ring” includes oxetanyl, tetrahydrofuranyl, tetrahydropyranyl, 2,3-dihydro-1,3-thiazolyl, 1,3-thiazolidinyl, 1,3-oxazolidinyl, oxepanyl, azetidinyl, pyrrolinyl, pyrrolidinyl, morpholinyl, thiamorpholinyl (perhydro-1,4-thiazinyl), (8-oxa-3-azabicyclo[3.2.1]octyl), (7-oxa-3-azabicyclo[3.1.1]heptyl), perhydroazepinyl, perhydrooxazepinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, or tetrahydroquinolyl each of which may be optionally substituted as previously described.

When two substituents on an amine together with the nitrogen atom to which they are attached represent a saturated or partially unsaturated 4 to 10 (or 4 to 8) membered heterocyclic ring optionally containing an additional oxygen, sulphur, SO, SO₂ or nitrogen O and/or optionally fused to a benz ring then such rings include azetidino, pyrrolidino, morpholino, piperidino, imidazolidinyl, imidazolinyl, piperazino, thiamorpholino (perhydro-1,4-thiazinyl), homopiperazino, perhydroazepino, perhydrooxazepino, (2,3-dihydro-1,3-thiazolyl, 1,3-thiazolidinyl, 1,3-oxazolidinyl, oxepanyl, oxazepanyl, dihydropyrimidinyl, tetrahydropyrimidinyl, and homopiperidinyl, each of which is optionally substituted as previously described.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl, t-butyl, pentyl, isopentyl, neopentyl, tert-pentyl, hexyl and isohexyl. Preferred alkyl groups are methyl, ethyl, propyl, isopropyl, butyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

An example of “C₁₋₆alkanoyloxy” is acetoxy. Examples of “C₁₋₆alkoxycarbonyl” include C₁₋₄alkoxycarbonyl, methoxycarbonyl, ethoxycarbonyl, n- and t-butoxycarbonyl. Examples of “C₁₋₆alkoxycarbonylamino” include methoxycarbonylamino, ethoxycarbonylamino, n- and t-butoxycarbonylamino. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ(O)_{b}— group in which a is 0, 1 or 2 and b is 0 except when a is 2 then b may also be 1” include C₁₋₄alkylsulfonyl, methylthio, ethylthio, methylsulfinyl, ethylsulfinyl, mesyl, ethylsulfonyl methylsulfonyloxy and where substituted by fluoro include trifluoromethylsulfonyloxy and trifluoropropylsulfonyloxy. Examples of “C₁₋₆alkylsulfonylamino” include methylsulfonylamino, ethylsulfonylamino and propylsulfonylamino. Examples of “C₁₋₆alkylsulfonyl-N—(C₁₋₆alkyl)amino” include methylsulfonyl-N-methylamino, ethylsulfonyl-N-methylamino and propylsulfonyl-N-ethylamino. Examples of “C₁₋₆alkanoyl” include C₁₋₄alkanoyl, propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂carbamoyl” are N,N—(C₁₋₄alkyl)carbamoyl, dimethylaminocarbonyl and methylethylaminocarbonyl. Examples of “(heterocyclic group)C₁₋₆alkyl” include pyridylmethyl, 3-morpholinopropyl and 2-pyrimid-2-ylethyl. Examples of “C₃₋₈cycloalkylC₁₋₆cycloalkyl” include cyclopropylmethyl and 2-cyclohexylpropyl. Examples of “N—(C₁₋₆alkyl)sulphamoylamino” are N-(methyl)sulphamoylamino and N-(ethyl)sulphamoylamino. Examples of “N—(C₁₋₆alkyl)₂sulphamoylamino” are N,N-(dimethyl)sulphamoylamino and N-(methyl)-N-(ethyl)sulphamoylamino. Examples of “C₁₋₆alkylsulphonylaminocarbonyl” include methylsulphonylaminocarbonyl, ethylsulphonylaminocarbonyl and propylsulphonylaminocarbonyl.

Specific compounds of the invention include one or more, for example from 1 to 418, of the following compounds below labelled as List 1:
1-butyl-3-[5-[4-(4-cyanophenyl)piperidine-1-carbonyl]-2-methyl-phenyl]urea;
3-[5-[4-(4-cyanophenyl)piperidine-1-carbonyl]-2-methyl-phenyl]-1-propan-2-yl-urea;
3-[5-[4-(4-cyanophenyl)piperidine-1-carbonyl]-2-(trifluoromethoxy)phenyl]-1-propan-2-yl-urea;
3-benzyl-1-[5-[4-(4-cyanophenyl)piperidine-1-carbonyl]-2-(trifluoromethoxy)phenyl]urea;
1-[5-[4-(4-cyanophenyl)piperidine-1-carbonyl]-2-methoxy-phenyl]-3-propan-2-yl-urea;
3-benzyl-1-[3-[4-(4-cyanophenyl)piperidine-1-carbonyl]-4-methyl-phenyl]urea;
3-benzyl-1-[5-[4-(4-cyanophenyl)piperidine-1-carbonyl]-2-fluoro-phenyl]urea;
N-[5-[4-(4-cyanophenyl)piperidine-1-carbonyl]-2-methyl-phenyl]morpholine-4-carboxamide;
3-benzyl-1-[5-[4-(4-cyanophenyl)piperidine-1-carbonyl]-2-methoxy-phenyl]urea;
N-[5-[4-(4-cyanophenyl)piperidine-1-carbonyl]-2-methyl-phenyl]pyrrolidine-1-carboxamide;
3-[5-[4-(4-cyanophenyl)piperidine-1-carbonyl]-2-fluoro-phenyl]-1-propan-2-yl-urea;
3-benzyl-1-[5-[4-(4-cyanophenyl)piperidine-1-carbonyl]-2,4-dimethyl-phenyl]urea;
3-[5-[4-(4-cyanophenyl)piperidine-1-carbonyl]-2,4-dimethyl-phenyl]-1-propan-2-yl-urea;
1-benzyl-3-[3-[4-(4-cyanophenyl)piperidine-1-carbonyl]phenyl]urea;
1-[5-[4-(4-cyanophenyl)piperidine-1-carbonyl]-2-methyl-phenyl]-3-cyclopentyl-urea;
1-[5-[4-(4-cyanophenyl)piperidine-1-carbonyl]-2-methyl-phenyl]-3-phenethyl-urea;
1-[5-[4-(4-cyanophenyl)piperidine-1-carbonyl]-2-methyl-phenyl]-3-(oxan-4-ylmethyl)urea;
3-[3-[4-(4-cyanophenyl)piperidine-1-carbonyl]-4-methyl-phenyl]-1-methyl-urea;
3-[3-[4-(4-cyanophenyl)piperidine-1-carbonyl]-4-methyl-phenyl]-1-ethyl-urea;
1-butyl-3-[3-[4-(4-cyanophenyl)piperidine-1-carbonyl]-4-methyl-phenyl]urea;
1-[3-[4-(4-cyanophenyl)piperidine-1-carbonyl]-4-methyl-phenyl]-3-cyclopentyl-urea;
3-[2-cyano-5-[4-(4-cyanophenyl)piperidine-1-carbonyl]phenyl]-1-propan-2-yl-urea;
1-[3-[4-(4-cyanophenyl)piperidine-1-carbonyl]-4-methyl-phenyl]-3-[(2-fluorophenyl)methyl]urea;
1-benzyl-3-[3-[4-(4-cyanophenyl)piperidine-1-carbonyl]-4-methyl-phenyl]-1-methyl-urea;
1-[3-[4-(4-cyanophenyl)piperidine-1-carbonyl]-4-methyl-phenyl]-3-(pyridin-3-ylmethyl)urea;
1-[3-[4-(4-cyanophenyl)piperidine-1-carbonyl]-4-methyl-phenyl]-3-phenethyl-urea;
1-[3-[4-(4-cyanophenyl)piperidine-1-carbonyl]-4-methyl-phenyl]-3-(oxan-4-ylmethyl)urea;
N-[3-[4-(4-cyanophenyl)piperidine-1-carbonyl]-4-methyl-phenyl]morpholine-4-carboxamide;
N-[3-[4-(4-cyanophenyl)piperidine-1-carbonyl]-4-methyl-phenyl]pyrrolidine-1-carboxamide;
3-[5-[4-(4-cyanophenyl)piperidine-1-carbonyl]-2-methyl-phenyl]-1-ethyl-urea;
1-[5-[4-(4-cyanophenyl)piperidine-1-carbonyl]-2-methyl-phenyl]-3-tert-butyl-urea;
3-benzyl-1-[5-[4-(4-cyanophenyl)piperidine-1-carbonyl]-2-methyl-phenyl]urea;
3-[(4-cyanophenyl)methyl]-1-[5-[4-(4-cyanophenyl)piperidine-1-carbonyl]-2-methyl-phenyl]urea;
1-[5-[4-(4-cyanophenyl)piperidine-1-carbonyl]-2-methyl-phenyl]-3-[(2-fluorophenyl)methyl]urea;
1-[5-[4-(4-cyanophenyl)piperidine-1-carbonyl]-2-methyl-phenyl]-3-(pyridin-3-ylmethyl)urea;
1-[5-[4-(4-cyanophenyl)piperidine-1-carbonyl]-2-methyl-phenyl]-3-(pyridin-4-ylmethyl)urea;
3-[3-[4-(4-cyanophenyl)piperidine-1-carbonyl]-4-methyl-phenyl]-1-[(1R)-1-phenylethyl]urea;
3-[3-[4-(4-cyanophenyl)piperidine-1-carbonyl]-4-methyl-phenyl]-1-[(1S)-1-phenylethyl]urea;
1-[5-[4-(4-bromophenyl)-4-hydroxy-piperidine-1-carbonyl]-2-methyl-phenyl]-3-propan-2-yl-urea;
3-benzyl-1-[5-[4-(4-bromophenyl)-4-hydroxy-piperidine-1-carbonyl]-2-methyl-phenyl]urea;
3-(1-benzyl-4-piperidyl)-1-[5-[4-(4-cyanophenyl)piperidine-1-carbonyl]-2-methyl-phenyl]urea;
1-[5-[4-(4-cyanophenyl)piperidine-1-carbonyl]-2-methyl-phenyl]-3-(2-methylpropyl)urea;
1-[5-[4-(4-cyanophenyl)piperidine-1-carbonyl]-2-methyl-phenyl]-3-prop-2-ynyl-urea;
1-[5-[4-(4-cyanophenyl)piperidine-1-carbonyl]-2-methyl-phenyl]-3-[(3,4,5-trimethoxyphenyl)methyl]urea;
methyl 3-[[5-[4-(4-cyanophenyl)piperidine-1-carbonyl]-2-methyl-phenyl]carbamoylamino]propanoate;
3-[3-[4-(4-cyanophenyl)piperidine-1-carbonyl]phenyl]-1-propan-2-yl-urea;
3-[(3-cyanophenyl)methyl]-1-[3-[4-(4-cyanophenyl)piperidine-1-carbonyl]-4-methyl-phenyl]urea
1-[3-[4-(4-cyanophenyl)piperidine-1-carbonyl]-4-methyl-phenyl]-3-(4-hydroxycyclohexyl)urea
1-[3-[4-(4-cyanophenyl)piperidine-1-carbonyl]-4-methyl-phenyl]-3-(4-hydroxycyclohexyl)urea
1-[5-[4-(4-cyanophenyl)-4-hydroxy-piperidine-1-carbonyl]-2-methyl-phenyl]-3-propan-2-yl-urea
tert-butyl 4-[[5-[4-(4-cyanophenyl)piperidine-1-carbonyl]-2-methyl-phenyl]carbamoylamino]butanoate
1-[5-[4-(4-cyanophenyl)piperidine-1-carbonyl]-2-methyl-phenyl]-3-(pyridin-2-ylmethyl)urea
3-[5-[4-(4-cyanophenyl)piperidine-1-carbonyl]-2-methyl-phenyl]-1-pentan-3-yl-urea
3-[5-[4-(4-cyanophenyl)piperidine-1-carbonyl]-2-methyl-phenyl]-1-[(3-methylphenyl)methyl]urea
N-[2-[[5-[4-(4-cyanophenyl)piperidine-1-carbonyl]-2-methyl-phenyl]carbamoylamino]ethyl]acetamide
1-[5-[4-(4-cyanophenyl)piperidine-1-carbonyl]-2-methyl-phenyl]-3-[(3,4-difluorophenyl)methyl]urea
1-[5-[4-(4-cyanophenyl)piperidine-1-carbonyl]-2-methyl-phenyl]-3-[2-(4-sulfamoylphenyl)ethyl]urea
2-[[5-[4-(4-cyanophenyl)piperidine-1-carbonyl]-2-methyl-phenyl]carbamoylamino]acetamide
3-(1-anilino-2-methyl-propan-2-yl)-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-(2-pyridin-2-ylethyl)urea
1-[5-[4-(4-cyanophenyl)piperidine-1-carbonyl]-2-methyl-phenyl]-3-[2-(2-methoxyphenyl)ethyl]urea
1-[5-[4-(4-cyanophenyl)piperidine-1-carbonyl]-2-methyl-phenyl]-3-(2-pyridin-4-ylethyl)urea
1-[5-[4-(4-cyanophenyl)piperidine-1-carbonyl]-2-methyl-phenyl]-3-(2-thiophen-2-ylethyl)urea
1-[5-[4-(4-cyanophenyl)piperidine-1-carbonyl]-2-methyl-phenyl]-3-[2-(4-methoxyphenyl)ethyl]urea
1-[5-[4-(4-cyanophenyl)piperidine-1-carbonyl]-2-methyl-phenyl]-3-[2-(3-methoxyphenyl)ethyl]urea
1-[5-[4-(4-cyanophenyl)piperidine-1-carbonyl]-2-methyl-phenyl]-3-(2-pyridin-3-ylethyl)urea
1-[5-[4-(4-cyanophenyl)piperidine-1-carbonyl]-2-methyl-phenyl]-3-[2-(3,5-dimethyl-1,2-oxazol-4-yl)ethyl]urea
1-[5-[4-(4-cyanophenyl)piperidine-1-carbonyl]-2-methyl-phenyl]-3-[2-(3-fluorophenyl)ethyl]urea
tert-butyl N-[4-[[5-[4-(4-cyanophenyl)piperidine-1-carbonyl]-2-methyl-phenyl]carbamoylamino]butyl]carbamate
1-[5-[4-(4-cyanophenyl)piperidine-1-carbonyl]-2-methyl-phenyl]-3-[1-(1H-indol-3-yl)propan-2-yl]urea
1-[5-[4-(4-cyanophenyl)piperidine-1-carbonyl]-2-methyl-phenyl]-3-(2,2-dimethylpropyl)urea
Methyl 2-[[5-[4-(4-cyanophenyl)piperidine-1-carbonyl]-2-methyl-phenyl]carbamoylamino]acetate
(2S)-2-[[5-[4-(4-cyanophenyl)piperidine-1-carbonyl]-2-methyl-phenyl]carbamoylamino]-4-methyl-pentanamide
1-[5-[4-(4-cyanophenyl)piperidine-1-carbonyl]-2-methyl-phenyl]-3-(3-pyrrolidin-1-ylpropyl)urea
1-[5-[4-(4-cyanophenyl)piperidine-1-carbonyl]-2-methyl-phenyl]-3-[[4-(thiadiazol-4-yl)phenyl]methyl]urea
1-[5-[4-(4-cyanophenyl)piperidine-1-carbonyl]-2-methyl-phenyl]-3-[2-(3,5-dimethylpyrazol-1-yl)ethyl]urea
1-[5-[4-(4-cyanophenyl)piperidine-1-carbonyl]-2-methyl-phenyl]-3-(2,2-dimethyloxan-4-yl)urea
(2S)-2-[[5-[4-(4-cyanophenyl)piperidine-1-carbonyl]-2-methyl-phenyl]carbamoylamino]3-[(2-methylpropan-2-yl)oxy]propanamide
3-[5-[4-(4-cyanophenyl)piperidine-1-carbonyl]-2-methyl-phenyl]-1-(1-propyl-4-piperidyl)urea
1-[5-[4-(4-cyanophenyl)piperidine-1-carbonyl]-2-methyl-phenyl]-3-[(1,1-dioxothiolan-3-yl)methyl]urea
Benzyl N-[2-[[5-[4-(4-cyanophenyl)piperidine-1-carbonyl]-2-methyl-phenyl]carbamoylamino]ethyl]carbamate
1-[5-[4-(4-cyanophenyl)piperidine-1-carbonyl]-2-methyl-phenyl]-3-(1H-tetrazol-5-ylmethyl)urea
1-[5-[4-(4-cyanophenyl)piperidine-1-carbonyl]-2-methyl-phenyl]-3-[2-(2-methoxyphenoxy)ethyl]urea
1-[5-[4-(4-cyanophenyl)piperidine-1-carbonyl]-2-methyl-phenyl]-3-[(1R)-1-(4-methoxyphenyl)ethyl]urea
3-[(3-aminophenyl)methyl]-1-[5-[4-(4-cyanophenyl)piperidine-1-carbonyl]-2-methyl-phenyl]urea
3-[2-(benzenesulfonamido)ethyl]-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-[2-[(4-nitrophenyl)amino]ethyl]urea
1-[5-[4-(4-cyanophenyl)piperidine-1-carbonyl]-2-methyl-phenyl]-3-[1-(4-fluorophenyl)ethyl]urea
1-[5-[4-(4-cyanophenyl)piperidine-1-carbonyl]-2-methyl-phenyl]-3-(3-furylmethyl)urea
1-[5-[4-(4-cyanophenyl)piperidine-1-carbonyl]-2-methyl-phenyl]-3-(2-hydroxyethyl)urea
N-[2-[[5-[4-(4-cyanophenyl)piperidine-1-carbonyl]-2-methyl-phenyl]carbamoylamino]ethyl]pyridine-2-carboxamide
1-[5-[4-(4-cyanophenyl)piperidine-1-carbonyl]-2-methyl-phenyl]-3-[2-(dimethylsulfamoylamino)ethyl]urea
N-[2-[[5-[4-(4-cyanophenyl)piperidine-1-carbonyl]-2-methyl-phenyl]carbamoylamino]-2-methyl-propyl]pyridine-3-carboxamide
3-[2-[(2-amino-5,6-dimethyl-pyrimidin-4-yl)amino]ethyl]-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-[2-(9H-purin-6-ylamino)ethyl]urea
1-[5-[4-(4-cyanophenyl)piperidine-1-carbonyl]-2-methyl-phenyl]-3-(3-methylbut-2-enyl)urea
tert-butyl 3-[[5-[4-(4-cyanophenyl)piperidine-1-carbonyl]-2-methyl-phenyl]carbamoylamino]azetidine-1-carboxylate
1-[5-[4-(4-cyanophenyl)piperidine-1-carbonyl]-2-methyl-phenyl]-3-[1-(4-methylsulfonylphenyl)ethyl]urea
1-[5-[4-(4-cyanophenyl)piperidine-1-carbonyl]-2-methyl-phenyl]-3-(2-oxo-3,4-dihydro-1H-1,7-naphthyridin-3-yl)urea
1-[5-[4-(4-cyanophenyl)piperidine-1-carbonyl]-2-methyl-phenyl]-3-[3-(3-methyl-1-piperidyl)propyl]urea
1-[5-[4-(4-cyanophenyl)piperidine-1-carbonyl]-2-methyl-phenyl]-3-[2-[(4-methoxyphenyl)amino]ethyl]urea
tert-butyl N-[2-[[5-[4-(4-cyanophenyl)piperidine-1-carbonyl]-2-methyl-phenyl]carbamoylamino]ethyl]carbamate
3-(2-aminoethyl)-1-[5-[4-(4-cyanophenyl)piperidine-1-carbonyl]-2-methyl-phenyl]urea
2-[[5-[4-(4-cyanophenyl)piperidine-1-carbonyl]-2-methyl-phenyl]carbamoylamino]ethylcarbamoylformic acid
2-[2-[[5-[4-(4-cyanophenyl)piperidine-1-carbonyl]-2-methyl-phenyl]carbamoylamino]ethylcarbamoyl]acetic acid
1-[5-[4-(4-cyanophenyl)piperidine-1-carbonyl]-2-methyl-phenyl]-3-[(1S)-1-phenylethyl]urea
1-[5-[4-(4-cyanophenyl)piperidine-1-carbonyl]-2-methyl-phenyl]-3-[(1R)-1-phenylethyl]urea
1-[5-[4-(4-cyanophenyl)piperidine-1-carbonyl]-2-methyl-phenyl]-3-(4-hydroxycyclohexyl)urea
3-[(3-cyanophenyl)methyl]-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-(2-methanesulfonamidoethyl)urea
3-[5-[4-(4-cyanophenyl)piperidine-1-carbonyl]-2-methyl-phenyl]-1-[2-(ethylsulfonylamino)ethyl]urea
N-[2-[[5-[4-(4-cyanophenyl)piperidine-1-carbonyl]-2-methyl-phenyl]carbamoylamino]ethyl]propanamide
N-[2-[[5-[4-(4-cyanophenyl)piperidine-1-carbonyl]-2-methyl-phenyl]carbamoylamino]ethyl]-2-methyl-propanamide
2-[[5-[4-(4-cyanophenyl)piperidine-1-carbonyl]-2-methyl-phenyl]carbamoylamino]ethylcarbamoylmethyl acetate
N-[2-[[5-[4-(4-cyanophenyl)piperidine-1-carbonyl]-2-methyl-phenyl]carbamoylamino]ethyl]-2-hydroxy-acetamide
tert-butyl N-[2-[[5-[4-(4-cyanophenyl)piperidine-1-carbonyl]-2-methyl-phenyl]carbamoylamino]ethyl]-N-methyl-carbamate
1-[5-[4-(4-cyanophenyl)piperidine-1-carbonyl]-2-methyl-phenyl]-3-(2-methylaminoethyl)urea
N-[2-[[5-[4-(4-cyanophenyl)piperidine-1-carbonyl]-2-methyl-phenyl]carbamoylamino]ethyl]thiophene-2-carboxamide
N-[2-[[5-[4-(4-cyanophenyl)piperidine-1-carbonyl]-2-methyl-phenyl]carbamoylamino]ethyl]-1-methyl-pyrrole-2-carboxamide
N-[2-[[5-[4-(4-cyanophenyl)piperidine-1-carbonyl]-2-methyl-phenyl]carbamoylamino]ethyl]-5-methyl-1,2-oxazole-4-carboxamide
N-[2-[[5-[4-(4-cyanophenyl)piperidine-1-carbonyl]-2-methyl-phenyl]carbamoylamino]ethyl]-N-methyl-acetamide
1-[5-[4-(4-cyanophenyl)piperidine-1-carbonyl]-2-methyl-phenyl]-3-[2-(methyl-methylsulfonyl-amino)ethyl]urea
1-[5-[4-(4-cyanophenyl)piperidine-1-carbonyl]-2-methyl-phenyl]-3-[2-(propan-2-ylsulfonylamino)ethyl]urea
4-[1-[3-(benzylcarbamoylamino)-4-methyl-benzoyl]-4-piperidyl]-N,N-dimethyl-benzamide
N,N-dimethyl-4-[1-[4-methyl-3-(propan-2-ylcarbamoylamino)benzoyl]-4-piperidyl]benzamide
3-[[5-[4-(4-cyanophenyl)piperidine-1-carbonyl]-2-methyl-phenyl]carbamoylamino]propanoic acid
3-[[5-[4-(4-cyanophenyl)piperidine-1-carbonyl]-2-methyl-phenyl]carbamoylamino]propanamide
1-[5-[4-(4-cyanophenyl)piperidine-1-carbonyl]-2-methyl-phenyl]-3-(3-morpholin-4-yl-3-oxo-propyl)urea
3-[[5-[4-(4-cyanophenyl)piperidine-1-carbonyl]-2-methyl-phenyl]carbamoylamino]-N-(2-methoxyethyl)propanamide
3-[[5-[4-(4-cyanophenyl)piperidine-1-carbonyl]-2-methyl-phenyl]carbamoylamino]-N-propan-2-yl-propanamide
3-[[5-[4-(4-cyanophenyl)piperidine-1-carbonyl]-2-methyl-phenyl]carbamoylamino]-N,N-dimethyl-propanamide
1-[5-[4-(4-cyanophenyl)piperidine-1-carbonyl]-2-methyl-phenyl]-3-[3-(2-oxopyrrolidin-1-yl)propyl]urea
ethyl 4-[[5-[4-(4-cyanophenyl)piperidine-1-carbonyl]-2-methyl-phenyl]carbamoylamino]piperidine-1-carboxylate
1-[5-[4-(4-cyanophenyl)piperidine-1-carbonyl]-2-methyl-phenyl]-3-(2-oxoazepan-3-yl)urea
(1S)-2-[[5-[4-(4-cyanophenyl)piperidine-1-carbonyl]-2-methyl-phenyl]carbamoylamino]cyclohexane-1-carboxamide
1-[5-[4-(4-cyanophenyl)piperidine-1-carbonyl]-2-methyl-phenyl]-3-[2-(2-oxoimidazolidin-1-yl)ethyl]urea
1-[5-[4-(4-cyanophenyl)piperidine-1-carbonyl]-2-methyl-phenyl]-3-[(3-oxo-1,2-oxazol-5-yl)methyl]urea
1-[5-[4-(4-cyanophenyl)piperidine-1-carbonyl]-2-methyl-phenyl]-3-(2-pyrrolidin-1-ylethyl)urea
1-[5-[4-(4-cyanophenyl)piperidine-1-carbonyl]-2-methyl-phenyl]-3-(3-dimethylaminopropyl)urea
1-[5-[4-(4-cyanophenyl)piperidine-1-carbonyl]-2-methyl-phenyl]-3-[2-(1H-imidazol-4-yl)ethyl]urea
1-[5-[4-(4-cyanophenyl)piperidine-1-carbonyl]-2-methyl-phenyl]-3-[3-(4-methylpiperazin-1-yl)propyl]urea
3-[3-(bis(2-hydroxyethyl)amino)propyl]-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-(4-dimethylaminobutyl)urea
3-(1-azabicyclo[2.2.2]oct-8-yl)-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-[2-(1-methylpyrrolidin-2-yl)ethyl]urea
1-[5-[4-(4-cyanophenyl)piperidine-1-carbonyl]-2-methyl-phenyl]-3-(2-dimethylaminoethyl)urea
1-[5-[4-(4-cyanophenyl)piperidine-1-carbonyl]-2-methyl-phenyl]-3-(3-morpholin-4-ylpropyl)urea
1-[5-[4-(4-cyanophenyl)piperidine-1-carbonyl]-2-methyl-phenyl]-3-[1-(2-methoxyethyl)-4-piperidyl]urea
1-[5-[4-(4-cyanophenyl)piperidine-1-carbonyl]-2-methyl-phenyl]-3-(1-methyl-4-piperidyl)urea
3-[5-[4-(4-cyanophenyl)piperidine-1-carbonyl]-2-methyl-phenyl]-1-[[(2S)-1-ethylpyrrolidin-2-yl]methyl]urea
1-[5-[4-(4-cyanophenyl)piperidine-1-carbonyl]-2-methyl-phenyl]-3-[(3-methylimidazol-4-yl)methyl]urea
1-[5-[4-(4-cyanophenyl)piperidine-1-carbonyl]-2-methyl-phenyl]-3-(oxolan-2-ylmethyl)urea
1-[5-[4-(4-cyanophenyl)piperidine-1-carbonyl]-2-methyl-phenyl]-3-(2-methoxyethyl)urea
1-[5-[4-(4-cyanophenyl)piperidine-1-carbonyl]-2-methyl-phenyl]-3-(3-propan-2-yloxypropyl)urea
1-[5-[4-(4-cyanophenyl)piperidine-1-carbonyl]-2-methyl-phenyl]-3-(3-methoxypropyl)urea
1-[5-[4-(4-cyanophenyl)piperidine-1-carbonyl]-2-methyl-phenyl]-3-(1-methoxypropan-2-yl)urea
1-[5-[4-(4-cyanophenyl)piperidine-1-carbonyl]-2-methyl-phenyl]-3-(2-methylsulfanylethyl)urea
1-[5-[4-(4-cyanophenyl)piperidine-1-carbonyl]-2-methyl-phenyl]-3-(3-methylsulfanylpropyl)urea
3-[5-[4-(4-cyanophenyl)piperidine-1-carbonyl]-2-methyl-phenyl]-1-(3-ethoxypropyl)urea
1-[5-[4-(4-cyanophenyl)piperidine-1-carbonyl]-2-methyl-phenyl]-3-(2-methoxy-2-methyl-propyl)urea
1-[5-[4-(4-cyanophenyl)piperidine-1-carbonyl]-2-methyl-phenyl]-3-(1,4-dioxan-2-ylmethyl)urea
1-[5-[4-(4-cyanophenyl)piperidine-1-carbonyl]-2-methyl-phenyl]-3-[3-(2-methoxyethoxy)propyl]urea
1-[5-[4-(4-cyanophenyl)piperidine-1-carbonyl]-2-methyl-phenyl]-3-(oxan-4-yl)urea
1-[5-[4-(4-cyanophenyl)piperidine-1-carbonyl]-2-methyl-phenyl]-3-(2,6-dioxabicyclo[5.4.0]undeca-8,10,12-trien-4-yl)urea
3-[5-[4-(4-cyanophenyl)piperidine-1-carbonyl]-2-methyl-phenyl]-1-(2-propoxyethyl)urea
1-[5-[4-(4-cyanophenyl)piperidine-1-carbonyl]-2-methyl-phenyl]-3-(7,10-dioxabicyclo[4.4.0]deca-2,4,11-trien-8-ylmethyl)urea
1-[5-[4-(4-cyanophenyl)piperidine-1-carbonyl]-2-methyl-phenyl]-3-(oxan-2-ylmethyl)urea
3-(cyanomethyl)-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-(2,2,2-trifluoroethyl)urea
1-[5-[4-(4-cyanophenyl)piperidine-1-carbonyl]-2-methyl-phenyl]-3-(1,1-dioxothiolan-3-yl)urea
3-[[5-[4-(4-cyanophenyl)piperidine-1-carbonyl]-2-methyl-phenyl]carbamoylamino]-N-pyridin-2-yl-propanamide
2-[[5-[4-(4-cyanophenyl)piperidine-1-carbonyl]-2-methyl-phenyl]carbamoylamino]-N-propan-2-yl-acetamide
1-butyl-3-[2-[[5-[4-(4-cyanophenyl)piperidine-1-carbonyl]-2-methyl-phenyl]carbamoylamino]ethyl]urea
N-[5-[[5-[4-(4-cyanophenyl)piperidine-1-carbonyl]-2-methyl-phenyl]carbamoylamino]pentyl]morpholine-4-carboxamide
3-[5-[4-(4-cyanophenyl)piperidine-1-carbonyl]-2-methyl-phenyl]-1-[2-(propylsulfonylamino)ethyl]urea
N-[2-[[5-[4-(4-cyanophenyl)piperidine-1-carbonyl]-2-methyl-phenyl]carbamoylamino]ethyl]cyclohexanecarboxamide
1-[5-[4-(4-cyanophenyl)piperidine-1-carbonyl]-2-methyl-phenyl]-3-[2-(2-oxopyrrolidin-1-yl)ethyl]urea
Methyl (2S)-3-[[5-[4-(4-cyanophenyl)piperidine-1-carbonyl]-2-methyl-phenyl]carbamoylamino]-2-[(2-methylpropan-2-yl)oxycarbonylamino]propanoate
1-[5-[4-(4-cyanophenyl)piperidine-1-carbonyl]-2-methyl-phenyl]-3-[3-(2-oxoazepan-1-yl)propyl]urea
3-[[5-[4-(4-cyanophenyl)piperidine-1-carbonyl]-2-methyl-phenyl]carbamoylamino]-N-methyl-propanamide
tert-butyl (2S)-2-[[5-[4-(4-cyanophenyl)piperidine-1-carbonyl]-2-methyl-phenyl]carbamoylamino]-4-methylsulfonyl-butanoate
Methyl 4-[[5-[4-(4-cyanophenyl)piperidine-1-carbonyl]-2-methyl-phenyl]carbamoylamino]cyclohexane-1-carboxylate
1-[5-[4-(4-cyanophenyl)piperidine-1-carbonyl]-2-methyl-phenyl]-3-(1-hydroxypropan-2-yl)urea
1-[5-[4-(4-cyanophenyl)piperidine-1-carbonyl]-2-methyl-phenyl]-3-(2-hydroxypropyl)urea
1-[5-[4-(4-cyanophenyl)piperidine-1-carbonyl]-2-methyl-phenyl]-3-(2,3-dihydroxypropyl)urea
1-[5-[4-(4-cyanophenyl)piperidine-1-carbonyl]-2-methyl-phenyl]-3-(4-hydroxybutyl)urea
1-[5-[4-(4-cyanophenyl)piperidine-1-carbonyl]-2-methyl-phenyl]-3-(1,3-dihydroxypropan-2-yl)urea
1-[5-[4-(4-cyanophenyl)piperidine-1-carbonyl]-2-methyl-phenyl]-3-(2-hydroxycyclohexyl)urea
1-[5-[4-(4-cyanophenyl)piperidine-1-carbonyl]-2-methyl-phenyl]-3-(3-hydroxy-2,2-dimethyl-propyl)urea
1-[5-[4-(4-cyanophenyl)piperidine-1-carbonyl]-2-methyl-phenyl]-3-[2-(2-hydroxyethoxy)ethyl]urea
1-[5-[4-(4-cyanophenyl)piperidine-1-carbonyl]-2-methyl-phenyl]-3-(1-hydroxy-2-methyl-propan-2-yl)urea
1-[5-[4-(4-cyanophenyl)piperidine-1-carbonyl]-2-methyl-phenyl]-3-(3-hydroxypropyl)urea
(2S)-2-[[5-[4-(4-cyanophenyl)piperidine-1-carbonyl]-2-methyl-phenyl]carbamoylamino]-3-hydroxy-propanamide
1-[5-[4-(4-cyanophenyl)piperidine-1-carbonyl]-2-methyl-phenyl]-3-(4-hydroxycyclohexyl)urea
1-[5-[4-(4-cyanophenyl)piperidine-1-carbonyl]-2-methyl-phenyl]-3-[1-(hydroxymethyl)cyclopentyl]urea
1-[5-[4-(4-cyanophenyl)piperidine-1-carbonyl]-2-methyl-phenyl]-3-(3,3,3-trifluoro-2-hydroxy-propyl)urea
3-[3-(2-chlorophenoxy)-2-hydroxy-propyl]-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-(3-hydroxy-1-adamantyl)urea
1-[5-[4-(4-cyanophenyl)piperidine-1-carbonyl]-2-methyl-phenyl]-3-[(2R)-1-hydroxy-3-methoxy-propan-2-yl]urea
1-[5-[4-(4-cyanophenyl)piperidine-1-carbonyl]-2-methyl-phenyl]-3-[(3R)-oxolan-3-yl]urea
1-[5-[4-(4-cyanophenyl)piperidine-1-carbonyl]-2-methyl-phenyl]-3-[3-(3-methyl-2H-pyrazol-4-yl)propyl]urea
1-[5-[4-(4-cyanophenyl)piperidine-1-carbonyl]-2-ethyl-phenyl]-3-propan-2-yl-urea
1-[5-[4-(4-cyanophenyl)piperidine-1-carbonyl]-2-methyl-phenyl]-3-(2-methanesulfonamido-2-methyl-propyl)urea
3-(2-amino-2-methyl-propyl)-1-[5-[4-(4-cyanophenyl)piperidine-1-carbonyl]-2-methyl-phenyl]urea
3-[5-[4-(4-cyanophenyl)piperidine-1-carbonyl]-2-methyl-phenyl]-1-[2-(ethylsulfonylamino)-2-methyl-propyl]urea
N-[1-[[5-[4-(4-cyanophenyl)piperidine-1-carbonyl]-2-methyl-phenyl]carbamoylamino]-2-methyl-propan-2-yl]-2,2-dimethyl-propanamide
tert-butyl N-[3-[[5-[4-(4-cyanophenyl)piperidine-1-carbonyl]-2-methyl-phenyl]carbamoylamino]propyl]carbamate
3-(3-aminopropyl)-1-[5-[4-(4-cyanophenyl)piperidine-1-carbonyl]-2-methyl-phenyl]urea
tert-butyl 4-[[5-[4-(4-cyanophenyl)piperidine-1-carbonyl]-2-methyl-phenyl]carbamoylamino]piperidine-1-carboxylate
tert-butyl (3R)-3-[[5-[4-(4-cyanophenyl)piperidine-1-carbonyl]-2-methyl-phenyl]carbamoylamino]pyrrolidine-1-carboxylate
tert-butyl (3S)-3-[[5-[4-(4-cyanophenyl)piperidine-1-carbonyl]-2-methyl-phenyl]carbamoylamino]pyrrolidine-1-carboxylate
1-[5-[4-(4-cyanophenyl)piperidine-1-carbonyl]-2-methyl-phenyl]-3-(4-piperidyl)urea
1-[5-[4-(4-cyanophenyl)piperidine-1-carbonyl]-2-methyl-phenyl]-3-[(3R)-pyrrolidin-3-yl]urea
1-[5-[4-(4-cyanophenyl)piperidine-1-carbonyl]-2-methyl-phenyl]-3-[(3S)-pyrrolidin-3-yl]urea
1-[5-[4-(4-cyanophenyl)piperidine-1-carbonyl]-2-methyl-phenyl]-3-[2-(methyl-propan-2-ylsulfonyl-amino)ethyl]urea
1-[5-[4-(4-cyanophenyl)piperidine-1-carbonyl]-2-methyl-phenyl]-3-(3-methanesulfonamidopropyl)urea
3-[5-[4-(4-cyanophenyl)piperidine-1-carbonyl]-2-methyl-phenyl]-1-[3-(ethylsulfonylamino)propyl]urea
1-[5-[4-(4-cyanophenyl)piperidine-1-carbonyl]-2-methyl-phenyl]-3-[3-(propan-2-ylsulfonylamino)propyl]urea
1-[5-[4-(4-cyanophenyl)piperidine-1-carbonyl]-2-methyl-phenyl]-3-[(3R)-1-methylsulfonylpyrrolidin-3-yl]urea
3-[5-[4-(4-cyanophenyl)piperidine-1-carbonyl]-2-methyl-phenyl]-1-[(3R)-1-ethylsulfonylpyrrolidin-3-yl]urea
3-[5-[4-(4-cyanophenyl)piperidine-1-carbonyl]-2-methyl-phenyl]-1-[(3S)-1-propanoylpyrrolidin-3-yl]urea
1-[5-[4-(4-cyanophenyl)piperidine-1-carbonyl]-2-methyl-phenyl]-3-[(3S)-1-methylsulfonylpyrrolidin-3-yl]urea
3-[5-[4-(4-cyanophenyl)piperidine-1-carbonyl]-2-methyl-phenyl]-1-[(3S)-1-ethylsulfonylpyrrolidin-3-yl]urea
1-[5-[4-(4-cyanophenyl)piperidine-1-carbonyl]-2-methyl-phenyl]-3-(3-methylsulfonylpropyl)urea
1-benzyl-3-[3-[4-(4-cyanophenyl)piperidine-1-carbonyl]-4-fluoro-phenyl]urea
1-[5-[4-(4-cyanophenyl)piperidine-1-carbonyl]-2-methyl-phenyl]-3-cyclopropyl-urea
1-butan-2-yl-3-[5-[4-(4-cyanophenyl)piperidine-1-carbonyl]-2-methyl-phenyl]urea
3-[5-[4-(4-cyanophenyl)piperidine-1-carbonyl]-2-methyl-phenyl]-1-propyl-urea
1-[5-[4-(4-cyanophenyl)piperidine-1-carbonyl]-2-methyl-phenyl]-3-cyclohexyl-urea
1-[5-[4-(4-cyanophenyl)piperidine-1-carbonyl]-2-methyl-phenyl]-3-(3-methylbutan-2-yl)urea
3-[[5-[4-(4-cyanophenyl)piperidine-1-carbonyl]-2-methyl-phenyl]carbamoylamino]-N-(2-hydroxyethyl)propanamide
N-[2-[[5-[4-(4-cyanophenyl)piperidine-1-carbonyl]-2-methyl-phenyl]carbamoylamino]ethyl]-N′,N′-dimethyl-propanediamide
N-[2-[[5-[4-(4-cyanophenyl)piperidine-1-carbonyl]-2-methyl-phenyl]carbamoylamino]propyl]acetamide
N-[3-[[5-[4-(4-cyanophenyl)piperidine-1-carbonyl]-2-methyl-phenyl]carbamoylamino]propyl]morpholine-4-carboxamide
3-[2-(carbamoylamino)ethyl]-1-[5-[4-(4-cyanophenyl)piperidine-1-carbonyl]-2-methyl-phenyl]urea
4-[[5-[4-(4-cyanophenyl)piperidine-1-carbonyl]-2-methyl-phenyl]carbamoylamino]-N-propyl-butanamide
1-[5-[4-(4-cyanophenyl)piperidine-1-carbonyl]-2-methyl-phenyl]-3-(2-oxo-3-piperidyl)urea
Methyl 2-[[2-[[5-[4-(4-cyanophenyl)piperidine-1-carbonyl]-2-methyl-phenyl]carbamoylamino]acetyl]amino]acetate
2-[[5-[4-(4-cyanophenyl)piperidine-1-carbonyl]-2-methyl-phenyl]carbamoylamino]-N,N-dimethyl-acetamide
1-[5-[4-(4-cyanophenyl)piperidine-1-carbonyl]-2-methyl-phenyl]-3-(2-oxo-3,4-dihydro-1H-1,8-naphthyridin-3-yl)urea
(2S)-2-[[5-[4-(4-cyanophenyl)piperidine-1-carbonyl]-2-methyl-phenyl]carbamoylamino]-4-methylsulfonyl-butanoic acid
3-[[5-[4-(4-cyanophenyl)piperidine-1-carbonyl]-2-methyl-phenyl]carbamoylamino]-N-(5-methyl-1,2-oxazol-4-yl)propanamide
3-[[5-[4-(4-cyanophenyl)piperidine-1-carbonyl]-2-methyl-phenyl]carbamoylamino]-N,N-bis(2-hydroxyethyl)propanamide
1-[5-[4-(4-cyanophenyl)piperidine-1-carbonyl]-2-methyl-phenyl]-3-[(4-methylsulfonylphenyl)methyl]urea
1-[5-[4-(4-cyanophenyl)piperidine-1-carbonyl]-2-methyl-phenyl]-3-(3-pyrazol-1-ylpropyl)urea
1-[5-[4-(4-cyanophenyl)piperidine-1-carbonyl]-2-methyl-phenyl]-3-[(3-methyl-1,2-oxazol-5-yl)methyl]urea
1-[5-[4-(4-cyanophenyl)piperidine-1-carbonyl]-2-methyl-phenyl]-3-[(5-methyl-1,2-oxazol-3-yl)methyl]urea
1-[5-[4-(4-cyanophenyl)piperidine-1-carbonyl]-2-methyl-phenyl]-3-[(3-methylsulfonylphenyl)methyl]urea
1-[5-[4-(4-cyanophenyl)piperidine-1-carbonyl]-2-methyl-phenyl]-3-(1,3-thiazol-2-ylmethyl)urea
1-[5-[4-(4-cyanophenyl)piperidine-1-carbonyl]-2-methyl-phenyl]-3-[(6-methylpyridin-2-yl)methyl]urea
3-[5-[4-(4-cyanophenyl)piperidine-1-carbonyl]-2-methyl-phenyl]-1-[(3-methylpyridin-2-yl)methyl]urea
1-[5-[4-(4-cyanophenyl)piperidine-1-carbonyl]-2-methyl-phenyl]-3-[[3-(2-methoxypyridin-3-yl)-1,2-oxazol-5-yl]methyl]urea
1-[5-[4-(4-cyanophenyl)piperidine-1-carbonyl]-2-methyl-phenyl]-3-(4-methoxybutyl)urea
1-[5-[4-(4-cyanophenyl)piperidine-1-carbonyl]-2-methyl-phenyl]-3-(1-phenoxypropan-2-yl)urea
1-[5-[4-(4-cyanophenyl)piperidine-1-carbonyl]-2-methyl-phenyl]-3-[2-(3,3-difluoropyrrolidin-1-yl)ethyl]urea
1-[5-[4-(4-cyanophenyl)piperidine-1-carbonyl]-2-methyl-phenyl]-3-(1-methyl-3-piperidyl)urea
1-[5-[4-(4-cyanophenyl)piperidine-1-carbonyl]-2-methyl-phenyl]-3-(4-dimethylaminocyclohexyl)urea
1-[5-[4-(4-cyanophenyl)piperidine-1-carbonyl]-2-methyl-phenyl]-3-[(1,5-dimethylpyrazol-3-yl)methyl]urea
1-[5-[4-(4-cyanophenyl)piperidine-1-carbonyl]-2-methyl-phenyl]-3-[(1,3-dimethylpyrazol-4-yl)methyl]urea
1-[5-[4-(4-cyanophenyl)piperidine-1-carbonyl]-2-ethyl-phenyl]-3-(1,4-dioxan-2-ylmethyl)urea
3-(1-amino-2-methyl-propan-2-yl)-1-[5-[4-(4-cyanophenyl)piperidine-1-carbonyl]-2-methyl-phenyl]urea
N-[1-[[5-[4-(4-cyanophenyl)piperidine-1-carbonyl]-2-methyl-phenyl]carbamoylamino]-2-methyl-propan-2-yl]acetamide
N-[1-[[5-[4-(4-cyanophenyl)piperidine-1-carbonyl]-2-methyl-phenyl]carbamoylamino]-2-methyl-propan-2-yl]propanamide
N-[1-[[5-[4-(4-cyanophenyl)piperidine-1-carbonyl]-2-methyl-phenyl]carbamoylamino]-2-methyl-propan-2-yl]-2-methyl-propanamide
1-[5-[4-(4-cyanophenyl)piperidine-1-carbonyl]-2-methyl-phenyl]-3-[(4-methyl-1,3-thiazol-2-yl)methyl]urea
1-[5-[4-(4-cyanophenyl)piperidine-1-carbonyl]-2-methyl-phenyl]-3-[(3R)-6-oxo-3-piperidyl]urea
1-[5-[4-(4-cyanophenyl)piperidine-1-carbonyl]-2-methyl-phenyl]-3-(pyrimidin-4-ylmethyl)urea
1-[5-[4-(4-cyanophenyl)piperidine-1-carbonyl]-2-methyl-phenyl]-3-[(1-methylimidazol-4-yl)methyl]urea
1-[5-[4-(4-cyanophenyl)piperidine-1-carbonyl]-2-methyl-phenyl]-3-[(1-methylpyrrolidin-3-yl)methyl]urea
1-[5-[4-(4-cyanophenyl)piperidine-1-carbonyl]-2-methyl-phenyl]-3-(1,3-oxazol-2-ylmethyl)urea
3-[(8S)-1-azabicyclo[2.2.2]oct-8-yl]-1-[5-[4-(4-cyanophenyl)piperidine-1-carbonyl]-2-methyl-phenyl]urea
N-[2-[[5-[4-(4-cyanophenyl)piperidine-1-carbonyl]-2-methyl-phenyl]carbamoylamino]ethyl]-N,2-dimethyl-propanamide
3-[5-[4-(4-cyanophenyl)piperidine-1-carbonyl]-2-methyl-phenyl]-1-[2-(ethylsulfonyl-methyl-amino)ethyl]urea
N-[3-[[5-[4-(4-cyanophenyl)piperidine-1-carbonyl]-2-methyl-phenyl]carbamoylamino]propyl]acetamide
N-[3-[[5-[4-(4-cyanophenyl)piperidine-1-carbonyl]-2-methyl-phenyl]carbamoylamino]propyl]-2-methyl-propanamide
3-[(3R)-1-acetylpyrrolidin-3-yl]-1-[5-[4-(4-cyanophenyl)piperidine-1-carbonyl]-2-methyl-phenyl]urea
3-[5-[4-(4-cyanophenyl)piperidine-1-carbonyl]-2-methyl-phenyl]-1-[(3R)-1-propanoylpyrrolidin-3-yl]urea
3-[(3S)-1-acetylpyrrolidin-3-yl]-1-[5-[4-(4-cyanophenyl)piperidine-1-carbonyl]-2-methyl-phenyl]urea
1-[3-[4-(4-cyanophenyl)piperidine-1-carbonyl]-4-methyl-phenyl]-3-(3-methylsulfonylpropyl)urea and
tert-butyl 3-[[5-[4-(4-cyanophenyl)piperidine-1-carbonyl]-2-methyl-phenyl]carbamoylamino]piperidine-1-carboxylate
1-benzyl-3-[5-[4-(4-cyanophenyl)piperidine-1-carbonyl]-2-(trifluoromethyl)phenyl]urea
1-[5-[4-(4-cyanophenyl)piperidine-1-carbonyl]-2-methyl-phenyl]-3-(1-methylsulfonyl-4-piperidyl)urea
3-(1-acetyl-4-piperidyl)-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-[1-(2-methylpropanoyl)-4-piperidyl]urea
4-[[5-[4-(4-cyanophenyl)piperidine-1-carbonyl]-2-methyl-phenyl]carbamoylamino]-N,N-dimethyl-piperidine-1-carboxamide
1-[5-[4-(4-cyanophenyl)piperidine-1-carbonyl]-2-methyl-phenyl]-3-[1-(dimethylsulfamoyl)-4-piperidyl]urea
1-[5-[4-(4-cyanophenyl)piperidine-1-carbonyl]-2-methyl-phenyl]-3-(3-piperidyl)urea
1-[5-[4-(4-cyanophenyl)piperidine-1-carbonyl]-2-methyl-phenyl]-3-cyclobutyl-urea
1-[5-[4-(4-cyanophenyl)piperidine-1-carbonyl]-2-methyl-phenyl]-3-(cyclopropylmethyl)urea
1-[5-[4-(4-cyanophenyl)piperidine-1-carbonyl]-2-methyl-phenyl]-3-(1H-pyrazol-3-ylmethyl)urea
1-[5-[4-(4-cyanophenyl)piperidine-1-carbonyl]-2-methyl-phenyl]-3-[(1-methylpyrazol-3-yl)methyl]urea
1-[5-[4-(4-cyanophenyl)piperidine-1-carbonyl]-2-methyl-phenyl]-3-(pyrimidin-2-ylmethyl)urea
1-[5-[4-(4-cyanophenyl)piperidine-1-carbonyl]-2-methyl-phenyl]-3-[(5-methyl-2H-pyrazol-3-yl)methyl]urea
1-[5-[4-(4-cyanophenyl)piperidine-1-carbonyl]-2-methyl-phenyl]-3-(pyrazin-2-ylmethyl)urea
1-[5-[4-(4-cyanophenyl)piperidine-1-carbonyl]-2,4-dimethyl-phenyl]-3-(3-methylsulfonylpropyl)urea
3-[5-[4-(4-cyanophenyl)piperidine-1-carbonyl]-2-methyl-phenyl]-1-(1-propanoyl-3-piperidyl)urea
1-[5-[4-(4-cyanophenyl)piperidine-1-carbonyl]-2-methyl-phenyl]-3-(1-methylsulfonyl-3-piperidyl)urea
3-[5-[4-(4-cyanophenyl)piperidine-1-carbonyl]-2-methyl-phenyl]-1-(1-ethylsulfonyl-3-piperidyl)urea
1-[5-[4-(4-cyanophenyl)piperidine-1-carbonyl]-2-ethyl-phenyl]-3-(oxetan-3-yl)urea
1-[5-[4-(4-cyanophenyl)piperidine-1-carbonyl]-2-methyl-phenyl]-3-(oxetan-3-yl)urea
3-(azetidin-3-yl)-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-[1-(2-methylpropanoyl)azetidin-3-yl]urea
1-[5-[4-(4-cyanophenyl)piperidine-1-carbonyl]-2-methyl-phenyl]-3-(1-methylsulfonylazetidin-3-yl)urea
1-[5-[4-(4-cyanophenyl)piperidine-1-carbonyl]-2-methyl-phenyl]-3-[1-(dimethylsulfamoyl)azetidin-3-yl]urea
3-[(cis)-2-aminocyclohexyl]-1-[5-[4-(4-cyanophenyl)piperidine-1-carbonyl]-2-methyl-phenyl]urea
3-[(trans)-2-aminocyclohexyl]-1-[5-[4-(4-cyanophenyl)piperidine-1-carbonyl]-2-methyl-phenyl]urea
3-amino-N-[5-[4-(4-cyanophenyl)piperidine-1-carbonyl]-2-methyl-phenyl]azetidine-1-carboxamide
1-[5-[4-(4-cyanophenyl)piperidine-1-carbonyl]-2-methyl-phenyl]-3-[(trans)-2-methanesulfonamidocyclohexyl]urea
N-[(trans)-2-[[5-[4-(4-cyanophenyl)piperidine-1-carbonyl]-2-methyl-phenyl]carbamoylamino]cyclohexyl]acetamide
3-[5-[4-(4-cyanophenyl)piperidine-1-carbonyl]-2-methyl-phenyl]-1-[(trans)-2-(ethylsulfonylamino)cyclohexyl]urea
3-(1-acetylazetidin-3-yl)-1-[5-[4-(4-cyanophenyl)piperidine-1-carbonyl]-2-methyl-phenyl]urea
3-[5-[4-(4-cyanophenyl)piperidine-1-carbonyl]-2-methyl-phenyl]-1-(1-ethylsulfonylazetidin-3-yl)urea
3-acetamido-N-[5-[4-(4-cyanophenyl)piperidine-1-carbonyl]-2-methyl-phenyl]azetidine-1-carboxamide
N-[5-[4-(4-cyanophenyl)piperidine-1-carbonyl]-2-methyl-phenyl]-3-methanesulfonamido-azetidine-1-carboxamide
N-[5-[4-(4-cyanophenyl)piperidine-1-carbonyl]-2-methyl-phenyl]-3-(dimethylsulfamoylamino)azetidine-1-carboxamide
1-[5-[4-(4-cyanophenyl)piperidine-1-carbonyl]-2-methyl-phenyl]-3-[(trans)-2-methanesulfonamidocyclohexyl]urea
tert-butyl N-[(1S,3S)-3-[[5-[4-(4-cyanophenyl)piperidine-1-carbonyl]-2-methyl-phenyl]carbamoylamino]cyclopentyl]carbamate
1-[5-[4-(4-cyanophenyl)piperidine-1-carbonyl]-2-methyl-phenyl]-3-[(cis)-2-(dimethylsulfamoylamino)cyclohexyl]urea
3-[(1S,3S)-3-aminocyclopentyl]-1-[5-[4-(4-cyanophenyl)piperidine-1-carbonyl]-2-methyl-phenyl]urea
1-[5-[4-(4-cyanophenyl)piperidine-1-carbonyl]-2-methyl-phenyl]-3-[(1R,2R)-2-(dimethylsulfamoylamino)cyclohexyl]urea
1-[5-[4-(4-cyanophenyl)piperidine-1-carbonyl]-2-methyl-phenyl]-3-[(1S,3S)-3-methanesulfonamidocyclopentyl]urea
N-[(1S,3S)-3-[[5-[4-(4-cyanophenyl)piperidine-1-carbonyl]-2-methyl-phenyl]carbamoylamino]cyclopentyl]acetamide
N-[5-[4-(4-cyanophenyl)piperidine-1-carbonyl]-2-methyl-phenyl]-4-methylsulfonyl-piperazine-1-carboxamide
1-[5-[4-(4-cyanophenyl)piperidine-1-carbonyl]-2-methyl-phenyl]-3-[(1S,3S)-3-(dimethylsulfamoylamino)cyclopentyl]urea
3-[5-[4-(4-cyanophenyl)piperidine-1-carbonyl]-2-methyl-phenyl]-1-[(1S,3S)-3-(ethylsulfonylamino)cyclopentyl]urea
1-[5-[4-(4-fluorophenyl)piperidine-1-carbonyl]-2-methyl-phenyl]-3-propan-2-yl-urea
N-[2-[[2-methyl-5-[4-[4-(trifluoromethyl)phenyl]piperidine-1-carbonyl]phenyl]carbamoylamino]ethyl]acetamide
1-[5-[4-(3-cyanophenyl)piperidine-1-carbonyl]-2-methyl-phenyl]-3-propan-2-yl-urea
1-[5-[4-(4-methoxyphenyl)piperidine-1-carbonyl]-2-methyl-phenyl]-3-propan-2-yl-urea
N-methyl-4-[1-[4-methyl-3-(propan-2-ylcarbamoylamino)benzoyl]-4-piperidyl]benzamide
Ethyl 4-[[5-[4-(4-cyanophenyl)piperidine-1-carbonyl]-2-methyl-phenyl]carbamoyl]piperazine-1-carboxylate
N-[5-[4-(4-cyanophenyl)piperidine-1-carbonyl]-2-methyl-phenyl]-4-(2-methylpropanoyl)piperazine-1-carboxamide
N-[5-[4-(4-cyanophenyl)piperidine-1-carbonyl]-2-methyl-phenyl]-N′,N′-dimethyl-piperazine-1,4-dicarboxamide
tert-butyl 3-[[2-methyl-5-[4-[4-(trifluoromethyl)phenyl]piperidine-1-carbonyl]phenyl]carbamoylamino]piperidine-1-carboxylate
3-(2-dimethylaminoethyl)-1-[2-methyl-5-[4-[4-(trifluoromethyl)phenyl]piperidine-1-carbonyl]phenyl]urea
1-[5-[4-(4-cyanophenyl)piperidine-1-carbonyl]-2-methyl-phenyl]-3-(3-methyl-1,1-dioxo-thiolan-3-yl)urea
tert-butyl N-[2-[[2-methyl-5-[4-[4-(trifluoromethyl)phenyl]piperidine-1-carbonyl]phenyl]carbamoylamino]ethyl]carbamate
1-[2-methyl-5-[4-[4-(trifluoromethyl)phenyl]piperidine-1-carbonyl]phenyl]-3-(3-piperidyl)urea
N-[2-[[5-[4-(4-fluorophenyl)piperidine-1-carbonyl]-2-methyl-phenyl]carbamoylamino]ethyl]acetamide
tert-butyl 3-[[5-[4-(4-fluorophenyl)piperidine-1-carbonyl]-2-methyl-phenyl]carbamoylamino]azetidine-1-carboxylate
3-[2-(dimethylsulfamoylamino)ethyl]-1-[2-methyl-5-[4-[4-(trifluoromethyl)phenyl]piperidine-1-carbonyl]phenyl]urea
3-(2-methanesulfonamidoethyl)-1-[2-methyl-5-[4-[4-(trifluoromethyl)phenyl]piperidine-1-carbonyl]phenyl]urea
3-(2-aminoethyl)-1-[2-methyl-5-[4-[4-(trifluoromethyl)phenyl]piperidine-1-carbonyl]phenyl]urea
tert-butyl 3-[[5-[4-(4-fluorophenyl)piperidine-1-carbonyl]-2-methyl-phenyl]carbamoylamino]pyrrolidine-1-carboxylate
N-[5-[4-(4-cyanophenyl)piperidine-1-carbonyl]-2-methyl-phenyl]-3-methylsulfonyl-pyrrolidine-1-carboxamide
N-[5-[4-(4-cyanophenyl)piperidine-1-carbonyl]-2-methyl-phenyl]-3-hydroxy-azetidine-1-carboxamide
N-[5-[4-(4-cyanophenyl)piperidine-1-carbonyl]-2-methyl-phenyl]azetidine-1-carboxamide
tert-butyl N-[2-[[5-[4-(4-fluorophenyl)piperidine-1-carbonyl]-2-methyl-phenyl]carbamoylamino]ethyl]-N-methyl-carbamate
3-(1-acetylpyrrolidin-3-yl)-1-[5-[4-(4-fluorophenyl)piperidine-1-carbonyl]-2-methyl-phenyl]urea
1-[5-[4-(4-fluorophenyl)piperidine-1-carbonyl]-2-methyl-phenyl]-3-pyrrolidin-3-yl-urea
1-[5-[4-(4-chlorophenyl)-4-hydroxy-piperidine-1-carbonyl]-2-methyl-phenyl]-3-propan-2-yl-urea
1-[5-[4-(4-fluorophenyl)piperidine-1-carbonyl]-2-methyl-phenyl]-3-[2-(methyl-methylsulfonyl-amino)ethyl]urea
N-[2-[[5-[4-(4-fluorophenyl)piperidine-1-carbonyl]-2-methyl-phenyl]carbamoylamino]ethyl]-N-methyl-acetamide
N-[2-[[5-[4-(4-cyanophenyl)piperidine-1-carbonyl]-2-methyl-phenyl]carbamoyl-methyl-amino]ethyl]propanamide
1-[5-[4-(4-cyanophenyl)piperidine-1-carbonyl]-2-methyl-phenyl]-3-(1-pyridin-3-ylethyl)urea
1-[5-[4-(4-chlorophenyl)piperidine-1-carbonyl]-2-methyl-phenyl]-3-(pyridin-2-ylmethyl)urea
1-[5-[4-(4-cyanophenyl)piperidine-1-carbonyl]-2-methyl-phenyl]-3-[(1-methylpyrazol-4-yl)methyl]urea
1-[5-[4-(4-cyanophenyl)piperidine-1-carbonyl]-2-methyl-phenyl]-3-(1-pyridin-4-ylethyl)urea
1-[5-[4-(4-cyanophenyl)piperidine-1-carbonyl]-2-methyl-phenyl]-3-[(6-morpholin-4-ylpyridin-2-yl)methyl]urea
1-[5-[4-(4-chlorophenyl)piperidine-1-carbonyl]-2-methyl-phenyl]-3-propan-2-yl-urea
1-[2-methyl-5-[4-(4-sulfamoylphenyl)piperidine-1-carbonyl]phenyl]-3-propan-2-yl-urea
1-[5-[4-(4-cyanophenyl)piperidine-1-carbonyl]-2-methyl-phenyl]-3-methyl-urea
3-[5-[4-(4-cyanophenyl)piperidine-1-carbonyl]-2-methylsulfanylphenyl]-1-propan-2-ylurea
3-benzyl-1-[5-[4-(4-cyanophenyl)piperidine-1-carbonyl]-2-methylsulfanylphenyl]urea
3-[5-[4-(4-cyanophenyl)piperidine-1-carbonyl]-2-methylsulfonylphenyl]-1-propan-2-ylurea
3-[5-[4-(4-cyanophenyl)piperidine-1-carbonyl]-2-(methoxymethyl)phenyl]-1-propan-2-ylurea
[5-[4-(4-cyanophenyl)piperidine-1-carbonyl]-2-methylphenyl]urea
1-[5-[4-(4-cyanophenyl)piperidine-1-carbonyl]-2-methylphenyl]-3-[(3,5-difluoropyridin-2-yl)methyl]urea
1-[5-[4-(4-cyanophenyl)piperidine-1-carbonyl]-2-methylphenyl]-3-(1-pyridin-2-ylethyl)urea
2-[[5-[4-(4-cyanophenyl)piperidine-1-carbonyl]-2-methylphenyl]carbamoylamino]-3-(4-fluorophenyl)propanoic acid
(2R)-3-[[5-[4-(4-cyanophenyl)piperidine-1-carbonyl]-2-methylphenyl]carbamoylamino]-2-[(2-methylpropan-2-yl)oxycarbonylamino]propanoic acid
2-[[5-[4-(4-cyanophenyl)piperidine-1-carbonyl]-2-methylphenyl]carbamoylamino]-3-methylbutanoic acid
4-[[5-[4-(4-cyanophenyl)piperidine-1-carbonyl]-2-methylphenyl]carbamoylamino]butanoic acid
2-[[5-[4-(4-cyanophenyl)piperidine-1-carbonyl]-2-methylphenyl]carbamoylamino]acetic acid
1-[2-methyl-5-[4-(4-methylsulfonylphenyl)piperidine-1-carbonyl]phenyl]-3-propan-2-ylurea
1-[3-[4-(4-cyanophenyl)piperidine-1-carbonyl]-4-methylphenyl]-3-propan-2-ylurea
3-tert-butyl-1-[3-[4-(4-cyanophenyl)piperidine-1-carbonyl]-4-methylphenyl]urea
3-[3-[4-(4-cyanophenyl)piperidine-1-carbonyl]-4-fluorophenyl]-1-propan-2-ylurea
3-[(4-cyanophenyl)methyl]-1-[3-[4-(4-cyanophenyl)piperidine-1-carbonyl]-4-methylphenyl]urea
1-[3-[4-(4-cyanophenyl)piperidine-1-carbonyl]-4-methylphenyl]-3-(pyridin-4-ylmethyl)urea
3-[3-[4-(4-cyanophenyl)piperidine-1-carbonyl]-4-methylphenyl]-1,1-dimethylurea
1-benzyl-3-[5-[4-(4-cyanophenyl)piperidine-1-carbonyl]-2-methylphenyl]-1-methylurea
1-[5-[4-(4-bromophenyl)-4-hydroxypiperidine-1-carbonyl]-2-methylphenyl]-3-propan-2-ylurea
1-[5-[4-(4-cyanophenyl)piperidine-1-carbonyl]-2-methylphenyl]-3-(3-oxo-1,2-oxazolidin-4-yl)urea
1-[5-[4-(4-cyanophenyl)piperidine-1-carbonyl]-2-methylphenyl]-3-(2-methylbut-3-yn-2-yl)urea
Ethyl 2-[[5-[4-(4-cyanophenyl)piperidine-1-carbonyl]-2-methylphenyl]carbamoylamino]-propanoate
3-[5-[4-(4-cyanophenyl)piperidine-1-carbonyl]-2-methylsulfinylphenyl]-1-propan-2-ylurea
3-benzyl-1-[5-[4-(4-cyanophenyl)piperidine-1-carbonyl]-2-methylsulfinylphenyl]urea
2-[[5-[4-(4-cyanophenyl)piperidine-1-carbonyl]-2-methylphenyl]carbamoylamino]cyclopentane-1-carboxylic acid
1-[5-[4-(4-cyanophenyl)piperidine-1-carbonyl]-2-methylphenyl]-3-(1-methylcyclopropyl)urea
3-(1-acetylpiperidin-3-yl)-1-[5-[4-(4-cyanophenyl)piperidine-1-carbonyl]-2-methylphenyl]urea
1-[2-methyl-5-(4-phenylpiperidine-1-carbonyl)phenyl]-3-propan-2-ylurea
3-[5-[4-(4-cyanophenyl)piperidine-1-carbonyl]-2-methylphenyl]-1-(2-hydroxyethyl)-1-methylurea
3-[5-[4-(4-cyanophenyl)piperidine-1-carbonyl]-2-methylphenyl]-1-methyl-1-(1-methylpiperidin-4-yl)urea
3-[5-[4-(4-cyanophenyl)piperidine-1-carbonyl]-2-methylphenyl]-1-(2-dimethylaminoethyl)-1-methylurea
3-[5-[4-(4-cyanophenyl)piperidine-1-carbonyl]-2-methylphenyl]-1-(2-hydroxyethyl)-1-propan-2-ylurea
3-[5-[4-(4-cyanophenyl)piperidine-1-carbonyl]-2-methylphenyl]-1-methyl-1-(oxan-4-yl)urea
3-[5-[4-(4-cyanophenyl)piperidine-1-carbonyl]-2-methylphenyl]-1-(1,1-dioxothiolan-3-yl)-1-propylurea
2-[[5-[4-(4-cyanophenyl)piperidine-1-carbonyl]-2-methylphenyl]carbamoyl-methylamino]-N-propan-2-ylacetamide
N-[5-[4-(4-cyanophenyl)piperidine-1-carbonyl]-2-methylphenyl]-2-(ethoxymethyl)pyrrolidine-1-carboxamide
1-[5-[4-(4-fluorophenyl)piperidine-1-carbonyl]-2-methylphenyl]-3-(2-methylaminoethyl)urea
N-[2-[[5-[4-(4-cyanophenyl)piperidine-1-carbonyl]-2-methylphenyl]carbamoyl-methylamino]ethyl]acetamide
3-[5-[4-(4-cyanophenyl)piperidine-1-carbonyl]-2-methylphenyl]-1-[2-(ethylsulfonylamino)ethyl]-1-methylurea
3-[5-[4-(4-cyanophenyl)piperidine-1-carbonyl]-2-(methylsulfonylmethyl)phenyl]-1-propan-2-ylurea
1-[3-[4-(4-cyanophenyl)piperidine-1-carbonyl]-4-methylphenyl]-3-propan-2-ylurea
3-[3-[4-(4-cyanophenyl)piperidine-1-carbonyl]-4-methylsulfanylphenyl]-1-propan-2-ylurea
4-[[5-[4-(4-cyanophenyl)piperidine-1-carbonyl]-2-methylphenyl]carbamoylamino]cyclohexane-1-carboxylic acid
4-[[5-[4-(4-cyanophenyl)piperidine-1-carbonyl]-2-methylphenyl]carbamoylamino]butanoic acid
2-[[5-[4-(4-cyanophenyl)piperidine-1-carbonyl]-2-methylphenyl]carbamoylamino]acetic acid
1-[5-[4-(4-cyanophenyl)piperidine-1-carbonyl]-2,4-dimethylphenyl]-3-(pyridin-2-ylmethyl)urea
3-[5-[4-(4-cyanophenyl)piperidine-1-carbonyl]-2,4-dimethylphenyl]-1-ethylurea
1-[5-[4-(4-cyanophenyl)piperidine-1-carbonyl]-2,4-dimethylphenyl]-3-cyclopropylurea
1-[5-[4-(4-cyanophenyl)piperidine-1-carbonyl]-2,4-dimethylphenyl]-3-(2-methoxyethyl)urea
1-[5-[4-(4-cyanophenyl)piperidine-1-carbonyl]-2,4-dimethylphenyl]-3-prop-2-ynylurea
1-[3-[4-(4-cyanophenyl)piperidine-1-carbonyl]-4-methylphenyl]-3-(pyridin-2-ylmethyl)urea
1-[3-[4-(4-cyanophenyl)piperidine-1-carbonyl]-4-methylphenyl]-3-cyclopropylurea
3-[3-[4-(4-cyanophenyl)piperidine-1-carbonyl]-4-methylphenyl]-1-(3-ethoxypropyl)urea
1-[3-[4-(4-cyanophenyl)piperidine-1-carbonyl]-4-methylphenyl]-3-(2-methoxyethyl)urea
1-[3-[4-(4-cyanophenyl)piperidine-1-carbonyl]-4-methylphenyl]-3-prop-2-ynylurea
   or a pharmaceutically-acceptable salt thereof.
   In another embodiment there is provided a compound or compounds selected from one or more of the following compounds labelled as List 2:
3-[5-[4-(4-cyanophenyl)piperidine-1-carbonyl]-2-methyl-phenyl]-1-propan-2-yl-urea;
3-[5-[4-(4-cyanophenyl)piperidine-1-carbonyl]-2-methyl-phenyl]-1-ethyl-urea;
1-[5-[4-(4-cyanophenyl)piperidine-1-carbonyl]-2-methyl-phenyl]-3-(pyridin-2-ylmethyl)urea;
1-[5-[4-(4-cyanophenyl)piperidine-1-carbonyl]-2-methyl-phenyl]-3-(2-methoxyethyl)urea;
1-[5-[4-(4-cyanophenyl)piperidine-1-carbonyl]-2-ethyl-phenyl]-3-propan-2-yl-urea;
1-[5-[4-(4-cyanophenyl)piperidine-1-carbonyl]-2-methyl-phenyl]-3-(3-methanesulfonamidopropyl)urea;
1-[5-[4-(4-cyanophenyl)piperidine-1-carbonyl]-2-methyl-phenyl]-3-[(3S)-1-methylsulfonylpyrrolidin-3-yl]urea;
3-[5-[4-(4-cyanophenyl)piperidine-1-carbonyl]-2-methyl-phenyl]-1-[(3S)-1-ethylsulfonylpyrrolidin-3-yl]urea;
1-[5-[4-(4-cyanophenyl)piperidine-1-carbonyl]-2-methyl-phenyl]-3-(4-dimethylaminocyclohexyl)urea;
3-(azetidin-3-yl)-1-[5-[4-(4-cyanophenyl)piperidine-1-carbonyl]-2-methyl-phenyl]urea; or
1-[5-[4-(4-cyanophenyl)piperidine-1-carbonyl]-2-methylphenyl]-3-[(3,5-difluoropyridin-2-yl)methyl]urea
   or a pharmaceutically-acceptable salt thereof

A compound of the Formula I, or a pharmaceutically-acceptable salt thereof, may be prepared by any process known to be applicable to the preparation of chemically-related compounds. Such processes, when used to prepare a compound of the formula I are provided as a further feature of the invention and are illustrated by the following representative process variants. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated that are within the ordinary skill of an organic chemist. Unless otherwise stated R¹, R^{a}, R², R^{b}, R³, R⁴, R⁵, R^{5′}, R⁶, R^{6′} and R⁷ are as described above.

According to a further aspect, the present invention provides a process for preparing a compound of formula I

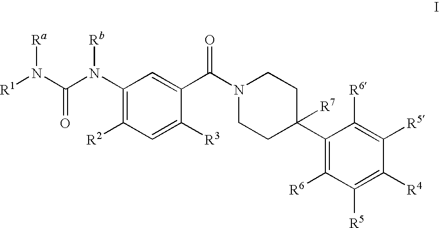

or a pharmaceutically acceptable salt thereof wherein R¹, R^{a}, R², R^{b}, R³, R⁴, R⁵, R^{5′}, R⁶, R^{6′} and R⁷ are, unless otherwise specified which comprises
(a) reacting a compound of formula VI

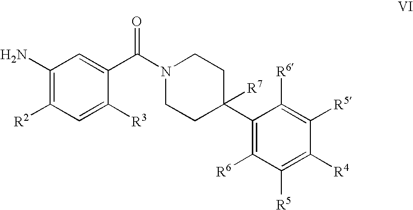

with an isocyanate of formula VII

R¹—N═C═O VII

to give compounds of formula I in which R^{a} is H or
b) reacting a compound of formula VI

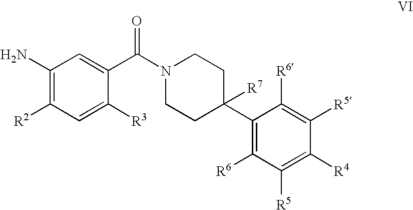

with phosgene or an equivalent thereof, for example triphosgene, and then further reacting the intermediate obtained with an amine of formula VIII

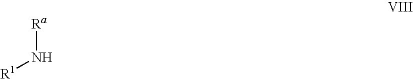

Compounds of formula I may also be prepared by reacting a compound of formula IX

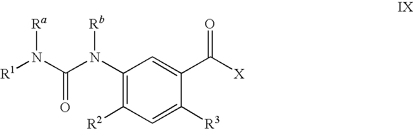

in which X represents a leaving group for example halo, e.g. chloro with a compound of formula X

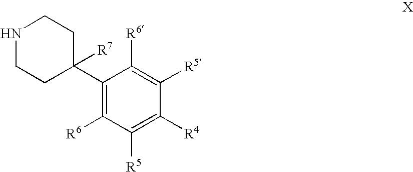

in the presence of a diluent for example a solvent e.g. dichloromethane and optionally in the presence of a base, for example an organic amine e.g. DIPEA, at a temperature in the range of 0-150° C.

Compounds of formula I may also be prepared by reacting a compound of formula XI

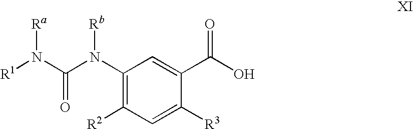

with a compound of formula X optionally in the presence of a coupling agent and optionally in the presence of a diluent for example a solvent at a temperature in the range of 0-150° C.

Compounds of formula I may also be prepared by reacting a compound of formula XII

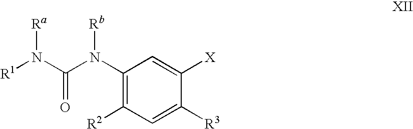

in which X represents a replaceable group, e.g. Cl, Br, I, OMesyl, or OTriflyl with a compound of formula X in the presence of carbon monoxide and in the presence of a metal catalyst, e.g. Pd or derivatives thereof, and in a solvent such as an alcohol, THF, toluene, or DMF, and in the temperature range 0-150° C. The carbon monoxide may be gaseous or in the form of a metal carbonyl, e.g. Molybdenum hexacarbonyl.

Compounds of formula I in which R^{b} is H may also be prepared by reacting a compound of formula XIII

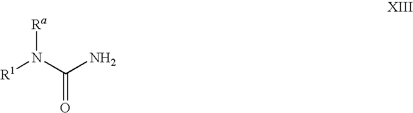

with a compound of formula XIV

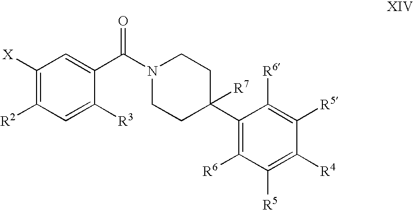

in which X represents a replaceable group, e.g. F, Cl, Br, I, OMesyl, or OTriflyl, in the presence of a metal catalyst, for example Pd (O), Pd (II) or CU (I), in an organic diluent for example, dioxan, DMF, NMP or DMA at a temperature in the range 0-150° C.

Examples of coupling agents are Dichlorotriphenyl phosphorane (DCTPP), 1-ethyl-3-(3-dimethylaminopropyl)carbodiimide hydrochloride (EDAC), O-benzotriazol-1-yl-N,N,N′,N′-tetramethyluronium hexafluorophosphate (HTBU), O-(7-azabenzotriazol-1-yl)-N,N,N′,N′-tetramethyluronium hexafluorophosphate (HATU) and 4-(4,6-dimethoxy-1,3,5-triazin-2-yl)-4-methylmorpholinium chloride (DMTMM).

Examples of optional additives are: 1-hydroxy benzotriazole (HOBt), 4-dimethylamino pyridine (DMAP), di-iso-propylethylamine (DIPEA), and triethylamine (TEA).

Examples of suitable solvents are: dimethyl formamide (DMF), chloroform, dichloromethane (DCM), and tetrahydrofuran (THF).

Certain compounds of formula I may be converted into other compounds of formula I by methods known to those skilled in the art. For example, compounds of formula I

Compounds of formula I in which R¹ represents an optionally substituted pyridyl-N-oxide may be prepared by reacting a compound of formula I in which R¹ represents an optionally substituted pyridyl with an oxidising agent for example urea hydrogen peroxide or 3-chloroperbenzoic acid, in the presence of a diluent for example dichloromethane or acetonitrile at a temperature in the range of 0-150° C.

In other processes compounds of formula I containing a sulphide group may be oxidised to SO or SO₂ for example by use of potassium peroxymonosulfate, nitriles may be reduce to aminomethyl compounds, amines may be acylated or sulphonated to give amides or sulphonamides, respectively, activated heteroaryl halides may be hydrolysed to hydroxy groups, esters may be hydrolysed to acids, and carboxylic acids may be esterified.
It will be appreciated by those skilled in the art that certain functional groups may require protection before certain transformations are attempted followed by deprotection after the particular transformation. Such methods are well known to those skilled in the art and are described in “Protective Groups in Organic Synthesis”, 2^{nd} Edition (1991) by Greene and Wuts.

Certain intermediates of formula VI are believed to be novel and are herein claimed as another aspect of the present invention.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation comprising a compound of formula I, or pharmaceutically acceptable salt thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating), dyslipidaemia and the treatment of type 2 diabetes mellitus.

The present compounds of formula (I) are useful for the prophylaxis and/or treatment of clinical conditions associated with inherent or induced reduced sensitivity to insulin (insulin resistance) and associated metabolic disorders (also known as the metabolic syndrome). These clinical conditions will include, but will not be limited to, general obesity, abdominal obesity, arterial hypertension, hyperinsulinaemia, hyperglycaemia, type 2 diabetes and the dyslipidaemia characteristically appearing with insulin resistance. This dyslipidaemia, also known as the atherogenic lipoprotein profile, is characterised by moderately elevated non-esterified fatty acids, elevated very low density lipoprotein (VLDL) triglyceride rich particles, high Apo B levels, low high density lipoprotein (HDL) levels associated with low apoAI particle levels and high Apo B levels in the presence of small, dense, low density lipoproteins (LDL) particles, phenotype B.

The compounds of the present invention are expected to be useful in treating patients with combined or mixed hyperlipidemias or various degrees of hypertriglyceridemias and postprandial dyslipidemia with or without other manifestations of the metabolic syndrome.

Treatment with the present compounds is expected to lower the cardiovascular morbidity and mortality associated with atherosclerosis due to their antidyslipidaemic as well as antiinflammatory properties. The cardiovascular disease conditions include macro-angiopathies of various internal organs causing myocardial infarction, congestive heart failure, cerebrovascular disease and peripheral arterial insufficiency of the lower extremities. Because of their insulin sensitizing effect the compounds of formula I are also expected to prevent or delay the development of type 2 diabetes from the metabolic syndrome and diabetes of pregnancy. Therefore the development of long-term complications associated with chronic hyperglycaemia in diabetes mellitus, such as the micro-angiopathies causing renal disease, retinal damage and peripheral vascular disease of the lower limbs, is expected to be delayed. Furthermore the compounds may be useful in treatment of various conditions outside the cardiovascular system whether or not associated with insulin resistance, like polycystic ovarian syndrome, obesity, cancer and states of inflammatory disease including neurodegenerative disorders such as mild cognitive impairment, Alzheimer's disease, Parkinson's disease and multiple sclerosis.

The compounds of formula I may also be useful in the treatment of metabolic syndrome and Prader-Willi syndrome.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating) and for the treatment or prophylaxis of dyslipidaemia and for the treatment or prophylaxis of type 2 diabetes mellitus.

In a still further aspect the present invention provides a method of treating obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating) dyslipidaemia and type 2 diabetes mellitus comprising administering a pharmacologically effective amount of a compound of formula I, to a patient in need thereof.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:
a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an aldose reductase inhibitor;
a glycogen phosphorylase inhibitor;
a glycogen synthase kinase inhibitors;
a glucokinase activator;
a haemostasis modulator;
an antithrombotic;
an activator of fibrinolysis;
an antiplatelet agent;
a thrombin antagonist;
a factor Xa inhibitor;
a factor VIIa inhibitor;
an antiplatelet agents;
a 5 HT transporter inhibitor;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator; for example an NPY agonist or an NPY2 agonist or an NPY5 antagonist;
an Mc4r modulator for example an Mc4r agonist;
an Mc3r modulator for example an Mc3r agonist;
an orexin receptor modulator for example an antagonist;
a phosphoinositide-dependent protein kinase (PDK) modulator; or
modulators of nuclear receptors for example LXR, FXR, RXR, GR, ERRα, β, PPARα, β, γ, δ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a CB1 receptor modulator for example an inverse agonist or an antagonist;
a GLK receptor modulator;
a DPP-IV inhibitor;
a cholesterol absorption inhibitor;
a GLP-1 agonist;
an SGLT-2 inhibitor;
a DGAT1 inhibitor;
a DGAT2 inhibitor;
a DGAT2 anti-sense oligonucleotide;
a ghrelin antibody;
a ghrelin antagonist;
an 11β HSD-1 inhibitor;
an UCP-1, 2 or 3 activator;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

The compounds of the invention may also be useful as anti-cell-proliferation (such as anti-cancer) agents and are therefore useful in methods of treatment of the human or animal body.

Such properties are expected to be of value in the treatment of disease states associated with cell cycle and cell proliferation such as cancers (solid tumors and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine, N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

The compounds of the present invention may also be useful as anti-infective agents or as anti-bacterial agents.

The compounds of the present invention may also be useful as in decreasing sebum production following topical application.

### Pharmacological Activity

The compounds of the present invention are Fatty Acid Synthase inhibitors. The activity of the compounds of the invention was demonstrated using the following assay.

### Human and Rat FAS Enzyme Assay.

Fatty acid synthase is an enzyme complex that harbours seven enzymatic activities catalysing the reductive synthesis of long chain fatty acids from acetyl CoA and malonyl CoA to palmitate. When acetyl CoA and malonyl CoA are forming palmitate NADPH is consumed forming NADP. Since NADPH is fluorescent but not NADP the reaction can be measured by analysing the decrease in fluorescence.

Compounds were added to a black 384 well plate (Matrix) in a volume of 5 μl consisting of 20% DMSO and 80% Tris buffer pH 7.5, at a top concentration of 1 mM. NADPH, 30 μl of 166.6 μM, formulated in assay buffer (0.1M Tris ph7.5, 0.1 mM EDTA, 1 mM glutathione, 0.05% BSA), was then added to all of the wells of the plate. Fatty acid synthase Human or rat enzyme (0.4 μg, produced in house), dissolved in 20 mM Tris/HCl pH 7.5, 5 mM BOG, 1 mM TCEP, 10% glycerol, 1 mM EDTA, 150 mM NaCl, was then added to the plate in a volume of 10 μl. Enzyme was added to all but the last two columns of the plate, to which, 10 μl of assay buffer was added (0.1M Tris ph7.5, 0.1 mM EDTA, 1 mM glutathione, 0.05% BSA) to provide a no enzyme assay control. Following a 15-minute incubation period, at room temperature, the plates were read on an Envision plate reader using 340 nm excitation and 460 nm emission filters. This served as a time zero background read. Substrates (an equal mix of both malonyl and acetyl CoA) were then added to the plates in a total volume of 5 μl. The concentrations of malonyl and acetyl CoA in the mixture were 500 μM and 150 μM respectively. Both were prepared as 10 mM stock solutions in distilled water and were subsequently diluted to working concentrations in assay buffer. Plates were then incubated for a further 60 minutes, at room temperature, before being read again on the Envision reader using the same parameters as previously used. The data was analysed by subtracting the background time zero data from that generated following the final 60 minute incubate and the percent inhibition compared to the maximum and minimum assay controls was determined. Sigmoid curves were fitted using Origin 7.5 Client software and IC50 values were determined.

The compounds of the present invention were found to inhibit the activation of human Fatty Acid Synthase with IC₅₀s in a range of about 0.0001 μM to about 30 μM. The examples of the present invention inhibited the activation of human Fatty Acid Synthase with IC₅₀s in a range of about 0.001 μM to about 30.0 μM. In another embodiment, the compounds inhibit the activation of Fatty Acid Synthase with IC₅₀ₛ in a range of about 0.0001 μM to about 0.1 μM.

The results obtained are given in Table 1 in which Ex No stands for Example Number and Inhib (%) stands for the % inhibition at a concentration of 100 μmolar.

TABLE 1

| Ex No | Inhib (%) |
|---|---|
| 1 | 95.6 |
| 2 | 79.7 |
| 3 | 95.6 |
| 4 | 86.1 |
| 5 | 78.8 |
| 6 | 79.6 |
| 7 | 79.1 |
| 8 | 82.6 |
| 9 | 70.4 |
| 10 | 83.5 |
| 11 | 68.4 |
| 12 | 84.6 |
| 13 | 84.8 |
| 14 | 90.1 |
| 15 | 91.3 |
| 16 | 98.3 |
| 17 | 90.1 |
| 18 | 75.9 |
| 19 | 79.9 |
| 20 | 79.2 |
| 21 | 78.8 |
| 22 | 86.8 |
| 23 | 77.0 |
| 24 | 78.1 |
| 25 | 79.7 |
| 26 | 82.9 |
| 27 | 80.7 |
| 28 | 83.1 |
| 29 | 79.6 |
| 30 | 91.3 |
| 31 | 83.3 |
| 32 | 95.5 |
| 33 | 91.8 |
| 34 | 86.5 |
| 35 | 91.6 |
| 36 | 93.8 |
| 37 | 74.3 |
| 38 | 76.6 |
| 39 | 79.1 |
| 40 | 71.5 |
| 41 | 94.4 |
| 42 | 83.8 |
| 43 | 88.3 |
| 44 | 87.9 |
| 45 | 91.8 |
| 46 | 90.3 |
| 47 | 77.4 |
| 48 | 93.7 |
| 49 | 98.1 |
| 50 | 81.2 |
| 51 | 100.6 |
| 52 | 94.4 |
| 53 | 90.8 |
| 54 | 92.2 |
| 55 | 93.2 |
| 56 | 105.2 |
| 57 | 96.8 |
| 58 | 89.9 |
| 59 | 87.2 |
| 60 | 95.3 |
| 61 | 84.8 |
| 62 | 101.0 |
| 63 | 93.4 |
| 64 | 94.0 |
| 65 | 99.8 |
| 66 | 91.5 |
| 67 | 96.9 |
| 68 | 102.5 |
| 69 | 87.9 |
| 70 | 87.7 |
| 71 | 94.2 |
| 72 | 80.1 |
| 73 | 78.2 |
| 74 | 89.6 |
| 75 | 87.4 |
| 76 | 97.5 |
| 77 | 89.6 |
| 78 | 85.7 |
| 79 | 95.4 |
| 80 | 96.1 |
| 81 | 88.2 |
| 82 | 82.0 |
| 83 | 102.2 |
| 84 | 92.3 |
| 85 | 93.6 |
| 86 | 94.0 |
| 87 | 92.5 |
| 88 | 87.6 |
| 89 | 93.1 |
| 90 | 93.6 |
| 91 | 95.9 |
| 92 | 101.7 |
| 93 | 90.1 |
| 94 | 89.6 |
| 95 | 94.4 |
| 96 | 102.9 |
| 97 | 95.5 |
| 98 | 91.9 |
| 99 | 90.2 |
| 100 | 77.8 |
| 101 | 90.7 |
| 102 | 91.9 |
| 103 | 91.8 |
| 104 | 74.5 |
| 104A | 79.0 |
| 105 | 92.7 |
| 106 | 90.3 |
| 107 | 97.7 |
| 108 | 91.8 |
| 109 | 98.3 |
| 110 | 94.2 |
| 111 | 95.6 |
| 112 | 94.3 |
| 113 | 92.7 |
| 114 | 89.4 |
| 115 | 92.9 |
| 116 | 86.2 |
| 117 | 84.5 |
| 118 | 96.2 |
| 119 | 89.2 |
| 120 | 68.3 |
| 121 | 86.0 |
| 122 | 96.1 |
| 123 | 79.7 |
| 124 | 78.0 |
| 125 | 91.7 |
| 126 | 87.4 |
| 127 | 93.0 |
| 128 | 87.5 |
| 129 | 99.1 |
| 130 | 90.5 |
| 131 | 89.8 |
| 132 | 89.5 |
| 133 | 81.2 |
| 134 | 84.6 |
| 135 | 81.1 |
| 136 | 80.9 |
| 137 | 87.9 |
| 138 | 83.7 |
| 139 | 87.0 |
| 140 | 79.2 |
| 141 | 82.1 |
| 142 | 80.3 |
| 143 | 84.4 |
| 144 | 81.7 |
| 145 | 79.3 |
| 146 | 89.7 |
| 147 | 79.5 |
| 148 | 79.5 |
| 149 | 80.2 |
| 150 | 85.0 |
| 151 | 92.1 |
| 152 | 95.1 |
| 153 | 88.2 |
| 154 | 93.0 |
| 155 | 90.1 |
| 156 | 92.2 |
| 157 | 92.7 |
| 158 | 89.1 |
| 159 | 88.0 |
| 160 | 94.9 |
| 161 | 93.7 |
| 162 | 84.2 |
| 163 | 101.6 |
| 164 | 95.0 |
| 165 | 92.4 |
| 166 | 94.7 |
| 167 | 90.3 |
| 168 | 84.6 |
| 169 | 95.6 |
| 170 | 94.6 |
| 171 | 96.3 |
| 172 | 91.8 |
| 173 | 95.5 |
| 174 | 97.4 |
| 175 | 96.1 |
| 176 | 92.6 |
| 177 | 94.8 |
| 178 | 95.9 |
| 179 | 89.2 |
| 180 | 92.0 |
| 181 | 93.1 |
| 182 | 96.2 |
| 183 | 89.6 |
| 184 | 81.7 |
| 185 | 92.6 |
| 186 | 86.4 |
| 187 | 90.4 |
| 188 | 89.0 |
| 189 | 85.6 |
| 190 | 82.9 |
| 191 | 92.2 |
| 192 | 85.1 |
| 193 | 85.8 |
| 194 | 84.3 |
| 195 | 88.6 |
| 196 | 91.7 |
| 197 | 84.4 |
| 198 | 87.9 |
| 199 | 96.3 |
| 200 | 91.8 |
| 201 | 94.0 |
| 202 | 97.3 |
| 203 | 91.0 |
| 204 | 91.0 |
| 205 | 88.2 |
| 206 | 83.8 |
| 207 | 89.7 |
| 208 | 84.9 |
| 209 | 89.7 |
| 210 | 97.4 |
| 211 | 87.4 |
| 212 | 97.0 |
| 213 | 88.6 |
| 214 | 89.8 |
| 215 | 100.4 |
| 216 | 92.4 |
| 217 | 87.0 |
| 218 | 84.4 |
| 219 | 88.8 |
| 220 | 96.7 |
| 221 | 102.4 |
| 222 | 90.7 |
| 223 | 92.8 |
| 224 | 71.9 |
| 225 | 89.9 |
| 226 | 84.2 |
| 227 | 90.1 |
| 228 | 82.1 |
| 229 | 88.7 |
| 230 | 94.3 |
| 231 | 90.3 |
| 232 | 94.2 |
| 233 | 86.5 |
| 234 | 91.5 |
| 235 | 95.8 |
| 236 | 77.1 |
| 237 | 90.9 |
| 238 | 89.9 |
| 239 | 86.5 |
| 240 | 99.3 |
| 241 | 92.4 |
| 242 | 90.6 |
| 243 | 99.9 |
| 244 | 96.2 |
| 245 | 94.3 |
| 246 | 105.1 |
| 247 | 100.6 |
| 248 | 100.0 |
| 249 | 84.2 |
| 250 | 104.1 |
| 251 | 88.4 |
| 252 | 99.4 |
| 253 | 90.9 |
| 254 | 104.1 |
| 255 | 101.9 |
| 256 | 98.7 |
| 257 | 86.2 |
| 258 | 89.0 |
| 259 | 102.5 |
| 260 | 77.4 |
| 261 | 85.9 |
| 262 | 98.7 |
| 263 | 79.0 |
| 264 | 90.1 |
| 265 | 90.6 |
| 266 | 102.9 |
| 267 | 90.9 |
| 268 | 90.1 |
| 269 | 89.9 |
| 270 | 89.4 |
| 271 | 93.4 |
| 272 | 93.9 |
| 273 | 87.0 |
| 274 | 86.0 |
| 275 | 105.9 |
| 276 | 98.2 |
| 277 | 96.8 |
| 278 | 95.8 |
| 279 | 99.4 |
| 280 | 90.6 |
| 281 | 99.7 |
| 282 | 95.0 |
| 283 | 101.2 |
| 284 | 106.4 |
| 285 | 98.7 |
| 286 | 99.6 |
| 287 | 92.4 |
| 288 | 99.8 |
| 289 | 91.2 |
| 290 | 102.1 |
| 291 | 89.6 |
| 292 | 95.6 |
| 293 | 92.5 |
| 294 | 92.2 |
| 295 | 97.2 |
| 296 | 103.0 |
| 297 | 97.9 |
| 298 | 95.8 |
| 299 | 95.1 |
| 300 | 84.3 |
| 301 | 96.6 |
| 302 | 93.9 |
| 303 | 93.5 |
| 304 | 92.8 |
| 305 | 78.8 |
| 306 | 84.7 |
| 307 | 96.1 |
| 308 | 91.5 |
| 309 | 96.7 |
| 310 | 94.5 |
| 311 | 105.0 |
| 312 | 88.8 |
| 313 | 90.5 |
| 314 | 87.4 |
| 315 | 104.4 |
| 316 | 99.6 |
| 317 | 94.9 |
| 318 | 93.9 |
| 319 | 84.3 |
| 320 | 94.4 |
| 321 | 96.7 |
| 322 | 90.5 |
| 323 | 94.0 |
| 324 | 97.1 |
| 325 | 72.6 |
| 326 | 91.8 |
| 327 | 93.1 |
| 328 | 86.7 |
| 329 | 84.3 |
| 330 | 82.4 |
| 331 | 84.2 |
| 332 | 96.5 |
| 333 | 90.9 |
| 334 | 92.6 |
| 335 | 78.2 |
| 336 | 86.9 |
| 337 | 90.1 |
| 338 | 81.3 |
| 339 | 81.3 |
| 340 | 96.0 |
| 341 | 95.4 |
| 342 | 82.8 |
| 343 | 93.6 |
| 344 | 112.1 |
| 345 | 96.5 |
| 346 | 75.5 |
| 347 | 86.3 |
| 348 | 84.5 |
| 349 | 88.7 |
| 350 | 71.4 |
| 351 | 88.9 |
| 352 | 75.6 |
| 353 | 78.4 |
| 354 | 91.3 |
| 355 | 82.2 |
| 356 | 92.4 |
| 357 | 102.4 |
| 358 | 93.2 |
| 359 | 92.1 |
| 360 | 97.3 |
| 361 | 101.2 |
| 362 | 87.9 |
| 363 | 84.7 |
| 364 | 84.1 |
| 365 | 82.5 |
| 366 | 101.1 |
| 367 | 107.3 |
| 368 | 109.9 |
| 369 | 88.5 |
| 370 | 106.0 |
| 371 | 79.0 |
| 374 | 71.8 |
| 375 | |
| 376 | 68.4 |
| 377 | 71.1 |
| 378 | 73.2 |
| 379 | 73.3 |
| 380 | 74.8 |
| 381 | 71.1 |
| 383 | 73.6 |
| 384 | 97.6 |
| 385 | 60.8 |
| 386 | 67.4 |
| 387 | 72.9 |
| 388 | 83.9 |
| 389 | 78.6 |
| 390 | 90.6 |
| 391 | 71.0 |
| 392 | 76.3 |
| 393 | 65.8 |
| 394 | 68.7 |
| 395 | 71.6 |
| 396 | 62.9 |
| 397 | 63.7 |
| 398 | 66.3 |
| 399 | 73.3 |
| 400 | 66.7 |
| 401 | 65.7 |
| 402 | 64.6 |
| 403 | 79.9 |
| 404 | 76.54 |
| 405 | 66.82 |
| 406 | 93.39 |
| 407 | 103.6 |
| 408 | 95.68 |
| 409 | 96.93 |
| 410 | 93.65 |
| 411 | 89.97 |
| 412 | 89.91 |
| 413 | 100.7 |
| 414 | 83.61 |
| 415 | 78.16 |
| 416 | 90.83 |
| 417 | 85.13 |
| 418 | 67.26 |

The following compounds do not have IC₅₀s in the range of about 0.001 μM to about 30 μM in the above assay:
3-[5-[4-(4-cyanophenyl)piperidine-1-carbonyl]-2-methylphenyl]-1,1-dimethylurea
3-[3-[4-(4-cyanophenyl)piperidine-1-carbonyl]-4-methoxyphenyl]-1-propan-2-ylurea
3-benzyl-1-[3-[4-(4-cyanophenyl)piperidine-1-carbonyl]-4-methoxyphenyl]urea
Methyl (2S)-2-[[5-[4-(4-cyanophenyl)piperidine-1-carbonyl]-2-methylphenyl]carbamoylamino]-3-methylbutanoate
Methyl (2S)-2-[[5-[4-(4-cyanophenyl)piperidine-1-carbonyl]-2-methylphenyl]carbamoylamino]-2-phenylacetate
Methyl 2-[[5-[4-(4-cyanophenyl)piperidine-1-carbonyl]-2-methylphenyl]carbamoylamino]-3-(4-fluorophenyl)propanoate
Methyl 2-[[5-[4-(4-cyanophenyl)piperidine-1-carbonyl]-2-methylphenyl]carbamoylamino]-4-(tetrazol-1-yl)butanoate
Methyl (2S)-2-[[5-[4-(4-cyanophenyl)piperidine-1-carbonyl]-2-methylphenyl]carbamoylamino]-4-(1H-tetrazol-5-yl)butanoate
Methyl (2S)-2-[[5-[4-(4-cyanophenyl)piperidine-1-carbonyl]-2-methylphenyl]carbamoylamino]-3-(1-methylimidazol-4-yl)propanoate
Methyl 2-[[5-[4-(4-cyanophenyl)piperidine-1-carbonyl]-2-methylphenyl]carbamoylamino]-2-methylpropanoate
Ethyl (2S)-2-[[5-[4-(4-cyanophenyl)piperidine-1-carbonyl]-2-methylphenyl]carbamoylamino]-3-hydroxypropanoate
3-benzyl-1-[5-[4-(4-cyanophenyl)piperidine-1-carbonyl]-2-methylsulfonylphenyl]urea
N-[(trans)-2-[[5-[4-(4-cyanophenyl)piperidine-1-carbonyl]-2-methylphenyl]carbamoylamino]cyclohexyl]acetamide
1-[5-(4-hydroxy-4-phenylpiperidine-1-carbonyl)-2-methylphenyl]-3-propan-2-ylurea
1-[5-[4-(2-methoxyphenyl)piperidine-1-carbonyl]-2-methylphenyl]-3-propan-2-ylurea
1-[5-[4-hydroxy-4-[3-(trifluoromethyl)phenyl]piperidine-1-carbonyl]-2-methylphenyl]-3-propan-2-ylurea
1-[5-[4-(2-fluorophenyl)-4-hydroxypiperidine-1-carbonyl]-2-methylphenyl]-3-propan-2-ylurea
3-[5-[4-(4-cyanophenyl)piperidine-1-carbonyl]-2-methylphenyl]-1-methyl-1-(oxolan-2-ylmethyl)urea
3-[5-[4-(4-cyanophenyl)piperidine-1-carbonyl]-2-methylphenyl]-1-(2-methoxyethyl)-1-methylurea
3-[5-[4-(4-cyanophenyl)piperidine-1-carbonyl]-2-methylphenyl]-1-(2-cyanopropan-2-yl)-1-methylurea
3-[5-[4-(4-cyanophenyl)piperidine-1-carbonyl]-2-methylphenyl]-1-cyclopropyl-1-(1,1-dioxothiolan-3-yl)urea
N-[2-[[5-[4-(4-cyanophenyl)piperidine-1-carbonyl]-2-methylphenyl]carbamoyl-methylamino]ethyl]-2-methylpropanamide
N-[5-[4-(4-cyanophenyl)piperidine-1-carbonyl]-2-methylphenyl]-1,1-dioxo-1,4-thiazinane-4-carboxamide
1-[5-[4-(3-fluorophenyl)piperidine-1-carbonyl]-2-methylphenyl]-3-propan-2-ylurea
1-[5-[4-(3-chlorophenyl)piperidine-1-carbonyl]-2-methylphenyl]-3-propan-2-ylurea
1-[5-[4-(3-methoxyphenyl)piperidine-1-carbonyl]-2-methylphenyl]-3-propan-2-ylurea
1-[5-[4-(2-cyanophenyl)piperidine-1-carbonyl]-2-methylphenyl]-3-propan-2-ylurea
3-[3-[4-(4-cyanophenyl)piperidine-1-carbonyl]-4-methylsulfinylphenyl]-1-propan-2-ylurea and
3-[3-[4-(4-cyanophenyl)piperidine-1-carbonyl]-4-methylsulfonylphenyl]-1-propan-2-ylurea.

In an alternative embodiment these compounds are excluded from the claims of the present application.

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C., unless otherwise stated;
(ii) organic solutions were dried over anhydrous magnesium sulfate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LC-MS, and reaction times are given for illustration only;
The following methods were used for liquid chromatography (LC)/mass spectral (MS) analysis:—

### HPLC: Agilent 1100 or Waters Alliance HT (2790 & 2795)

### Mass Spectrometer Waters ZQ ESCi

(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard when the solvent is CDCl₃ (when the solvent is d₆-DMSO, it locks on to the 2.49 DMSO peak), determined at 300 MHz unless otherwise indicated; the following abbreviations have been used: s, singlet; d, doublet; t, triplet; q, quartet; m, multiplet; b, broad;
(viii) chemical symbols have their usual meanings; SI units and symbols are used;
(ix) solvent ratios are given in volume:volume (v/v) terms; and
(x) mass spectra (MS) were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is MH⁺;
[A] When Cl is present in the molecule, the m/z value for the (M+H)⁺ molecular ion is based on the ³⁵Cl isotope. When there are multiple chlorine atoms in the molecule, the m/z is based on the first peak of the isotope pattern.
[B] When Br is present in the molecule, the m/z value for the (M+H)⁺ and/or (M−H)⁻ molecular ions may be based either on the ⁷⁹Br isotope or the ⁸¹Br isotope. As the isotopes are of approximately equal abundance, in many cases both isotopes are seen in the spectrum, but only one is reported.
(xi) unless stated otherwise compounds containing an asymmetrically substituted carbon and/or sulphur atom have not been resolved;
(xii) where a synthesis is described as being analogous to that described in a previous example the amounts used are the millimolar ratio equivalents to those used in the previous example;
(xvi) the following abbreviations have been used:

### Abbreviations

ACN Acetonitrile
DIPEA Di-iso-propylethylamine
DMA Dimethyl acetamide
DMAP 4-dimethylamino pyridine
DMTMM 4-(4,6-Dimethoxy-1,3,5-Triazin-2-yl)-4-Methylmorpholinium Chloride
DMSO (dmso)dimethyl sulphoxide (in NMR data the solvent is d₆-deuterioDMSO)
EDAC N-ethyl-N′-(3-dimethylaminopropyl)-carbodiimide hydrochloride
EtOAc Ethyl acetate
EtOH Ethanol
HATU O-(7-Azabenzotriazol-1-yl)-N,N,N′,N′-Tetramethyluronium Hexafluoro-phosphate
HOBT 1-Hydroxybenzotriazole
hrs hours
HTBU O-benzotriazol-1-yl-N,N,N′,N′-tetramethyluronium hexafluorophosphate
MeOH Methanol
mins minutes
TEA Triethylamine
TFAA Trifluoroacetic Anhydride
THF Tetrahydrofuran

### Method 1

### EXAMPLE 1

### 1-butyl-3-[5-[4-(4-cyanophenyl)piperidine-1-carbonyl]-2-methyl-phenyl]urea

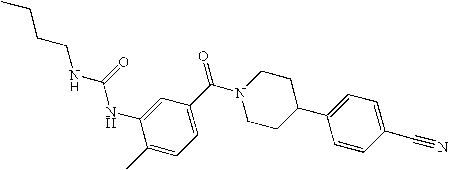

A suspension of 4-[1-(3-amino-4-methyl-benzoyl)-4-piperidyl]benzonitrile (Intermediate A, 200 mg, 0.63 mmol) in DCM (5 mL) was treated with n-butyl isocyanate (0.28 mL, 2.5 mmol), and the reaction stirred at ambient temperature for 24 hrs. Analysis of the reaction mixture indicated only partial reaction so extra isocyanate was added and stirring was continued; triethylamine (0.1 mL) was also added and stirring for a further 24 hrs. A parallel experiment was carried out on the same scale as the above, using acetonitrile (5 mL) as solvent, and using Microwave heating (10 mins at 100° C., 30 mins at 120° C. and 60 mins at 130° C.)

The reaction mixtures from the two experiments were combined and reduced in vacuo. EtOAc (30 ml) was added and the solution was washed sequentially with water (30 ml) and brine (30 ml), dried (MgSO₄), filtered and reduced in vacuo to give a brown oil which was chromatographed (Optix, 12 g silica column, eluting with a gradient consisting of 40-100% EtOAc in isohexane) to give the title compound as a colourless solid (201 mg), ¹H NMR (300.072 MHz, CDCl₃) δ 0.93 (3H, t), 1.30-1.42 (2H, m), 1.44-1.54 (2H, m), 1.57-1.99 (4H, m), 2.04 (3H, s), 2.79-2.92 (2H, m), 2.99-3.13 (1H, m), 3.20 (2H, t), 3.90-4.04 (1H, m), 4.74-4.95 (1H, m), 5.55 (1H, s), 6.69 (1H, s), 6.92-7.07 (2H, m), 7.32 (2H, d), 7.51 (1H, s), 7.60 (2H, d), m/z 419 (M+H)⁺.

### Method 2

### EXAMPLE 2

### N-[5-[4-(4-cyanophenyl)piperidine-1-carbonyl]-2-methyl-phenyl]morpholine-4-carboxamide

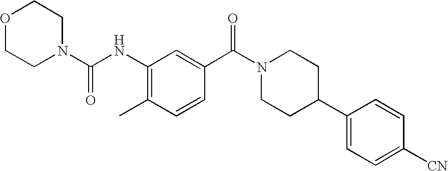

A suspension of 4-[1-(3-amino-4-methyl-benzoyl)-4-piperidyl]benzonitrile (Intermediate A, 200 mg, 0.63 mmol) in THF (15 mL) was blanketed with nitrogen and treated with triphosgene (63 mg, 0.31 mmol, 0.5 eq) and DIPEA (218 μL, 1.25 mmol, 2 eq), and the reaction stirred at ambient temperature for 0.5 hr. Morpholine (274 μL, 3.13 mmol, 5 eq) was added and the reaction mixture stirred for a further four hours. The reaction mixture was then concentrated and the solid residue dissolved in DCM; the suspension was filtered and the filtrate purified by column chromatography (4 g silica column, eluting with a gradient consisting of 0-10% methanol in DCM) to give the title compound as a colourless solid (91 mg), ¹H NMR (300.073 MHz, d₆-DMSO) δ 1.50-1.91 (m, 4H), 2.19 (s, 3H), 2.85-3.02 (m, 3H), 3.38-3.65 (m, 8H), 3.72-3.86 (m, 1H), 4.42-4.78 (m, 1H), 7.10 (d, J=7.7 Hz, 1H), 7.23 (d, J=7.8 Hz, 1H), 7.29 (s, 1H), 7.50 (d, J=8.3 Hz, 2H), 7.76 (d, J=8.3 Hz, 2H), 8.10 (s, 1H), m/z 433 (M+H)⁺.

It will be appreciated that alternative solvents, reagents, additives and conditions may be used in the above reactions. Examples of solvents are THF, DCM, other; examples of additives are TEA, DIPEA and pyridine, and the reactions may be performed at temperatures between 0° C. and the boiling point of the solvent.

The following examples were prepared using the method indicated, and starting from the appropriate intermediate and reagents:

### EXAMPLE 3

### 3-[5-[4-(4-cyanophenyl)piperidine-1-carbonyl]-2-methyl-phenyl]-1-propan-2-yl-urea

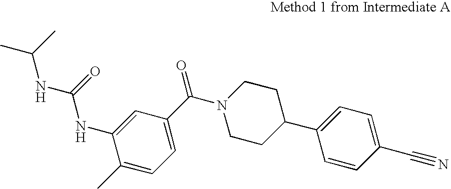

¹H NMR (300.072 MHz, CDCl₃) δ 1.16 (6H, d), 1.60-1.98 (4H, m), 2.01 (3H, s), 2.75-2.89 (2H, m), 2.97-3.22 (1H, m), 3.85-4.07 (2H, m), 4.77-4.94 (1H, m), 5.49 (1H, s), 6.71 (1H, s), 6.90-7.08 (2H, m), 7.31 (2H, d), 7.53 (1H, s) 7.61 (2H, d), m/z 405 (M+H)⁺.

### EXAMPLE 4

### 3-[5-[4-(4-cyanophenyl)piperidine-1-carbonyl]-2-(trifluoromethoxy)phenyl]-1-propan-2-yl-urea

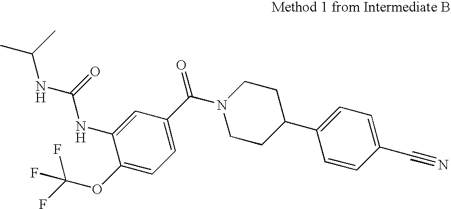

¹H NMR (300.072 MHz, CDCl₃) δ 1.21 (6H, d), 1.60-1.95 (4H, m), 2.77-2.91 (2H, m), 3.10-3.26 (1H, m), 3.86-4.02 (2H, m), 4.79-4.98 (1H, m), 5.05 (1H, d), 6.81 (1H, s), 7.07-7.13 (1H, m), 7.22-7.25 (1H, m), 7.33 (2H, d), 7.61 (2H, d), 8.29 (1H, s), m/z 475 (M+H)⁺.

### EXAMPLE 5

### 3-benzyl-1-[5-[4-(4-cyanophenyl)piperidine-1-carbonyl]-2-(trifluoromethoxy)phenyl]urea

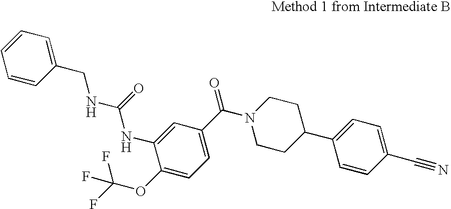

¹H NMR (300.072 MHz, CDCl₃) δ 1.73-2.02 (4H, m), 2.73-2.90 (2H, m), 3.02-3.26 (1H, m), 3.84-3.99 (1H, m), 4.39 (2H, d), 4.70-4.88 (1H, m), 5.91 (1H, t), 6.94-7.00 (1H, m), 7.11-7.17 (1H, m), 7.19-7.22 (1H, m), 7.26-7.34 (7H, m), 7.60 (2H, d), 8.25 (1H, d), m/z 523 (M+H)⁺.

### EXAMPLE 6

### 1-[5-[4-(4-cyanophenyl)piperidine-1-carbonyl]-2-methoxy-phenyl]-3-propan-2-yl-urea

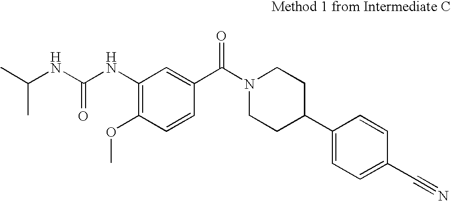

¹H NMR (300.072 MHz, CDCl₃, 30° C.) δ 1.21 (6H, dJ=7.9 Hz), 1.50-1.99 (4H, m), range 2.75-3.21 (3H, m, br s, br s), 3.87 (3H, s), 3.90-4.03 (1H, m), 4.06-4.40 (1H, m), 4.50-4.98 (1H, m), 6.80-6.93 (2H, m), 7.07-7.15 (1H, m), 7.33 (2H, dJ=7.9 Hz), 7.61 (2H, dJ=8.6 Hz), 8.20 (1H, dJ=3.0 Hz) (NB. Integration is imprecise as spectrum contains signals due to presence of water and also displays extensive peak broadening due to rotational isomerism), m/z 421 (M+H)⁺.

### EXAMPLE 7

### 3-benzyl-1-[3-[4-(4-cyanophenyl)piperidine-1-carbonyl]-4-methyl-phenyl]urea

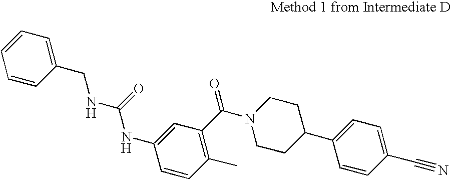

¹H NMR (400.132 MHz, CDCl₃) δ 1.37-1.74 (3H, m), 1.89-1.97 (1H, m), 2.14 (3H, d), 2.68-2.85 (2H, m), 2.95-3.08 (1H, m), 3.54-3.63 (1H, m), 4.33 (2H, d), 4.72-4.79 (1H, m), 6.02 (1H, t), 6.92-7.12 (3H, m), 7.17-7.32 (7H, m), 7.58-7.70 (3H, m), m/z 453 (M+H)⁺.

### EXAMPLE 8

### 3-benzyl-1-[5-[4-(4-cyanophenyl)piperidine-1-carbonyl]-2-fluoro-phenyl]urea

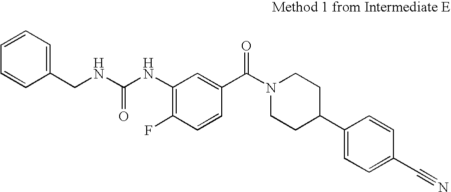

¹H NMR (400.132 MHz, d₆-DMSO) δ 1.47-1.94 (m, 4H), 2.76-3.26 (m, 3H), 3.62-3.85 (m, 1H), 4.32 (d, 2H), 4.48-4.74 (m, 1H), 6.98-7.06 (m, 1H), 7.13 (t, 1H), 7.21-7.39 (m, 6H), 7.51 (d, 2H), 7.78 (d, 2H), 8.21-8.29 (m, 1H), 8.56 (d, 1H), m/z 457 (M+H)⁺.

### EXAMPLE 9

### 3-benzyl-1-[5-[4-(4-cyanophenyl)piperidine-1-carbonyl]-2-methoxy-phenyl]urea

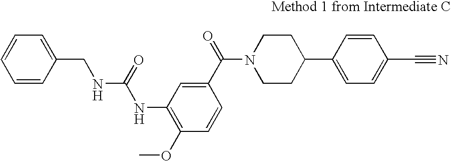

¹H NMR (300.073 MHz, d₆-DMSO, 30° C.) δ 1.49-1.70 (2H, m), 1.71-1.88 (2H, m), 2.79-3.19 (3H, m), 3.87 (3H, s), 3.93-4.21 (1H, m), 4.29 (2H, dJ=5.6 Hz), 4.35-4.98 (1H, m), 6.94-7.04 (2H, m), 7.18-7.38 (6H, m), 7.49 (2H, dJ=7.1 Hz), 7.75 (2H, dJ=7.9 Hz), 8.11 (1H, s), 8.22 (1H, s) (NB. Integration is imprecise as spectrum displays extensive peak broadening due to rotational isomerism), m/z 469 (M+H)⁺.

### EXAMPLE 10

### N-[5-[4-(4-cyanophenyl)piperidine-1-carbonyl]-2-methyl-phenyl]pyrrolidine-1-carboxamide

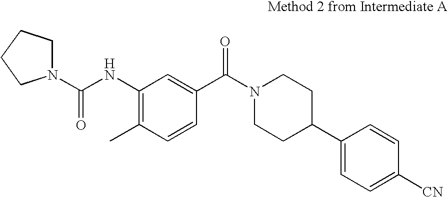

¹H NMR (300.073 MHz, d₆-DMSO) δ 1.54-1.71 (m, 2H), 1.79-1.95 (m, 6H), 2.25 (s, 3H), 2.88-3.10 (m, 3H), 3.36-3.46 (m, 4H), 4.15-4.31 (m, 2H), 7.04 (d, J=7.6 Hz, 1H), 7.16-7.28 (m, 2H), 7.44-7.58 (m, 3H), 7.71 (d, J=8.3 Hz, 2H), m/z 417 (M+H)⁺.

### EXAMPLE 11

### 3-[5-[4-(4-cyanophenyl)piperidine-1-carbonyl]-2-fluoro-phenyl]-1-propan-2-yl-urea

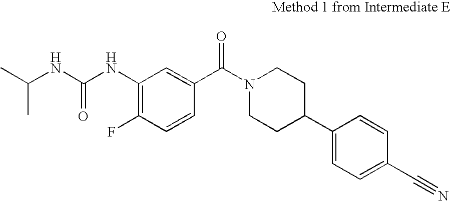

¹H NMR (300.073 MHz, d₆-DMSO) δ 1.09 (d, 6H), 1.44-1.94 (m, 4H), 2.77-3.30 (m, 3H), 3.67-3.90 (m, 2H), 4.34-4.82 (m, 1H), 6.51-6.64 (m, 1H), 6.93-7.05 (m, 1H), 7.22 (t, 1H), 7.50 (d, 2H), 7.76 (d, 2H), 8.18-8.38 (m, 2H), m/z 409 (M+H)⁺.

### EXAMPLE 12

### 3-benzyl-1-[5-[4-(4-cyanophenyl)piperidine-1-carbonyl]-2,4-dimethyl-phenyl]urea

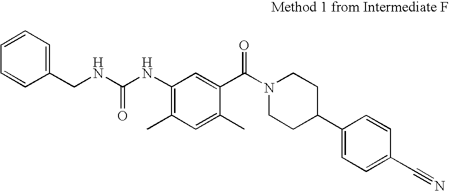

¹H NMR (300.072 MHz, CDCl₃) δ1.65 (2H, m), 1.93 (1H, m), 2.06 (3H, s), 2.19-2.26 (3H, m), 2.77 (2H, m), 3.06 (1H, m), 3.67 (1H, m), 4.42 (2H, m), 4.84 (1H, m), 5.40 (1H, m), 6.35 (1H, m), 6.94 (1H, s), 7.31 (7H, s), 7.59-7.62 (2H, d), m/z 467 (M+H)⁺.

### EXAMPLE 13

### 3-[5-[4-(4-cyanophenyl)piperidine-1-carbonyl]-2,4-dimethyl-phenyl]-1-propan-2-yl-urea

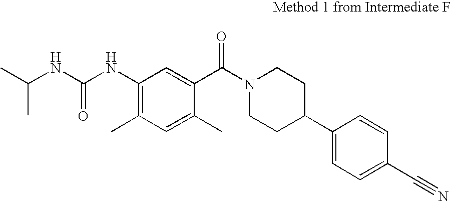

¹H NMR (300.072 MHz, CDCl₃) δ1.13-1.16 (6H, m), 1.73 (2H, m), 1.98-2.05 (1H, m), 2.08 (3H, s), 2.25 (3H, d), 2.84 (2H, m), 3.13 (1H, m), 3.71 (1H, d), 3.92-3.99 (1H, m), 4.94 (2H, m), 6.11-6.22 (1H, m), 6.95 (1H, s), 7.31 (2H, d), 7.61 (2H, d), m/z 419 (M+H)⁺.

### EXAMPLE 14

### 1-benzyl-3-[3-[4-(4-cyanophenyl)piperidine-1-carbonyl]phenyl]urea

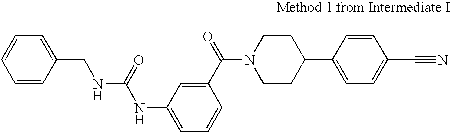

¹H NMR (300.073 MHz, dmso, 30° C.) δ 1.48-1.97 (4H, m), 2.69-3.25 (3H, m), 3.60-3.90 (1H, m), 4.29 (2H, dJ=5.3 Hz), 4.48-4.76 (1H, m), 6.60-6.70 (1H, m), 6.94 (1H, dJ=6.7 Hz), 7.18-7.42 (7H, m), 7.45-7.56 (3H, m), 7.76 (2H, dJ=8.0 Hz), 8.67 (1H, s), m/z 439 (M+H)⁺.

### EXAMPLE 15

### 1-[5-[4-(4-cyanophenyl)piperidine-1-carbonyl]-2-methyl-phenyl]-3-cyclopentyl-urea

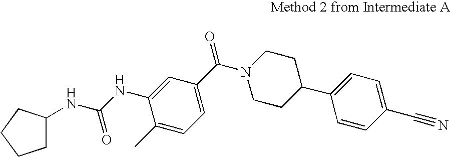

¹H NMR (300.072 MHz, CDCl₃) δ1.35-1.45 (2H, m), 1.53-2.01 (13H, m), 2.79-3.20 (3H, m), 3.90-4.12 (2H, m), 4.85 (1H, m), 5.64 (1H, d), 6.71 (1H, s), 6.90-6.94 (1H, m), 7.03 (1H, d), 7.32 (2H, d), 7.54 (1H, s), 7.60 (2H, d), m/z 431 (M+H)⁺.

### EXAMPLE 16

### 1-[5-[4-(4-cyanophenyl)piperidine-1-carbonyl]-2-methyl-phenyl]-3-phenethyl-urea

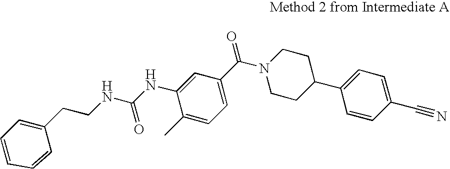

¹H NMR (300.072 MHz, CDCl₃) δ1.64-2.01 (4H, m), 2.01 (3H, s), 2.75 (1H, t), 2.83 (3H, t), 3.1 (1H, m), 3.34-3.49 (2H, m), 3.90 (1H, m), 4.77 (1H, m), 5.58 (1H, t), 6.78 (1H, s), 6.88-6.91 (1H, m), 7.02 (1H, d), 7.14-7.23 (1H, m), 7.19-7.23 (3H, m), 7.30 (3H, q), 7.47 (1H, d), 7.58-7.61 (2H, m), m/z 467 (M+H)⁺.

### EXAMPLE 17

### 1-[5-[4-(4-cyanophenyl)piperidine-1-carbonyl]-2-methyl-phenyl]-3-(oxan-4-ylmethyl)urea

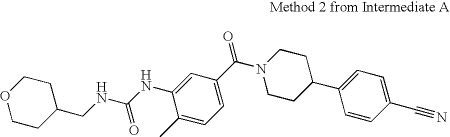

¹H NMR (300.072 MHz, CDCl₃) δ1.24-1.30 (1H, m), 1.33-1.38 (1H, m), 1.55-2.00 (7H, m), 2.00 (3H, s), 2.80-2.88 (2H, m), 3.10 (3H, t), 3.34 (1H, d), 3.38 (1H, d), 3.94-3.98 (3H, m), 4.84 (1H, m), 5.86 (1H, t), 6.79 (1H, s), 6.90-6.93 (1H, m), 7.02 (1H, d), 7.31 (2H, d), 7.50 (1H, d), 7.61 (2H, d), m/z 461 (M+H)⁺.

### EXAMPLE 18

### 3-[3-[4-(4-cyanophenyl)piperidine-1-carbonyl]-4-methyl-phenyl]-1-methyl-urea

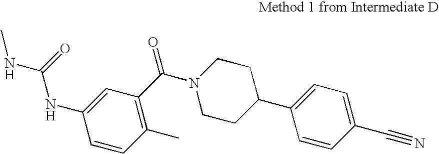

¹H NMR (400.132 MHz, CDCl₃) [symbol] 1.49-1.82 (m, 3H), 1.97-2.03 (m, 1H), 2.24 (d, 3H), 2.75 (d, 3H), 2.79-2.93 (m, 2H), 3.04-3.14 (m, 1H), 3.62-3.69 (m, 1H), 4.88-4.99 (m, 1H), 5.34 (s, 1H), 6.98 (d, 1H), 7.06 (d, 1H), 7.12-7.24 (m, 2H), 7.29-7.33 (m, 2H), 7.61 (d, 2H), m/z 377 (M+H)⁺.

### EXAMPLE 19

### 3-[3-[4-(4-cyanophenyl)piperidine-1-carbonyl]-4-methyl-phenyl]-1-ethyl-urea

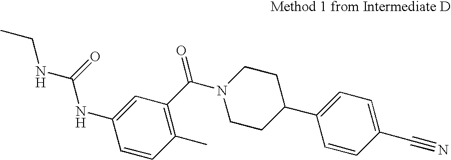

¹H NMR (400.132 MHz, CDCl₃) [symbol] 1.11 (t, 3H), 1.48-1.81 (m, 3H), 1.97-2.03 (m, 1H), 2.24 (d, 3H), 2.79-2.92 (m, 2H), 3.04-3.15 (m, 1H), 3.19-3.25 (m, 2H), 3.62-3.68 (m, 1H), 4.92-4.99 (m, 1H), 5.39 (s, 1H), 6.96 (d, 1H), 7.06 (d, 1H), 7.15 (d, 1H), 7.21 (t, 1H), 7.31 (d, 2H), 7.62 (d, 2H), m/z 377 (M+H)⁺.

### EXAMPLE 20

### 1-butyl-3-[3-[4-(4-cyanophenyl)piperidine-1-carbonyl]-4-methyl-phenyl]urea

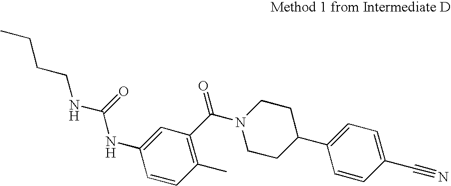

¹H NMR (400.132 MHz, CDCl₃) [symbol] 0.90 (t, 3H), 1.32 (sextet, 2H), 1.43 (quintet, 2H), 1.53-1.80 (m, 3H), 1.95-2.01 (m, 1H), 2.22 (d, 3H), 2.78-2.92 (m, 2H), 3.04-3.18 (m, 3H), 3.60-3.66 (m, 1H), 4.91-4.97 (m, 1H), 5.64 (t, 1H), 6.98-7.18 (m, 3H), 7.31 (d, 2H), 7.52-7.63 (m, 3H), m/z 419 (M+H)⁺.

### EXAMPLE 21

### 1-[3-[4-(4-cyanophenyl)piperidine-1-carbonyl]-4-methyl-phenyl]-3-cyclopentyl-urea

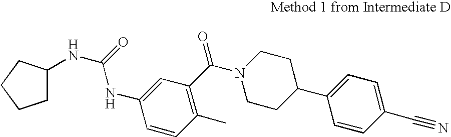

¹H NMR (400.132 MHz, CDCl₃) [symbol] 1.30-1.39 (m, 2H), 1.49-1.81 (m, 7H), 1.87-1.96 (m, 2H), 1.99-2.03 (m, 1H), 2.23 (d, 3H), 2.78-2.92 (m, 2H), 3.02-3.13 (m, 1H), 3.61-3.68 (m, 1H), 4.00-4.08 (m, 1H), 4.89-4.94 (m, 1H), 5.65 (s, 1H), 6.91-7.04 (m, 2H), 7.08-7.21 (m, 1H), 7.31 (d, 2H), 7.42 (d, 1H), 7.61 (d, 2H), m/z 431 (M+H)⁺.

### EXAMPLE 22

### 3-[2-cyano-5-[4-(4-cyanophenyl)piperidine-1-carbonyl]phenyl]-1-propan-2-yl-urea

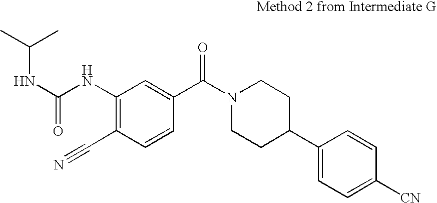

¹H NMR (400.132 MHz, CDCl₃) [symbol] 1.22 (d, 6H), 1.69-1.84 (m, 3H), 1.95-2.03 (m, 1H), 2.80-2.93 (m, 2H), 3.15-3.25 (m, 1H), 3.81-3.89 (m, 1H), 3.97 (octet, 1H), 4.84-4.93 (m, 1H), 5.32 (d, 1H), 7.07-7.10 (m, 1H), 7.24 (s, 1H), 7.33 (d, 2H), 7.51 (d, 1H), 7.62 (d, 2H), 8.30 (d, 1H), m/z 416 (M+H)⁺.

### EXAMPLE 23

### 1-[3-[4-(4-cyanophenyl)piperidine-1-carbonyl]-4-methyl-phenyl]-3-[(2-fluorophenyl)methyl]urea

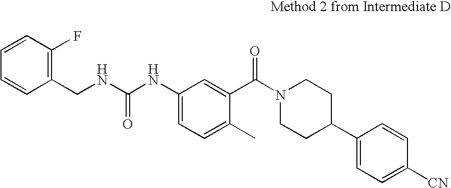

¹H NMR (300.072 MHz, cdcl3) [symbol] 1.45-1.80 (m, 3H), 1.91-1.99 (m, 1H), 2.16 (d, 3H), 2.72-2.87 (m, 2H), 3.00-3.12 (m, 1H), 3.57-3.66 (m, 1H), 4.41 (d, 2H), 4.82-4.89 (m, 1H), 5.91-5.97 (m, 1H), 6.87-7.08 (m, 4H), 7.11-7.23 (m, 2H), 7.27-7.36 (m, 3H), 7.49-7.63 (m, 3H), m/z 471 (M+H)⁺.

### EXAMPLE 24

### 1-benzyl-3-[3-[4-(4-cyanophenyl)piperidine-1-carbonyl]-4-methyl-phenyl]-1-methyl-urea

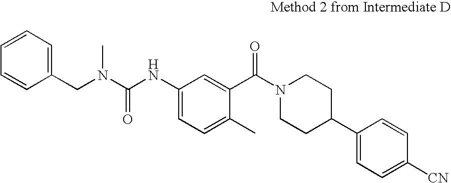

¹H NMR (300.072 MHz, CDCl₃) [symbol] 1.62-1.82 (m, 3H), 1.93-2.02 (m, 1H), 2.27 (d, 3H), 2.75-2.91 (m, 2H), 3.02 (s, 3H), 3.06-3.12 (m, 1H), 3.66-3.72 (m, 1H), 4.53-4.60 (m, 2H), 4.90-4.99 (m, 1H), 6.41 (s, 1H), 7.09-7.14 (m, 2H), 7.27-7.39 (m, 8H), 7.60 (d, 2H), m/z 467 (M+H)⁺.

### EXAMPLE 25

### 1-[3-[4-(4-cyanophenyl)piperidine-1-carbonyl]-4-methyl-phenyl]-3-(pyridin-3-ylmethyl)urea

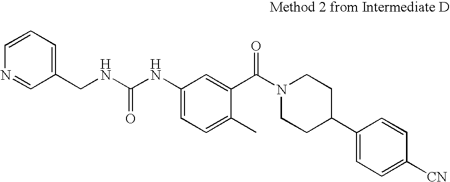

¹H NMR (300.072 MHz, CDCl₃) [symbol] 1.48-1.82 (m, 3H), 1.91-2.00 (m, 1H), 2.17 (d, 3H), 2.73-2.87 (m, 2H), 3.00-3.13 (m, 1H), 3.57-3.66 (m, 1H), 4.38 (d, 2H), 4.79-4.86 (m, 1H), 5.99-6.09 (m, 1H), 6.90-7.06 (m, 2H), 7.15-7.34 (m, 4H), 7.51 (d, 1H), 7.60-7.67 (m, 3H), 8.45-8.55 (m, 2H), m/z 454 (M+H)⁺.

### EXAMPLE 26

### 1-[3-[4-(4-cyanophenyl)piperidine-1-carbonyl]-4-methyl-phenyl]-3-phenethyl-urea

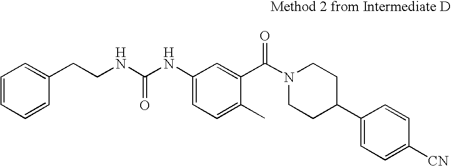

¹H NMR (300.072 MHz, CDCl₃) [symbol] 1.42-1.69 (m, 3H), 1.91-2.00 (m, 1H), 2.19 (d, 3H), 2.74-2.85 (m, 4H), 2.97-3.12 (m, 1H), 3.37-3.46 (m, 2H), 3.57-3.65 (m, 1H), 4.77-4.87 (m, 1H), 5.44 (s, 1H), 6.91-7.07 (m, 3H), 7.16-7.31 (m, 8H), 7.60 (d, 2H), m/z 467 (M+H)⁺.

### EXAMPLE 27

### 1-[3-[4-(4-cyanophenyl)piperidine-1-carbonyl]-4-methyl-phenyl]-3-(oxan-4-ylmethyl)urea

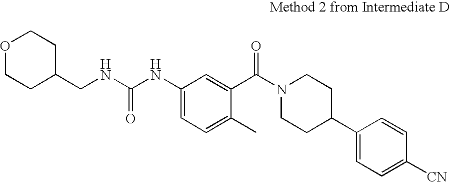

¹H NMR (300.072 MHz, CDCl₃) [symbol] 1.21-1.37 (m, 3H) 1.42-1.85 (m, 5H), 1.91-2.01 (m, 1H), 2.23 (d, 3H), 2.77-2.90 (m, 2H), 3.03-3.18 (m, 3H), 3.30-3.40 (m, 2H), 3.63-3.67 (m, 1H), 3.91-3.98 (dd, 2H), 4.90-4.99 (m, 1H), 5.62 (s, 1H), 6.96 (d, 1H), 7.03 (d, 1H), 7.10-7.27 (m, 2H), 7.30 (d, 2H), 7.61 (d, 2H), m/z 461 (M+H)⁺.

### EXAMPLE 28

### N-[3-[4-(4-cyanophenyl)piperidine-1-carbonyl]-4-methyl-phenyl]morpholine-4-carboxamide

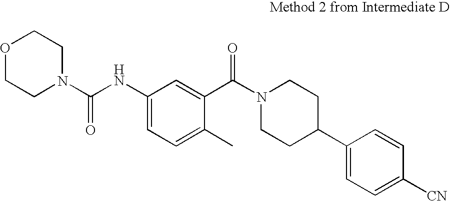

¹H NMR (300.072 MHz, CDCl₃) [symbol] 1.63-1.82 (m, 3H), 1.91-2.01 (m, 1H), 2.26 (d, 3H), 2.77-2.90 (m, 2H), 3.03-3.15 (m, 1H), 3.47 (t, 4H), 3.63-3.67 (m, 1H), 3.71 (t, 4H), 4.90-4.99 (m, 1H), 6.82 (s, 1H), 7.11 (d, 1H), 7.17-7.22 (m, 1H), 7.27-7.34 (m, 3H), 7.61 (d, 2H), m/z 432 (M+H)⁺.

### EXAMPLE 29

### N-[3-[4-(4-cyanophenyl)piperidine-1-carbonyl]-4-methyl-phenyl]pyrrolidine-1-carboxamide

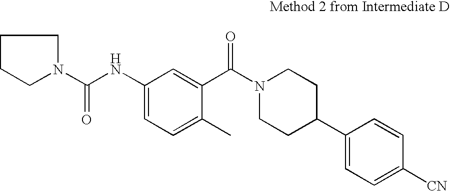

¹H NMR (300.072 MHz, CDCl₃) [symbol] 1.45-1.81 (m, 3H), 1.92-2.00 (m, 5H), 2.27 (d, 3H), 2.77-2.88 (m, 2H), 3.03-3.15 (m, 1H), 3.39-3.49 (m, 4H), 3.65-3.75 (m, 1H), 4.91-5.00 (m, 1H), 6.23 (s, 1H), 7.11 (d, 1H), 7.23-7.37 (m, 4H), 7.60 (d, 2H), m/z 417 (M+H)⁺.

### EXAMPLE 30

### 3-[5-[4-(4-cyanophenyl)piperidine-1-carbonyl]-2-methyl-phenyl]-1-ethyl-urea

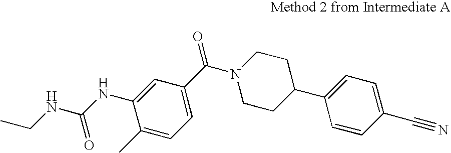

¹H NMR (300.072 MHz, CDCl₃) δ1.05-1.11 (3H, m), 1.14-1.67 (3H, m), 2.03 (4H, d), 2.79-2.88 (2H, m), 3.11-3.18 (1H, m), 3.20-3.22 (2H, m), 3.95 (1H, m), 4.85 (1H, m), 5.77 (1H, s), 6.91-6.94 (2H, m), 7.03 (1H, d), 7.31 (2H, d), 7.58 (2H, q), 7.61 (1H, s), m/z 391 (M+H)⁺.

### EXAMPLE 31

### 1-[5-[4-(4-cyanophenyl)piperidine-1-carbonyl]-2-methyl-phenyl]-3-tert-butyl-urea

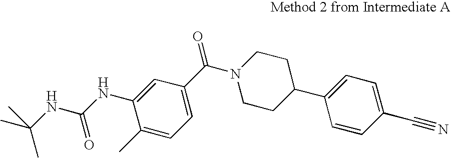

¹H NMR (300.072 MHz, CDCl₃) δ1.35 (9H, s), 1.66-1.90 (4H, m), 1.96 (3H, s), 2.79-2.88 (2H, m), 3.10 (1H, m), 3.95 (1H, m), 4.85 (1H, m), 5.74 (1H, s), 6.81-6.85 (1H, m), 6.86 (1H, s), 6.96 (1H, d), 7.31-7.34 (2H, m), 7.57-7.59 (2H, m), 7.61 (1H, s), m/z 419 (M+H)⁺.

### EXAMPLE 32

### 3-benzyl-1-[5-[4-(4-cyanophenyl)piperidine-1-carbonyl]-2-methyl-phenyl]urea

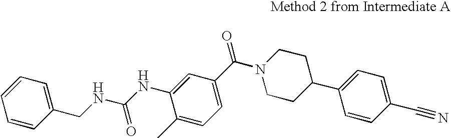

¹H NMR (300.072 MHz, CDCl₃) δ1.50-1.90 (4H, m), 1.97 (3H, s), 2.68-2.81 (2H, m), 3.05 (1H, s), 3.90 (1H, s), 4.34 (2H, d), 4.69 (1H, s), 6.21 (1H, t), 6.85-6.89 (1H, m), 6.98 (1H, d), 7.07-7.11 (1H, m), 7.16-7.28 (7H, m), 7.56 (1H, s), 7.58 (2H, t), m/z 453 (M+H)⁺.

### EXAMPLE 33

### 3-[(4-cyanophenyl)methyl]-1-[5-[4-(4-cyanophenyl)piperidine-1-carbonyl]-2-methyl-phenyl]urea

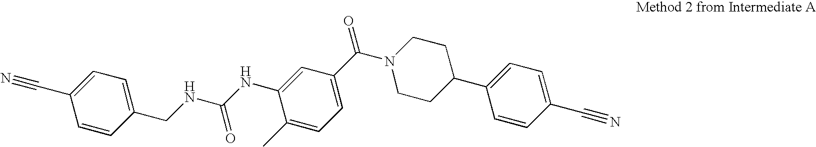

¹H NMR (300.072 MHz, CDCl₃) δ1.76-1.97 (4H, m), 1.98 (3H, s), 2.78-2.82 (2H, m), 3.11 (1H, s), 3.92 (1H, m), 4.42 (2H, d), 4.74 (1H, m), 6.51 (1H, t), 6.86-6.89 (1H, m), 7.00 (1H, d), 7.12 (1H, d), 7.26-7.30 (2H, m), 7.37 (2H, d), 7.55-7.61 (5H, m), m/z 478 (M+H)⁺.

### EXAMPLE 34

### 1-[5-[4-(4-cyanophenyl)piperidine-1-carbonyl]-2-methyl-phenyl]-3-[(2-fluorophenyl)methyl]urea

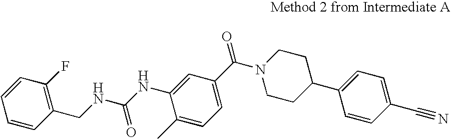

¹H NMR (300.072 MHz, CDCl₃) δ1.74-1.90 (4H, m), 1.96 (3H, s), 2.70-2.86 (2H, m), 3.02-3.08 (1H, m), 3.91 (1H, s), 4.39 (2H, d), 4.75 (1H, s), 6.29 (1H, t), 6.85-7.10 (4H, m), 7.12-7.21 (2H, m), 7.29-7.35 (3H, m), 7.50-7.60 (1H, m), 7.55-7.58 (2H, m), m/z 471 (M+H)⁺.

### EXAMPLE 35

### 1-[5-[4-(4-cyanophenyl)piperidine-1-carbonyl]-2-methyl-phenyl]-3-(pyridin-3-ylmethyl)urea

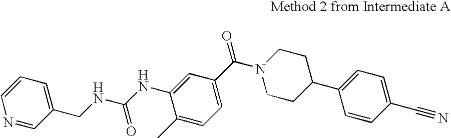

¹H NMR (300.072 MHz, CDCl₃) δ1.50-1.99 (4H, m), 1.99 (3H, s), 2.77-2.81 (1H, m), 2.84 (1H, t), 3.11 (1H, s), 3.92 (1H, s), 4.38 (2H, d), 4.74 (1H, s), 6.45 (1H, t), 6.87-6.90 (1H, m), 7.02 (1H, t), 7.12 (1H, d), 7.21-7.25 (1H, m), 7.30 (3H, d), 7.51-7.67 (4H, m), 8.45-8.47 (1H, m), 8.51-8.52 (1H, m), m/z 454 (M+H)⁺.

### EXAMPLE 36

### 1-[5-[4-(4-cyanophenyl)piperidine-1-carbonyl]-2-methyl-phenyl]-3-(pyridin-4-ylmethyl)urea

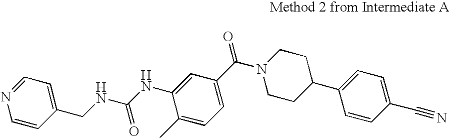

¹H NMR (300.072 MHz, CDCl₃) δ1.61 (2H, s), 1.75-1.91 (1H, m), 2.02 (4H, m), 2.77-2.85 (2H, m), 2.99 (1H, d), 3.11 (1H, s), 3.92 (1H, s), 4.36 (2H, d), 4.76 (1H, s), 6.66 (1H, t), 6.88-6.91 (1H, m), 7.01 (1H, d), 7.18 (2H, q), 7.29 (1H, d), 7.33 (1H, s), 7.57-7.60 (2H, m), 7.70 (1H, d), 8.47-8.49 (2H, m), m/z 454 (M+H)⁺.

### EXAMPLE 37

### 3-[3-[4-(4-cyanophenyl)piperidine-1-carbonyl]-4-methyl-phenyl]-1-[(1R)-1-phenylethyl]urea

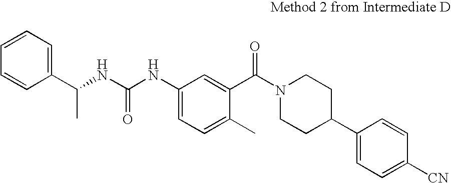

¹H NMR (300.072 MHz, CDCl₃) δ 1.44 (3H, d), 1.48-1.78 (3H, m), 1.87-2.00 (1H, m), 2.09-2.27 (3H, m), 2.73-2.84 (2H, m), 2.96-3.09 (1H, m), 3.58-3.62 (1H, m), 4.77-4.95 (2H, m), 5.69-5.84 (1H, m), 6.82-7.05 (2H, m), 7.15-7.35 (9H, m), 7.61 (2H, d), m/z 467 M+H)⁺.

### EXAMPLE 38

### 3-[3-[4-(4-cyanophenyl)piperidine-1-carbonyl]-4-methyl-phenyl]-1-[(1S)-1-phenylethyl]urea

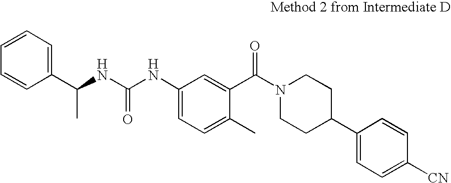

¹H NMR (300.072 MHz, CDCl₃) [symbol] 1.42 (d, 3H), 1.47-1.83 (m, 3H), 1.83-2.00 (m, 1H), 2.08-2.26 (m, 3H), 2.64-2.87 (m, 2H), 2.93-3.11 (m, 1H), 3.53-3.65 (m, 1H), 4.75-4.99 (m, 2H), 5.86-5.94 (m, 1H), 6.84-7.05 (m, 2H), 7.17-7.42 (m, 9H), 7.61 (d, 2H), m/z 467 M+H)⁺.

### EXAMPLE 39

### 1-[5-[4-(4-bromophenyl)-4-hydroxy-piperidine-1-carbonyl]-2-methyl-phenyl]-3-propan-2-yl-urea

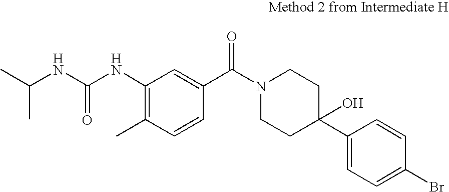

¹H NMR (300.072 MHz, CDCl₃) [symbol] 1.15 (d, 6H), 1.45-1.56 (m, 1H), 1.76-1.90 (m, 3H), 1.96 (s, 3H), 3.04 (s, 1H), 3.17-3.31 (m, 2H), 3.40-3.51 (m, 1H), 3.89 (octet, 1H), 4.47-4.60 (m, 1H), 5.73 (d, 1H), 6.85 (d, 1H), 6.95-7.00 (m, 2H), 7.35 (d, 2H), 7.46 (d, 3H), m/z 474 and 476 M+H)⁺ [B].

### EXAMPLE 40

### 3-benzyl-1-[5-[4-(4-bromophenyl)-4-hydroxy-piperidine-1-carbonyl]-2-methyl-phenyl]urea

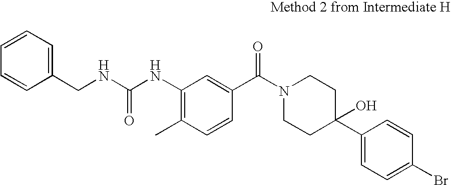

¹H NMR (300.072 MHz, CDCl₃) [symbol] 1.41-1.51 (m, 1H), 1.67-1.83 (m, 3H), 1.88 (s, 3H), 2.97 (s, 1H), 3.06-3.24 (m, 2H), 3.32-3.45 (m, 1H), 4.25 (d, 2H), 4.35-4.44 (m, 1H), 6.26 (t, 1H), 6.79-6.82 (m, 1H), 6.94 (d, 1H), 7.18-7.33 (m, 8H), 7.44 (d, 3H), m/z 522 and 524 M+H)⁺ [B].

### EXAMPLE 41

### 3-(1-benzyl-4-piperidyl)-1-[5-[4-(4-cyanophenyl)piperidine-1-carbonyl]-2-methyl-phenyl]urea

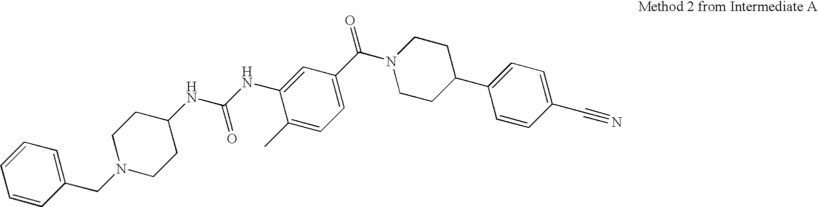

¹H NMR (300.072 MHz, CDCl₃) δ1.41 (2H, d), 1.38-1.50 (1H, m), 1.67 (1H, s), 1.74-1.78 (1H, m), 1.87-1.91 (3H, m), 1.99 (3H, s), 2.08 (1H, d), 2.04-2.13 (1H, m), 2.82 (4H, t), 3.10 (1H, m), 3.48 (2H, s), 3.60 (1H, m), 3.95 (1H, m), 4.85 (1H, m), 5.69 (1H, d), 6.89-6.93 (2H, m), 7.00 (1H, d), 7.29-7.33 (7H, m), 7.45-7.51 (1H, m), 7.57-7.60 (2H, m), m/z 536 (M+H)⁺.

### EXAMPLE 42

### 1-[5-[4-(4-cyanophenyl)piperidine-1-carbonyl]-2-methyl-phenyl]-3-(2-methylpropyl)urea

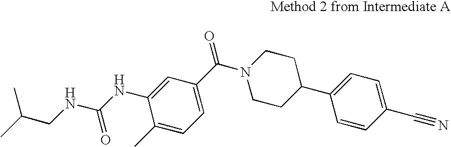

¹H NMR (300.072 MHz, CDCl₃) δ0.86-0.93 (6H, m), 1.62-1.79 (4H, m), 1.87 (1H, s), 2.02 (3H, d), 2.79-2.87 (2H, m), 2.92-2.96 (1H, m), 3.02 (2H, t), 3.95 (1H, m), 4.60-4.84 (1H, m), 5.83 (1H, t), 6.90-6.93 (1H, m), 6.93 (1H, d), 7.02 (1H, d), 7.31 (2H, d), 7.54-7.62 (3H, m), m/z 419 (M+H)⁺.

### EXAMPLE 43

### 1-[5-[4-(4-cyanophenyl)piperidine-1-carbonyl]-2-methyl-phenyl]-3-prop-2-ynyl-urea

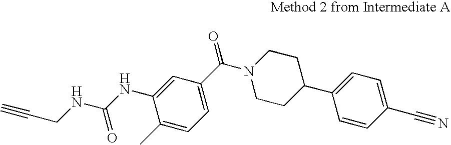

¹H NMR (300.072 MHz, CDCl₃) δ1.50-2.00 (4H, m), 2.00 (3H, s), 2.20 (1H, m), 2.84-3.20 (3H, m), 3.96-3.99 (3H, m), 4.86 (1H, s), 6.12 (1H, t), 6.93-6.96 (1H, m), 7.03 (1H, d), 7.17 (1H, s), 7.32 (2H, d), 7.54-7.59 (2H, m), 7.61 (1H, s), m/z 401 (M+H)⁺.

### EXAMPLE 44

### 1-[5-[4-(4-cyanophenyl)piperidine-1-carbonyl]-2-methyl-phenyl]-3-[(3,4,5-trimethoxyphenyl)methyl]urea

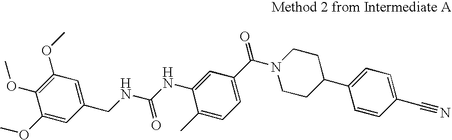

¹H NMR (300.072 MHz, CDCl₃) δ1.50-2.00 (4H, m), 2.04 (3H, s), 2.77-3.10 (3H, m), 3.80-3.83 (9H, m), 3.95 (1H, m), 4.26-4.33 (2H, m), 4.70 (1H, m), 6.07 (1H, t), 6.53-6.54 (2H, m), 6.90-6.93 (1H, m), 6.97 (1H, s), 7.02-7.10 (1H, m), 7.30 (2H, d), 7.59 (2H, d), 7.60 (1H, s), m/z 543 (M+H)⁺.

### EXAMPLE 45

### methyl 3-[[5-[4-(4-cyanophenyl)piperidine-1-carbonyl]-2-methyl-phenyl]carbamoylamino]propanoate

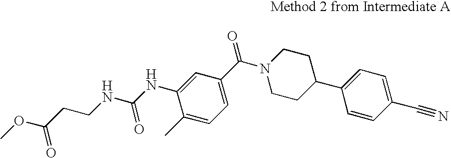

¹H NMR (300.072 MHz, CDCl₃) δ1.50-2.04 (4H, m), 2.04 (3H, d), 2.48-2.58 (2H, m), 2.80-3.20 (3H, m), 3.41-3.48 (2H, m), 3.68 (3H, s), 3.95 (1H, m), 4.88 (1H, s), 5.99 (1H, t), 6.92-6.95 (1H, m), 7.04 (2H, d), 7.32 (2H, d), 7.46-7.52 (1H, m), 7.58-7.61 (2H, m), m/z 449 (M+H)⁺.

### EXAMPLE 46

### 3-[3-[4-(4-cyanophenyl)piperidine-1-carbonyl]phenyl]-1-propan-2-yl-urea

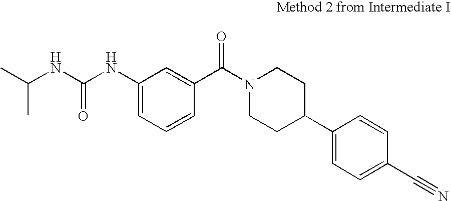

¹H NMR (300.073 MHz, d₆-DMSO, 30° C.) δ 1.09 (6H, dJ=6.1 Hz), 1.48-1.95 (4H, m), 2.69-3.22 (3H, m), 3.63-3.84 (2H, m), 4.43-4.75 (1H, m), 6.03 (1H, dJ=6.8 Hz), 6.92 (1H, dJ=7.5 Hz), 7.21-7.38 (2H, m), 7.45-7.54 (3H, m), 7.76 (2H, dJ=9.6 Hz), 8.41 (1H, s), m/z 391 (M+H)⁺.

### EXAMPLE 47

### 3-[(3-cyanophenyl)methyl]-1-[3-[4-(4-cyanophenyl)piperidine-1-carbonyl]-4-methyl-phenyl]urea

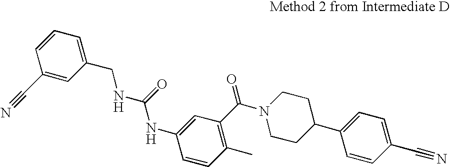

¹H NMR (300.072 MHz, CDCl₃) [symbol] 1.46-1.83 (m, 3H), 1.94-2.00 (m, 1H), 2.19 (d, 3H), 2.76-2.93 (m, 2H), 3.03-3.19 (m, 1H), 3.60-3.66 (m, 1H), 4.38 (d, 2H), 4.82-4.90 (m, 1H), 6.15-6.25 (m, 1H), 6.90-7.07 (m, 2H), 7.13-7.24 (m, 1H), 7.30 (d, 2H), 7.41 (d, 1H), 7.50-7.62 (m, 6H), m/z 478 (M+H)⁺.

### EXAMPLE 48

### 1-[3-[4-(4-cyanophenyl)piperidine-1-carbonyl]-4-methyl-phenyl]-3-cis-(4-hydroxycyclohexyl)urea

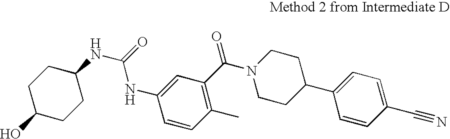

¹H NMR (300.072 MHz, CDCl₃) [symbol] 1.55-1.83 (m, 11H), 1.96-2.01 (m, 1H), 2.11 (s, 1H), 2.25 (d, 3H), 2.79-2.91 (m, 2H), 3.03-3.20 (m, 1H), 3.62-3.77 (m, 2H), 3.83-3.88 (m, 1H), 4.91-4.99 (m, 1H), 5.44-5.51 (m, 1H), 7.04-7.20 (m, 3H), 7.31 (d, 2H), 7.41 (s, 1H), 7.61 (d, 2H) (probably cis), m/z 461 (M+H)⁺.

### EXAMPLE 49

### 1-[3-[4-(4-cyanophenyl)piperidine-1-carbonyl]-4-methyl-phenyl]-3-trans-(4-hydroxycyclohexyl)urea

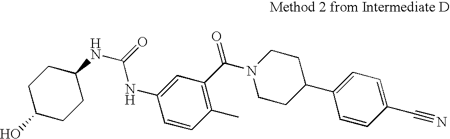

¹H NMR (300.072 MHz, CDCl₃) [symbol] 1.04-2.02 (m, 13H), 2.24 (d, 3H), 2.76-2.93 (m, 2H), 3.05-3.15 (m, 1H), 3.61-3.67 (m, 1H), 4.88-4.97 (m, 1H), 5.33-5.40 (m, 1H), 7.02-7.18 (m, 3H), 7.29-7.34 (m, 2H), 7.39 (s, 1H), 7.61 (d, 2H) (probably trans), m/z 461 (M+H)⁺.

### EXAMPLE 50

### 1-[5-[4-(4-cyanophenyl)-4-hydroxy-piperidine-1-carbonyl]-2-methyl-phenyl]-3-propan-2-yl-urea

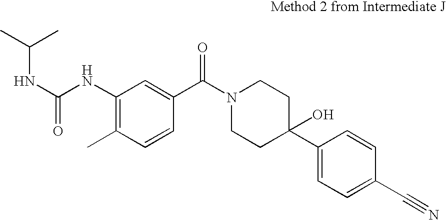

¹H NMR (300.072 MHz, CDCl₃) δ1.14 (d, 6H), 1.42-1.54 (m, 1H), 1.75-1.85 (m, 3H), 1.94 (s, 3H), 3.10-3.24 (m, 2H), 3.34-3.43 (m, 1H), 3.80 (s, 1H), 3.85-3.89 (m, 1H), 4.47-4.61 (m, 1H), 5.88 (d, 1H), 6.82 (d, 1H), 6.98 (d, 1H), 7.12 (s, 1H), 7.51 (s, 1H), 7.57-7.64 (m, 4H), m/z 421 (M+H)⁺.

### EXAMPLE 51

### tert-butyl 4-[[5-[4-(4-cyanophenyl)piperidine-1-carbonyl]-2-methyl-phenyl]carbamoylamino]butanoate

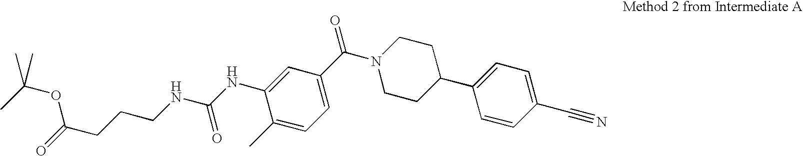

¹H NMR (300.072 MHz, CDCl₃) δ1.72-2.04 (9H, m), 2.29 (2H, t), 2.83 (1H, m), 3.20 (4H, m), 3.95 (1H, m), 4.85 (1H, m), 5.78 (1H, s), 6.88 (1H, s), 6.92-6.96 (1H, m), 7.05 (1H, d), 7.30-7.33 (2H, m), 7.52 (1H, d), 7.58-7.61 (2H, m), m/z 505 (M+H)⁺.

### EXAMPLE 52

### 1-[5-[4-(4-cyanophenyl)piperidine-1-carbonyl]-2-methyl-phenyl]-3-(pyridin-2-ylmethyl)urea

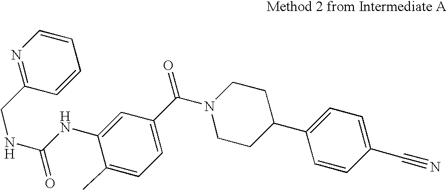

¹H NMR (300.072 MHz, CDCl₃) δ1.55-2.00 (4H, m), 2.04-2.09 (3H, m), 2.77-3.20 (3H, m), 4.00 (1H, m), 4.51 (2H, d), 4.85 (1H, m), 6.58 (1H, t), 6.95-6.98 (1H, m), 7.04 (1H, d), 7.12-7.16 (1H, m), 7.30 (3H, d), 7.48 (1H, s), 7.57-7.60 (2H, m), 7.62-7.65 (1H, m), 7.69 (1H, d), 8.46-8.48 (1H, m), m/z 454 (M+H)⁺.

### EXAMPLE 53

### 3-[5-[4-(4-cyanophenyl)piperidine-1-carbonyl]-2-methyl-phenyl]-1-pentan-3-yl-urea

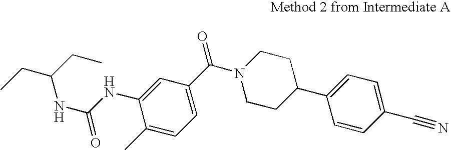

¹H NMR (300.072 MHz, CDCl₃) δ0.90 (6H, t), 1.44-2.00 (11H, m), 2.82 (2H, t), 3.10 (1H, m), 3.60 (1H, m), 3.94 (1H, m), 4.83 (1H, m), 5.55-5.58 (1H, d), 6.90 (2H, d), 7.00 (1H, d), 7.31 (2H, d), 7.51 (1H, d), 7.59 (2H, d), m/z 433 (M+H)⁺.

### EXAMPLE 54

### 3-[5-[4-(4-cyanophenyl)piperidine-1-carbonyl]-2-methyl-phenyl]-1-[(3-methylphenyl)methyl]urea

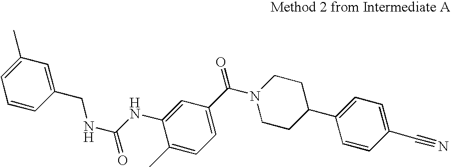

¹H NMR (300.072 MHz, CDCl₃) δ0.90 (6H, t), 1.44-2.00 (11H, m), 2.82 (2H, t), 3.10 (1H, m), 3.60 (1H, m), 3.94 (1H, m), 4.83 (1H, m), 5.55-5.58 (1H, d), 6.90 (2H, d), 7.00 (1H, d), 7.31 (2H, d), 7.51 (1H, d), 7.59 (2H, d), m/z 467 (M+H)⁺.

### EXAMPLE 55

### N-[2-[[5-[4-(4-cyanophenyl)piperidine-1-carbonyl]-2-methyl-phenyl]carbamoylamino]ethyl]acetamide

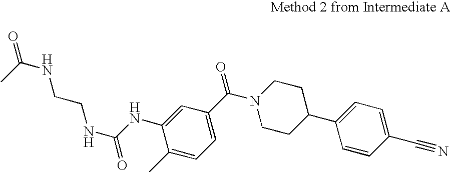

¹H NMR (300.072 MHz, CDCl₃) δ0.50-2.00 (7H, m), 2.02 (3H, s), 2.70-3.22 (3H, m), 3.20 (4H, m), 3.94 (1H, m), 4.75 (1H, m), 6.38 (1H, m), 6.84-6.87 (1H, m), 7.01 (1H, t), 7.11 (1H, s), 7.27 (3H, m), 7.53 (2H, d), 7.73-7.78 (1H, m), m/z 448 (M+H)⁺.

### EXAMPLE 56

### 1-[5-[4-(4-cyanophenyl)piperidine-1-carbonyl]-2-methyl-phenyl]-3-[(3,4-difluorophenyl)methyl]urea

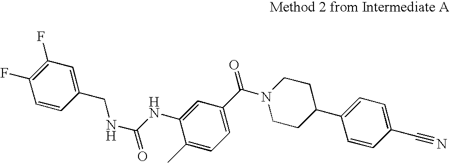

¹H NMR (300.072 MHz, CDCl₃) δ1.50-2.00 (7H, m), 2.81 (2H, m), 3.05-3.10 (1H, m), 3.92 (1H, s), 4.30 (2H, d), 4.75 (1H, s), 6.39 (1H, t), 6.86-6.89 (1H, m), 7.00 (2H, d), 7.07 (2H, d), 7.04-7.12 (1H, m), 7.29 (2H, d), 7.51-7.57 (1H, m), 7.56-7.61 (2H, m), m/z 489 (M+H)⁺.

### EXAMPLE 57

### 1-[5-[4-(4-cyanophenyl)piperidine-1-carbonyl]-2-methyl-phenyl]-3-[2-(4-sulfamoylphenyl)ethyl]urea

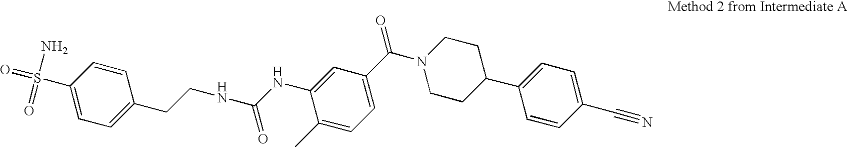

¹H NMR (400.132 MHz, d₆-DMSO) δ 1.47-1.96 (m, 4H), 2.18 (s, 3H), 2.84 (t, 2H), 2.88-3.24 (m, 3H), 3.35-3.44 (m, 2H), 3.67-3.88 (m, 1H), 4.50-4.73 (m, 1H), 6.67 (t, 1H), 6.89-6.97 (m, 1H), 7.18 (d, 1H), 7.32 (s, 2H), 7.44 (d, 2H), 7.51 (d, 2H), 7.73-7.81 (m, 5H), 7.96 (s, 1H), m/z 546 (M+H)⁺.

### EXAMPLE 58

### 2-[[5-[4-(4-cyanophenyl)piperidine-1-carbonyl]-2-methyl-phenyl]carbamoylamino]acetamide

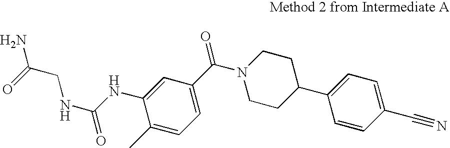

¹H NMR (300.072 MHz, CDCl₃) δ0.50-2.20 (7H, m), 2.73 (2H, m), 3.02 (1H, m), 3.53-3.62 (2H, m), 3.72 (2H, s), 3.80-6.67 (1H, m), 6.85 (1H, d), 7.00 (2H, t), 7.22 (3H, d), 7.48 (2H, d), 7.82 (1H, s), 8.08-10.77 (2H, m), m/z 420 (M+H)⁺.

### EXAMPLE 59

### 3-(1-anilino-2-methyl-propan-2-yl)-1-[5-[4-(4-cyanophenyl)piperidine-1-carbonyl]-2-methyl-phenyl]urea

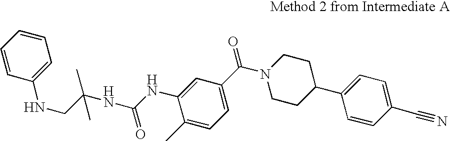

¹H NMR (300.072 MHz, CDCl₃) δ1.39-1.45 (6H, m), 1.50-1.90 (7H, m), 2.73 (2H, t), 3.09 (1H, s), 3.43 (2H, d), 3.92 (1H, d), 4.80 (1H, m), 5.00 (1H, m), 6.00 (1H, d), 6.62-6.66 (1H, m), 6.72-6.77 (3H, m), 6.90 (1H, d), 7.01 (1H, d), 7.09-7.14 (2H, m), 7.20 (2H, t), 7.56 (2H, d), 7.85 (1H, d), m/z 510 (M+H)⁺.

### EXAMPLE 60

### 1-[5-[4-(4-cyanophenyl)piperidine-1-carbonyl]-2-methyl-phenyl]-3-(2-pyridin-2-ylethyl)urea

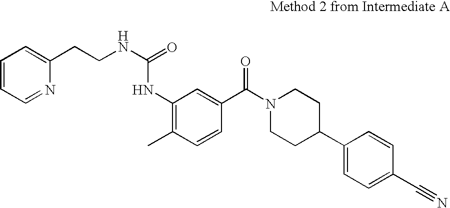

¹H NMR (400.132 MHz, d₆-DMSO) δ 1.54-2.02 (m, 4H), 2.23 (s, 3H), 2.82-3.28 (m, 5H), 3.55 (q, 2H), 3.75-3.96 (m, 1H), 4.58-4.78 (m, 1H), 6.74 (t, 1H), 6.95-7.01 (m, 1H), 7.23 (d, 1H), 7.26-7.33 (m, 1H), 7.35 (d, 1H), 7.57 (d, 2H), 7.75-7.81 (m, 1H), 7.84 (d, 3H), 8.01 (s, 1H), 8.56-8.61 (m, 1H), m/z 468 (M+H)⁺.

### EXAMPLE 61

### 1-[5-[4-(4-cyanophenyl)piperidine-1-carbonyl]-2-methyl-phenyl]-3-[2-(2-methoxyphenyl)ethyl]urea

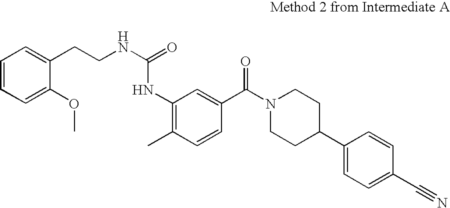

¹H NMR (400.132 MHz, DMSO) δ 1.49-1.95 (m, 4H), 2.19 (s, 3H), 2.73 (t, 2H), 2.78-3.21 (m, 3H), 3.25-3.33 (m, 2H), 3.65-3.88 (m, 4H), 4.51-4.73 (m, 1H), 6.63 (t, 1H), 6.85-7.00 (m, 3H), 7.12-7.26 (m, 3H), 7.51 (d, 2H), 7.71 (s, 1H), 7.78 (d, 2H), 7.97 (s, 1H), m/z 497 (M+H)⁺.

### EXAMPLE 62

### 1-[5-[4-(4-cyanophenyl)piperidine-1-carbonyl]-2-methyl-phenyl]-3-(2-pyridin-4-ylethyl)urea

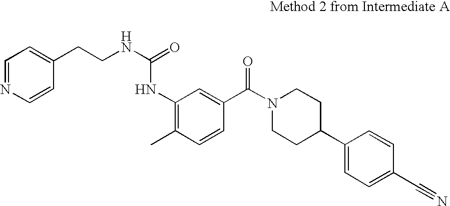

¹H NMR (400.132 MHz, d₆-DMSO) δ 1.46-1.96 (m, 4H), 2.18 (s, 3H), 2.78 (t, 2H), 2.82-3.25 (m, 3H), 3.40 (q, 2H), 3.67-3.89 (m, 1H), 4.49-4.74 (m, 1H), 6.66 (t, 1H), 6.90-6.97 (m, 1H), 7.18 (d, 1H), 7.28 (d, 2H), 7.51 (d, 2H), 7.77 (t, 3H), 7.94 (s, 1H), 8.49 (d, 2H), m/z 468 (M+H)⁺.

### EXAMPLE 63

### 1-[5-[4-(4-cyanophenyl)piperidine-1-carbonyl]-2-methyl-phenyl]-3-(2-thiophen-2-ylethyl)urea

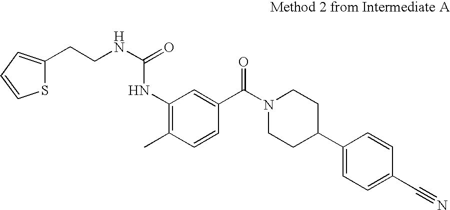

¹H NMR (400.132 MHz, d₆-DMSO) δ 1.48-1.95 (m, 4H), 2.20 (s, 3H), 2.71-3.25 (m, 5H), 3.29-3.42 (m, 2H), 3.67-3.90 (m, 1H), 4.49-4.74 (m, 1H), 6.75 (t, 1H), 6.89-7.00 (m, 3H), 7.18 (d, 1H), 7.33-7.39 (m, 1H), 7.51 (d, 2H), 7.77 (d, 2H), 7.82 (s, 1H), 7.96 (s, 1H), m/z 473 (M+H)⁺.

### EXAMPLE 64

### 1-[5-[4-(4-cyanophenyl)piperidine-1-carbonyl]-2-methyl-phenyl]-3-[2-(4-methoxyphenyl)ethyl]urea

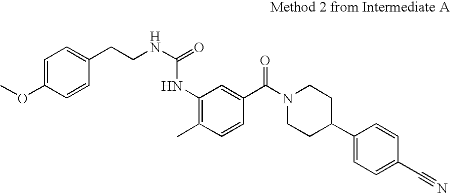

¹H NMR (400.132 MHz, d₆-DMSO) δ 1.47-1.95 (m, 4H), 2.19 (s, 3H), 2.62-2.72 (m, 2H), 2.73-3.20 (m, 3H), 3.22-3.33 (m, 2H), 3.65-3.92 (m, 4H), 4.51-4.76 (m, 1H), 6.62 (t, 1H), 6.81-6.96 (m, 4H), 7.09-7.21 (m, 4H), 7.51 (d, 2H), 7.71-7.84 (m, 2H), 7.93-8.00 (m, 1H) Some base line imps.; m/z 497 (M+H)⁺.

### EXAMPLE 65

### 1-[5-[4-(4-cyanophenyl)piperidine-1-carbonyl]-2-methyl-phenyl]-3-[2-(3-methoxyphenyl)ethyl]urea

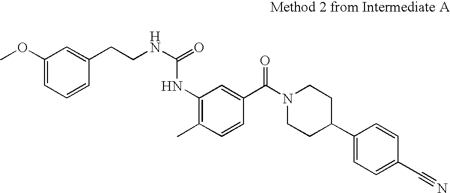

¹H NMR (400.132 MHz, d₆-DMSO) δ 1.49-1.95 (m, 4H), 2.18 (s, 3H), 2.73 (t, 2H), 2.78-3.24 (m, 3H), 3.27-3.33 (m, 2H), 3.67-3.87 (m, 4H), 4.52-4.74 (m, 1H), 6.64 (t, 1H), 6.73-6.85 (m, 3H), 6.93 (d, 1H), 7.14-7.26 (m, 2H), 7.51 (d, 2H), 7.78 (d, 3H), 7.93-8.02 (m, 1H), m/z 497 (M+H)⁺.

### EXAMPLE 66

### 1-[5-[4-(4-cyanophenyl)piperidine-1-carbonyl]-2-methyl-phenyl]-3-(2-pyridin-3-ylethyl)urea

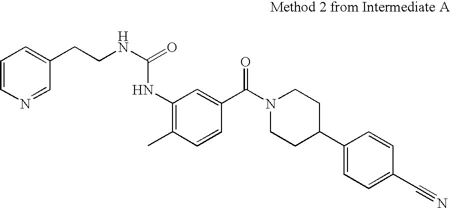

¹H NMR (400.132 MHz, d₆-DMSO) δ 1.46-1.94 (m, 4H), 2.18 (s, 3H), 2.64-3.27 (m, 5H), 3.35-3.43 (m, 2H), 3.66-3.93 (m, 1H), 4.48-4.75 (m, 1H), 6.66 (t, 1H), 6.93 (d, 1H), 7.18 (d, 1H), 7.31-7.38 (m, 1H), 7.51 (d, 2H), 7.68 (d, 1H), 7.72-7.82 (m, 3H), 7.93 (s, 1H), 8.39-8.50 (m, 2H), m/z 468 (M+H)⁺.

### EXAMPLE 67

### 1-[5-[4-(4-cyanophenyl)piperidine-1-carbonyl]-2-methyl-phenyl]-3-[2-(3,5-dimethyl-1,2-oxazol-4-yl)ethyl]urea

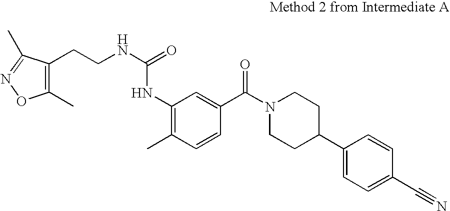

¹H NMR (400.132 MHz, d₆-DMSO) δ 2.16 (s, 3H), 2.19 (s, 3H), 2.29 (s, 3H), 2.46 (t, 2H), 2.71-3.27 (m, 5H), 3.69-3.88 (m, 1H), 4.53-4.72 (m, 1H), 6.62 (t, 1H), 6.90-6.97 (m, 1H), 7.18 (d, 1H), 7.51 (d, 2H), 7.73-7.81 (m, 3H), 7.89-7.95 (m, 1H), m/z 486 (M+H)⁺.

### EXAMPLE 68

### 1-[5-[4-(4-cyanophenyl)piperidine-1-carbonyl]-2-methyl-phenyl]-3-[2-(3-fluorophenyl)ethyl]urea

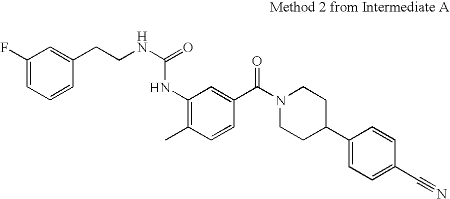

¹H NMR (400.132 MHz, d₆-DMSO) δ 1.49-1.94 (m, 4H), 2.18 (s, 3H), 2.78 (t, 2H), 2.82-3.25 (m, 3H), 3.30-3.42 (m, 2H), 3.68-3.91 (m, 1H), 4.52-4.73 (m, 1H), 6.65 (t, 1H), 6.89-6.96 (m, 1H), 7.00-7.13 (m, 3H), 7.18 (d, 1H), 7.31-7.40 (m, 1H), 7.51 (d, 2H), 7.72-7.81 (m, 3H), 7.92-7.98 (m, 1H), m/z 485 (M+H)⁺.

### EXAMPLE 69

### tert-butyl N-[4-[[5-[4-(4-cyanophenyl)piperidine-1-carbonyl]-2-methyl-phenyl]carbamoylamino]butyl]carbamate

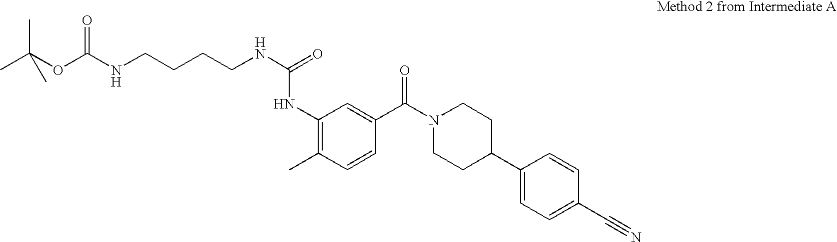

¹H NMR (300.072 MHz, CDCl₃) δ1.37 (4H, s), 1.44 (9H, s), 1.65-1.68 (2H, m), 1.97 (2H, s), 2.14 (3H, s), 2.84 (2H, t), 3.01-3.03 (4H, m), 4.00 (1H, m), 4.85 (1H, m), 5.02 (1H, s), 5.29 (3H, s), 6.05 (1H, s), 6.94-6.97 (1H, m), 7.10 (1H, d), 7.32 (3H, m), 7.59 (2H, d), 7.83 (1H, s), m/z 534 (M+H)⁺.

### EXAMPLE 70

### (RS)-1-[5-[4-(4-cyanophenyl)piperidine-1-carbonyl]-2-methyl-phenyl]-3-[1-(1H-indol-3-yl)propan-2-yl]urea

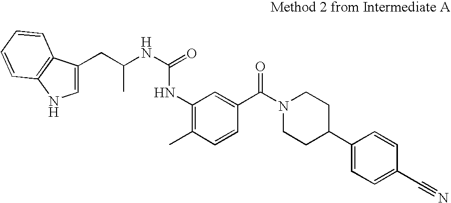

¹H NMR (300.072 MHz, CDCl₃) δ1.25-1.95 (10H, m), 2.85-3.20 (3H, m), 3.40 (1H, m), 3.75 (1H, m), 4.38 (2H, m), 4.70 (1H, m), 5.71 (1H, d), 6.62-7.60 (13H, m), 9.88 (1H, s), m/z 520 (M+H)⁺.

### EXAMPLE 71

### 1-[5-[4-(4-cyanophenyl)piperidine-1-carbonyl]-2-methyl-phenyl]-3-(2,2-dimethylpropyl)urea

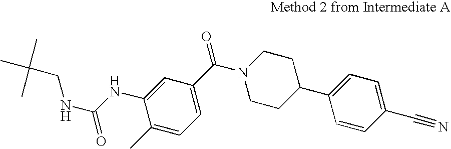

¹H NMR (300.072 MHz, CDCl₃) δ0.92 (9H, s), 1.50-1.98 (7H, m), 2.79-2.87 (2H, m), 3.01 (2H, d), 3.00-3.20 (1H, m), 3.95 (1H, m), 4.85 (1H, m), 5.88 (1H, t), 6.87-6.90 (1H, m), 7.00 (2H, d), 7.31 (2H, d), 7.50 (1H, d), 7.60 (2H, d), m/z 433 (M+H)⁺.

### EXAMPLE 72

### Methyl 2-[[5-[4-(4-cyanophenyl)piperidine-1-carbonyl]-2-methyl-phenyl]carbamoylamino]acetate

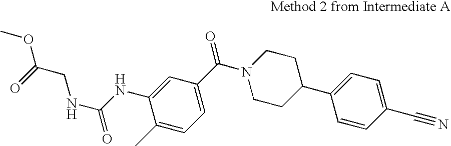

¹H NMR (300.072 MHz, CDCl₃) δ1.50-2.00 (4H, m), 2.04 (3H, s), 2.74-2.83 (1H, m), 2.83-2.87 (1H, m), 3.11 (1H, s), 3.72 (3H, s), 3.92-3.99 (3H, m), 4.90 (1H, m), 6.17 (1H, t), 6.96-6.99 (1H, m), 7.04 (1H, d), 7.32 (3H, m), 7.54-7.61 (3H, m), m/z 435 (M+H)⁺.

### EXAMPLE 73

### (2S)-2-[[5-[4-(4-cyanophenyl)piperidine-1-carbonyl]-2-methyl-phenyl]carbamoylamino]-4-methyl-pentanamide

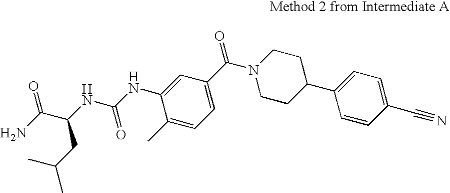

¹H NMR (300.072 MHz, CDCl₃) δ0.91-0.94 (6H, m), 1.48-1.58 (1H, m), 1.62-1.68 (2H, m), 1.72-1.83 (3H, m), 2.05 (3H, s), 2.81-2.85 (2H, m), 3.09 (1H, s), 3.90 (1H, m), 4.40-4.48 (1H, m), 4.84 (1H, s), 6.77 (1H, d), 6.85 (2H, d), 6.98 (1H, d), 7.31 (3H, d), 7.58 (3H, d), 7.76 (1H, s), m/z 476 (M+H)⁺.

### EXAMPLE 74

### 1-[5-[4-(4-cyanophenyl)piperidine-1-carbonyl]-2-methyl-phenyl]-3-(3-pyrrolidin-1-ylpropyl)urea

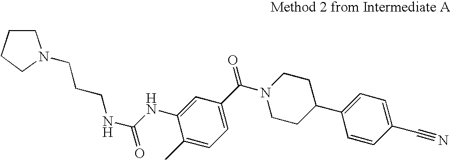

¹H NMR (300.072 MHz, CDCl₃) δ1.60-2.10 (10H, m), 2.14 (3H, s), 2.47-2.60 (6H, m), 2.84-3.20 (3H, m), 3.29 (2H, t), 4.00 (1H, m), 4.85 (1H, m), 6.00 (1H, m), 6.99-7.11 (3H, m), 7.31-7.33 (2H, m), 7.59-7.62 (3H, m), m/z 474 (M+H)⁺.

### EXAMPLE 75

### 1-[5-[4-(4-cyanophenyl)piperidine-1-carbonyl]-2-methyl-phenyl]-3-[[4-(thiadiazol-4-yl)phenyl]methyl]urea

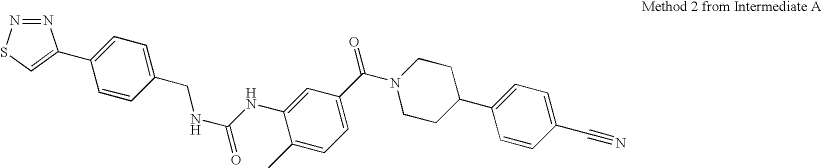

¹H NMR (300.072 MHz, CDCl₃) δ1.50-2.00 (4H, m), 2.00 (3H, s), 2.75 (2H, m), 3.07 (1H, m), 3.92 (1H, s), 4.40 (2H, d), 4.74 (1H, m), 6.43 (1H, t), 6.89-6.92 (1H, m), 7.01 (1H, d), 7.23-7.27 (3H, m), 7.35 (2H, d), 7.54 (2H, d), 7.68 (1H, d), 7.91 (2H, d), 8.63 (1H, s), m/z 537 (M+H)⁺.

### EXAMPLE 76

### 1-[5-[4-(4-cyanophenyl)piperidine-1-carbonyl]-2-methyl-phenyl]-3-[2-(3,5-dimethylpyrazol-1-yl)ethyl]urea

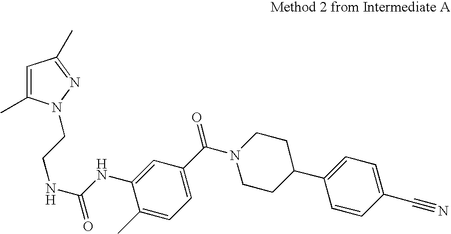

¹H NMR (300.072 MHz, CDCl₃) δ1.60-2.00 (4H, m), 2.05-2.22 (9H, m), 2.80-3.20 (3H, m), 3.53 (1H, q), 4.07 (3H, m), 4.85 (1H, m), 5.77 (1H, s), 6.05 (1H, t), 6.98-7.01 (1H, m), 7.11 (1H, d), 7.33 (3H, d), 7.61 (3H, d), m/z 485 (M+H)⁺.

### EXAMPLE 77

### 1-[5-[4-(4-cyanophenyl)piperidine-1-carbonyl]-2-methyl-phenyl]-3-(2,2-dimethyloxan-4-yl)urea

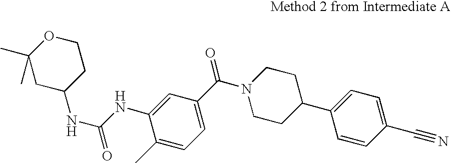

¹H NMR (300.072 MHz, CDCl₃) δ1.18-1.32 (10H, m), 1.50-2.00 (4H, m), 2.01 (3H, s), 2.81-3.20 (3H, m), 3.66-3.79 (2H, m), 3.95-4.02 (1H, m), 5.69 (1H, d), 6.86 (1H, s), 6.90-6.93 (1H, m), 7.03 (1H, d), 7.31-7.34 (2H, m), 7.54 (1H, d), 7.59-7.62 (2H, m), m/z 475 (M+H)⁺.

### EXAMPLE 78

### (2S)-2-[[5-[4-(4-cyanophenyl)piperidine-1-carbonyl]-2-methyl-phenyl]carbamoylamino]-3-[(2-methylpropan-2-yl)oxy]propanamide

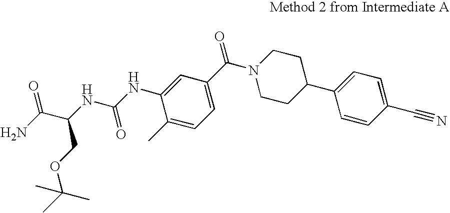

¹H NMR (300.072 MHz, CDCl₃) δ1.20 (9H, s), 1.50-2.00 (4H, m), 2.11 (3H, s), 2.83-3.20 (3H, m), 3.45 (1H, m), 3.79-3.83 (1H, m), 3.95 (1H, m), 4.43-4.46 (1H, m), 4.85 (1H, m), 6.43 (1H, s), 6.60 (1H, d), 6.93-6.97 (2H, m), 7.04 (1H, d), 7.32 (2H, d), 7.60 (2H, d), 7.69 (1H, s), 7.75 (1H, s), m/z 506 (M+H)⁺.

### EXAMPLE 79

### 3-[5-[4-(4-cyanophenyl)piperidine-1-carbonyl]-2-methyl-phenyl]-1-(1-propyl-4-piperidyl)urea

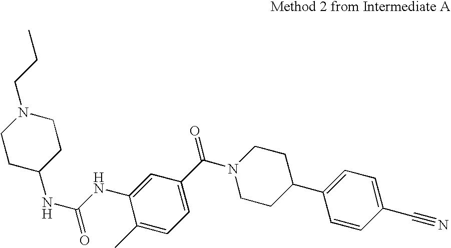

¹H NMR (300.072 MHz, CDCl₃) δ0.83-0.94 (4H, m), 1.04 (3H, t), 1.40-2.10 (10H, m), 2.23-2.35 (2H, m), 2.42 (1H, s), 2.81 (4H, m), 3.06 (1H, m), 3.65 (1H, m), 3.95 (1H, m), 4.85 (1H, m), 5.74 (1H, d), 6.89-6.92 (1H, m), 6.94 (1H, s), 7.02 (1H, d), 7.32 (2H, d), 7.52 (1H, d), 7.60 (2H, d), m/z 488 (M+H)⁺.

### EXAMPLE 80

### 1-[5-[4-(4-cyanophenyl)piperidine-1-carbonyl]-2-methyl-phenyl]-3-[(1,1-dioxothiolan-3-yl)methyl]urea

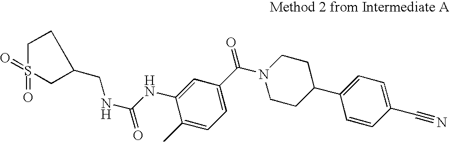

¹H NMR (300.072 MHz, CDCl₃) δ1.55-2.10 (8H, m), 2.30 (1H, m), 2.60-3.40 (10H, m), 3.98 (1H, m), 4.85 (1H, m), 6.39 (1H, t), 6.93 (2H, d), 7.08 (1H, d), 7.33 (2H, d), 7.61 (2H, d), 7.67 (1H, d), m/z 495 (M+H)⁺.

### EXAMPLE 81

### Benzyl N-[2-[[5-[4-(4-cyanophenyl)piperidine-1-carbonyl]-2-methyl-phenyl]carbamoylamino]ethyl]carbamate

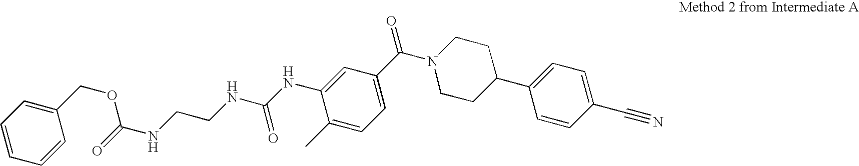

¹H NMR (300.072 MHz, CDCl₃) δ1.50-2.00 (7H, m), 2.74-3.20 (3H, m), 3.29 (4H, s), 3.95 (1H, m), 4.80 (1H, m), 5.07 (2H, s), 5.75 (1H, s), 6.15 (1H, s), 6.85 (1H, s), 6.92 (1H, d), 7.02 (1H, d), 7.26-7.30 (7H, m), 7.58 (2H, d), 7.62 (1H, s), m/z 540 (M+H)⁺.

### EXAMPLE 82

### 1-[5-[4-(4-cyanophenyl)piperidine-1-carbonyl]-2-methyl-phenyl]-3-(1H-tetrazol-5-ylmethyl)urea

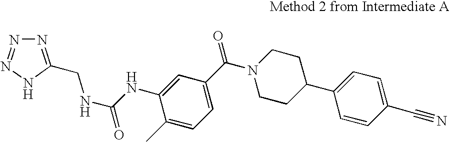

¹H NMR (300.072 MHz, CDCl₃) δ1.50-2.20 (7H, m), 2.75-3.20 (2H, m), 3.64 (2H, t), 4.00 (1H, m), 4.75-4.76 (2H, m), 6.96 (1H, d), 7.08 (1H, d), 7.33 (2H, d), 7.60 (2H, d), 7.80 (1H, m), 7.89 (1H, s), 8.37 (1H, s), m/z 445 (M+H)⁺.

### EXAMPLE 83

### 1-[5-[4-(4-cyanophenyl)piperidine-1-carbonyl]-2-methyl-phenyl]-3-[2-(2-methoxyphenoxy)ethyl]urea

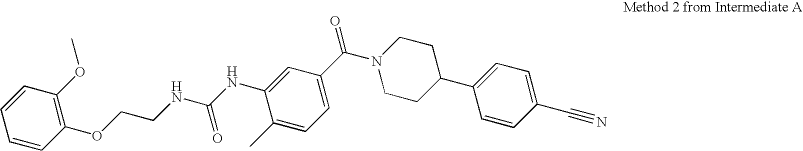

### EXAMPLE 84

### 1-[5-[4-(4-cyanophenyl)piperidine-1-carbonyl]-2-methyl-phenyl]-3-[(1R)-1-(4-methoxyphenyl)ethyl]urea

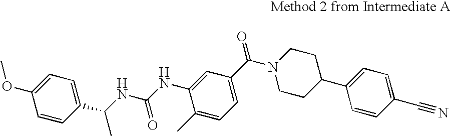

¹H NMR (300.072 MHz, CDCl₃) δ1.42 (3H, d), 1.50-2.00 (7H, m), 2.75-3.20 (3H, m), 3.74 (3H, s), 3.92 (1H, s), 4.75-4.81 (1H, m), 4.85 (1H, t), 6.04 (1H, d), 6.80-6.88 (4H, m), 6.97-6.99 (1H, m), 7.23 (1H, d), 7.26-7.30 (3H, m), 7.57-7.60 (3H, m), m/z 497 (M+H)⁺.

### EXAMPLE 85

### 3-[(3-aminophenyl)methyl]-1-[5-[4-(4-cyanophenyl)piperidine-1-carbonyl]-2-methyl-phenyl]urea

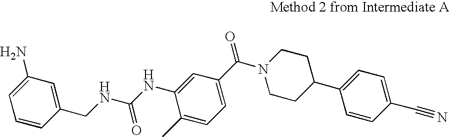

¹H NMR (300.072 MHz, CDCl₃) δ1.40-1.90 (4H, m), 1.97 (3H, d), 2.67-2.75 (2H, m), 3.05 (1H, s), 3.89 (1H, m), 4.30 (2H, d), 4.68 (1H, m), 6.18-7.75 (13H, m), m/z 468 (M+H)⁺.

### EXAMPLE 86

### 3-[2-(benzenesulfonamido)ethyl]-1-[5-[4-(4-cyanophenyl)piperidine-1-carbonyl]-2-methyl-phenyl]urea

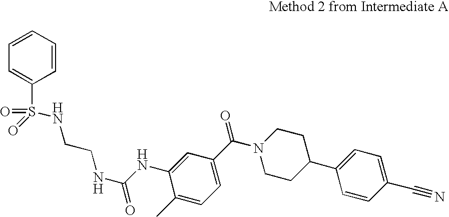

¹H NMR (300.072 MHz, CDCl₃) δ1.73 (2H, s), 1.86 (2H, d), 1.94 (3H, s), 2.79-2.91 (2H, m), 2.96-3.01 (3H, m), 3.25 (2H, m), 3.94-4.00 (1H, m), 4.87 (1H, d), 6.46 (1H, t), 6.55 (1H, t), 6.84-6.87 (1H, m), 6.97 (1H, d), 7.16 (1H, s), 7.33 (2H, d), 7.45-7.48 (2H, m), 7.52 (2H, d), 7.53 (1H, d), 7.83-7.87 (3H, m), m/z 546 (M+H)⁺.

### EXAMPLE 87

### 1-[5-[4-(4-cyanophenyl)piperidine-1-carbonyl]-2-methyl-phenyl]-3-[2-[(4-nitrophenyl)amino]ethyl]urea

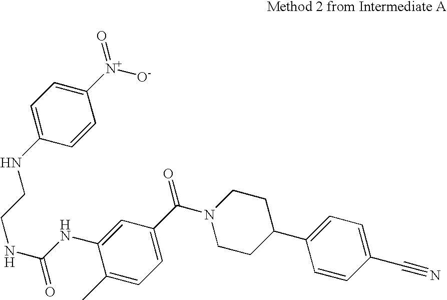

¹H NMR (300.072 MHz, CDCl₃) δ1.50-2.00 (7H, m), 2.63 (1H, m), 2.77 (1H, m), 3.00 (1H, m), 3.37 (2H, d), 3.51-3.55 (2H, m), 3.93 (1H, d), 4.70 (1H, d), 6.33 (1H, t), 6.52 (1H, t), 6.56-6.61 (2H, m), 6.80-6.83 (1H, m), 6.94-6.96 (2H, m), 7.30 (2H, d), 7.59 (2H, d), 7.86 (1H, d), 7.98-8.02 (2H, m), m/z 527 (M+H)⁺.

### EXAMPLE 88

### 1-[5-[4-(4-cyanophenyl)piperidine-1-carbonyl]-2-methyl-phenyl]-3-[1-(4-fluorophenyl)ethyl]urea

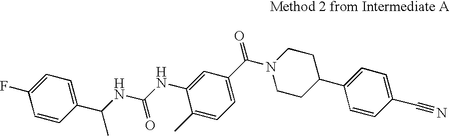

¹H NMR (300.072 MHz, CDCl₃) δ1.41-1.49 (3H, d), 1.50-2.00 (7H, m), 2.77-2.81 (2H, m), 3.09 (1H, m), 3.88 (1H, m), 4.73-4.80 (1H, m), 4.86-4.95 (1H, m), 5.99 (1H, d), 6.73 (1H, s), 6.86-6.89 (1H, m), 6.93-7.00 (3H, m), 7.28 (1H, s), 7.30-7.32 (3H, m), 7.52 (1H, d), 7.59-7.61 (2H, m), m/z 485 (M+H)⁺.

### EXAMPLE 89

### 1-[5-[4-(4-cyanophenyl)piperidine-1-carbonyl]-2-methyl-phenyl]-3-(3-furylmethyl)urea

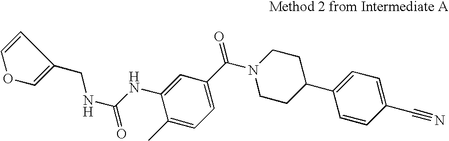

¹H NMR (300.072 MHz, CDCl₃) δ1.50-2.00 (7H, m), 2.78-3.20 (3H, m), 3.95 (1H, m), 4.24 (2H, d), 4.80 (1H, m), 5.82 (1H, t), 6.39-6.40 (1H, m), 6.82 (1H, s), 6.90-6.93 (1H, m), 7.02 (1H, d), 7.29 (1H, s), 7.32-7.38 (3H, m), 7.51 (1H, d), 7.59-7.62 (2H, m), m/z 443 (M+H)⁺.

### EXAMPLE 90

### 1-[5-[4-(4-cyanophenyl)piperidine-1-carbonyl]-2-methyl-phenyl]-3-(2-hydroxyethyl)urea

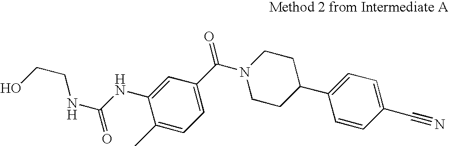

¹H NMR (300.072 MHz, CDCl₃) δ1.60-2.02 (7H, m), 2.80-2.88 (2H, m), 3.13 (1H, m), 3.35 (2H, q), 3.70 (2H, t), 3.96 (1H, m), 4.82-4.88 (1H, m), 6.20 (1H, t), 6.89-6.92 (1H, m), 7.02 (1H, d), 7.11 (1H, s), 7.33 (2H, d), 7.60 (2H, d), 7.74 (1H, d), m/z 407 (M+H)⁺.

### EXAMPLE 91

### N-[2-[[5-[4-(4-cyanophenyl)piperidine-1-carbonyl]-2-methyl-phenyl]carbamoylamino]ethyl]pyridine-2-carboxamide

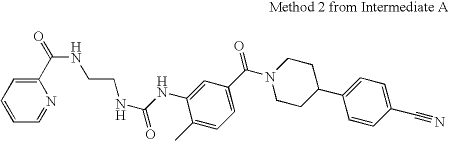

¹H NMR (300.072 MHz, CDCl₃) δ1.50-2.00 (4H, m), 2.10 (3H, s), 2.78-3.20 (3H, m), 3.47 (2H, q), 3.54-3.66 (2H, m), 4.00 (1H, m), 5.95 (1H, t), 6.84 (1H, s), 6.99-7.03 (1H, m), 7.10 (1H, d), 7.31 (2H, d), 7.37-7.42 (1H, m), 7.60 (3H, d), 7.78-7.84 (1H, m), 8.11-8.15 (1H, m), 8.41 (1H, t), 8.52-8.57 (1H, m), m/z 511 (M+H)⁺.

### EXAMPLE 92

### 1-[5-[4-(4-cyanophenyl)piperidine-1-carbonyl]-2-methyl-phenyl]-3-[2-(dimethylsulfamoylamino)ethyl]urea

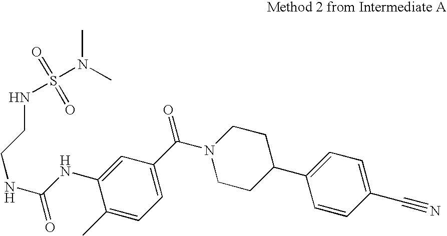

¹H NMR (300.072 MHz, CDCl₃) δ1.60-2.04 (7H, m), 2.80 (8H, m), 3.20-3.26 (3H, m), 3.38 (2H, q), 3.98 (1H, m), 4.90 (1H, m), 5.93 (1H, t), 6.33 (1H, t), 6.87 (1H, d), 6.96-7.00 (2H, m), 7.35 (2H, d), 7.60 (2H, d), 7.78 (1H, s), m/z 513 (M+H)⁺.

### EXAMPLE 93

### N-[2-[[5-[4-(4-cyanophenyl)piperidine-1-carbonyl]-2-methyl-phenyl]carbamoylamino]-2-methyl-propyl]pyridine-3-carboxamide

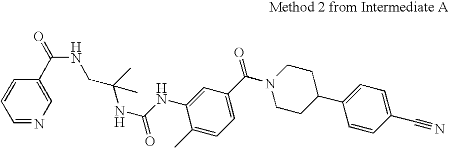

¹H NMR (300.072 MHz, CDCl₃) δ1.36 (6H, s), 1.40-2.00 (7H, m), 2.81 (2H, m), 2.98 (1H, m), 3.73 (2H, d), 3.90-3.96 (1H, m), 4.83 (1H, d), 6.14 (1H, s), 6.79-6.82 (1H, m), 6.91-6.95 (2H, m), 7.21 (2H, d), 7.25-7.30 (1H, m), 7.57 (2H, d), 7.78 (1H, d), 8.31-8.35 (1H, m), 8.57-8.59 (1H, m), 8.95 (1H, t), 9.21 (1H, d), m/z 539 (M+H)⁺.

### EXAMPLE 94

### 3-[2-[(2-amino-5,6-dimethyl-pyrimidin-4-yl)amino]ethyl]-1-[5-[4-(4-cyanophenyl)piperidine-1-carbonyl]-2-methyl-phenyl]urea

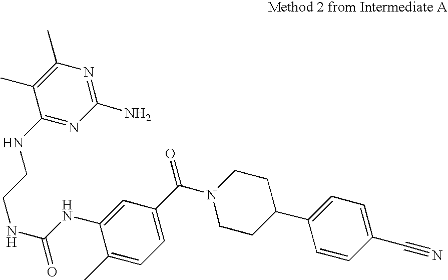

¹H NMR (300.072 MHz, CDCl₃) δ1.50-2.05 (7H, m), 2.06 (3H, s), 2.18 (3H, s), 2.79-3.20 (3H, m), 3.43-3.55 (4H, m), 3.95 1H, m), 4.63 (2H, s), 4.85 (1H, m), 5.47 (1H, t), 6.14 (1H, t), 6.81 (1H, s), 6.95-6.98 (1H, m), 7.05 (1H, d), 7.31 (2H, d), 7.61 (3H, d), m/z 527 (M+H)⁺.

### EXAMPLE 95

### 1-[5-[4-(4-cyanophenyl)piperidine-1-carbonyl]-2-methyl-phenyl]-3-[2-(9H-purin-6-ylamino)ethyl]urea

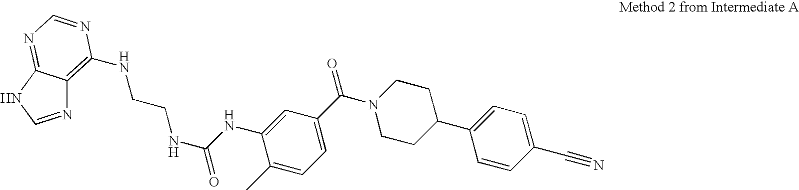

¹H NMR (300.072 MHz, CDCl₃) δ1.43-2.05 (7H, m), 2.61-3.20 (5H, m), 3.20-4.00 (3H, m), 4.75 (1H, m), 6.87-7.00 (4H, m), 7.07-7.31 (3H, m), 7.51-7.61 (3H, m), 7.60-8.00 (2H, m), 8.22 (1H, s), m/z 524 (M+H)⁺.

### EXAMPLE 96

### 1-[5-[4-(4-cyanophenyl)piperidine-1-carbonyl]-2-methyl-phenyl]-3-(3-methylbut-2-enyl)urea

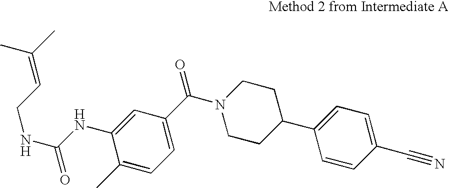

¹H NMR (300.072 MHz, CDCl₃) δ1.50-2.04 (13H, m), 2.79-2.87 (2H, m), 3.09-3.14 (1H, m), 3.80-4.82 (2H, m), 3.95 (1H, m), 4.85 (1H, m), 5.17-5.22 (1H, m), 5.57 (1H, t), 6.86 (1H, s), 6.93-6.96 (1H, m), 7.04 (1H, d), 7.32 (2H, d), 7.57 (1H, d), 7.60 (2H, d), m/z 431 (M+H)⁺.

### EXAMPLE 97

### tert-butyl 3-[[5-[4-(4-cyanophenyl)piperidine-1-carbonyl]-2-methyl-phenyl]carbamoylamino]azetidine-1-carboxylate

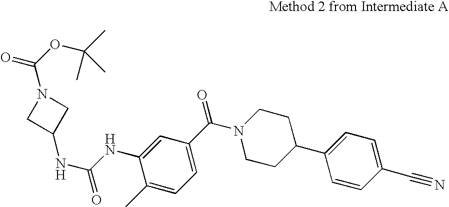

¹H NMR (300.072 MHz, CDCl₃) δ1.44 (9H, d), 1.50-2.00 (7H, s), 2.85-3.25 (3H, m), 3.70-3.75 (2H, m), 3.95 (1H, m), 4.22 (2H, m), 4.49 (1H, m), 4.85 (1H, m), 6.51 (1H, d), 6.95 (2H, d), 7.05-7.07 (1H, m), 7.33 (2H, d), 7.52-7.52 (1H, m), 7.61 (2H, d), m/z 516 (M−H)⁻.

### EXAMPLE 98

### 1-[5-[4-(4-cyanophenyl)piperidine-1-carbonyl]-2-methyl-phenyl]-3-[1-(4-methylsulfonylphenyl)ethyl]urea

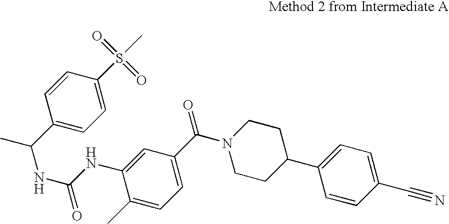

¹H NMR (300.072 MHz, CDCl₃) δ1.42-2.00 (10H, m), 2.79-2.87 (2H, m), 3.00-3.09 (4H, m), 3.90 (1H, m), 4.80-5.05 (2H, m), 6.47 (1H, d), 6.88-6.91 (1H, m), 7.00 (2H, d), 7.30 (2H, d), 7.50 (2H, d), 7.60 (2H, d), 7.56-7.65 (1H, m), 7.81 (2H, q), m/z 545 (M+H)⁺.

### EXAMPLE 99

### 1-[5-[4-(4-cyanophenyl)piperidine-1-carbonyl]-2-methyl-phenyl]-3-(2-oxo-3,4-dihydro-1H-1,7-naphthyridin-3-yl)urea

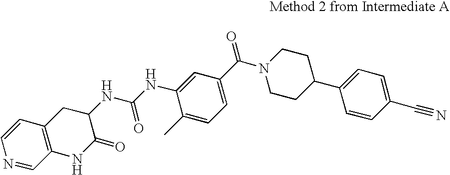

¹H NMR (300.073 MHz, DMSO-d₆) δ1.50-1.90 (4H, m), 2.23 (3H, s), 2.70-3.40 (5H, m), 3.60-4.00 (1H, m), 4.35-4.80 (2H, m), 6.91-6.94 (1H, m), 7.15-7.20 (3H, m), 7.46 (2H, d), 7.71 (2H, d), 7.97 (1H, d), 8.14 (2H, m), 10.55 (1H, s), m/z 509 (M+H)⁺.

### EXAMPLE 100

### 1-[5-[4-(4-cyanophenyl)piperidine-1-carbonyl]-2-methyl-phenyl]-3-[3-(3-methyl-1-piperidyl)propyl]urea

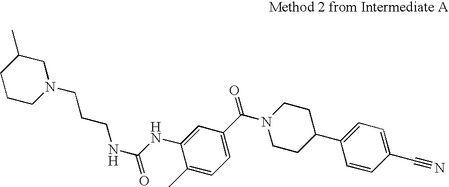

¹H NMR (300.072 MHz, CDCl₃) δ0.84 (4H, m), 1.41-2.00 (9H, m), 2.12 (3H, s), 2.39 (3H, m), 2.79 (3H, d), 2.85 (2H, t), 3.23-3.29 (2H, m), 4.00 (1H, m), 4.85 (1H, s), 6.16 (1H, m), 6.89 (1H, s), 6.98-7.01 (1H, m), 7.09 (1H, d), 7.32 (2H, d), 7.59-7.63 (3H, m), m/z 502 (M+H)⁺.

### EXAMPLE 101

### 1-[5-[4-(4-cyanophenyl)piperidine-1-carbonyl]-2-methyl-phenyl]-3-[2-[(4-methoxyphenyl)amino]ethyl]urea

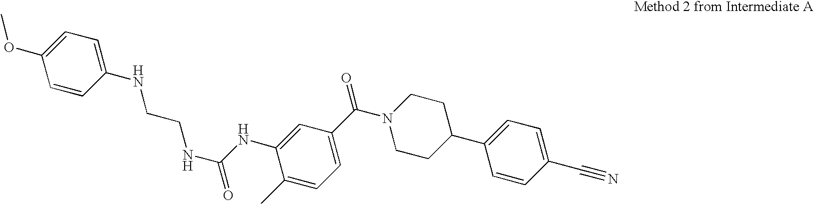

¹H NMR (300.072 MHz, CDCl₃) δ1.50-2.00 (7H, m), 2.77-3.30 (5H, m), 3.41-3.48 (2H, m), 3.70 (3H, s), 3.95 (1H, m), 4.76 (1H, d), 6.13 (1H, t), 6.51-6.65 (3H, m), 6.72-6.75 (3H, m), 6.87-7.00 (3H, m), 7.29 (1H, d), 7.58 (2H, d), 7.65 (1H, d), m/z 512 (M+H)⁺.

### EXAMPLE 102

### tert-butyl N-[2-[[5-[4-(4-cyanophenyl)piperidine-1-carbonyl]-2-methyl-phenyl]carbamoylamino]ethyl]carbamate

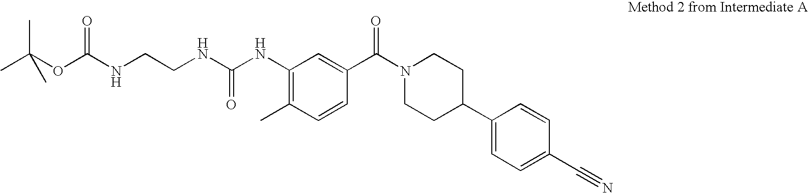

¹H NMR (300.072 MHz, CDCl₃) [symbol] 1.42 (s, 9H), 1.62-2.01 (m, 4H), 2.05 (s, 3H), 2.78-2.93 (m, 2H), 3.06-3.15 (m, 1H), 3.24 (t, 2H), 3.29 (t, 2H), 3.89-4.06 (m, 1H), 4.78-4.97 (m, 1H), 5.21 (s, 1H), 5.92 (s, 1H), 6.81 (s, 1H), 6.94-7.11 (m, 2H), 7.33 (d, 2H), 7.56-7.63 (m, 3H), m/z 506 (M+H)⁺.

### EXAMPLE 103

### 3-(2-aminoethyl)-1-[5-[4-(4-cyanophenyl)piperidine-1-carbonyl]-2-methyl-phenyl]urea

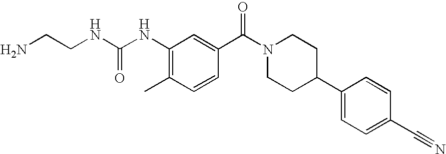

### Method 3

A solution of tert-butyl N-[2-[[5-[4-(4-cyanophenyl)piperidine-1-carbonyl]-2-methyl-phenyl]carbamoylamino]ethyl]carbamate (Example 102) (3.5 g, 6.92 mmol) in DCM (35 mL) was treated with hydrogen chloride (35 mL of a saturated solution in EtOAc), and the reaction stirred at ambient temperature for 2 hrs. The reaction mixture was reduced in vacuo and the residue triturated with ether. The residue was dried in vacuo to give the title compound as a white solid (3.05 g), ¹H NMR (300.073 MHz, d₆-DMSO) [symbol] 1.47-1.83 (m, 4H), 2.24 (s, 3H), 2.82-2.98 (m, 4H), 3.03-3.12 (m, 1H), 3.32-3.42 (m, 2H), 3.68-3.89 (m, 1H), 4.21-4.65 (m, 3H), 6.93 (d, 1H), 7.17 (d, 1H), 7.37 (s, 1H), 7.48 (d, 2H), 7.75 (d, 2H), 7.92 (s, 1H), 8.24 (s, 1H), m/z 406 (M+H)⁺.

### EXAMPLE 104

### 2-[[5-[4-(4-cyanophenyl)piperidine-1-carbonyl]-2-methyl-phenyl]carbamoylamino]ethylcarbamoylformic acid

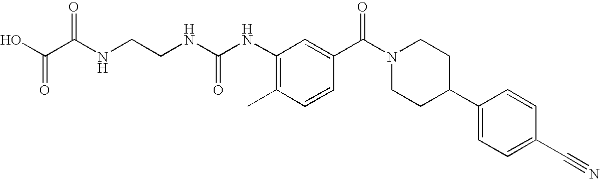

### Method 4

Pyridine (0.15 mL, 1.81 mmol) was added to a solution of 3-(2-aminoethyl)-1-[5-[4-(4-cyanophenyl)piperidine-1-carbonyl]-2-methyl-phenyl]urea (Example 103) (0.2 g, 0.45 mmol) and methyl oxalyl chloride (42 μL, 0.45 mmol) in DCM (3 mL), and the reaction mixture stirred at ambient temperature for 24 hrs. It was then diluted with DCM (10 mL) and the resulting solution washed sequentially with dilute hydrochloric acid (10 mL of 1M), saturated sodium bicarbonate solution (10 mL), brine (10 mL), dried (MgSO₄) and evaporated in vacuo to give a brown oil. This was dissolved in MeOH (4 mL) and a solution of potassium carbonate (240 mg, 5 eq) in water (4 mL) added. The reaction mixture was heated to reflux for 5 mins, and the MeOH then removed in vacuo. The resulting aqueous portion was acidified and extracted with ethyl acetate (30 mL) and the organic extracts washed with brine (30 mL), dried (MgSO₄), and evaporated in vacuo to give a beige solid which was purified by chromatography on silica, eluting with 0-40% MeOH in DCM, to give the title compound as a colourless solid, ¹H NMR (300.073 MHz) δ 1.48-1.86 (m, 4H), 2.19 (s, 3H), 2.84-2.96 (m, 2H), 3.01-3.09 (m, 1H), 3.20-3.24 (m, 4H), 3.62-3.89 (m, 1H), 4.49-4.75 (m, 1H), 6.86 (s, 2H), 6.91-6.94 (m, 1H), 7.15 (d, 1H), 7.49 (d, 2H), 7.75 (d, 2H), 7.91 (s, 1H), 7.95 (s, 1H), m/z 478 (M+H)⁺.

### EXAMPLE 104A

### 2-[2-[[5-[4-(4-cyanophenyl)piperidine-1-carbonyl]-2-methyl-phenyl]carbamoylamino]ethylcarbamoyl]acetic acid

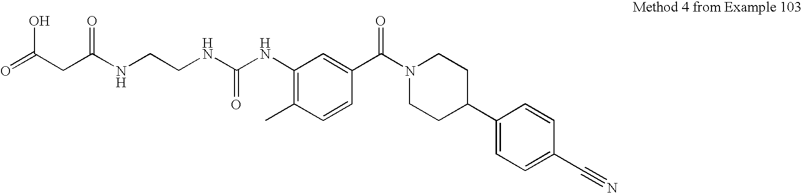

¹H NMR (300.073 MHz, d₆-DMSO) δ 1.48-1.87 (m, 4H), 2.20 (s, 3H), 2.79-2.94 (m, 2H), 3.00 (s, 2H), 3.06-3.12 (m, 1H), 3.17-3.23 (m, 4H), 3.71-3.92 (m, 1H), 4.40-4.72 (m, 1H), 6.88-6.93 (m, 1H), 7.14 (d, 2H), 7.49 (d, 2H), 7.75 (d, 2H), 7.91 (s, 1H), 8.10 (s, 1H), 8.62 (s, 1H), m/z 492 (M+H)⁺.

### EXAMPLE 105

### 1-[5-[4-(4-cyanophenyl)piperidine-1-carbonyl]-2-methyl-phenyl]-3-[(1S)-1-phenylethyl]urea

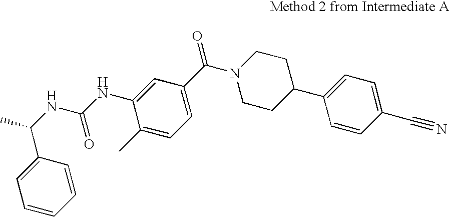

¹H NMR (300.072 MHz, CDCl₃) δ1.44 (3H, d), 1.67-2.00 (7H, m), 2.74-3.20 (3H, m), 3.90 (1H, m), 4.76 (1H, m), 4.86-4.95 (1H, m), 6.07 (1H, d), 6.87 (2H, d), 6.98 (1H, d), 7.17-7.23 (1H, m), 7.28 (3H, d), 7.30-7.34 (3H, m), 7.57 (1H, d), 7.59 (2H, d), m/z 467 (M+H)⁺.

### EXAMPLE 106

### 1-[5-[4-(4-cyanophenyl)piperidine-1-carbonyl]-2-methyl-phenyl]-3-[(1R)-1-phenylethyl]urea

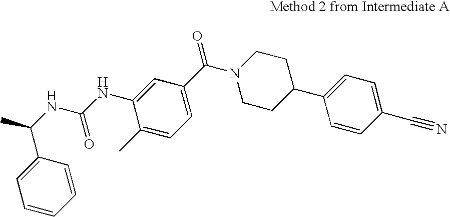

¹H NMR (300.072 MHz, CDCl₃) δ1.41-1.49 (3H, d), 1.67-2.00 (7H, m), 2.74-3.07 (3H, m), 3.87-3.91 (1H, m), 4.73 (1H, m), 4.85-4.94 (1H, m), 6.19 (1H, d), 6.85-6.96 (3H, m), 7.11-7.31 (7H, m), 7.56-7.59 (3H, m), m/z 467 (M+H)⁺.

### EXAMPLE 107

### 1-[5-[4-(4-cyanophenyl)piperidine-1-carbonyl]-2-methyl-phenyl]-3-(4-hydroxycyclohexyl)urea

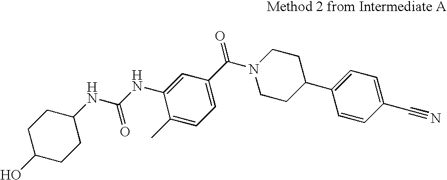

¹H NMR (300.072 MHz, CDCl₃) δ1.09-2.10 (15H, m), 2.83-3.20 (3H, t), 3.51-3.56 (1H, m), 3.73-4.10 (2H, s), 4.83 (1H, s), 5.68-5.91 (1H, m), 6.85-7.10 (3H, m), 7.32 (2H, d), 7.58-7.71 (3H, m), m/z 461 (M+H)⁺.

### EXAMPLE 108

### 3-[(3-cyanophenyl)methyl]-1-[5-[4-(4-cyanophenyl)piperidine-1-carbonyl]-2-methyl-phenyl]urea

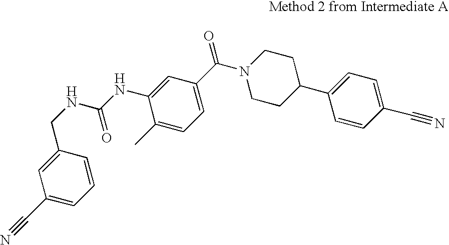

¹H NMR (300.072 MHz, CDCl₃) δ1.63-2.02 (7H, m), 2.80-3.20 (3H, m), 3.91 (1H, m), 4.38 (2H, d), 4.77 (1H, m), 6.51 (1H, t), 6.89-6.92 (1H, m), 7.02-7.08 (2H, m), 7.29-7.32 (2H, m), 7.41 (1H, q), 7.49-7.52 (2H, m), 7.58 (4H, t), m/z 478 (M+H)⁺.

### EXAMPLE 109

### 1-[5-[4-(4-cyanophenyl)piperidine-1-carbonyl]-2-methyl-phenyl]-3-(2-methanesulfonamidoethyl)urea

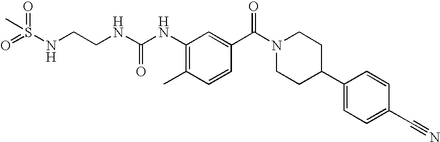

### Method 5

Pyridine (0.15 mL, 1.81 mmol) was added to a solution of 3-(2-aminoethyl)-1-[5-[4-(4-cyanophenyl)piperidine-1-carbonyl]-2-methyl-phenyl]urea (Example 103) (0.2 g, 0.45 mmol) and methane sulfonyl chloride (43 μL, 0.54 mmol) in DCM (3 mL), and the reaction mixture stirred at ambient temperature for 48 hrs. It was then diluted with DCM (10 mL) and the resulting solution washed sequentially with dilute hydrochloric acid (10 mL of 1M), saturated sodium bicarbonate solution (10 mL), brine (10 mL), dried (MgSO4) and evaporated in vacuo to give a brown oil. This was purified by chromatography on silica, eluting with 0-10% MeOH in EtOAc, to give the title compound as a colourless solid, ¹H NMR (300.072 MHz, CDCl₃) [symbol] 1.63-1.83 (m, 4H), 1.95 (s, 3H), 2.80-2.90 (m, 2H), 2.97 (s, 3H), 3.11-3.20 (m, 1H), 3.25-3.32 (m, 2H), 3.37-3.44 (m, 2H), 3.90-4.05 (m, 1H), 4.81-4.92 (m, 1H), 6.02 (t, 1H), 6.38 (t, 1H), 6.85 (d, 1H), 6.96 (s, 1H), 6.97 (d, 1H), 7.35 (d, 2H), 7.61 (d, 2H), 7.82 (s, 1H), m/z 484 (M+H)⁺.

### EXAMPLE 110

### 3-[5-[4-(4-cyanophenyl)piperidine-1-carbonyl]-2-methyl-phenyl]-1-[2-(ethylsulfonylamino)ethyl]urea

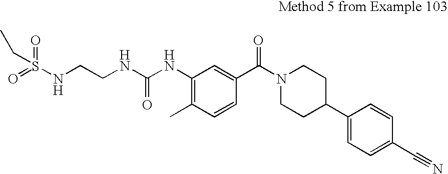

¹H NMR (300.072 MHz, CDCl₃) [symbol] 1.36 (t, 3H), 1.66-1.91 (m, 4H), 2.00 (s, 3H), 2.81-2.92 (m, 2H), 3.05 (q, 2H), 3.14-3.20 (m, 1H), 3.23-3.29 (m, 2H), 3.33-3.39 (m, 2H), 3.93-4.03 (m, 1H), 4.75-4.96 (m, 1H), 5.96-6.04 (m, 1H), 6.42-6.50 (m, 1H), 6.87 (d, 1H), 7.00 (d, 1H), 7.09 (s, 1H), 7.35 (d, 2H), 7.60 (d, 2H), 7.80 (s, 1H), m/z 498 (M+H)⁺. ¹H NMR (300.072 MHz, CDCl₃) [symbol] 1.10 (t, 3H), 1.52-1.87 (m, 4H), 2.09 (s, 3H), 2.19 (q, 2H), 2.79-2.90 (m, 2H), 3.02-3.23 (m, 1H), 3.28-3.37 (m, 4H), 3.90-4.06 (m, 1H), 4.73-4.92 (m, 1H), 6.34-6.38 (m, 1H), 6.92-6.98 (m, 2H), 7.07 (d, 1H), 7.26 (s, 1H), 7.33 (d, 2H), 7.61 (d, 2H), 7.78-7.80 (m, 1H), m/z 462 (M+H)⁺.

### EXAMPLE 111

### N-[2-[[5-[4-(4-cyanophenyl)piperidine-1-carbonyl]-2-methyl-phenyl]carbamoylamino]ethyl]propanamide

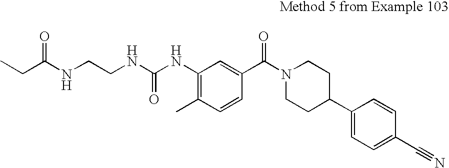

¹H NMR (300.072 MHz, CDCl₃) δ 1.10 (t, 3H), 1.52-1.87 (m, 4H), 2.09 (s, 3H), 2.19 (q, 2H), 2.79-2.90 (m, 2H), 3.02-3.23 (m, 1H), 3.28-3.37 (m, 4H), 3.90-4.06 (m, 1H), 4.73-4.92 (m, 1H), 6.34-6.38 (m, 1H), 6.92-6.98 (m, 2H), 7.07 (d, 1H), 7.26 (s, 1H), 7.33 (d, 2H), 7.61 (d, 2H), 7.78-7.80 (m, 1H), m/z 462 (M+H)⁺.

### EXAMPLE 112

### N-[2-[[5-[4-(4-cyanophenyl)piperidine-1-carbonyl]-2-methyl-phenyl]carbamoylamino]ethyl]-2-methyl-propanamide

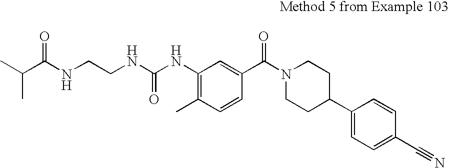

¹H NMR (300.072 MHz, CDCl₃) [symbol] 11111 (d, 6H), 1.61-1.97 (m, 4H), 2.10 (s, 3H), 2.38 (septet, 1H), 2.81-2.89 (m, 2H), 2.99-3.23 (m, 1H), 3.30-3.37 (m, 4H), 3.90-4.02 (m, 1H), 4.72-4.91 (m, 1H), 6.29 (s, 1H), 6.84 (s, 1H), 6.96 (d, 1H), 7.08 (d, 1H), 7.21 (s, 1H), 7.33 (d, 2H), 7.61 (d, 2H), 7.76 (s, 1H), m/z 476 (M+H)⁺.

### EXAMPLE 113

### 2-[[5-[4-(4-cyanophenyl)piperidine-1-carbonyl]-2-methyl-phenyl]carbamoylamino]ethylcarbamoylmethyl acetate

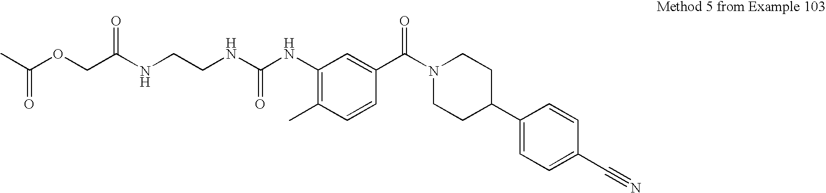

¹H NMR (300.072 MHz, CDCl₃) δ1.59-1.88 (m, 4H), 2.05 (s, 3H), 2.08 (s, 3H), 2.78-2.89 (m, 2H), 3.00-3.18 (m, 1H), 3.34-3.43 (m, 4H), 3.89-4.06 (m, 1H), 4.53 (s, 2H), 4.74-4.93 (m, 1H), 6.10-6.16 (m, 1H), 6.93-6.97 (m, 1H), 7.04-7.10 (m, 2H), 7.33 (d, 2H), 7.47-7.52 (m, 1H), 7.60 (d, 2H), 7.68-7.72 (m, 1H), m/z 506 (M+H)⁺.

### EXAMPLE 114

### N-[2-[[5-[4-(4-cyanophenyl)piperidine-1-carbonyl]-2-methyl-phenyl]carbamoylamino]ethyl]-2-hydroxy-acetamide

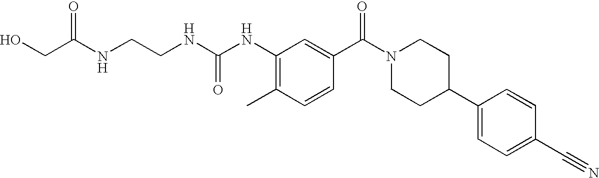

### Method 6

DIPEA (0.31 mL, 1.81 mmol) was added to a mixture of 3-(2-aminoethyl)-1-[5-[4-(4-cyanophenyl)piperidine-1-carbonyl]-2-methyl-phenyl]urea (Example 103) (0.2 g, 0.45 mmol), glycolic acid (104 mg, 1.36 mmol) and HATU (0.36 g 0.95 mmol) in DMF (3 mL) and stirred at ambient temperature for 24 hrs. It was then diluted with EtOAc (30 mL) and the resulting solution washed sequentially with water and brine (30 mL of each), dried (MgSO₄) and evaporated in vacuo to give a brown oil. This was purified by chromatography on silica, eluting with 0-20% MeOH in EtOAc, to give the title compound as a colourless solid, ¹H NMR (300.072 MHz, CDCl₃) [symbol] 1.71-1.86 (m, 4H), 1.98 (s, 3H), 2.80-2.99 (m, 2H), 3.09-3.25 (m, 1H), 3.36-3.48 (m, 4H), 3.91-3.97 (m, 1H), 4.00-4.05 (m, 2H), 4.78-4.90 (m, 1H), 4.97-5.04 (m, 1H), 6.12-6.17 (m, 1H), 6.83-6.87 (m, 1H), 6.99 (d, 1H), 7.14 (s, 1H), 7.30-7.37 (m, 3H), 7.61 (d, 2H), 7.69 (d, 1H), m/z 462 (M−H)⁻.

### EXAMPLE 115

### tert-butyl N-[2-[[5-[4-(4-cyanophenyl)piperidine-1-carbonyl]-2-methyl-phenyl]carbamoylamino]ethyl]-N-methyl-carbamate

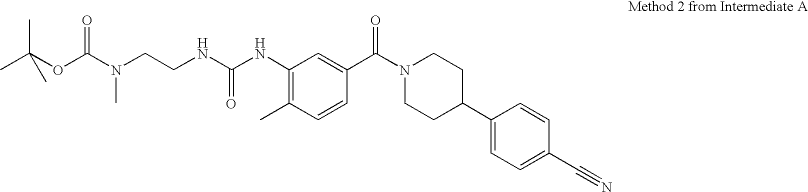

¹H NMR (300.072 MHz, CDCl₃) δ 1.42 (s, 9H), 1.64-1.97 (m, 4H), 2.19 (s, 3H), 2.78-2.86 (m, 2H), 2.89 (s, 3H), 3.02-3.22 (m, 1H), 3.34-3.41 (m, 4H), 3.91-4.07 (m, 1H), 4.75-4.99 (m, 1H), 5.48 (s, 1H), 6.38 (s, 1H), 7.06-7.19 (m, 2H), 7.32 (d, 2H), 7.61 (d, 2H), 7.65 (s, 1H), m/z 518 (M−H)⁻.

### EXAMPLE 116

### 1-[5-[4-(4-cyanophenyl)piperidine-1-carbonyl]-2-methyl-phenyl]-3-(2-methylaminoethyl)urea

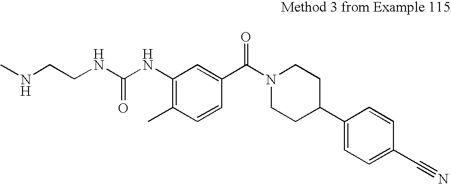

¹H NMR (300.073 MHz, d₆-DMSO) [symbol] 1.51-1.86 (m, 4H), 2.24 (s, 3H), 2.53-2.57 (m, 3H), 2.88-3.03 (m, 4H), 3.09-3.16 (m, 1H), 3.36-3.41 (m, 2H), 3.76-3.85 (m, 1H), 4.44-4.71 (m, 1H), 6.92-6.96 (m, 1H), 7.18 (d, 2H), 7.49 (d, 2H), 7.76 (d, 2H), 7.89-7.92 (m, 1H), 8.15 (s, 1H), 8.96 (s, 1H), m/z 420 (M+H)⁺.

### EXAMPLE 117

### N-[2-[[5-[4-(4-cyanophenyl)piperidine-1-carbonyl]-2-methyl-phenyl]carbamoylamino]ethyl]thiophene-2-carboxamide

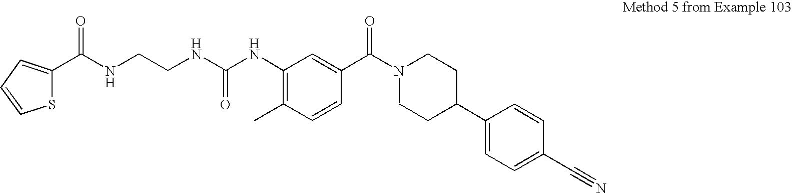

¹H NMR (300.072 MHz, CDCl₃) [symbol] 1.45-1.81 (m, 4H), 1.97 (s, 3H), 2.70-2.98 (m, 3H), 3.41-3.49 (m, 2H), 3.52-3.59 (m, 2H), 3.81-3.95 (m, 1H), 4.70-4.91 (m, 1H), 6.52 (s, 1H), 6.82-6.87 (m, 1H), 6.91-6.94 (m, 1H), 6.98 (d, 1H), 7.14 (s, 1H), 7.29 (d, 2H), 7.36 (d, 1H), 7.59 (d, 2H), 7.66 (d, 1H), 7.85 (s, 1H), 7.91 (t, 1H), m/z 516 (M+H)⁺.

### EXAMPLE 118

### N-[2-[[5-[4-(4-cyanophenyl)piperidine-1-carbonyl]-2-methyl-phenyl]carbamoylamino]ethyl]-1-methyl-pyrrole-2-carboxamide

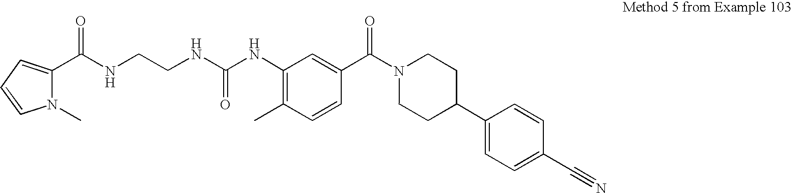

¹H NMR (300.072 MHz, CDCl₃) [symbol] 1.58-1.93 (m, 4H), 1.98 (s, 3H), 2.75-2.85 (m, 2H), 2.98-3.18 (m, 1H), 3.35-3.54 (m, 4H), 3.91 (s, 3H), 3.98-4.08 (m, 1H), 4.68-4.83 (m, 1H), 5.94-5.97 (m, 1H), 6.25 (t, 1H), 6.63-6.68 (m, 2H), 6.89-6.93 (m, 1H), 6.98-7.03 (m, 2H), 7.20 (t, 1H), 7.30 (d, 2H), 7.59 (d, 2H), 7.69 (s, 1H), m/z 513 (M+H)⁺.

### EXAMPLE 119

### N-[2-[[5-[4-(4-cyanophenyl)piperidine-1-carbonyl]-2-methyl-phenyl]carbamoylamino]ethyl]-5-methyl-1,2-oxazole-4-carboxamide

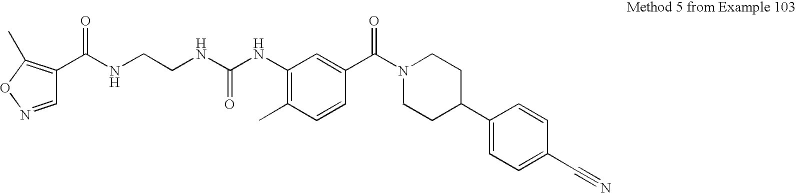

¹H NMR (300.072 MHz, CDCl₃) δ 1.47-1.81 (m, 4H), 1.90 (s, 3H), 2.69 (s, 3H), 2.75-2.86 (m, 2H), 2.93-3.07 (m, 1H), 3.40-3.56 (m, 4H), 3.82-3.98 (m, 1H), 4.75-4.88 (m, 1H), 6.46-6.51 (m, 1H), 6.84 (d, 1H), 6.94 (d, 1H), 7.05 (s, 1H), 7.32 (d, 2H), 7.62 (d, 2H), 7.70 (s, 1H), 7.98 (t, 1H), 8.67 (s, 1H), m/z 515 (M+H)⁺.

### EXAMPLE 120

### N-[2-[[5-[4-(4-cyanophenyl)piperidine-1-carbonyl]-2-methyl-phenyl]carbamoylamino]ethyl]-N-methyl-acetamide

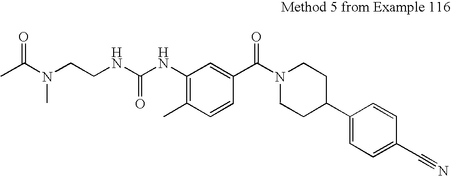

¹H NMR (300.072 MHz, CDCl₃) δ 1.63-1.95 (m, 4H), 2.10 & 2.12 (2×s, 3H), 2.13 & 2.20 (2×s, 3H), 2.78-2.90 (m, 2H), 2.93 & 3.07 (2×s, 3H), 3.11-3.19 (m, 1H), 3.36-3.54 (m, 4H), 3.87-4.14 (m, 1H), 4.79-4.93 (m, 1H), 5.85 & 6.26 (2×t, 1H), 6.88 (s, 1H), 7.01-7.17 (m, 2H), 7.32 (d, 2H), 7.60 (d, 2H), 7.66-7.72 (m, 1H) (NB: spectrum is complicated due to rotamers), m/z 462 (M+H)⁺.

### EXAMPLE 121

### 1-[5-[4-(4-cyanophenyl)piperidine-1-carbonyl]-2-methyl-phenyl]-3-[2-(methyl-methylsulfonyl-amino)ethyl]urea

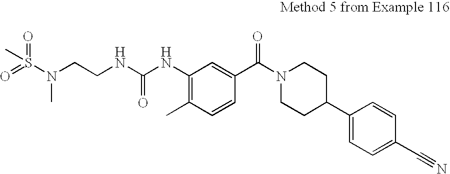

¹H NMR (300.072 MHz, CDCl₃) δ1.62-1.98 (m, 4H), 2.13 (s, 3H), 2.81 (s, 3H), 2.82-2.87 (m, 2H), 2.90 (s, 3H), 3.05-3.19 (m, 1H), 3.23-3.28 (m, 2H), 3.38-3.46 (m, 2H), 3.88-4.05 (m, 1H), 4.75-4.97 (m, 1H), 5.72 (s, 1H), 6.81 (s, 1H), 7.01-7.13 (m, 2H), 7.34 (d, 2H), 7.53 (s, 1H), 7.62 (d, 2H), m/z 498 (M+H)⁺.

### EXAMPLE 122

### 1-[5-[4-(4-cyanophenyl)piperidine-1-carbonyl]-2-methyl-phenyl]-3-[2-(propan-2-ylsulfonylamino)ethyl]urea

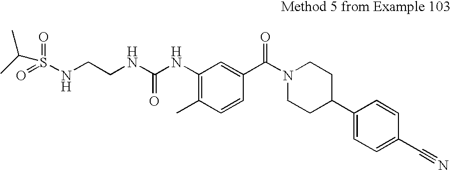

¹H NMR (300.072 MHz, CDCl₃) [symbol] 1.37 (d, 6H), 1.54-1.82 (m, 4H), 1.98 (s, 3H), 2.79-2.88 (m, 2H), 2.97-3.09 (m, 1H), 3.19 (septet, 1H), 3.27-3.32 (m, 2H), 3.36-3.43 (m, 2H), 3.92-4.04 (m, 1H), 4.81-4.95 (m, 1H), 5.72 (t, 1H), 6.24 (s, 1H), 6.84 (s, 1H), 6.87-6.91 (m, 1H), 6.97-7.02 (m, 1H), 7.36 (d, 2H), 7.60 (d, 2H), 7.76-7.80 (m, 1H), m/z 512 (M+H)⁺.

### EXAMPLE 123

### 4-[1-[3-(benzylcarbamoylamino)-4-methyl-benzoyl]-4-piperidyl]-N,N-dimethyl-benzamide

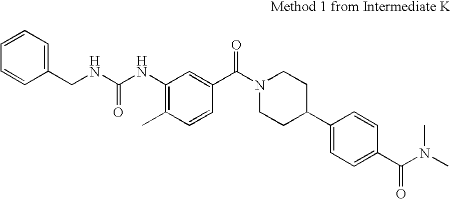

¹H NMR (300.073 MHz, d₆-DMSO) δ 1.47-1.88 (m, 4H), 2.21 (s, 3H), 2.74-3.02 (m, 9H), 3.67-3.90 (m, 1H), 4.30 (d, J=5.7 Hz, 2H), 4.47-4.70 (m, 1H), 6.93 (d, J=7.5 Hz, 1H), 7.02-7.09 (m, 1H), 7.18 (d, J=7.7 Hz, 1H), 7.22-7.27 (m, 1H), 7.32 (s, 8H), 7.81 (s, 1H), 7.98 (s, 1H), m/z 499 (M+H)⁺.

### EXAMPLE 124

### N,N-dimethyl-4-[1-[4-methyl-3-(propan-2-ylcarbamoylamino)benzoyl]-4-piperidyl]benzamide

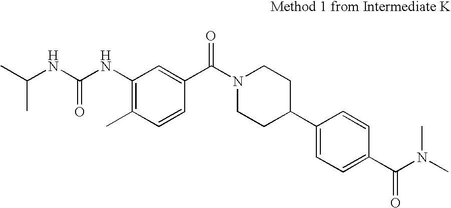

¹H NMR (300.073 MHz, d₆-DMSO) δ 1.10 (d, J=6.5 Hz, 6H), 1.47-1.87 (m, 4H), 1.98 (s, 1H), 2.19 (s, 3H), 2.76-3.04 (m, 9H), 3.67-3.81 (m, 1H), 4.47-4.71 (m, 1H), 6.52 (d, J=7.3 Hz, 1H), 6.89 (d, J=6.2 Hz, 1H), 7.16 (d, J=7.7 Hz, 1H), 7.32 (s, 4H), 7.58 (s, 1H), 8.00 (s, 1H), m/z 451 (M+H)⁺.

### EXAMPLE 125

### 3-[[5-[4-(4-cyanophenyl)piperidine-1-carbonyl]-2-methyl-phenyl]carbamoylamino]propanoic acid

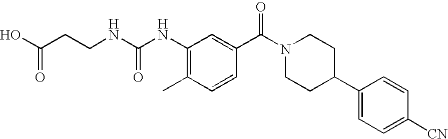

### Method 7

A solution of methyl 3-[[5-[4-(4-cyanophenyl)piperidine-1-carbonyl]-2-methyl-phenyl]carbamoylamino]propanoate (Example 45) (1.6 g, 3.56 mmol) in THF (10 mL) was treated with a solution of lithium hydroxide (299 mg, 7.12 mmol) in water (5 mL), and the reaction mixture was stirred for 16 hours at ambient temperature. It was then evaporated in vacuo to remove the THF and the aqueous phase was diluted with water (10 mL) and washed with EtOAc (10 mL). Citric acid solution (10% aqueous) was added to the aqueous phase until the pH was 4-5. The precipitate thus formed was extracted into EtOAc (3×15 mL), the combined organic phases dried (MgSO₄) and concentrated in vacuo to yield a colourless foam, ¹H NMR (300.073 MHz, d₆-DMSO) δ 1.5-1.9 (4H, m), 2.1 (3H, s), 2.4 (2H, t), 2.8-3.2 (2H, m), 3.3 (2H, m), 3.6-3.9 (1H, m), 4.4-4.7 (1H, m), 6.7 (1H, t), 6.9 (1H, dd), 7.1 (1H, d), 7.5 (2H, d), 7.7 (2H, d), 7.9 (1H, s), 8.0 (1H, s), m/z 435 (M+H)⁺.

### EXAMPLE 126

### 3-[[5-[4-(4-cyanophenyl)piperidine-1-carbonyl]-2-methyl-phenyl]carbamoylamino]propanamide

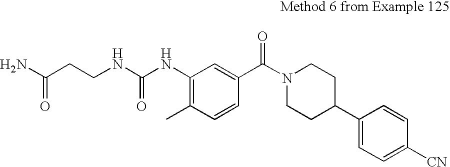

¹H NMR (300.073 MHz, d₆-DMSO) δ 1.4-2.0 (4H, m), 2.2 (3H, s), 2.3 (2H, t), 2.8-3.2 (2H, m), 3.3 (2H, m), 3.6-4.0 (1H, m), 4.4-4.6 (1H, m), 6.7 (1H, t), 6.8 (1H, bs), 6.9 (1H, dd), 7.1 (1H, d), 7.3 (1H, bs), 7.5 (2H, d), 7.8 (2H, d), 7.8 (1H, s), 8.0 (1H, s), m/z 434 (M+H)⁺.

### EXAMPLE 127

### 1-[5-[4-(4-cyanophenyl)piperidine-1-carbonyl]-2-methyl-phenyl]-3-(3-morpholin-4-yl-3-oxo-propyl)urea

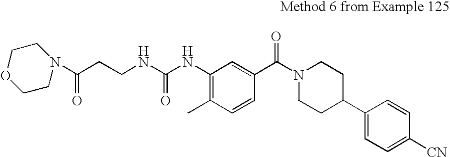

¹H NMR (300.073 MHz, d₆-DMSO) δ 1.50-1.80 (4H, m), 2.26 (3H, s), 2.27-2.87 (1H, m), 2.67-2.72 (1H, m), 2.88-3.18 (3H, m), 3.30-3.33 (2H, m), 3.43-3.54 (8H, m), 3.65-3.98 (1H, m), 4.58-4.62 (1H, m), 6.74 (1H, bt), 6.90 (1H, d, J7.7), 7.15 (1H, d, J7.9), 7.49 (2H, d, J8.3), 7.75 (1H, d, J8.3), 7.87 (1H, s), 7.95 (1H, s), m/z 504 (M+H)⁺.

### EXAMPLE 128

### 3-[[5-[4-(4-cyanophenyl)piperidine-1-carbonyl]-2-methyl-phenyl]carbamoylamino]-N-(2-methoxyethyl)propanamide

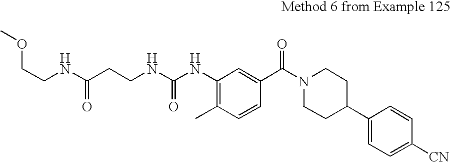

¹H NMR (300.073 MHz, d₆-DMSO) δ 1.57-1.78 (4H, m), 2.18 (3H, s), 2.28 (2H, t, J6.4), 2.68-3.02 (3H, m), 3.21-3.36 (6H, m), 3.23 (3H, s), 3.73-3.83 (1H, m), 4.50-4.61 (1H, m), 6.68 (1H, t, J5.7), 6.90 (1H, dd, J8.0, 1.7), 7.15 (1H, d, J8.0), 7.49 (2H, d, J8.3), 7.76 (2H, d, J8.3), 7.81 (1H, s), 7.96 (2H, bs), m/z 492 (M+H)⁺.

### EXAMPLE 129

### 3-[[5-[4-(4-cyanophenyl)piperidine-1-carbonyl]-2-methyl-phenyl]carbamoylamino]-N-propan-2-yl-propanamide

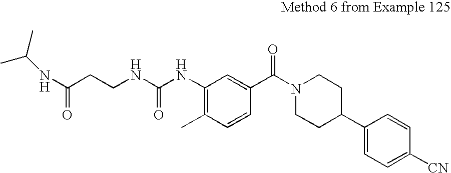

¹H NMR (300.073 MHz, d₆-DMSO) δ 1.04 (3H, s), 1.06 (3H, s), 1.36-1.89 (4H, m), 2.19 (3H, s), 2.24 (2H, t, J6.3), 2.74-3.24 (3H, m), 3.24-3.29 (2H, m), 3.69-3.80 (1H, m), 3.87 (1H, p, J6.6), 4.64-4.78 (1H, m), 6.72 (1H, t, J6.0), 6.92 (1H, dd, J7.6, 1.5), 7.17 (1H, d, J7.8), 7.51 (2H, d, J8.3), 7.78 (3H, d, J8.3), 7.85 (1H, s), 7.97 (1H, d, J1.5), m/z 476 (M+H)⁺.

### EXAMPLE 130

### 3-[[5-[4-(4-cyanophenyl)piperidine-1-carbonyl]-2-methyl-phenyl]carbamoylamino]-N,N-dimethyl-propanamide

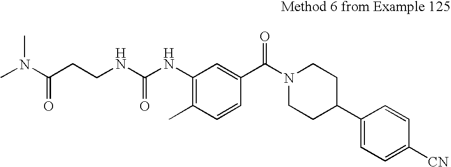

¹H NMR (300.073 MHz, d₆-DMSO) δ 1.58-1.99 (4H, m), 2.19 (3H, s), 2.45-2.50 (2H, m), 2.85 (3H, s), 2.94 (3H, s), 2.70-2.99 (3H, m), 3.30 (2H, m), 3.75-3.93 (1H, m), 4.5-4.7 (1H, m), 6.78 (1H, t, J6.3), 6.91 (1H, dd, J7.6, 1.5), 7.17 (1H, d, J8.3), 7.51 (2H, d, J8.3), 7.78 (2H, d, J8.3), 7.91 (1H, s), 7.98 (1H, d, J1.5), m/z 462 (M+H)⁺.

### EXAMPLE 131

### 1-[5-[4-(4-cyanophenyl)piperidine-1-carbonyl]-2-methyl-phenyl]-3-[3-(2-oxopyrrolidin-1-yl)propyl]urea

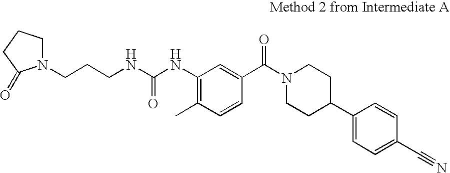

¹H NMR (300.073 MHz, d₆-DMSO) δ 1.49-2.01 (m, 8H), 2.14-2.29 (m, 5H), 2.76-3.12 (m, 5H), 3.21 (t, 2H), 3.25-3.38 (m, 2H), 3.57-4.00 (m, 1H), 4.38-4.80 (m, 1H), 6.62 (t, 1H), 6.91 (d, 1H), 7.17 (d, 1H), 7.49 (d, 2H), 7.71-7.83 (m, 3H), 7.93 (s, 1H), m/z 488 (M+H)⁺.

### EXAMPLE 132

### ethyl 4-[[5-[4-(4-cyanophenyl)piperidine-1-carbonyl]-2-methyl-phenyl]carbamoylamino]piperidine-1-carboxylate

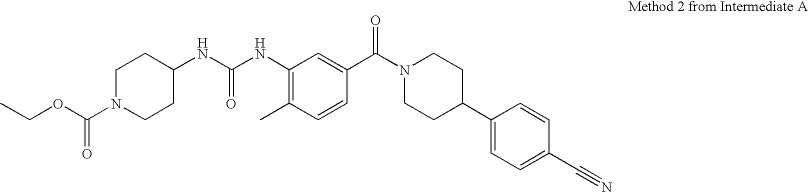

¹H NMR (300.073 MHz, d₆-DMSO) δ 1.06-1.36 (m, 5H), 1.48-1.93 (m, 6H), 2.19 (s, 3H), 2.73-3.21 (m, 5H), 3.52-3.92 (m, 4H), 4.03 (q, 2H), 4.34-4.83 (m, 1H), 6.67 (d, 1H), 6.91 (d, 1H), 7.17 (d, 1H), 7.49 (d, 2H), 7.65 (s, 1H), 7.76 (d, 2H), 7.97 (s, 1H), m/z 518 (M+H)⁺.

### EXAMPLE 133

### 1-[5-[4-(4-cyanophenyl)piperidine-1-carbonyl]-2-methyl-phenyl]-3-(2-oxoazepan-3-yl)urea

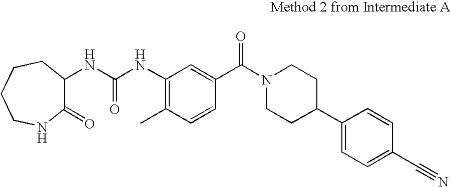

¹H NMR (300.073 MHz, d₆-DMSO) δ 1.09-1.45 (m, 2H), 1.48-1.99 (m, 8H), 2.21 (s, 3H), 2.75-3.24 (m, 5H), 3.55-4.05 (m, 1H), 4.25-4.39 (m, 1H), 4.44-4.79 (m, 1H), 6.91 (d, 1H), 7.16 (d, 2H), 7.49 (d, 2H), 7.75 (d, 2H), 7.83 (t, 2H), 7.94 (s, 1H), 8.22 (s, 1H), m/z 474 (M+H)⁺.

### EXAMPLE 134

### (1S)-2-[[5-[4-(4-cyanophenyl)piperidine-1-carbonyl]-2-methyl-phenyl]carbamoylamino]cyclohexane-1-carboxamide

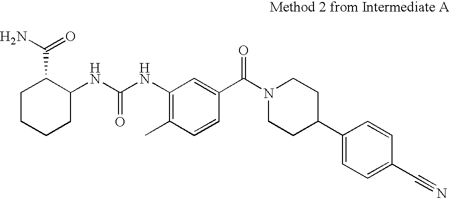

¹H NMR (300.073 MHz, d₆-DMSO) δ 1.19-2.00 (m, 12H), 2.20 (s, 3H), 2.66-3.20 (m, 4H), 3.58-3.93 (m, 1H), 3.96-4.09 (m, 1H), 4.45-4.76 (m, 1H), 6.65-6.77 (m, 2H), 6.89 (d, 1H), 7.15 (d, 1H), 7.23 (s, 1H), 7.49 (d, 2H), 7.76 (d, 2H), 7.96 (d, 2H), m/z 488 (M+H)⁺.

### EXAMPLE 135

### 1-[5-[4-(4-cyanophenyl)piperidine-1-carbonyl]-2-methyl-phenyl]-3-[2-(2-oxoimidazolidin-1-yl)ethyl]urea

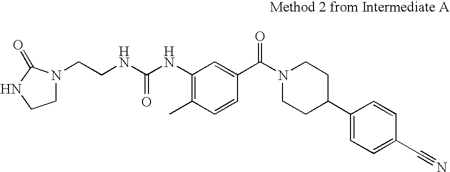

¹H NMR (300.073 MHz, d₆-DMSO) δ 1.46-1.95 (m, 4H), 2.19 (s, 3H), 2.66-3.04 (m, 3H), 3.08-3.16 (m, 2H), 3.16-3.26 (m, 4H), 3.31-3.43 (m, 2H), 3.60-3.98 (m, 1H), 4.45-4.76 (m, 1H), 6.29 (s, 1H), 6.61 (t, 1H), 6.92 (d, 1H), 7.17 (d, 1H), 7.50 (d, 2H), 7.71-7.82 (m, 3H), 7.93 (s, 1H), m/z 475 (M+H)⁺.

### EXAMPLE 136

### 1-[5-[4-(4-cyanophenyl)piperidine-1-carbonyl]-2-methyl-phenyl]-3-[(3-oxo-1,2-oxazol-5-yl)methyl]urea

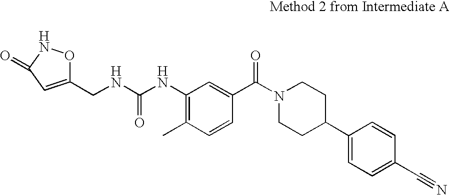

¹H NMR (300.073 MHz, d₆-DMSO) δ 1.44-2.00 (m, 4H), 2.21 (s, 3H), 2.77-3.14 (m, 3H), 3.66-3.99 (m, 1H), 4.30 (d, 2H), 4.44-4.82 (m, 1H), 5.85 (s, 1H), 6.96 (d, 1H), 7.13 (t, 1H), 7.19 (d, 1H), 7.49 (d, 2H), 7.76 (d, 2H), 7.91 (s, 2H), 11.11 (s, 1H), m/z 460 (M+H)⁺.

### EXAMPLE 137

### 1-[5-[4-(4-cyanophenyl)piperidine-1-carbonyl]-2-methyl-phenyl]-3-(2-pyrrolidin-1-ylethyl)urea

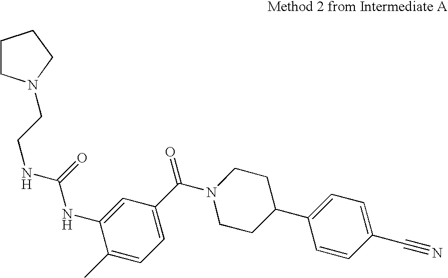

¹H NMR (300.072 MHz, CDCl₃) δ1.50-2.05 (6H, m), 2.15 (3H, s), 2.53-2.59 (2H, m), 2.55-2.61 (4H, m), 2.64 (2H, d), 2.79-3.25 (3H, m), 3.35 (2H, q), 4.00 (1H, m), 4.85 (1H, m), 5.95 (1H, t), 6.98-7.02 (1H, m), 7.11 (1H, d), 7.31 (3H, t), 7.59-7.61 (2H, m), 7.68 (1H, d), m/z 460 (M+H)⁺.

### EXAMPLE 138

### 1-[5-[4-(4-cyanophenyl)piperidine-1-carbonyl]-2-methyl-phenyl]-3-(3-dimethylaminopropyl)urea

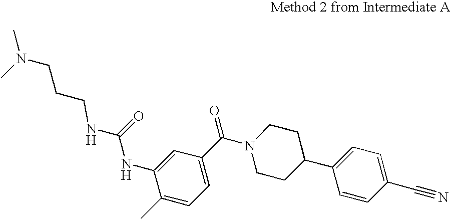

¹H NMR (300.072 MHz, CDCl₃) δ1.55-2.10 (6H, m), 2.17 (9H, d), 2.35 (2H, t), 2.48 (2H, s), 2.80-2.88 (1H, m), 3.00-3.28 (3H, m), 4.00 (1H, m), 4.85 (1H, m), 6.15 (1H, m), 7.00-7.03 (1H, m), 7.12 (2H, d), 7.32 (2H, d), 7.59-7.64 (3H, m), m/z 448 (M+H)⁺.

### EXAMPLE 139

### 1-[5-[4-(4-cyanophenyl)piperidine-1-carbonyl]-2-methyl-phenyl]-3-[2-(1H-imidazol-4-yl)ethyl]urea

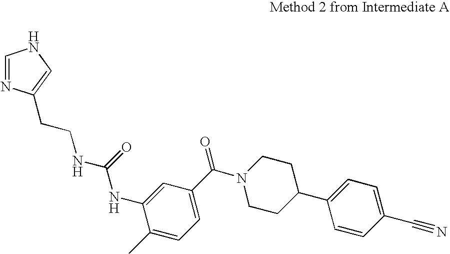

¹H NMR (300.072 MHz, CDCl₃) δ1.50-2.10 (4H, m), 2.05 (3H, s), 2.70 (2H, t), 2.83-3.20 (3H, m), 3.41-3.44 (2H, m), 3.95 (1H, m), 4.80 (1H, m), 5.20 (1H, s), 6.56 (1H, t), 6.64 (1H, s), 6.89-6.92 (1H, m), 7.05 (1H, d), 7.31 (3H, d), 7.58 (2H, d), 7.85 (1H, s), 7.89 (1H, d), m/z 457 (M+H)⁺.

### EXAMPLE 140

### 1-[5-[4-(4-cyanophenyl)piperidine-1-carbonyl]-2-methyl-phenyl]-3-[3-(4-methylpiperazin-1-yl)propyl]urea

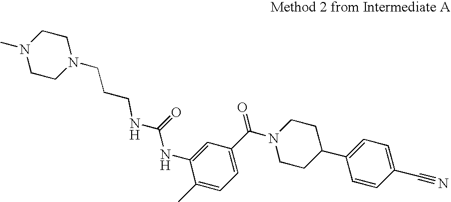

¹H NMR (300.072 MHz, CDCl₃) δ1.50-2.10 (6H, m), 2.16 (3H, s), 2.25 (3H, s), 2.30-2.55 (10H, m), 2.80-3.20 (3H, m), 3.29 (2H, q), 4.00 (1H, m), 4.85 (1H, m), 5.98 (1H, s), 6.61 (1H, s), 7.01-7.04 (1H, m), 7.13 (1H, d), 7.32 (2H, d), 7.59-7.62 (3H, m), m/z 503 (M+H)⁺.

### EXAMPLE 141

### 3-[3-(bis(2-hydroxyethyl)amino)propyl]-1-[5-[4-(4-cyanophenyl)piperidine-1-carbonyl]-2-methyl-phenyl]urea

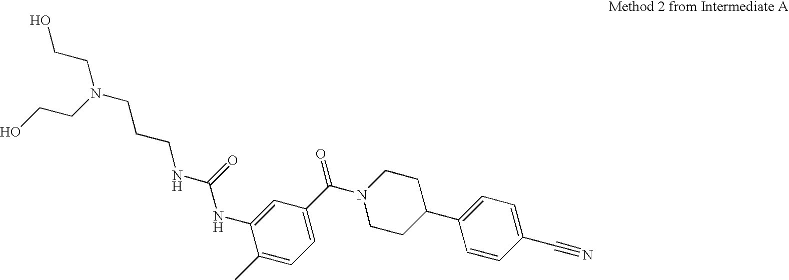

¹H NMR (300.072 MHz, CDCl₃) δ1.55-2.05 (6H, m), 2.06 (3H, s), 2.58 (4H, d), 2.60 (2H, s), 2.83-3.25 (3H, m), 3.30-3.32 (2H, m), 3.64 (4H, t), 4.00 (1H, m), 4.85 (1H, m), 6.81 (1H, s), 6.91-6.94 (1H, m), 7.06 (1H, d), 7.12 (1H, s), 7.33 (2H, d), 7.58-7.61 (2H, m), 7.84 (1H, d), m/z 508 (M+H)⁺.

### EXAMPLE 142

### 1-[5-[4-(4-cyanophenyl)piperidine-1-carbonyl]-2-methyl-phenyl]-3-(4-dimethylaminobutyl)urea

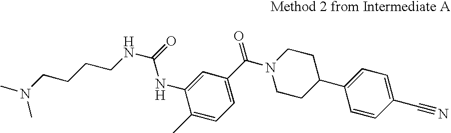

¹H NMR (300.072 MHz, CDCl₃) δ1.47-2.10 (6H, m), 2.11 (3H, s), 2.21 (6H, s), 2.26-2.29 (2H, m), 2.45 (2H, s), 2.79-3.23 (5H, m), 4.00 (1H, m), 4.85 (1H, m), 6.05 (1H, s), 6.96 (1H, d), 6.96 (1H, d), 7.08 (1H, d), 7.32 (2H, d), 7.59-7.61 (2H, m), 7.67 (1H, d), m/z 462 (M+H)⁺.

### EXAMPLE 143

### 3-(1-azabicyclo[2.2.2]oct-8-yl)-1-[5-[4-(4-cyanophenyl)piperidine-1-carbonyl]-2-methyl-phenyl]urea

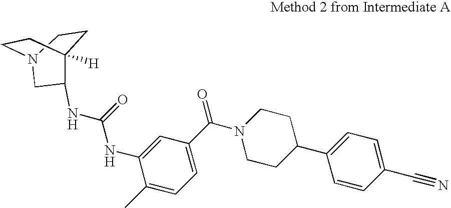

¹H NMR (300.072 MHz, CDCl₃) δ1.69-2.05 (6H, m), 2.13 (7H, d), 2.55 (1H, m), 2.75-3.25 (7H, m), 3.26-3.34 (1H, m), 3.80-4.10 (2H, m), 4.85 (1H,), 6.45 (1H, d), 6.92-6.95 (1H, m), 7.10 (2H, t), 7.27-7.33 (2H, m), 7.60-7.62 (2H, m), 7.68 (1H, d), m/z 472 (M+H)⁺.

### EXAMPLE 144

### 1-[5-[4-(4-cyanophenyl)piperidine-1-carbonyl]-2-methyl-phenyl]-3-[2-(1-methylpyrrolidin-2-yl)ethyl]urea

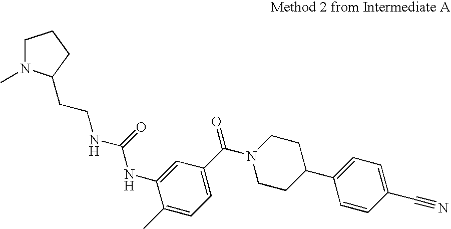

¹H NMR (300.072 MHz, CDCl₃) δ1.46-2.05 (6H, m), 2.15 (5H, m), 2.27 (4H, m), 2.50 (3H, s), 2.80-3.30 (6H, m), 4.00 (1H, m), 4.85 (1H, m), 6.17 (1H, s), 6.98 (2H, d), 7.10 (1H, d), 7.33 (2H, d), 7.60 (3H, d), m/z 474 (M+H)⁺.

### EXAMPLE 145

### 1-[5-[4-(4-cyanophenyl)piperidine-1-carbonyl]-2-methyl-phenyl]-3-(2-dimethylaminoethyl)urea

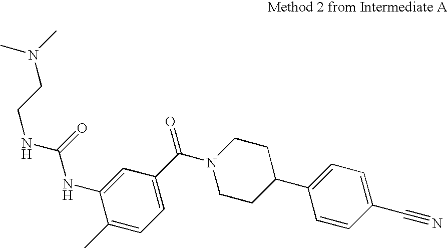

¹H NMR (300.072 MHz, CDCl₃) δ1.55-2.05 (4H, m), 2.15 (3H, s), 2.25 (6H, s), 2.45 (2H, t), 2.79-3.25 (3H, m), 3.31 (2H, q), 4.00 (1H, m), 4.85 (1H, m), 5.97 (1H, t), 6.99-7.02 (1H, m), 7.11 (1H, d), 7.31-7.45 (3H, m), 7.59-7.61 (2H, m), 7.70 (1H, d), m/z 434 (M+H)⁺.

### EXAMPLE 146

### 1-[5-[4-(4-cyanophenyl)piperidine-1-carbonyl]-2-methyl-phenyl]-3-(3-morpholin-4-ylpropyl)urea

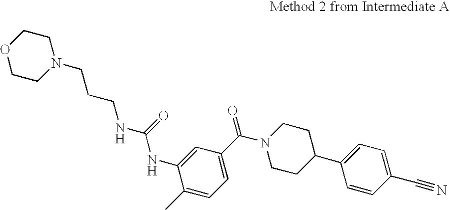

¹H NMR (300.072 MHz, CDCl₃) δ1.50-2.05 (6H, m), 2.09 (3H, s), 2.42 (2H, d), 2.42 (4H, d), 2.80-3.20 (3H, m), 3.24-3.30 (2H, m), 3.66 (4H, t), 4.00 (1H, m), 4.85 (1H, m), 5.97 (1H, s), 6.82 (1H, s), 6.95-6.98 (1H, m), 7.08 (1H, d), 7.32 (2H, d), 7.57 (1H, d), 7.61 (2H, d), m/z 490 (M+H)⁺.

### EXAMPLE 147

### 1-[5-[4-(4-cyanophenyl)piperidine-1-carbonyl]-2-methyl-phenyl]-3-[1-(2-methoxyethyl)-4-piperidyl]urea

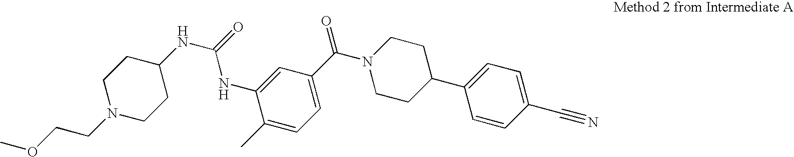

¹H NMR (300.072 MHz, CDCl₃) δ1.20-2.15 (10H, m), 2.15 (3H, s), 2.55 (2H, t), 2.79-3.25 (5H, m), 3.39 (3H, s), 3.52 (2H, t), 3.58-3.63 (1H, m), 4.00 (1H, m), 4.85 (1H, m), 5.74 (1H, d), 6.93-6.96 (1H, m), 7.10 (1H, d), 7.31 (2H, d), 7.60 (2H, d), 7.75 (1H, d), m/z 504 (M+H)⁺.

### EXAMPLE 148

### 1-[5-[4-(4-cyanophenyl)piperidine-1-carbonyl]-2-methyl-phenyl]-3-(1-methyl-4-piperidyl)urea

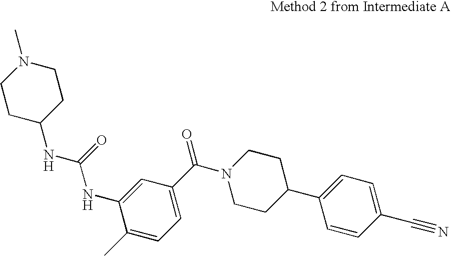

¹H NMR (300.072 MHz, CDCl₃) δ1.40-2.20 (11H, m), 2.28 (3H, s), 2.64-3.25 (7H, m), 3.59-3.67 (1H, m), 3.95 (1H, m), 4.85 (1H, m), 5.85 (1H, d), 6.90-6.93 (1H, m), 7.02 (2H, d), 7.32 (2H, d), 7.57-7.62 (3H, m), m/z 460 (M+H)⁺.

### EXAMPLE 149

### 3-[5-[4-(4-cyanophenyl)piperidine-1-carbonyl]-2-methyl-phenyl]-1-[[(2S)-1-ethylpyrrolidin-2-yl]methyl]urea

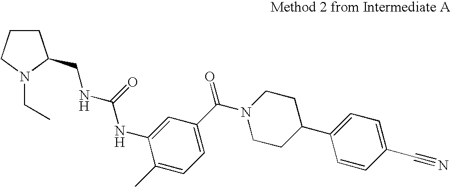

¹H NMR (300.072 MHz, CDCl₃) δ1.08 (3H, t), 1.55-2.10 (6H, m), 2.15-2.34 (5H, m), 2.51-2.59 (1H, m), 2.56-2.62 (1H, m), 2.80-3.20 (6H, m), 3.37-3.45 (1H, m), 4.00 (1H, m), 4.85 (1H, m), 5.87 (1H, s), 6.99-7.02 (1H, m), 7.11 (1H, d), 7.32 (2H, d), 7.60 (2H, d), 7.66 (1H, d), m/z 474 (M+H)⁺.

### EXAMPLE 150

### 1-[5-[4-(4-cyanophenyl)piperidine-1-carbonyl]-2-methyl-phenyl]-3-[(3-methylimidazol-4-yl)methyl]urea

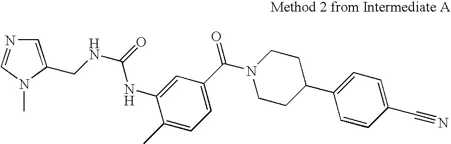

¹H NMR (300.072 MHz, CDCl₃) δ1.50-2.05 (4H, m), 2.07 (3H, s), 2.80-2.88 (2H, m), 3.14 (1H, s), 3.61 (3H, s), 4.00 (1H, m), 4.37 (2H, d), 4.77 (1H, s), 6.38 (1H, t), 6.81 (1H, s), 6.91-6.94 (1H, m), 7.06 (1H, d), 7.32 (2H, d), 7.37 (1H, s), 7.46 (1H, s), 7.59-7.62 (2H, m), 7.72 (1H, d), m/z 457 (M+H)⁺.

### EXAMPLE 151

### 1-[5-[4-(4-cyanophenyl)piperidine-1-carbonyl]-2-methyl-phenyl]-3-(oxolan-2-ylmethyl)urea

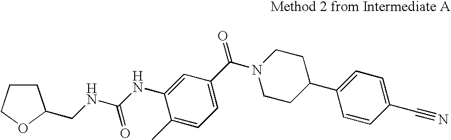

¹H NMR (300.072 MHz, CDCl₃) [symbol] 1.58-2.03 (m, 8H), 2.19 (s, 3H), 2.78-2.87 (m, 2H), 2.96-3.10 (m, 1H), 3.10-3.20 (m, 1H), 3.47-3.58 (m, 1H), 3.71-3.78 (m, 1H), 3.82-3.89 (m, 1H), 3.96-4.03 (m, 1H), 4.04-4.11 (m, 1H), 4.76-5.00 (m, 1H), 5.32-5.38 (m, 1H), 6.94 (s, 1H), 7.04-7.16 (m, 2H), 7.32 (d, 2H), 7.61 (d, 2H), 7.65 (s, 1H), m/z 447 (M+H)⁺.

### EXAMPLE 152

### 1-[5-[4-(4-cyanophenyl)piperidine-1-carbonyl]-2-methyl-phenyl]-3-(2-methoxyethyl)urea

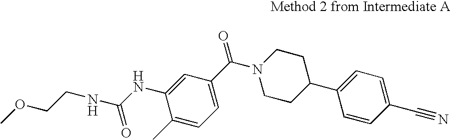

¹H NMR (300.072 MHz, CDCl₃) [symbol] 1.62-1.98 (m, 4H), 2.20 (s, 3H), 2.79-2.90 (m, 2H), 2.96-3.17 (m, 1H), 3.37 (s, 3H), 3.40-3.45 (m, 2H), 3.48-3.51 (m, 2H), 3.90-4.19 (m, 1H), 4.71-5.00 (m, 1H), 5.20 (s, 1H), 6.62 (s, 1H), 7.05-7.17 (m, 2H), 7.32 (d, 2H), 7.57-7.65 (m, 3H), m/z 421 (M+H)⁺.

### EXAMPLE 153

### 1-[5-[4-(4-cyanophenyl)piperidine-1-carbonyl]-2-methyl-phenyl]-3-(3-propan-2-yloxypropyl)urea

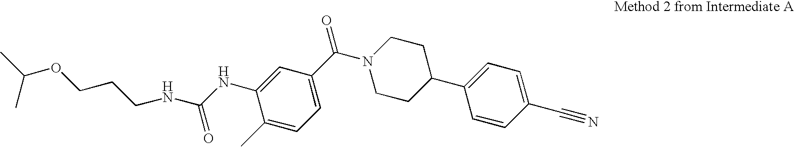

¹H NMR (300.072 MHz, CDCl₃) [symbol] 1.09 (d, 6H), 1.61-1.96 (m, 4H), 1.77 (quintet, 2H), 2.18 (s, 3H), 2.79-2.90 (m, 2H), 2.95-3.20 (m, 1H), 3.34 (q, 2H), 3.49 (t, 2H), 3.53 (septet, 1H), 3.89-4.15 (m, 1H), 4.72-4.98 (m, 1H), 5.43 (t, 1H), 6.33 (s, 1H), 7.04-7.17 (m, 2H), 7.32 (d, 2H), 7.54-7.56 (m, 1H), 7.61 (d, 2H), m/z 463 (M+H)⁺.

### EXAMPLE 154

### 1-[5-[4-(4-cyanophenyl)piperidine-1-carbonyl]-2-methyl-phenyl]-3-(3-methoxypropyl)urea

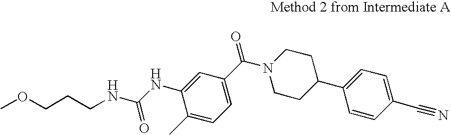

¹H NMR (300.072 MHz, CDCl₃) [symbol] 1.64-1.97 (m, 4H), 1.78 (quintet, 2H), 2.19 (s, 3H), 2.79-2.90 (m, 2H), 3.05-3.16 (m, 1H), 3.28 (s, 3H), 3.34 (q, 2H), 3.46 (t, 2H), 3.89-4.09 (m, 1H), 4.76-4.94 (m, 1H), 5.33-5.41 (m, 1H), 6.33 (s, 1H), 7.05-7.19 (m, 2H), 7.33 (d, 2H), 7.53-7.56 (m, 1H), 7.61 (d, 2H), m/z 435 (M+H)⁺.

### EXAMPLE 155

### 1-[5-[4-(4-cyanophenyl)piperidine-1-carbonyl]-2-methyl-phenyl]-3-(1-methoxypropan-2-yl)urea

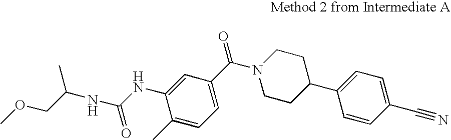

¹H NMR (300.072 MHz, CDCl₃) [symbol] 1.19 (d, 3H), 1.53-1.96 (m, 4H), 2.12 (s, 3H), 2.78-2.88 (m, 2H), 3.08-3.23 (m, 1H), 3.33-3.43 (m, 2H), 3.37 (s, 3H), 3.91-4.10 (m, 2H), 4.75-4.94 (m, 1H), 5.37 (d, 1H), 6.93 (s, 1H), 6.98-7.10 (m, 2H), 7.32 (d, 2H), 7.57-7.63 (m, 3H), m/z 435 (M+H)⁺.

### EXAMPLE 156

### 1-[5-[4-(4-cyanophenyl)piperidine-1-carbonyl]-2-methyl-phenyl]-3-(2-methylsulfanylethyl)urea

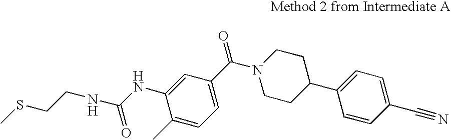

¹H NMR (300.072 MHz, CDCl₃) [symbol] 1.64-1.80 (m, 4H), 2.09 (s, 3H), 2.12 (s, 3H), 2.66 (t, 2H), 2.79-2.90 (m, 2H), 3.10-3.19 (m, 1H), 3.44 (q, 2H), 3.86-4.07 (m, 1H), 4.81-4.99 (m, 1H), 5.68 (t, 1H), 6.68 (s, 1H), 6.98-7.11 (m, 2H), 7.32 (d, 2H), 7.49-7.52 (m, 1H), 7.61 (d, 2H), m/z 437 (M+H)⁺.

### EXAMPLE 157

### 1-[5-[4-(4-cyanophenyl)piperidine-1-carbonyl]-2-methyl-phenyl]-3-(3-methylsulfanylpropyl)urea

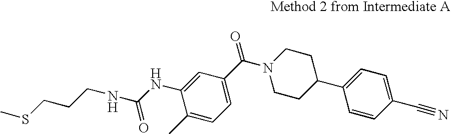

¹H NMR (300.072 MHz, CDCl₃) [symbol] 1.63-1.96 (m, 4H), 1.83 (quintet, 2H), 2.10 (s, 3H), 2.13 (s, 3H), 2.55 (t, 2H), 2.77-2.88 (m, 2H), 2.93-3.05 (m, 1H), 3.34 (q, 2H), 3.89-4.07 (m, 1H), 4.77-4.98 (m, 1H), 5.33 (t, 1H), 6.36 (s, 1H), 7.01-7.15 (m, 2H), 7.33 (d, 2H), 7.49-7.53 (m, 1H), 7.62 (d, 2H), m/z 451 (M+H)⁺.

### EXAMPLE 158

### 3-[5-[4-(4-cyanophenyl)piperidine-1-carbonyl]-2-methyl-phenyl]-1-(3-ethoxypropyl)urea

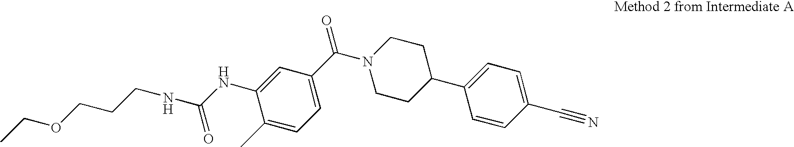

¹H NMR (300.072 MHz, CDCl₃) δ 1.12 (t, 3H), 1.68-2.04 (m, 4H), 1.79 (quintet, 2H), 2.17 (s, 3H), 2.80-2.88 (m, 2H), 2.95-3.13 (m, 1H), 3.34 (q, 2H), 3.40-3.52 (m, 4H), 3.86-4.11 (m, 1H), 4.67-5.00 (m, 1H), 5.43 (t, 1H), 6.38 (s, 1H), 7.04-7.16 (m, 2H), 7.32 (d, 2H), 7.53-7.54 (m, 1H), 7.61 (d, 2H), m/z 449 (M+H)⁺.

### EXAMPLE 159

### 1-[5-[4-(4-cyanophenyl)piperidine-1-carbonyl]-2-methyl-phenyl]-3-(2-methoxy-2-methyl-propyl)urea

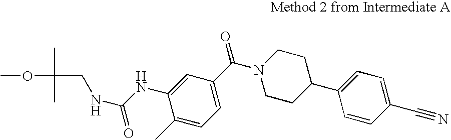

¹H NMR (300.072 MHz, CDCl₃) [symbol] 1.19 (s, 6H), 1.63-1.95 (m, 4H), 2.16 (s, 3H), 2.79-2.87 (m, 2H), 2.97-3.08 (m, 1H), 3.21 (s, 3H), 3.27 (d, 2H), 3.93-4.11 (m, 1H), 4.80-5.00 (m, 1H), 5.35-5.41 (m, 1H), 6.93 (s, 1H), 7.01-7.14 (m, 2H), 7.32 (d, 2H), 7.57-7.65 (m, 3H), m/z 449 (M+H)⁺.

### EXAMPLE 160

### 1-[5-[4-(4-cyanophenyl)piperidine-1-carbonyl]-2-methyl-phenyl]-3-(1,4-dioxan-2-ylmethyl)urea

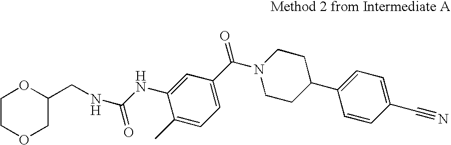

¹H NMR (300.072 MHz, CDCl₃) [symbol] 1.65-1.94 (m, 4H), 2.15 (s, 3H), 2.78-2.91 (m, 2H), 3.06-3.21 (m, 2H), 3.32-3.83 (m, 8H), 3.91-4.10 (m, 1H), 4.76-4.96 (m, 1H), 5.39 (t, 1H), 6.70 (s, 1H), 7.02-7.15 (m, 2H), 7.32 (d, 2H), 7.53-7.55 (m, 1H), 7.61 (d, 2H), m/z 463 (M+H)⁺.

### EXAMPLE 161

### 1-[5-[4-(4-cyanophenyl)piperidine-1-carbonyl]-2-methyl-phenyl]-3-[3-(2-methoxyethoxy)propyl]urea

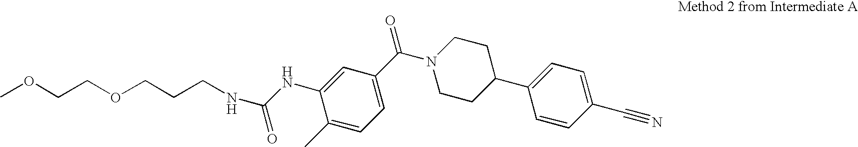

¹H NMR (300.072 MHz, CDCl₃) δ 1.62-1.96 (m, 4H), 1.80 (quintet, 2H), 2.23 (s, 3H), 2.77-2.89 (m, 2H), 3.04-3.18 (m, 1H), 3.35-3.41 (m, 2H), 3.38 (s, 3H), 3.54-3.65 (m, 6H), 3.93-4.21 (m, 1H), 4.69-5.00 (m, 1H), 5.72-5.80 (m, 1H), 6.44 (s, 1H), 7.06-7.20 (m, 2H), 7.32 (d, 2H), 7.60 (d, 2H), 7.72-7.77 (m, 1H), m/z 479 (M+H)⁺.

### EXAMPLE 162

### 1-[5-[4-(4-cyanophenyl)piperidine-1-carbonyl]-2-methyl-phenyl]-3-(oxan-4-yl)urea

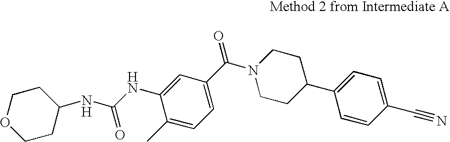

¹H NMR (400.132 MHz, CDCl₃) [symbol] 1.44-1.55 (m, 2H), 1.58-1.80 (m, 4H), 1.89-1.96 (m, 2H), 1.99 (s, 3H), 2.80-2.93 (m, 2H), 3.10-3.21 (m, 1H), 3.49 (t, 2H), 3.81-3.87 (m, 1H), 3.94-3.99 (m, 3H), 4.83-4.95 (m, 1H), 5.67 (d, 1H), 6.64 (s, 1H), 6.92-7.06 (m, 2H), 7.33 (d, 2H), 7.45-7.50 (m, 1H), 7.62 (d, 2H), m/z 447 (M+H)⁺.

### EXAMPLE 163

### 1-[5-[4-(4-cyanophenyl)piperidine-1-carbonyl]-2-methyl-phenyl]-3-(2,6-dioxabicyclo[5.4.0]undeca-8,10,12-trien-4-yl)urea

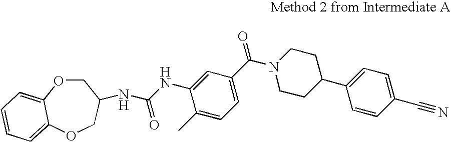

¹H NMR (400.132 MHz, CDCl₃) δ 1.48-1.83 (m, 4H), 2.05 (s, 3H), 2.59-2.81 (m, 2H), 2.99-3.17 (m, 1H), 3.89-3.96 (m, 1H), 4.08-4.12 (m, 2H), 4.25-4.30 (m, 2H), 4.34-4.43 (m, 1H), 4.72-4.82 (m, 1H), 6.48 (d, 1H), 6.91-7.05 (m, 6H), 7.20 (s, 1H), 7.27 (d, 2H), 7.47-7.50 (m, 1H), 7.60 (d, 2H), m/z 511 (M+H)⁺.

### EXAMPLE 164

### 3-[5-[4-(4-cyanophenyl)piperidine-1-carbonyl]-2-methyl-phenyl]-1-(2-propoxyethyl)urea

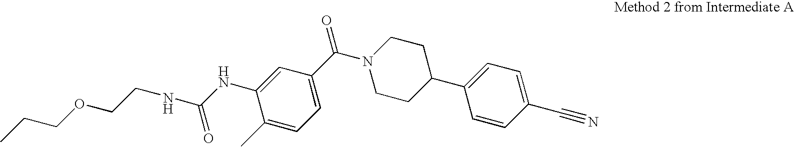

¹H NMR (400.132 MHz, CDCl₃) [symbol] 0.89 (t, 3H), 1.52-1.58 (m, 2H), 1.71-2.03 (m, 4H), 2.23 (s, 3H), 2.80-2.88 (m, 2H), 3.08-3.22 (m, 1H), 3.39-3.45 (m, 4H), 3.53 (t, 2H), 3.95-4.09 (m, 1H), 4.79-4.94 (m, 1H), 5.15 (t, 1H), 6.52 (s, 1H), 7.09-7.20 (m, 2H), 7.33 (d, 2H), 7.59-7.63 (m, 3H), m/z 449 (M+H)⁺.

### EXAMPLE 165

### 1-[5-[4-(4-cyanophenyl)piperidine-1-carbonyl]-2-methyl-phenyl]-3-(7,10-dioxabicyclo[4.4.0]deca-2,4,11-trien-8-ylmethyl)urea

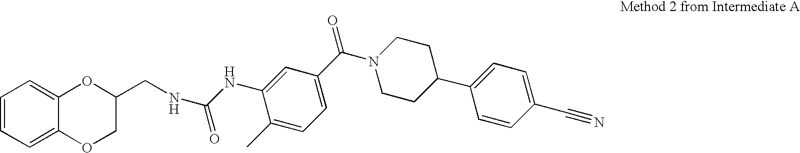

¹H NMR (400.132 MHz, CDCl₃) [symbol] 1.70-1.98 (m, 4H), 2.09 (s, 3H), 2.76-2.88 (m, 2H), 3.08-3.24 (m, 1H), 3.46-3.64 (m, 2H), 3.92-4.01 (m, 2H), 4.26-4.34 (m, 2H), 4.80-4.91 (m, 1H), 5.77 (t, 1H), 6.77 (s, 1H), 6.82-6.90 (m, 4H), 6.97-7.08 (m, 2H), 7.31 (d, 2H), 7.41 (s, 1H), 7.62 (d, 2H), m/z 511 (M+H)⁺.

### EXAMPLE 166

### 1-[5-[4-(4-cyanophenyl)piperidine-1-carbonyl]-2-methyl-phenyl]-3-(oxan-2-ylmethyl)urea

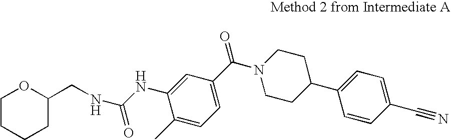

¹H NMR (400.132 MHz, CDCl₃) [symbol] 1.22-1.61 (m, 6H), 1.76-2.01 (m, 4H), 2.18 (s, 3H), 2.80-2.91 (m, 2H), 3.04-3.19 (m, 2H), 3.40-3.49 (m, 3H), 3.93-4.05 (m, 2H), 4.82-4.93 (m, 1H), 5.40 (s, 1H), 6.96 (s, 1H), 7.03-7.15 (m, 2H), 7.32 (d, 2H), 7.62 (d, 2H), 7.66 (s, 1H), m/z 461 (M+H)⁺.

### EXAMPLE 167

### 3-(cyanomethyl)-1-[5-[4-(4-cyanophenyl)piperidine-1-carbonyl]-2-methyl-phenyl]urea

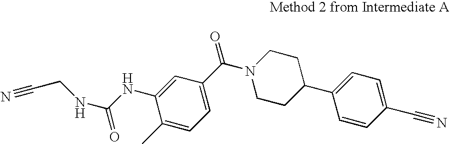

¹H NMR (400.132 MHz, CDCl₃) [symbol] 1.59-1.81 (m, 3H), 1.96 (s, 3H), 1.99-2.07 (m, 1H), 2.82-2.94 (m, 2H), 3.11-3.21 (m, 1H), 3.92-3.99 (m, 1H), 4.12 (d, 2H), 4.86-4.89 (m, 1H), 6.57 (t, 1H), 6.94-7.09 (m, 2H), 7.18 (s, 1H), 7.34 (d, 2H), 7.45-7.47 (m, 1H), 7.62 (d, 2H), m/z 402 (M+H)⁺.

### EXAMPLE 168

### 1-[5-[4-(4-cyanophenyl)piperidine-1-carbonyl]-2-methyl-phenyl]-3-(2,2,2-trifluoroethyl)urea

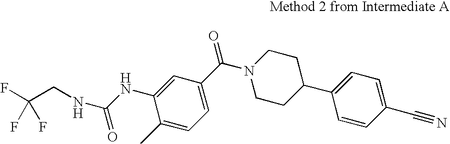

¹H NMR (400.132 MHz, CDCl₃) [symbol] 1.53-1.80 (m, 3H), 1.95 (s, 3H), 1.98-2.03 (m, 1H), 2.80-2.92 (m, 2H), 3.10-3.18 (m, 1H), 3.85 (quintet, 2H), 3.90-3.97 (m, 1H), 4.81-4.89 (m, 1H), 6.43 (t, 1H), 6.90-7.05 (m, 2H), 7.25 (s, 1H), 7.32 (d, 2H), 7.39-7.41 (m, 1H), 7.62 (d, 2H), m/z 445 (M+H)⁺.

### EXAMPLE 169

### 1-[5-[4-(4-cyanophenyl)piperidine-1-carbonyl]-2-methyl-phenyl]-3-(1,1-dioxothiolan-3-yl)urea

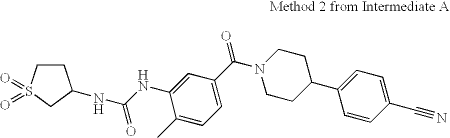

¹H NMR (400.132 MHz, CDCl₃) [symbol] 1.70-1.85 (m, 3H), 1.94 (s, 3H), 1.98-2.04 (m, 1H), 2.26-2.35 (m, 1H), 2.48-2.57 (m, 1H), 2.81-2.95 (m, 2H), 3.05-3.21 (m, 3H), 3.27-3.35 (m, 1H), 3.39-3.44 (m, 1H), 3.95-4.00 (m, 1H), 4.64 (sextet, 1H), 4.83-4.94 (m, 1H), 6.62 (d, 1H), 6.77 (s, 1H), 6.92-7.06 (m, 2H), 7.34 (d, 2H), 7.52 (s, 1H), 7.63 (d, 2H), m/z 481 (M+H)⁺.

### EXAMPLE 170

### 3-[[5-[4-(4-cyanophenyl)piperidine-1-carbonyl]-2-methyl-phenyl]carbamoylamino]-N-pyridin-2-yl-propanamide

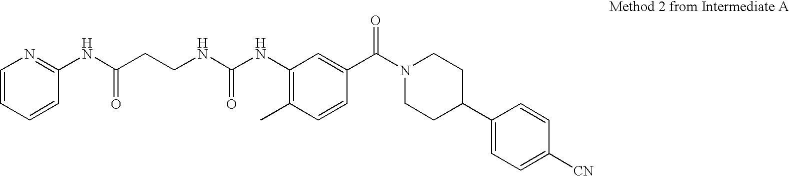

¹H NMR (300.073 MHz, d₆-DMSO) δ 1.54-1.96 (4H, m), 2.17 (3H, s), 2.61 (2H, t, J5.9), 2.84-3.10 (3H, m), 3.38-3.40 (2H, m), 3.62-3.80 (1H, m), 4.49-4.75 (1H, m), 6.76 (1H, t, J5.5), 6.90 (1H, d, J7.1), 7.07 (1H, t, J5.3), 7.15 (1H, d, J7.7), 7.49 (2H, d, J7.9), 7.76 (2H, d, 7.9), 7.70-7.80 (1H, m), 7.80 (1H, s), 7.97 (1H, s), 8.10 (1H, d, J8.2), 8.29 (1H, d, J3.7), 10.49 (1H, bs), m/z 511 (M+H)⁺.

### EXAMPLE 171

### 2-[[5-[4-(4-cyanophenyl)piperidine-1-carbonyl]-2-methyl-phenyl]carbamoylamino]-N-propan-2-yl-acetamide

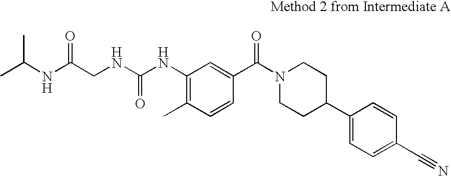

¹H NMR (300.073 MHz, d₆-DMSO, 30° C.) δ 1.05 (6H, d, J=7.3 Hz), 1.46-1.93 (4H, m), 2.20 (3H, s), 2.60-3.19 (3H, m), 3.51-3.95 (4H, m), 4.40-4.70 (1H, m), 6.82-6.96 (2H, m), 7.17 (1H, d J=6.8 Hz), 7.49 (2H, d J=7.3 Hz), 7.70-7.85 (3H, m), 7.94 (1H, s), 8.03 (1H, s), m/z 462 (M+H)⁺.

### EXAMPLE 172

### 1-butyl-3-[2-[[5-[4-(4-cyanophenyl)piperidine-1-carbonyl]-2-methyl-phenyl]carbamoylamino]ethyl]urea

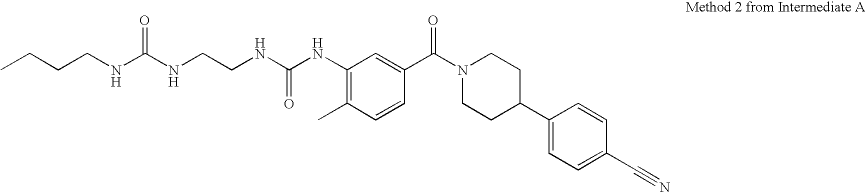

¹H NMR (300.073 MHz, d₆-DMSO, 30° C.) δ 0.85 (3H, t J=8.4 Hz), 1.16-1.40 (4H, m), 1.47-1.95 (4H, m), 2.20 (3H, s), 2.68-3.23 (9H, m), 3.60-4.01 (1H, m appears as a broad flat singlet), 4.37-4.71 (1H, m appears as a broad flat singlet), 5.73-5.91 (2H, m), 6.59-6.70 (1H, m), 6.92 (1H, d J=8.6 Hz), 7.17 (1H, d J=8.1 Hz), 7.49 (2H, d J=8.1 Hz), 7.72-7.82 (3H, m), 7.93 (1H, s), m/z 505 (M+H)⁺.

### EXAMPLE 173

### N-[5-[[5-[4-(4-cyanophenyl)piperidine-1-carbonyl]-2-methyl-phenyl]carbamoylamino]pentyl]morpholine-4-carboxamide

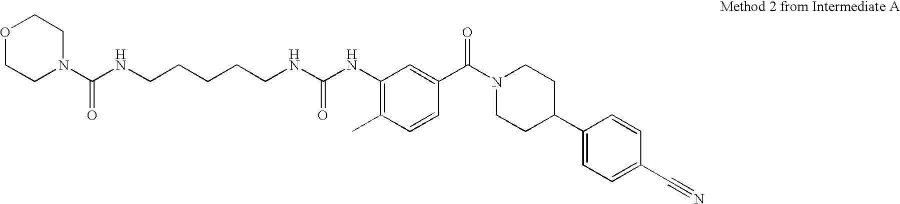

¹H NMR (300.073 MHz, d₆-DMSO, 30° C.) δ 1.21-1.35 (2H, m), 1.36-1.50 (4H, m), 1.50-1.69 (2H, m), 1.69-1.91 (2H, m), 2.20 (3H, s), 2.69-3.15 (7H, m), 3.16-3.25 (4H, m), 3.45-3.56 (4H, m), 3.64-3.96 (1H, m appears as a broad flat singlet), 4.40-4.80 (1H, m appears as a broad flat singlet), 6.45 (1H, t J=5.1 Hz), 6.59 (1H, t J=5.1 Hz), 6.91 (1H, d J=6.8 Hz), 7.16 (1H, d J=7.7 Hz), 7.49 (2H, d J=8.6 Hz), 7.66 (1H, s), 7.76 (2H, d J=8.5 Hz), 7.95 (1H, s), m/z 561 (M+H)⁺.

### EXAMPLE 174

### 3-[5-[4-(4-cyanophenyl)piperidine-1-carbonyl]-2-methyl-phenyl]-1-[2-(propylsulfonylamino)ethyl]urea

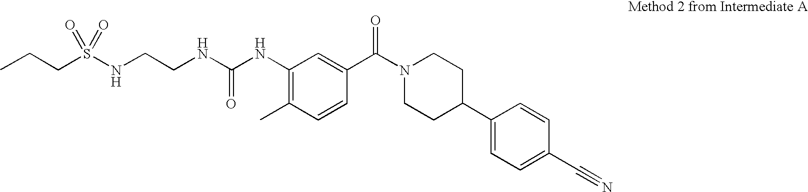

¹H NMR (300.073 MHz, d₆-DMSO, 30° C.) δ 0.95 (3H, t J=7.6 Hz), 1.49-1.93 (6H, m), 2.20 (3H, s), 2.75-3.08 (7H, m), 3.14-3.23 (2H, m), 3.58-3.95 (1H, m appears as a broad flat singlet), 4.34-4.78 (1H, m appears as a broad flat singlet), 6.73 (1H, t J=5.7 Hz), 6.93 (1H, d J=6.7 Hz), 7.08 (1H, t J=5.8 Hz), 7.17 (1H, d J=9.1 Hz), 7.49 (2H, d J=8.5 Hz), 7.76 (2H, d J=8.5 Hz), 7.84 (1H, s), 7.91 (1H, s), m/z 512 (M+H)⁺.

### EXAMPLE 175

### N-[2-[[5-[4-(4-cyanophenyl)piperidine-1-carbonyl]-2-methyl-phenyl]carbamoylamino]ethyl]cyclohexanecarboxamide

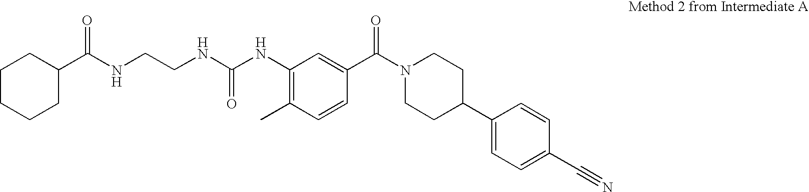

¹H NMR (300.073 MHz, d₆-DMSO, 30° C.) δ 1.02-1.40 (6H, m), 1.45-1.92 (10H, m), 1.96-2.12 (1H, m), 2.19 (3H, s), 2.68-3.20 (7H, m), 3.59-3.96 (1H, m), 4.40-4.80 (1H, m), 6.54-6.65 (1H, m), 6.92 (1H, d J=8.2 Hz), 7.17 (1H, d J=8.9 Hz), 7.49 (2H, d J=8.2 Hz), 7.59-7.73 (1H, m), 7.73-7.83 (3H, m), 7.91 (1H, s), m/z 516 (M+H)⁺.

### EXAMPLE 176

### 1-[5-[4-(4-cyanophenyl)piperidine-1-carbonyl]-2-methyl-phenyl]-3-[2-(2-oxopyrrolidin-1-yl)ethyl]urea

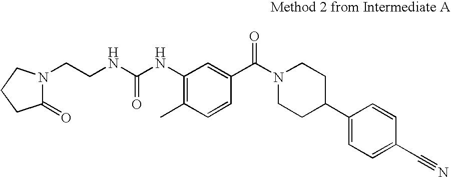

¹H NMR (300.073 MHz, d₆-DMSO, 30° C.) δ 1.46-1.69 (2H, m), 1.70-1.98 (4H, m), 2.08-2.29 (5H, m), 2.67-3.18 (3H, m), 3.18-3.27 (4H, m), 3.32-3.42 (2H, m), 3.60-3.94 (1H, m), 4.41-4.81 (1H, m), 6.53-6.62 (1H, m), 6.93 (1H, d J=7.5 Hz), 7.17 (1H, d J=8.1 Hz), 7.50 (2H, d J=8.1 Hz), 7.72-7.79 (3H, m), 7.88 (1H, s), m/z (M+H)⁺.

### EXAMPLE 177

### Methyl (2S)-3-[[5-[4-(4-cyanophenyl)piperidine-1-carbonyl]-2-methyl-phenyl]carbamoylamino]-2-[(2-methylpropan-2-yl)oxycarbonylamino]propanoate

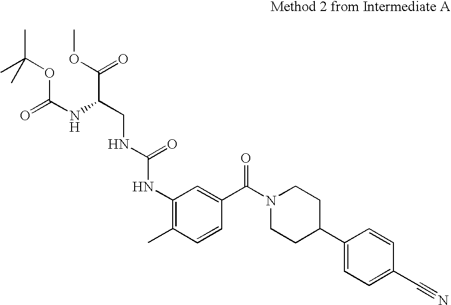

¹H NMR (300.073 MHz, d₆-DMSO, 30° C.) δ 1.37 (9H, s), 1.49-1.94 (4H, m), 2.20 (3H, s), 2.67-3.23 (5H, m), 3.47-3.94 (5H, m), 3.96-4.18 (2H, m), 4.32-4.76 (1H, m), 6.67-6.83 (1H, m), 6.93-6.96 (1H, m), 7.13-7.28 (2H, m), 7.50 (2H, d J=7.2 Hz), 7.74-7.77 (2H, m), 7.88-7.93 (2H, m), m/z 564 (M+H)⁺.

### EXAMPLE 178

### 1-[5-[4-(4-cyanophenyl)piperidine-1-carbonyl]-2-methyl-phenyl]-3-[3-(2-oxoazepan-1-yl)propyl]urea

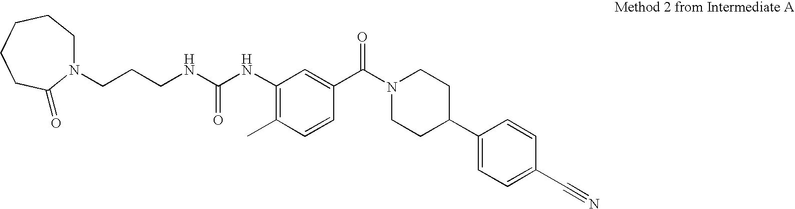

¹H NMR (300.073 MHz, d₆-DMSO, 30° C.) δ 1.42-1.95 (12H, m), 2.20 (3H, s), 2.33-2.45 (2H, m), 2.69-3.22 (6H, m), 3.24-3.40 (3H, m), 3.61-3.95 (1H, m), 4.39-4.79 (1H, m), 6.61 (1H, t J=5.3 Hz), 6.92 (1H, d J=9.7 Hz), 7.17 (1H, d J=8.1 Hz), 7.50 (2H, d J=8.8 Hz), 7.71-7.85 (3H, m), 7.92 (1H, s), m/z 516 (M+H)⁺.

### EXAMPLE 179

### 3-[[5-[4-(4-cyanophenyl)piperidine-1-carbonyl]-2-methyl-phenyl]carbamoylamino]-N-methyl-propanamide

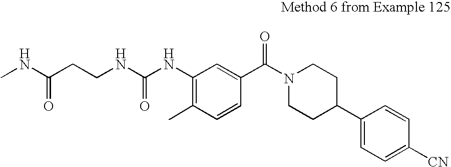

¹H NMR (400 MHz, d₆-DMSO, 30° C.) 1.50-1.80 (4H, m), 2.19 (3H, s), 2.27 (2H, t, J6.0), 2.83-3.17 (3H, m), 3.30 (2H, bs), 3.54 (3H, s), 3.78-3.86 (1H, m), 4.55-4.70 (1H, m), 6.73 (1H, bs), 6.92 (1H, d, J7.6), 7.17 (1H, d, J7.6), 7.51 (2H, d, J7.8), 7.78 (2H, d, J7.8), 7.84 (1H, s), 7.77-7.84 (1H, m), 7.98 (1H, s), m/z 448 (M+H)⁺.

### EXAMPLE 180

### tert-butyl (2S)-2-[[5-[4-(4-cyanophenyl)piperidine-1-carbonyl]-2-methyl-phenyl]carbamoylamino]-4-methylsulfonyl-butanoate

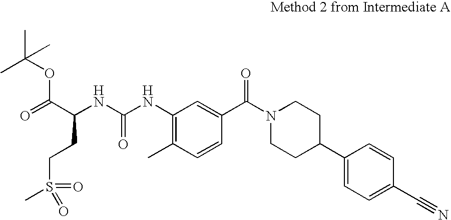

¹H NMR (300.073 MHz, d₆-DMSO) δ 1.42 (s, 9H), 1.51-1.87 (m, 4H), 1.89-2.08 (m, 1H), 2.10-2.28 (m, 4H), 2.76-3.26 (m, 8H), 3.63-3.95 (m, 1H), 4.17-4.31 (m, 1H), 4.40-4.75 (m, 1H), 6.91-6.98 (m, 1H), 7.08 (d, 1H), 7.19 (d, 1H), 7.49 (d, 2H), 7.76 (d, 2H), 7.89-7.98 (m, 2H), m/z 583 (M+H)⁺.

### EXAMPLE 181

### Methyl 4-[[5-[4-(4-cyanophenyl)piperidine-1-carbonyl]-2-methyl-phenyl]carbamoylamino]cyclohexane-1-carboxylate

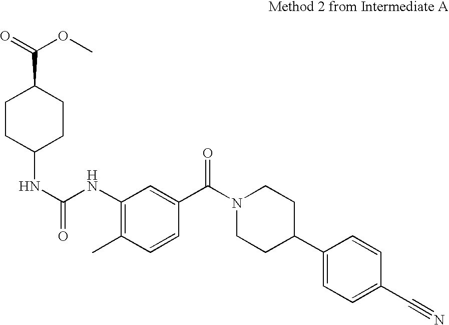

¹H NMR (300.073 MHz, d₆-DMSO) δ 1.08-1.27 (m, 2H), 1.32-1.49 (m, 2H), 1.51-1.84 (m, 4H), 1.86-2.01 (m, 4H), 2.18 (s, 3H), 2.24-2.38 (m, 1H), 2.75-3.24 (m, 3H), 3.31-3.47 (m, 1H), 3.59 (s, 3H), 3.67-3.96 (m, 1H), 4.36-4.81 (m, 1H), 6.57 (d, 1H), 6.90 (d, 1H), 7.16 (d, 1H), 7.49 (d, 2H), 7.60 (s, 1H), 7.76 (d, 2H), 7.98 (s, 1H), m/z 503 (M+H)⁺.

### EXAMPLE 182

### 1-[5-[4-(4-cyanophenyl)piperidine-1-carbonyl]-2-methyl-phenyl]-3-(1-hydroxypropan-2-yl)urea

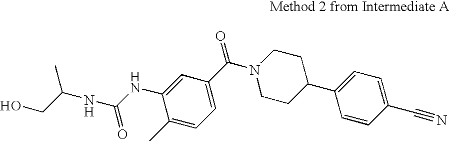

¹H NMR (400.132 MHz, CDCl₃) [symbol] 11.19 (d, 3H), 1.59-1.86 (m, 4H), 1.96 (s, 3H), 2.82-2.92 (m, 2H), 3.09-3.22 (m, 1H), 3.47-3.55 (m, 1H), 3.75 (s, 1H), 3.89-4.02 (m, 3H), 4.81-4.92 (m, 1H), 5.86 (s, 1H), 6.87-6.93 (m, 2H), 7.00 (d, 1H), 7.34 (d, 2H), 7.62 (d, 2H), 7.68-7.72 (m, 1H), m/z 421 (M+H)⁺.

### EXAMPLE 183

### 1-[5-[4-(4-cyanophenyl)piperidine-1-carbonyl]-2-methyl-phenyl]-3-(2-hydroxypropyl)urea

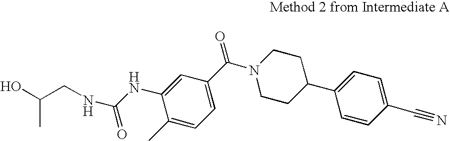

¹H NMR (400.132 MHz, CDCl₃) [symbol] 11.19 (d, 3H), 1.56-1.84 (m, 4H), 2.00 (s, 3H), 2.82-2.90 (m, 2H), 3.02-3.22 (m, 2H), 3.34-3.41 (m, 1H), 3.80 (s, 1H), 3.92-4.03 (m, 2H), 4.78-4.93 (m, 1H), 6.08 (s, 1H), 6.92 (d, 1H), 6.98 (s, 1H), 7.03 (d, 1H), 7.34 (d, 2H), 7.62 (d, 2H), 7.67 (s, 1H), m/z 421 (M+H)⁺.

### EXAMPLE 184

### 1-[5-[4-(4-cyanophenyl)piperidine-1-carbonyl]-2-methyl-phenyl]-3-(2,3-dihydroxypropyl)urea

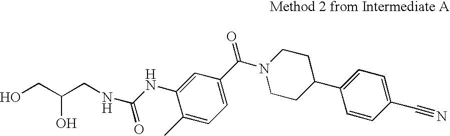

¹H NMR (400.132 MHz, CDCl₃) [symbol] 1.66-2.00 (m, 4H), 2.07 (s, 3H), 2.82-2.93 (m, 2H), 3.12-3.20 (m, 1H), 3.29-3.44 (m, 2H), 3.57-3.63 (m, 3H), 3.70 (s, 1H), 3.78-3.83 (m, 1H), 3.94-4.01 (m, 1H), 4.84-4.91 (m, 1H), 5.96 (s, 1H), 6.86 (s, 1H), 6.97 (d, 1H), 7.08 (d, 1H), 7.34 (d, 2H), 7.60-7.64 (m, 3H), m/z 437 (M+H)⁺.

### EXAMPLE 185

### 1-[5-[4-(4-cyanophenyl)piperidine-1-carbonyl]-2-methyl-phenyl]-3-(4-hydroxybutyl)urea

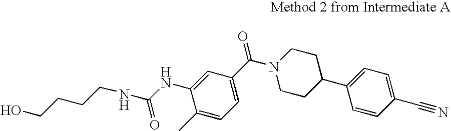

¹H NMR (400.132 MHz, CDCl₃) [symbol] 1.60-1.66 (m, 4H), 1.71-2.01 (m, 4H), 2.05 (s, 3H), 2.80-2.92 (m, 2H), 3.10-3.20 (m, 1H), 3.25-3.31 (m, 2H), 3.55 (s, 1H), 3.64-3.70 (m, 2H), 3.94-4.04 (m, 1H), 4.86-4.91 (m, 1H), 6.06 (t, 1H), 6.66 (s, 1H), 6.91 (d, 1H), 7.05 (d, 1H), 7.34 (d, 2H), 7.60-7.64 (m, 3H), m/z 435 (M+H)⁺.

### EXAMPLE 186

### 1-[5-[4-(4-cyanophenyl)piperidine-1-carbonyl]-2-methyl-phenyl]-3-(1,3-dihydroxypropan-2-yl)urea

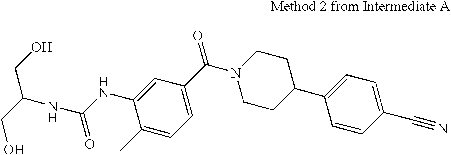

¹H NMR (400.132 MHz, CDCl₃) [symbol] 1.63-1.96 (m, 4H), 2.04 (s, 3H), 2.80-2.91 (m, 2H), 3.12-3.22 (m, 1H), 3.58 (s, 2H), 3.78-3.91 (m, 5H), 3.95-4.02 (m, 1H), 4.79-4.89 (m, 1H), 6.25 (s, 1H), 6.89 (s, 1H), 6.94 (d, 1H), 7.06 (d, 1H), 7.33 (d, 2H), 7.62 (d, 2H), 7.81-7.83 (m, 1H), m/z 437 (M+H)⁺.

### EXAMPLE 187

### (RS) 1-[5-[4-(4-cyanophenyl)piperidine-1-carbonyl]-2-methyl-phenyl]-3-(2-hydroxycyclohexyl)urea

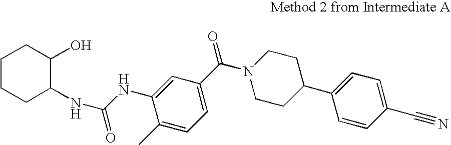

¹H NMR (400.132 MHz, CDCl₃) [symbol] 1.16-1.37 (m, 4H), 1.60-1.82 (m, 6H), 1.98 (s, 3H), 1.98-2.05 (m, 2H), 2.78-2.90 (m, 2H), 3.08-3.16 (m, 1H), 3.28-3.36 (m, 1H), 3.41-3.45 (m, 1H), 3.93-4.01 (m, 1H), 4.53 (s, 1H), 4.81-4.90 (m, 1H), 5.74 (s, 1H), 6.92 (d, 1H), 7.02 (d, 1H), 7.18 (s, 1H), 7.34 (d, 2H), 7.61 (d, 2H), 7.63-7.64 (m, 1H), m/z 461 (M+H)⁺.

### EXAMPLE 188

### 1-[5-[4-(4-cyanophenyl)piperidine-1-carbonyl]-2-methyl-phenyl]-3-(3-hydroxy-2,2-dimethyl-propyl)urea

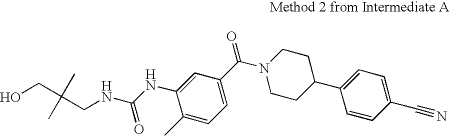

¹H NMR (400.132 MHz, CDCl₃) [symbol] 0.90 (s, 6H), 1.62-2.04 (m, 4H), 2.08 (s, 3H), 2.81-2.91 (m, 2H), 3.09 (d, 2H), 3.13-3.20 (m, 1H), 3.27 (d, 2H), 3.95-4.04 (m, 1H), 4.38 (s, 1H), 4.83-4.90 (m, 1H), 5.98 (s, 1H), 6.60 (s, 1H), 7.01 (d, 1H), 7.11 (d, 1H), 7.33 (d, 2H), 7.40-7.43 (m, 1H), 7.63 (d, 2H), m/z 449 (M+H)⁺.

### EXAMPLE 189

### 1-[5-[4-(4-cyanophenyl)piperidine-1-carbonyl]-2-methyl-phenyl]-3-[2-(2-hydroxyethoxy)ethyl]urea

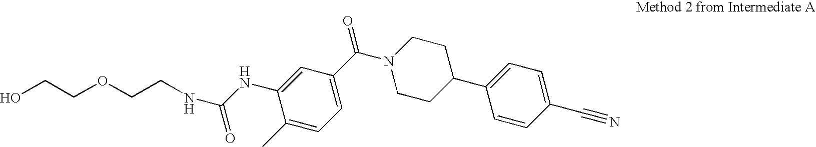

¹H NMR (400.132 MHz, CDCl₃) [symbol] 1.72-2.02 (m, 4H), 2.14 (s, 3H), 2.78-2.93 (m, 2H), 3.10-3.22 (m, 1H), 3.41-3.47 (m, 3H), 3.58-3.61 (m, 4H), 3.70-3.75 (m, 2H), 3.93-4.03 (m, 1H), 4.81-4.95 (m, 1H), 5.91 (s, 1H), 6.45 (s, 1H), 7.01 (d, 1H), 7.14 (d, 1H), 7.33 (d, 2H), 7.48-7.51 (m, 1H), 7.62 (d, 2H), m/z 451 (M+H)⁺.

### EXAMPLE 190

### 1-[5-[4-(4-cyanophenyl)piperidine-1-carbonyl]-2-methyl-phenyl]-3-(1-hydroxy-2-methyl-propan-2-yl)urea

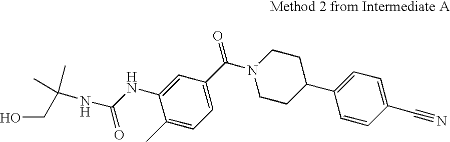

¹H NMR (400.132 MHz, CDCl₃) [symbol] 1.32 (s, 6H), 1.69-1.86 (m, 4H), 1.94 (s, 3H), 2.81-2.94 (m, 2H), 3.10-3.22 (m, 1H), 3.64-3.72 (m, 2H), 3.92-4.02 (m, 1H), 4.85-4.94 (m, 1H), 5.09 (t, 1H), 5.92 (s, 1H), 6.85 (d, 1H), 6.90 (s, 1H), 6.96 (d, 1H), 7.34 (d, 2H), 7.59 (s, 1H), 7.63 (d, 2H), m/z 435 (M+H)⁺.

### EXAMPLE 191

### 1-[5-[4-(4-cyanophenyl)piperidine-1-carbonyl]-2-methyl-phenyl]-3-(3-hydroxypropyl)urea

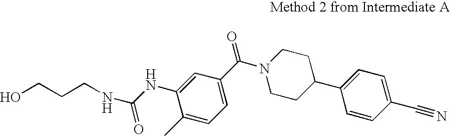

¹H NMR (400.132 MHz, CDCl₃) [symbol] 1.69-1.72 (m, 2H), 1.77-2.02 (m, 4H), 2.07 (s, 3H), 2.81-2.94 (m, 2H), 3.08-3.21 (m, 1H), 3.41 (q, 2H), 3.71 (q, 2H), 3.94-4.03 (m, 1H), 4.09 (t, 1H), 4.82-4.93 (m, 1H), 6.00 (t, 1H), 6.67 (s, 1H), 6.96 (d, 1H), 7.08 (d, 1H), 7.34 (d, 2H), 7.48 (s, 1H), 7.62 (d, 2H), m/z 421 (M+H)⁺.

### EXAMPLE 192

### (2S)-2-[[5-[4-(4-cyanophenyl)piperidine-1-carbonyl]-2-methyl-phenyl]carbamoylamino]-3-hydroxy-propanamide

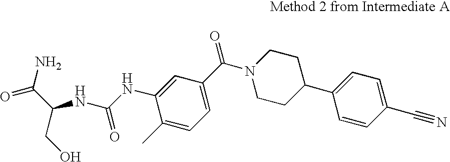

¹H NMR (400.132 MHz, CDCl₃) [symbol] 1.55-2.02 (m, 4H), 2.08 (s, 3H), 2.70-2.86 (m, 2H), 3.01-3.15 (m, 1H), 3.62-3.73 (m, 1H), 3.85-4.00 (m, 2H), 4.37 (s, 1H), 4.66-4.91 (m, 2H), 6.82-7.10 (m, 4H), 7.29-7.36 (m, 3H), 7.56 (d, 2H), 7.80 (s, 1H), 7.92 (s, 1H), m/z 450 (M+H)⁺.

### EXAMPLE 193

### 1-[5-[4-(4-cyanophenyl)piperidine-1-carbonyl]-2-methyl-phenyl]-3-(4-hydroxycyclohexyl)urea

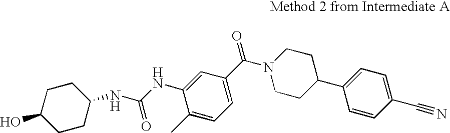

¹H NMR (400.132 MHz, CDCl₃) [symbol] 1.15-1.26 (m, 2H), 1.35-1.45 (m, 2H), 1.53 (d, 1H), 1.66-1.86 (m, 4H), 1.93-2.04 (m, 4H), 2.08 (s, 3H), 2.80-2.93 (m, 2H), 3.10-3.22 (m, 1H), 3.58-3.66 (m, 2H), 3.91-4.01 (m, 1H), 4.82-4.90 (m, 1H), 5.18 (d, 1H), 6.42 (s, 1H), 6.99 (d, 1H), 7.09 (d, 1H), 7.32 (d, 2H), 7.49-7.51 (m, 1H), 7.62 (d, 2H), m/z 461 (M+H)⁺.

### EXAMPLE 194

### 1-[5-[4-(4-cyanophenyl)piperidine-1-carbonyl]-2-methyl-phenyl]-3-[1-(hydroxymethyl)cyclopentyl]urea

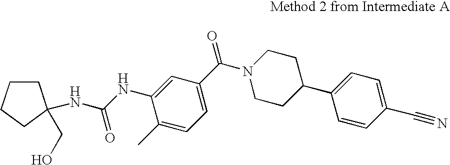

¹H NMR (400.132 MHz, CDCl₃) [symbol] 1.64-1.86 (m, 11H), 1.92 (s, 3H), 1.97-2.02 (m, 1H), 2.81-2.93 (m, 2H), 3.10-3.19 (m, 1H), 3.72-3.80 (m, 2H), 3.93-3.98 (m, 1H), 4.85-4.91 (m, 1H), 5.09 (t, 1H), 6.10 (s, 1H), 6.82 (d, 1H), 6.93 (d, 1H), 7.04 (s, 1H), 7.35 (d, 2H), 7.60-7.67 (m, 3H), m/z 461 (M+H)⁺.

### EXAMPLE 195

### (RS)-1-[5-[4-(4-cyanophenyl)piperidine-1-carbonyl]-2-methyl-phenyl]-3-(3,3,3-trifluoro-2-hydroxy-propyl)urea

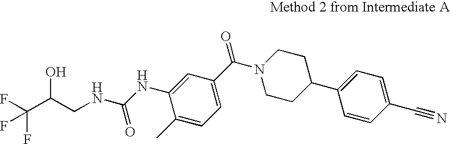

¹H NMR (400.132 MHz, CDCl₃) [symbol] 1.61-1.87 (m, 4H), 2.04 (s, 3H), 2.79-2.95 (m, 2H), 3.14-3.19 (m, 1H), 3.66-3.74 (m, 1H), 3.87 (t, 2H), 3.95-4.15 (m, 1H), 4.86-4.95 (m, 2H), 6.22-6.25 (m, 1H), 6.36-6.39 (m, 1H), 6.91-7.08 (m, 2H), 7.35 (d, 2H), 7.61-7.63 (m, 3H), m/z 475 (M+H)⁺.

### EXAMPLE 196

### 3-[3-(2-chlorophenoxy)-2-hydroxy-propyl]-1-[5-[4-(4-cyanophenyl)piperidine-1-carbonyl]-2-methyl-phenyl]urea

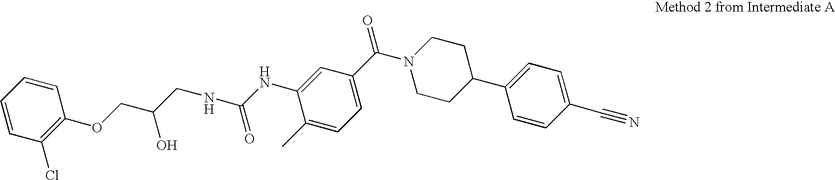

¹H NMR (400.132 MHz, CDCl₃) [symbol] 1.70-2.00 (m, 4H), 2.05 (s, 3H), 2.72-2.86 (m, 2H), 3.08-3.17 (m, 1H), 3.40-3.50 (m, 1H), 3.64-3.71 (m, 1H), 3.93-4.09 (m, 3H), 4.16-4.23 (m, 1H), 4.39 (s, 1H), 4.79-4.88 (m, 1H), 6.02 (s, 1H), 6.89-7.06 (m, 5H), 7.19-7.24 (m, 1H), 7.30-7.36 (m, 3H), 7.58-7.61 (m, 3H), m/z 548 (M+H)⁺.

### EXAMPLE 197

### 1-[5-[4-(4-cyanophenyl)piperidine-1-carbonyl]-2-methyl-phenyl]-3-(3-hydroxy-1-adamantyl)urea

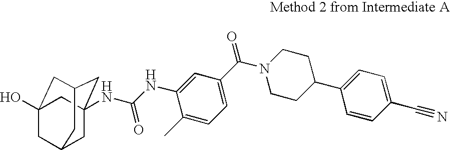

¹H NMR (400.132 MHz, CDCl₃) [symbol] 1.50-1.75 (m, 11H), 1.83-1.88 (m, 2H), 1.97 (s, 3H), 1.98-2.03 (m, 2H), 2.10-2.15 (m, 2H), 2.25-2.29 (m, 2H), 2.79-2.91 (m, 2H), 3.07-3.21 (m, 1H), 3.91-4.02 (m, 1H), 4.82-4.92 (m, 1H), 5.58 (s, 1H), 6.71 (s, 1H), 6.82 (d, 1H), 6.95 (d, 1H), 7.34 (d, 2H), 7.60-7.67 (m, 3H), m/z 513 (M+H)⁺.

### EXAMPLE 198

### 1-[5-[4-(4-cyanophenyl)piperidine-1-carbonyl]-2-methyl-phenyl]-3-[(2R)-1-hydroxy-3-methoxy-propan-2-yl]urea

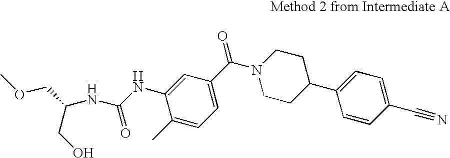

¹H NMR (400.132 MHz, CDCl₃) [symbol] 1.69-2.03 (m, 4H), 2.11 (s, 3H), 2.80-2.87 (m, 2H), 3.07-3.20 (m, 1H), 3.34-3.38 (m, 1H), 3.38 (s, 3H), 3.59 (d, 2H), 3.75-3.85 (m, 2H), 3.96-4.03 (m, 2H), 4.82-4.93 (m, 1H), 5.83 (d, 1H), 6.78 (s, 1H), 6.99 (d, 1H), 7.09 (d, 1H), 7.33 (d, 2H), 7.62 (d, 2H), 7.70 (s, 1H), m/z 451 (M+H)⁺.

### EXAMPLE 199

### 1-[5-[4-(4-cyanophenyl)piperidine-1-carbonyl]-2-methyl-phenyl]-3-[(3R)-oxolan-3-yl]urea

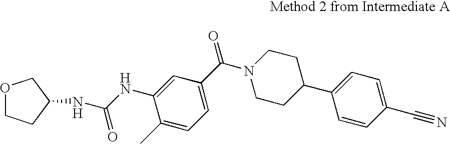

¹H NMR (400.132 MHz, CDCl₃) [symbol] 1.73-1.89 (m, 4H), 1.95-1.99 (m, 1H), 2.02 (s, 3H), 2.21-2.30 (m, 1H), 2.81-2.88 (m, 2H), 3.10-3.16 (m, 1H), 3.66-3.70 (m, 1H), 3.80-4.03 (m, 4H), 4.38-4.45 (m, 1H), 4.81-4.92 (m, 1H), 5.83 (d, 1H), 6.63 (s, 1H), 6.95 (d, 1H), 7.06 (d, 1H), 7.33 (d, 2H), 7.48 (s, 1H), 7.62 (d, 2H), m/z 433 (M+H)⁺.

### EXAMPLE 200

### 1-[5-[4-(4-cyanophenyl)piperidine-1-carbonyl]-2-methyl-phenyl]-3-[3-(3-methyl-2H-pyrazol-4-yl)propyl]urea

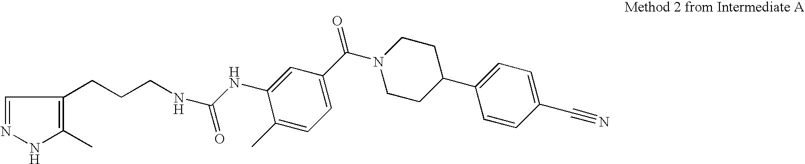

¹H NMR (300.072 MHz, CDCl₃) δ 1.60-1.93 (m, 6H), 2.04 (s, 3H), 2.15 (s, 3H), 2.38 (t, 2H), 2.75-2.89 (m, 2H), 3.08-3.24 (m, 3H), 3.86-4.06 (m, 1H), 4.76-4.91 (m, 1H), 5.82-5.93 (m, 1H), 6.93-7.08 (m, 3H), 7.21 (s, 1H), 7.29 (d, 2H), 7.58 (d, 2H), 7.67 (s, 1H) [NB the signal due to the imidazole was not apparent], m/z 485 (M+H)⁺.

### EXAMPLE 201

### 1-[5-[4-(4-cyanophenyl)piperidine-1-carbonyl]-2-ethyl-phenyl]-3-propan-2-yl-urea

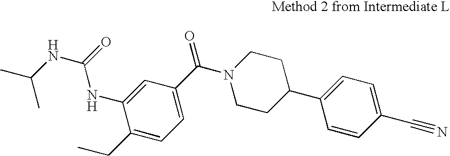

¹H NMR (300.073 MHz, d₆-DMSO, 30° C.) δ 1.03-1.21 (9H, m), 1.46-1.93 (4H, m), 2.42-2.62 (2H, m, partially obscured by DMSO signal), 2.66-3.20 (3H, m), 3.55-3.98 (2H, m, appears as a broad flat singlet containing a multiplet), 4.39-4.76 (1H, m), 6.52 (1H, dJ=7.2 Hz), 6.95 (1H, dJ=7.2 Hz), 7.16 (1H, dJ=8.4 Hz), 7.49 (2H, dJ=8.3 Hz), 7.57 (1H, s), 7.75 (2H, dJ=7.7 Hz), 7.95 (1H, s), m/z 419 (M+H)⁺.

### EXAMPLE 202

### 1-[5-[4-(4-cyanophenyl)piperidine-1-carbonyl]-2-methyl-phenyl]-3-(2-methanesulfonamido-2-methyl-propyl)urea

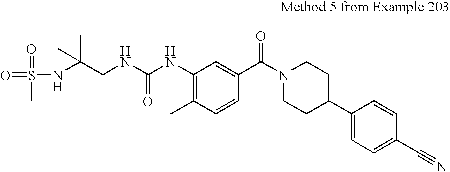

¹H NMR (300.072 MHz, CDCl₃) δ 1.38 (s, 6H), 1.69-1.84 (m, 4H), 2.00 (s, 3H), 2.77-2.93 (m, 2H), 3.01 (s, 3H), 3.08-3.19 (m, 1H), 3.31 (d, 2H), 3.89-4.00 (m, 1H), 4.76-4.98 (m, 1H), 5.64 (s, 1H), 6.24-6.32 (m, 1H), 6.92 (d, 1H), 6.99-7.05 (m, 2H), 7.34 (d, 2H), 7.58-7.64 (m, 3H), m/z 512 (M+H)⁺.

### EXAMPLE 203

### 3-(2-amino-2-methyl-propyl)-1-[5-[4-(4-cyanophenyl)piperidine-1-carbonyl]-2-methyl-phenyl]urea

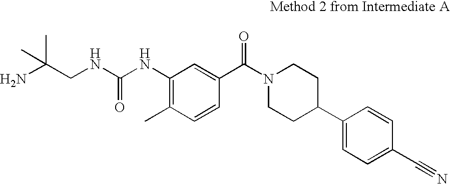

¹H NMR (300.072 MHz, CDCl₃) δ 1.12 (s, 6H), 1.56-2.03 (m, 4H), 2.20 (s, 3H), 2.77-2.96 (m, 4H), 3.03-3.09 (m, 1H), 3.15 (d, 2H), 3.93-4.09 (m, 1H), 4.70-5.03 (m, 1H), 6.45-6.53 (m, 1H), 6.97 (d, 1H), 7.11 (d, 1H), 7.31 (d, 2H), 7.60 (d, 2H), 7.71 (m, 2H), m/z 434 (M+H)⁺.

### EXAMPLE 204

### 3-[5-[4-(4-cyanophenyl)piperidine-1-carbonyl]-2-methyl-phenyl]-1-[2-(ethylsulfonylamino)-2-methyl-propyl]urea

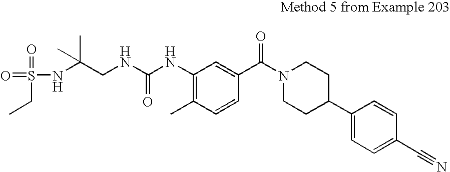

¹H NMR (300.072 MHz, CDCl₃) [symbol] 1.32-1.38 (m, 9H), 1.63-1.83 (m, 4H), 2.03 (s, 3H), 2.80-2.88 (m, 2H), 3.05 (q, 2H), 3.12-3.22 (m, 1H), 3.32 (d, 2H), 3.89-4.09 (m, 1H), 4.77-5.01 (m, 1H), 5.40 (s, 1H), 6.23 (t, 1H), 6.88 (s, 1H), 6.92-7.08 (m, 2H), 7.34 (d, 2H), 7.57-7.63 (m, 3H), m/z 526 (M+H)⁺.

### EXAMPLE 205

### N-[1-[[5-[4-(4-cyanophenyl)piperidine-1-carbonyl]-2-methyl-phenyl]carbamoylamino]-2-methyl-propan-2-yl]-2,2-dimethyl-propanamide

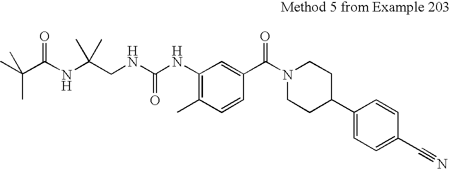

¹H NMR (300.072 MHz, CDCl₃) [symbol] 1.13 (s, 9H), 1.33 (s, 6H), 1.57-1.78 (m, 4H), 2.15 (s, 3H), 2.79-2.91 (m, 2H), 3.00-3.18 (m, 1H), 3.28 (d, 2H), 3.89-4.08 (m, 1H), 4.71-4.96 (m, 1H), 6.16 (t, 1H), 6.63 (s, 1H), 6.79 (s, 1H), 7.03-7.16 (m, 2H), 7.31 (d, 2H), 7.53-7.55 (m, 1H), 7.61 (d, 2H), m/z 518 (M+H)⁺.

### EXAMPLE 206

### tert-butyl N-[3-[[5-[4-(4-cyanophenyl)piperidine-1-carbonyl]-2-methyl-phenyl]carbamoylamino]propyl]carbamate

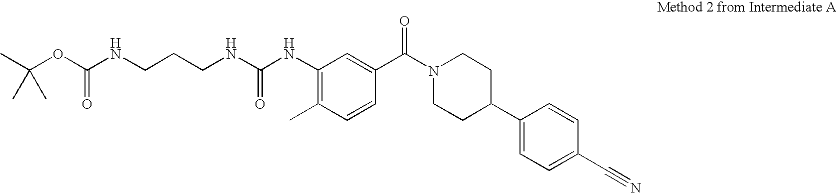

1H NMR (300.072 MHz, CDCl₃) δ 1.42 (s, 9H), 1.59-1.70 (m, 2H), 1.76-2.00 (m, 4H), 2.12 (s, 3H), 2.78-2.91 (m, 2H), 2.97-3.07 (m, 1H), 3.17 (q, 2H), 3.26 (q, 2H), 3.92-4.05 (m, 1H), 4.77-4.94 (m, 1H), 5.06 (s, 1H), 5.73 (s, 1H), 6.57 (s, 1H), 6.97-7.14 (m, 2H), 7.32 (d, 2H), 7.58-7.64 (m, 3H), m/z (ESI+) (M+H)+=520; HPLC tR=2.32 min

### EXAMPLE 207

### 3-(3-aminopropyl)-1-[5-[4-(4-cyanophenyl)piperidine-1-carbonyl]-2-methyl-phenyl]urea

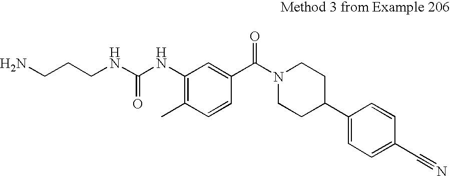

1H NMR (300.073 MHz, d₆-DMSO) δ1.56-1.85 (m, 6H), 2.23 (s, 3H), 2.76-3.00 (m, 4H), 3.07-3.22 (m, 3H), 3.63-3.93 (m, 1H), 4.57 (s, 3H), 6.92 (d, 1H), 7.16 (d, 1H), 7.22 (s, 1H), 7.49 (d, 2H), 7.76 (d, 2H), 7.93 (s, 1H), 8.12 (s, 1H), m/z (ESI+) (M+H)+=420; HPLC tR=1.24 min.

### EXAMPLE 208

### tert-butyl 4-[[5-[4-(4-cyanophenyl)piperidine-1-carbonyl]-2-methyl-phenyl]carbamoylamino]piperidine-1-carboxylate

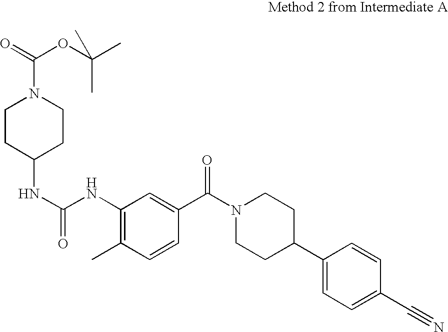

¹H NMR (300.073 MHz, d₆-DMSO) δ 1.20-1.33 (m, 2H), 1.39 (s, 9H), 1.48-1.94 (m, 6H), 2.19 (s, 3H), 2.65-3.22 (m, 5H), 3.50-3.95 (m, 4H), 4.37-4.83 (m, 1H), 6.66 (d, 1H), 6.91 (d, 1H), 7.16 (d, 1H), 7.49 (d, 2H), 7.64 (s, 1H), 7.75 (d, 2H), 7.97 (s, 1H), [also contains signals due to EtOAc], m/z 546 (M+H)⁺.

### EXAMPLE 209

### tert-butyl (3R)-3-[[5-[4-(4-cyanophenyl)piperidine-1-carbonyl]-2-methyl-phenyl]carbamoylamino]pyrrolidine-1-carboxylate

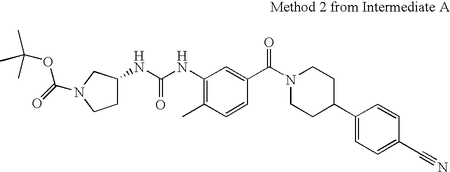

### EXAMPLE 210

### tert-butyl (3S)-3-[[5-[4-(4-cyanophenyl)piperidine-1-carbonyl]-2-methyl-phenyl]carbamoylamino]pyrrolidine-1-carboxylate

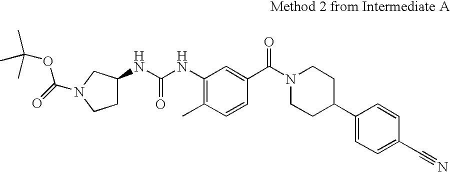

### EXAMPLE 211

### 1-[5-[4-(4-cyanophenyl)piperidine-1-carbonyl]-2-methyl-phenyl]-3-(4-piperidyl)urea

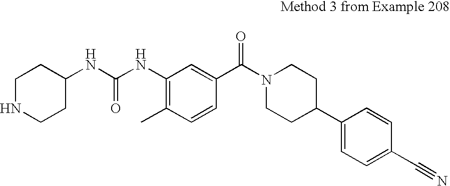

¹H NMR (300.073 MHz, d₆-DMSO) δ 1.24-1.94 (m, 8H), 2.21 (s, 3H), 2.58-3.25 (m, 6H), 3.38-3.96 (m, 2H), 4.11-4.85 (m, 2H), 6.90 (d, 1H), 6.96 (d, 1H), 7.15 (d, 1H), 7.48 (d, 2H), 7.75 (d, 2H), 7.81 (s, 1H), 7.99 (s, 1H), m/z 446 (M+H)⁺.

### EXAMPLE 212

### 1-[5-[4-(4-cyanophenyl)piperidine-1-carbonyl]-2-methyl-phenyl]-3-[(3R)-pyrrolidin-3-yl]urea

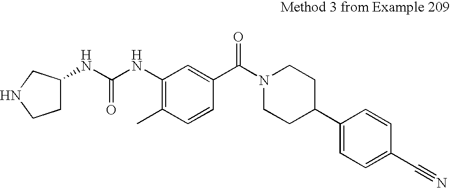

m/z (ESI+) (M+H)+=432; HPLC tR=1.24 min.

### EXAMPLE 213

### 1-[5-[4-(4-cyanophenyl)piperidine-1-carbonyl]-2-methyl-phenyl]-3-[(3S)-pyrrolidin-3-yl]urea

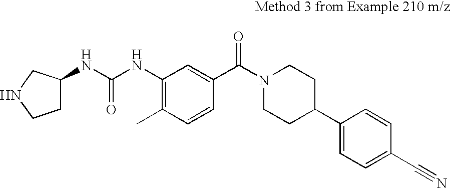

(ESI+) (M+H)+=432; HPLC tR=1.24 min.

### EXAMPLE 214

### 1-[5-[4-(4-cyanophenyl)piperidine-1-carbonyl]-2-methyl-phenyl]-3-[2-(methyl-propan-2-ylsulfonyl-amino)ethyl]urea

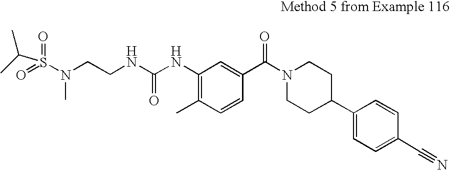

m/z (ESI+) (M+H)+=526; HPLC tR=2.14 min.

### EXAMPLE 215

### 1-[5-[4-(4-cyanophenyl)piperidine-1-carbonyl]-2-methyl-phenyl]-3-(3-methanesulfonamidopropyl)urea

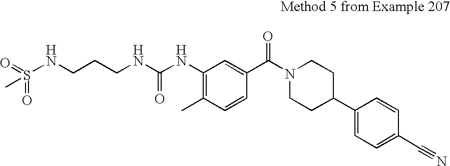

m/z (ESI+) (M+H)+=498; HPLC tR=1.93 min.

### EXAMPLE 216

### 3-[5-[4-(4-cyanophenyl)piperidine-1-carbonyl]-2-methyl-phenyl]-1-[3-(ethylsulfonylamino)propyl]urea

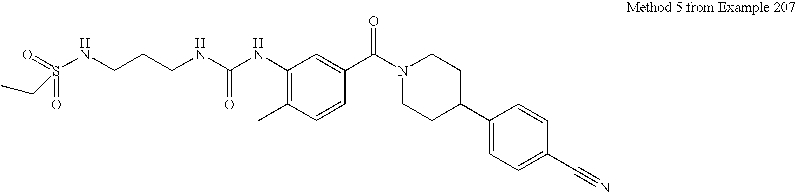

m/z (ESI+) (M+H)+=512; HPLC tR=2.00 min.

### EXAMPLE 217

### 1-[5-[4-(4-cyanophenyl)piperidine-1-carbonyl]-2-methyl-phenyl]-3-[3-(propan-2-ylsulfonylamino)propyl]urea

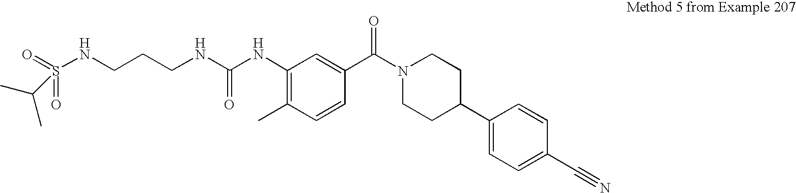

m/z (ESI+) (M+H)+=526; HPLC tR=2.08 min.

### EXAMPLE 218

### 1-[5-[4-(4-cyanophenyl)piperidine-1-carbonyl]-2-methyl-phenyl]-3-[(3R)-1-methylsulfonylpyrrolidin-3-yl]urea

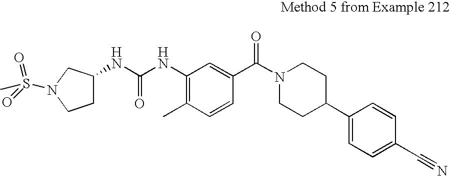

### EXAMPLE 219

### 3-[5-[4-(4-cyanophenyl)piperidine-1-carbonyl]-2-methyl-phenyl]-1-[(3R)-1-ethylsulfonylpyrrolidin-3-yl]urea

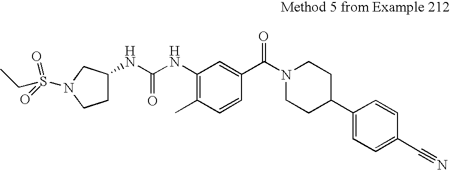

### EXAMPLE 220

### 3-[5-[4-(4-cyanophenyl)piperidine-1-carbonyl]-2-methyl-phenyl]-1-[(3S)-1-propanoylpyrrolidin-3-yl]urea

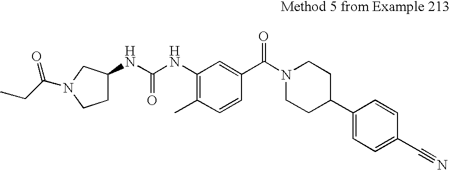

### EXAMPLE 221

### 1-[5-[4-(4-cyanophenyl)piperidine-1-carbonyl]-2-methyl-phenyl]-3-[(3S)-1-methylsulfonylpyrrolidin-3-yl]urea

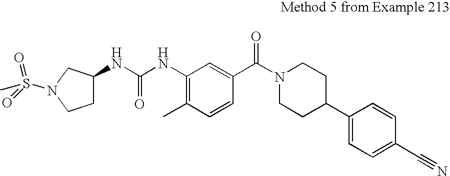

### EXAMPLE 222

### 3-[5-[4-(4-cyanophenyl)piperidine-1-carbonyl]-2-methyl-phenyl]-1-[(3S)-1-ethylsulfonylpyrrolidin-3-yl]urea

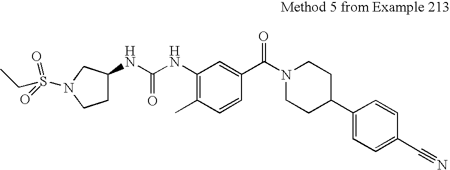

### EXAMPLE 223

### 1-[5-[4-(4-cyanophenyl)piperidine-1-carbonyl]-2-methyl-phenyl]-3-(3-methylsulfonylpropyl)urea

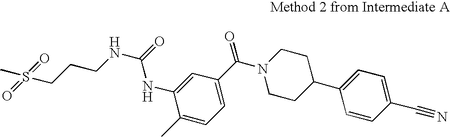

¹H NMR (300.073 MHz, d₆-DMSO, 30° C.) δ 1.44-2.03 (6H, m), 2.21 (3H, s), 2.70-3.25 (10H, m), 3.57-4.01 (1H, m appears as a broad flat singlet), 4.32-4.78 (1H, m appears as abroad flat singlet), 6.72 (1H, tJ=5.4 Hz), 6.93 (1H, dJ=6.7 Hz), 7.17 (1H, dJ=7.5 Hz), 7.49 (2H, dJ=8.3 Hz), 7.70-7.80 (3H, m), 7.93 (1H, s), m/z 483 (M+H)⁺.

### EXAMPLE 224

### 1-benzyl-3-[3-[4-(4-cyanophenyl)piperidine-1-carbonyl]-4-fluoro-phenyl]urea

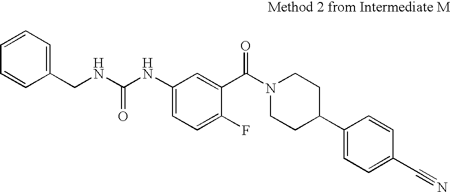

¹H NMR (300.073 MHz, d₆-DMSO, 30° C.) δ 1.38-1.95 (4H, m), 2.77-3.25 (3H, m), 3.46-3.60 (1H, m), 4.28 (2H, dJ=6.6 Hz), 4.59-4.72 (1H, m), 6.66 (1H, tJ=5.9 Hz), 7.09-7.42 (7H, m), 7.43-7.56 (3H, m), 7.75 (2H, dJ=9.6 Hz), 8.67 (1H, s), m/z 457 (M+H)⁺.

### EXAMPLE 225

### 1-[5-[4-(4-cyanophenyl)piperidine-1-carbonyl]-2-methyl-phenyl]-3-cyclopropyl-urea

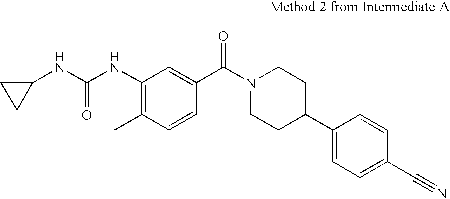

¹H NMR (300.073 MHz, d₆-DMSO) δ−0.01-0.39 (m, 4H), 1.21-1.61 (m, 4H), 1.90 (s, 3H), 2.32-2.96 (m, 4H), 3.39-3.68 (m, 1H), 4.18-4.49 (m, 1H), 6.49-6.54 (m, 1H), 6.65 (d, J=7.6 Hz, 1H), 6.89 (d, J=7.8 Hz, 1H), 7.21 (d, J=8.3 Hz, 2H), 7.30 (s, 1H), 7.48 (d, J=8.2 Hz, 2H), 7.65 (s, 1H), m/z 403 (M+H)⁺.

### EXAMPLE 226

### 1-butan-2-yl-3-[5-[4-(4-cyanophenyl)piperidine-1-carbonyl]-2-methyl-phenyl]urea

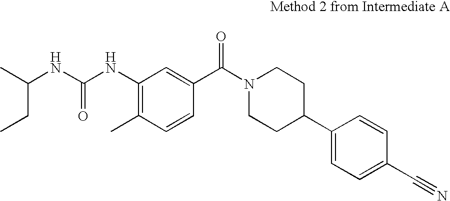

¹H NMR (300.073 MHz, d₆-DMSO) δ 0.75-1.49 (m, 8H), 1.52-1.68 (m, 2H), 1.70-1.90 (m, 2H), 2.19 (s, 3H), 2.60-3.18 (m, 3H), 3.52-3.65 (m, 1H), 3.68-3.93 (m, 1H), 4.43-4.72 (m, 1H), 6.48 (d, J=7.9 Hz, 1H), 6.89 (d, J=6.1 Hz, 1H), 7.16 (d, J=7.7 Hz, 1H), 7.49 (d, J=8.3 Hz, 2H), 7.60 (s, 1H), 7.76 (d, J=8.2 Hz, 2H), 8.00 (s, 1H), m/z 419 (M+H)⁺.

### EXAMPLE 227

### 3-[5-[4-(4-cyanophenyl)piperidine-1-carbonyl]-2-methyl-phenyl]-1-propyl-urea

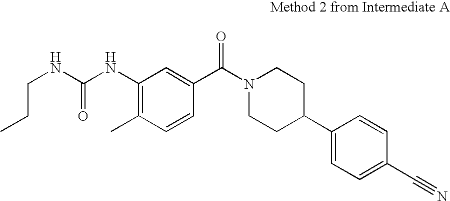

¹H NMR (300.073 MHz, d₆-DMSO) δ 0.77-1.49 (m, 5H), 1.51-1.66 (m, 2H), 1.69-1.91 (m, 2H), 2.20 (s, 3H), 2.78-3.14 (m, 5H), 3.66-3.97 (m, 1H), 4.42-4.79 (m, 1H), 6.56-6.64 (m, 1H), 6.90 (d, J=7.7 Hz, 1H), 7.16 (d, J=7.7 Hz, 1H), 7.49 (d, J=8.2 Hz, 2H), 7.66 (s, 1H), 7.76 (d, J=8.1 Hz, 2H), 7.96 (s, 1H), m/z 405 (M+H)⁺.

### EXAMPLE 228

### 1-[5-[4-(4-cyanophenyl)piperidine-1-carbonyl]-2-methyl-phenyl]-3-cyclohexyl-urea

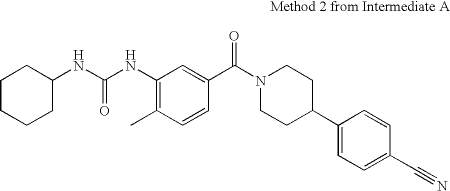

¹H NMR (300.073 MHz, d₆-DMSO) δ 0.92-1.88 (m, 14H), 2.19 (s, 3H), 2.77-3.09 (m, 3H), 3.38-3.53 (m, 1H), 3.65-3.98 (m, 1H), 4.42-4.75 (m, 1H), 6.58 (d, J=7.7 Hz, 1H), 6.89 (d, J=9.2 Hz, 1H), 7.16 (d, J=7.8 Hz, 1H), 7.49 (d, J=8.3 Hz, 2H), 7.61 (s, 1H), 7.76 (d, J=8.2 Hz, 2H), 7.99 (s, 1H), m/z 445 (M+H)⁺.

### EXAMPLE 229

### 1-[5-[4-(4-cyanophenyl)piperidine-1-carbonyl]-2-methyl-phenyl]-3-(3-methylbutan-2-yl)urea

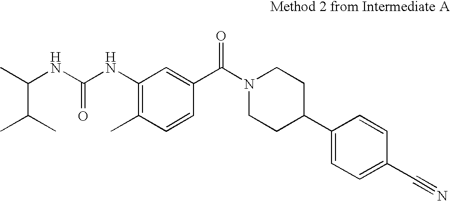

¹H NMR (300.073 MHz, d₆-DMSO) δ 0.75-1.92 (m, 14H), 2.20 (s, 3H), 2.71-3.17 (m, 3H), 3.49-3.65 (m, 1H), 3.67-3.95 (m, 1H), 4.46-4.75 (m, 1H), 6.50 (d, J=8.4 Hz, 1H), 6.89 (d, J=8.9 Hz, 1H), 7.16 (d, J=7.7 Hz, 1H), 7.49 (d, J=8.2 Hz, 2H), 7.63 (s, 1H), 7.76 (d, J=8.1 Hz, 2H), 8.01 (s, 1H), m/z 433 (M+H)⁺.

### EXAMPLE 230

### 3-[[5-[4-(4-cyanophenyl)piperidine-1-carbonyl]-2-methyl-phenyl]carbamoylamino]-N-(2-hydroxyethyl)propanamide

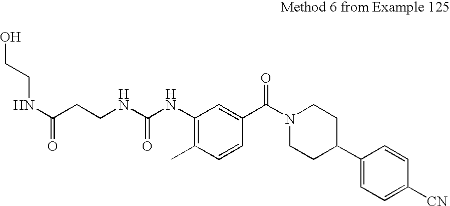

The title compound was prepared by the procedure described in Method 6, starting from 3-[[5-[4-(4-cyanophenyl)piperidine-1-carbonyl]-2-methyl-phenyl]carbamoylamino]propanoic acid (Example 125), ¹H NMR (300.073 MHz, d₆-DMSO) 1.45-1.92 (4H, m), 2.18 (3H, s), 2.29 (2H, t, J6.4), 2.72-3.08 (4H, m), 3.67-3.42 (2H, m), 3.79 (1H, bs), 4.05 (2H, q, J5.3), 4.62 (2H, t, J5.4), 6.68 (1H, t, J5.7), 6.90 (1H, dd, J7.5, 1.4), 7.15 (1H, d, J7.7), 7.49 (2H, d, J8.3), 7.76 (2H, d, J8.3), 7.81 (1H, s), 7.86 (1H, t, J5.5), 7.96 (1H, s), m/z 477 (M+H)⁺.

### EXAMPLE 231

### N-[2-[[5-[4-(4-cyanophenyl)piperidine-1-carbonyl]-2-methyl-phenyl]carbamoylamino]ethyl]-N′,N′-dimethyl-propanediamide

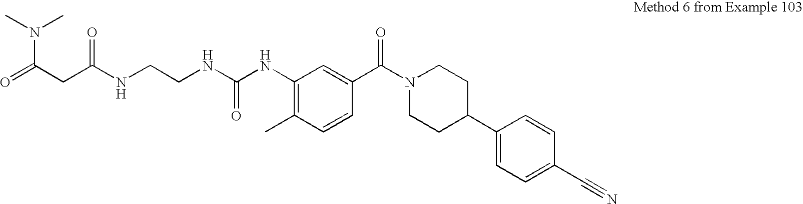

¹H NMR (300.073 MHz, d₆-DMSO) [symbol] 1.54-1.86 (m, 4H), 2.19 (s, 3H), 2.68 (s, 3H), 2.80 (s, 3H), 2.90-2.93 (m, 2H), 2.95 (s, 2H), 2.99-3.04 (m, 1H), 3.12-3.18 (m, 4H), 3.67-3.94 (m, 1H), 4.44-4.71 (m, 1H), 6.65 (s, 1H), 6.91-6.94 (m, 1H), 7.17 (d, 1H), 7.49 (d, 2H), 7.72-7.79 (m, 3H), 7.91 (s, 1H), 8.03-8.09 (m, 1H), m/z 519 (M+H)⁺.

### EXAMPLE 232

### N-[2-[[5-[4-(4-cyanophenyl)piperidine-1-carbonyl]-2-methyl-phenyl]carbamoylamino]propyl]acetamide

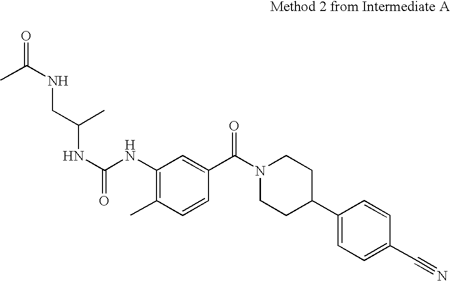

¹H NMR (300.073 MHz, d₆-DMSO, 30° C.) δ 1.04 (3H, d J=6.5 Hz), 1.21-1.30 (2H, m possible impurity), 1.48-1.69 (2H, m 1.69-1.90), (5H, m contains a singlet at 1.81), 2.20 (3H, s), 2.70-3.20 (5H, m), 3.51-4.00 (2H, m), 4.40-4.80 (1H, m appears as a broad flat singlet), 6.53 (1H, d J=7.4 Hz), 6.91 (1H, d J=8.1 Hz), 7.17 (1H, d J=8.1 Hz), 7.49 (2H, d J=8.8 Hz), 7.66-7.80 (3H, m), 7.81-7.89 (1H, m), 7.95 (1H, s), m/z 462 (M+H)⁺.

### EXAMPLE 233

### N-[3-[[5-[4-(4-cyanophenyl)piperidine-1-carbonyl]-2-methyl-phenyl]carbamoylamino]propyl]morpholine-4-carboxamide

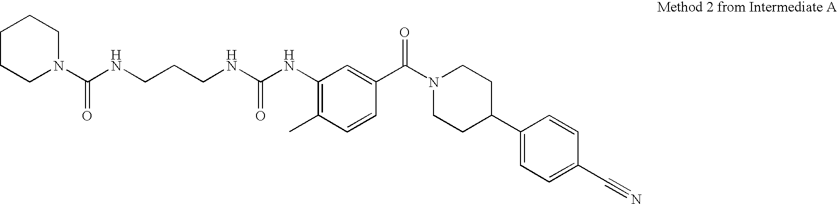

¹H NMR (300.073 MHz, d₆-DMSO, 30° C.) δ 1.20-1.30 (2H, m, possible impurity), 1.47-1.68 (4H, m), 1.70-1.90 (2H, m), 2.20 (3H, s), 2.70-3.00 (2H, m), 3.01-3.16 (5H, m), 3.19-3.26 (4H, m), 3.47-3.55 (4H, m), 3.67-3.91 (1H, m), 4.40-4.74 (1H, m), 6.50 (1H, t J=5.3 Hz), 6.64 (1H, t J=5.6 Hz), 6.92 (1H, d J=7.6 Hz), 7.17 (1H, d J=7.6 Hz), 7.49 (2H, d J=7.1 Hz), 7.72-7.80 (3H, m), 7.92 (1H, s), m/z 533 (M+H)⁺.

### EXAMPLE 234

### 3-[2-(carbamoylamino)ethyl]-1-[5-[4-(4-cyanophenyl)piperidine-1-carbonyl]-2-methyl-phenyl]urea

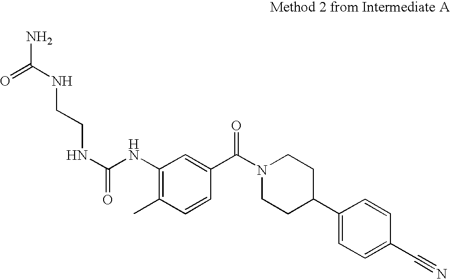

¹H NMR (300.073 MHz, d₆-DMSO, 30° C.) δ 1.44-1.95 (4H, m), 2.20 (3H, s), 2.66-3.21 (7H, m), 3.58-4.00 (1H, m appears as a broad flat singlet), 4.40-4.78 (1H, m appears as a broad flat singlet), 5.46 (2H, s), 5.93-6.03 (1H, m), 6.60-6.71 (1H, m), 6.92 (1H, d J=7.4 Hz), 7.17 (1H, d J=8.8 Hz), 7.50 (2H, d J=8.8 Hz), 7.71-7.82 (3H, m), 7.94 (1H, s), m/z 449 (M+H)⁺.

### EXAMPLE 235

### 4-[[5-[4-(4-cyanophenyl)piperidine-1-carbonyl]-2-methyl-phenyl]carbamoylamino]-N-propyl-butanamide

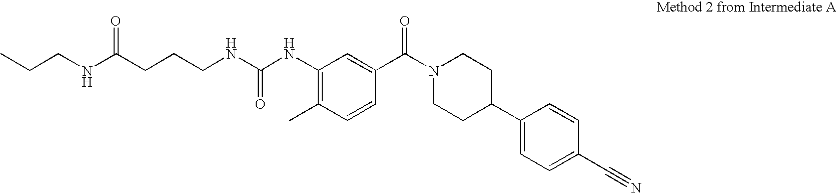

¹H NMR (300.073 MHz, d₆-DMSO, 30° C.) δ 0.82 (3H, t J=7.5 Hz), 1.30-1.46 (2H, m), 1.49-1.93 (6H, m), 2.10 (2H, t J=7.5 Hz), 2.19 (3H, s), 2.69-3.15 (7H, m), 3.63-3.93 (1H, m appears as a broad flat singlet), 4.39-4.78 (1H, m appears as a broad flat singlet), 6.62 (1H, t J=5.2 Hz), 6.91 (1H, d J=8.4 Hz), 7.16 (1H, d J=8.5 Hz), 7.49 (2H, d J=8.4 Hz), 7.69 (1H, s), 7.71-7.82 (3H, m), 7.95 (1H, s), m/z 490 (M+H)⁺.

### EXAMPLE 236

### (RS)-1-[5-[4-(4-cyanophenyl)piperidine-1-carbonyl]-2-methyl-phenyl]-3-(2-oxo-3-piperidyl)urea

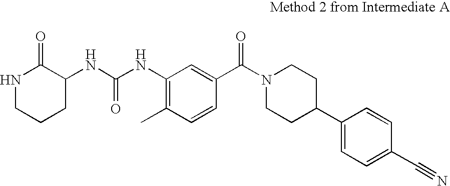

¹H NMR (300.073 MHz, d₆-DMSO, 30° C.) δ 1.43-1.94 (7H, m), 2.13-2.30 (4H, m), 2.76-3.22 (5H, m), 3.61-3.93 (1H, m), 3.95-4.09 (1H, m), 4.40-4.80 (1H, m), 6.87-7.02 (2H, m), 7.17 (1H, d J=8.5 Hz), 7.49 (2H, d J=7.7 Hz), 7.63 (1H, s), 7.75 (2H, d J=6.2 Hz), 7.97 (1H, s), 8.02 (1H, s), m/z 460 (M+H)⁺.

### EXAMPLE 237

### Methyl 2-[[2-[[5-[4-(4-cyanophenyl)piperidine-1-carbonyl]-2-methyl-phenyl]carbamoylamino]acetyl]amino]acetate

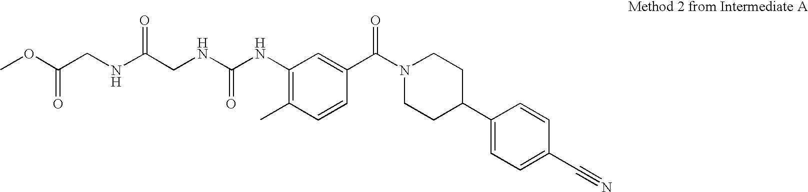

¹H NMR (300.073 MHz, d₆-DMSO, 30° C.) δ 1.42-1.93 (4H, m), 2.21 (3H, s), 2.68-3.11 (3H, m), 3.63 (3H, s), 3.69-3.96 (5H, m, consists of 3.80, d J=5.5 Hz, 3.87 d J=6.1 Hz) plus br s, 4.43-4.74 (1H, m appears as a broad flat singlet), 6.93 (2H, d J=6.7 Hz), 7.18 (1H, d J=7.3 Hz), 7.50 (2H, d J=8.5 Hz), 7.76 (2H, d J=9.7 Hz), 7.95 (1H, s), 8.01 (1H, s), 8.37 (1H, t J=5.8 Hz); the spectrum also contains signals due to MeOH and DCM, m/z 492 (M+H)⁺.

### EXAMPLE 238

### 2-[[5-[4-(4-cyanophenyl)piperidine-1-carbonyl]-2-methyl-phenyl]carbamoylamino]-N,N-dimethyl-acetamide

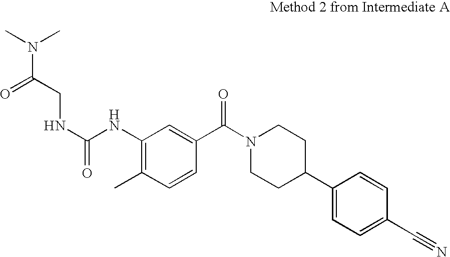

¹H NMR (300.073 MHz, d₆-DMSO, 30°. C) δ 1.46-1.93 (4H, m), 2.21 (3H, s), 2.67-3.20 (9H, m), 3.63-3.92 (1H, m), 3.96 (2H, d J=6.7 Hz), 4.40-4.74 (1H, m), 6.83-6.97 (2H, m), 7.18 (1H, d J=7.7 Hz), 7.49 (2H, d J=9.3 Hz), 7.76 (2H, d J=7.0 Hz), 7.93 (1H, s), 8.16 (1H, s), m/z 448 (M+H)⁺.

### EXAMPLE 239

### (RS)-1-[5-[4-(4-cyanophenyl)piperidine-1-carbonyl]-2-methyl-phenyl]-3-(2-oxo-3,4-dihydro-1H-1,8-naphthyridin-3-yl)urea

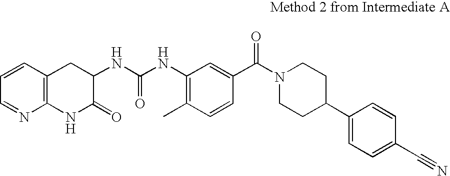

¹H NMR (300.073 MHz, d₆-DMSO, 30° C.) δ 1.43-1.95 (4H, m), 2.24 (3H, s), 2.64-3.20 (4H, m), 3.29 (1H, s, obscured by HOD signal), 3.59-3.98 (1H, m), 4.32-4.48 (1H, m), 4.49-4.74 (1H, m), 6.89-7.06 (2H, m), 7.19 (2H, d J=6.8 Hz), 7.50 (2H, d J=7.7 Hz), 7.64 (1H, d J=6.0 Hz), 7.75 (2H, d J=6.0 Hz), 7.97 (1H, s), 8.09-8.24 (2H, m), 10.76 (1H, s), m/z 507 (M−H)⁻ 509 (M+H)⁺.

### EXAMPLE 240

### (2S)-2-[[5-[4-(4-cyanophenyl)piperidine-1-carbonyl]-2-methyl-phenyl]carbamoylamino]-4-methylsulfonyl-butanoic acid

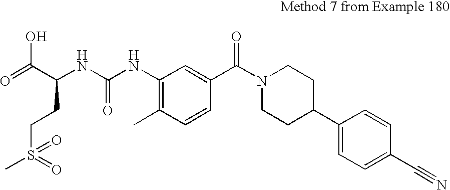

The title compound was prepared by hydrolysis as described in Method 7 (Example 125), starting from tert-butyl (2S)-2-[[5-[4-(4-cyanophenyl)piperidine-1-carbonyl]-2-methyl-phenyl]carbamoylamino]-4-methylsulfonyl-butanoate (Example 180), but using NaOH instead of LiOH, ¹H NMR (300.073 MHz, d₆-DMSO) δ 1.47-1.89 (m, 4H), 1.92-2.11 (m, 1H), 2.13-2.34 (m, 4H), 2.76-3.28 (m, 8H), 3.62-3.95 (m, 1H), 4.26-4.38 (m, 1H), 4.45-4.79 (m, 1H), 6.94 (d, 1H), 7.11 (d, 1H), 7.19 (d, 1H), 7.49 (d, 2H), 7.76 (d, 2H), 7.96 (d, 2H), 12.69-13.23 (m, 1H), m/z 527 (M+H)⁺.

### EXAMPLE 241

### 3-[[5-[4-(4-cyanophenyl)piperidine-1-carbonyl]-2-methyl-phenyl]carbamoylamino]-N-(5-methyl-1,2-oxazol-4-yl)propanamide

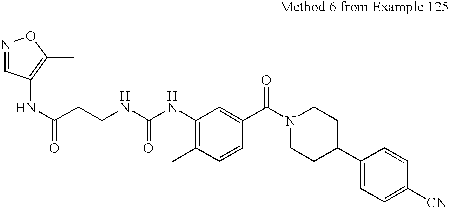

The title compound was prepared by the process described in Method 6, starting from 3-[[5-[4-(4-cyanophenyl)piperidine-1-carbonyl]-2-methyl-phenyl]carbamoylamino]propanoic acid (Example 125), (d₆-DMSO) 1.57-1.90 (4H, m), 2.18 (3H, s), 2.36 (3H, s), 2.52-2.54 (2H, m), 2.71-2.96 (3H, m), 3.38 (2H, q, J6.1), 3.70-4.04 (1H, m), 4.57-4.72 (1H, m), 6.77 (1H, t, J5.9), 6.91 (1H, dd, J7.7, 1.6), 7.16 (1H, d, J7.7), 7.49 (2H, d, J8.2), 7.76 (2H, d, J8.2), 7.82 (1H, s), 7.96 (1H, d, J1.6), 8.70 (1H, s), 9.70 (1H, s), m/z 513 (M−H)⁻ 515 (M+H)⁺.

### EXAMPLE 242

### 3-[[5-[4-(4-cyanophenyl)piperidine-1-carbonyl]-2-methyl-phenyl]carbamoylamino]-N,N-bis(2-hydroxyethyl)propanamide

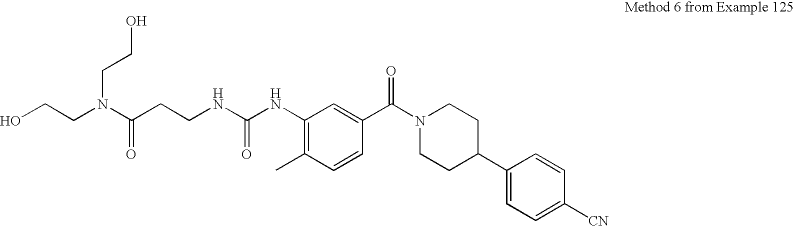

The title compound was prepared by the process described in Method 6, starting from 3-[[5-[4-(4-cyanophenyl)piperidine-1-carbonyl]-2-methyl-phenyl]carbamoylamino]-propanoic acid (Example 125), (d₆-DMSO) 1.57-1.97 (4H, m), 2.18 (3H, s), 2.50-2.57 (2H, m), 2.69-3.12 (3H, m), 3.30-3.40 (4H, m), 3.46-3.51 (4H, m), 3.73-3.95 (1H, m), 4.39-4.65 (1H, m), 4.63 (1H, t, J5.3), 4.78 (1H, t, J5.3), 6.71 (1H, t, J5.9), 6.90 (1H, dd, J7.5, 1.4), 7.15 (1H, d, J7.9), 7.49 (2H, d, J8.1), 7.76 (2H, d, J8.1), 7.87 (1H, s), 7.95 (1H, s), m/z 522 (M+H)⁺.

### EXAMPLE 243

### 1-[5-[4-(4-cyanophenyl)piperidine-1-carbonyl]-2-methyl-phenyl]-3-[(4-methylsulfonylphenyl)methyl]urea

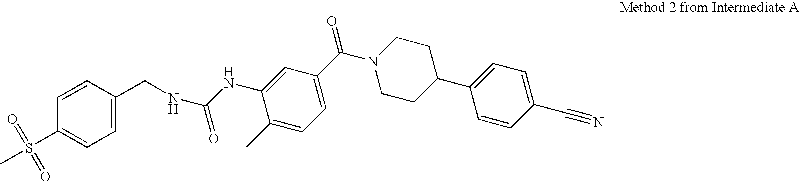

¹H NMR (300.072 MHz, CDCl₃) [symbol] 1.66-1.93 (m, 4H), 2.05 (s, 3H), 2.79-2.86 (m, 2H), 3.03 (s, 3H), 3.10-3.16 (m, 1H), 3.88-3.98 (m, 1H), 4.48 (d, 2H), 4.69-4.83 (m, 1H), 6.34 (t, 1H), 6.89-6.98 (m, 2H), 7.05 (d, 1H), 7.29-7.32 (m, 2H), 7.47 (d, 2H), 7.54-7.64 (m, 3H), 7.85 (d, 2H), m/z 531 (M+H)⁺.

### EXAMPLE 244

### 1-[5-[4-(4-cyanophenyl)piperidine-1-carbonyl]-2-methyl-phenyl]-3-(3-pyrazol-1-ylpropyl)urea

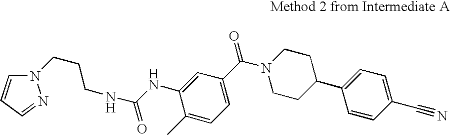

¹H NMR (300.072 MHz, CDCl₃) δ 1.64-1.90 (m, 4H), 2.06 (quintet, 2H), 2.16 (s, 3H), 2.76-2.90 (m, 2H), 3.05-3.18 (m, 1H), 3.20-3.27 (m, 2H), 3.89-4.03 (m, 1H), 4.22 (t, 2H), 4.69-4.99 (m, 1H), 5.48 (t, 1H), 6.23 (s, 1H), 6.44 (s, 1H), 7.05 (d, 1H), 7.15 (d, 1H), 7.31 (d, 2H), 7.42-7.46 (m, 2H), 7.58-7.64 (m, 3H), m/z 471 (M+H)⁺.

### EXAMPLE 245

### 1-[5-[4-(4-cyanophenyl)piperidine-1-carbonyl]-2-methyl-phenyl]-3-[(3-methyl-1,2-oxazol-5-yl)methyl]urea

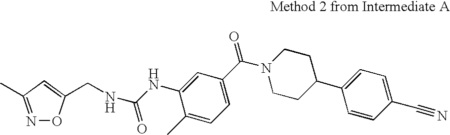

¹H NMR (300.072 MHz, CDCl₃) [symbol] 1.70-1.85 (m, 4H), 2.01 (s, 3H), 2.26 (s, 3H), 2.76-2.88 (m, 2H), 3.07-3.20 (m, 1H), 3.85-4.04 (m, 1H), 4.46 (d, 2H), 4.79-4.90 (m, 1H), 6.04 (s, 1H), 6.20 (t, 1H), 6.90-7.04 (m, 3H), 7.33 (d, 2H), 7.46 (s, 1H), 7.61 (d, 2H), m/z 458 (M+H)⁺.

### EXAMPLE 246

### 1-[5-[4-(4-cyanophenyl)piperidine-1-carbonyl]-2-methyl-phenyl]-3-[(5-methyl-1,2-oxazol-3-yl)methyl]urea

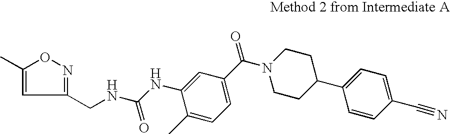

¹H NMR (300.072 MHz, CDCl₃) [symbol] 1.64-2.00 (m, 4H), 2.09 (s, 3H), 2.38 (s, 3H), 2.77-2.89 (m, 2H), 3.08-3.19 (m, 1H), 3.86-4.10 (m, 1H), 4.44 (d, 2H), 4.73-4.94 (m, 1H), 5.92 (t, 1H), 6.02 (s, 1H), 6.88 (s, 1H), 6.96-7.10 (m, 2H), 7.33 (d, 2H), 7.52-7.55 (m, 1H), 7.61 (d, 2H), m/z 458 (M+H)⁺.

### EXAMPLE 247

### 1-[5-[4-(4-cyanophenyl)piperidine-1-carbonyl]-2-methyl-phenyl]-3-[(3-methylsulfonylphenyl)methyl]urea

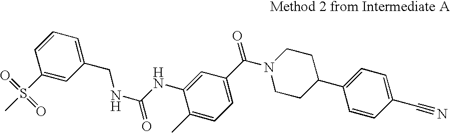

¹H NMR (300.072 MHz, CDCl₃) δ 1.56-1.97 (m, 4H), 2.04 (s, 3H), 2.75-2.89 (m, 2H), 3.04 (s, 3H), 3.11-3.25 (m, 1H), 3.87-4.07 (m, 1H), 4.45 (d, 2H), 4.66-4.86 (m, 1H), 6.31 (t, 1H), 6.92-6.97 (m, 2H), 7.05 (d, 1H), 7.31 (d, 2H), 7.50 (t, 1H), 7.57-7.62 (m, 4H), 7.79 (d, 1H), 7.85 (s, 1H), m/z 531 (M+H)⁺.

### EXAMPLE 248

### 1-[5-[4-(4-cyanophenyl)piperidine-1-carbonyl]-2-methyl-phenyl]-3-(1,3-thiazol-2-ylmethyl)urea

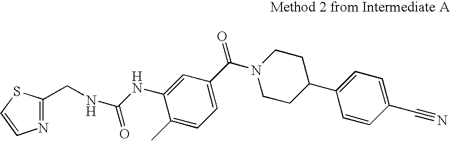

¹H NMR (400.132 MHz, CDCl₃) δ 1.65-1.98 (m, 4H), 2.10 (s, 3H), 2.79-2.88 (m, 2H), 2.99-3.19 (m, 1H), 3.83-4.08 (m, 1H), 4.73 (d, 2H), 4.77-4.90 (m, 1H), 6.20 (t, 1H), 6.96-7.01 (m, 2H), 7.07 (d, 1H), 7.25 (s, 1H), 7.31 (d, 2H), 7.54-7.56 (m, 1H), 7.60 (d, 2H), 7.69 (d, 1H), m/z 460 (M+H)⁺.

### EXAMPLE 249

### 1-[5-[4-(4-cyanophenyl)piperidine-1-carbonyl]-2-methyl-phenyl]-3-[(6-methylpyridin-2-yl)methyl]urea

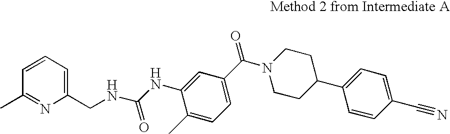

¹H NMR (400.132 MHz, CDCl₃) [symbol] 1.56-1.93 (m, 4H), 2.22 (s, 3H), 2.50 (s, 3H), 2.77-2.87 (m, 2H), 2.94-3.14 (m, 1H), 3.87-4.13 (m, 1H), 4.50 (d, 2H), 4.71-4.88 (m, 1H), 6.01-6.05 (m, 1H), 6.82-6.86 (m, 1H), 7.03 (d, 1H), 7.09 (t, 2H), 7.16 (d, 1H), 7.30 (d, 2H), 7.54 (t, 1H), 7.60 (d, 2H), 7.67-7.68 (m, 1H), m/z 468 (M+H)⁺.

### EXAMPLE 250

### 3-[5-[4-(4-cyanophenyl)piperidine-1-carbonyl]-2-methyl-phenyl]-1-[(3-methylpyridin-2-yl)methyl]urea

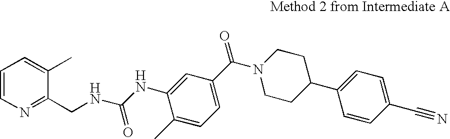

¹H NMR (400.132 MHz, CDCl₃) [symbol] 1.66-1.96 (m, 4H), 2.26 (s, 3H), 2.31 (s, 3H), 2.78-2.86 (m, 2H), 2.95-3.19 (m, 1H), 3.95-4.15 (m, 1H), 4.53 (d, 2H), 4.73-4.93 (m, 1H), 6.64 (s, 1H), 6.87 (s, 1H), 7.09-7.12 (m, 2H), 7.19 (d, 1H), 7.30 (d, 2H), 7.46 (d, 1H), 7.59 (d, 2H), 7.72-7.76 (m, 1H), 8.29 (d, 1H), m/z 468 (M+H)⁺.

### EXAMPLE 251

### 1-[5-[4-(4-cyanophenyl)piperidine-1-carbonyl]-2-methyl-phenyl]-3-[[3-(2-methoxypyridin-3-yl)-1,2-oxazol-5-yl]methyl]urea

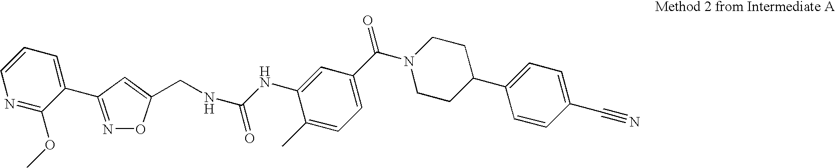

¹H NMR (400.132 MHz, CDCl₃) [symbol] 1.56-1.99 (m, 4H), 2.12 (s, 3H), 2.77-2.86 (m, 2H), 3.02-3.19 (m, 1H), 3.88-3.98 (m, 1H), 4.02 (s, 3H), 4.58 (s, 2H), 4.76-4.97 (m, 1H), 5.86 (s, 1H), 6.75 (s, 2H), 6.95-6.99 (m, 1H), 7.04 (d, 1H), 7.13 (d, 1H), 7.30 (d, 2H), 7.46 (s, 1H), 7.58 (d, 2H), 8.15-8.19 (m, 1H), 8.23-8.27 (m, 1H), m/z 551 (M+H)⁺.

### EXAMPLE 252

### 1-[5-[4-(4-cyanophenyl)piperidine-1-carbonyl]-2-methyl-phenyl]-3-(4-methoxybutyl)urea

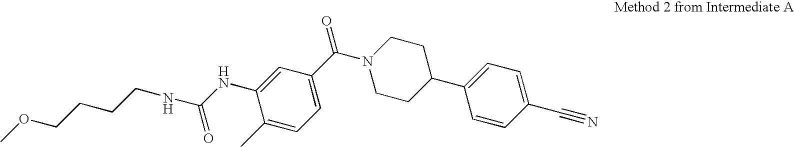

¹H NMR (400.132 MHz, CDCl₃) [symbol] 1.57-1.63 (m, 4H), 1.69-2.03 (m, 4H), 2.18 (s, 3H), 2.78-2.90 (m, 2H), 3.01-3.12 (m, 1H), 3.26 (q, 2H), 3.30 (s, 3H), 3.39 (t, 2H), 3.88-4.10 (m, 1H), 4.79-4.93 (m, 1H), 5.07 (t, 1H), 6.19 (s, 1H), 7.07 (d, 1H), 7.17 (d, 1H), 7.32 (d, 2H), 7.53-7.55 (m, 1H), 7.62 (d, 2H), m/z 449 (M+H)⁺.

### EXAMPLE 253

### (RS)-1-[5-[4-(4-cyanophenyl)piperidine-1-carbonyl]-2-methyl-phenyl]-3-(1-phenoxypropan-2-yl)urea

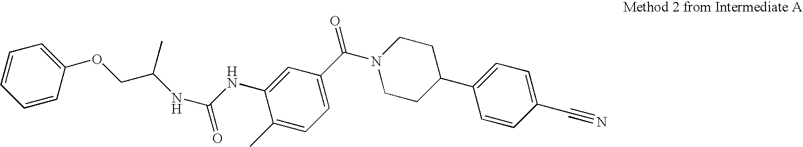

¹H NMR (400.132 MHz, CDCl₃) [symbol] 1.33 (d, 3H), 1.59-1.88 (m, 4H), 2.13 (s, 3H), 2.73-2.84 (m, 2H), 2.98-3.13 (m, 1H), 3.89-3.95 (m, 1H), 3.97-4.00 (m, 2H), 4.19-4.29 (m, 1H), 4.60-4.87 (m, 1H), 5.34 (d, 1H), 6.48 (s, 1H), 6.89-6.97 (m, 4H), 7.03 (d, 1H), 7.11 (d, 1H), 7.27-7.31 (m, 3H), 7.54 (s, 1H), 7.60 (d, 2H), m/z 497 (M+H)⁺.

### EXAMPLE 254

### 1-[5-[4-(4-cyanophenyl)piperidine-1-carbonyl]-2-methyl-phenyl]-3-[2-(3,3-difluoropyrrolidin-1-yl)ethyl]urea

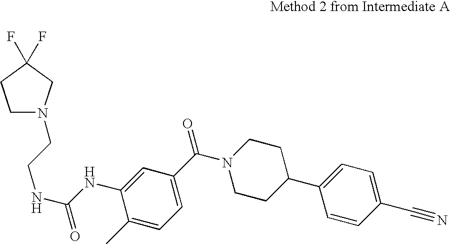

¹H NMR (300.072 MHz, CDCl₃) δ 1.55-2.05 (4H, m), 2.05 (3H, s), 2.18-2.33 (2H, m), 2.57-2.60 (1H, m), 2.62-2.68 (1H, m), 2.77-3.20 (7H, t), 3.30-3.39 (2H, m), 3.86-3.99 (1H, m), 4.85 (1H, s), 5.91 (1H, t), 6.93-6.96 (1H, m), 7.02 (1H, s), 7.06 (1H, t), 7.32 (2H, d), 7.54 (1H, d), 7.59-7.62 (2H, m), m/z 496 (M+H)⁺.

### EXAMPLE 255

### (RS)-1-[5-[4-(4-cyanophenyl)piperidine-1-carbonyl]-2-methyl-phenyl]-3-(1-methyl-3-piperidyl)urea

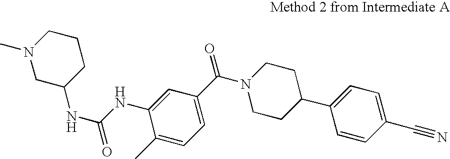

¹H NMR (300.072 MHz, CDCl₃) δ 1.12 (2H, d), 1.41 (1H, d), 1.50-2.40 (13H, m), 2.54-3.25 (5H, m), 3.92 (2H, m), 4.86 (1H, s), 5.98 (1H, d), 6.96-6.99 (1H, m), 7.07-7.10 (2H, m), 7.31 (2H, t), 7.60 (2H, d), 7.67 (1H, d), m/z 460 (M+H)⁺.

### EXAMPLE 256

### 1-[5-[4-(4-cyanophenyl)piperidine-1-carbonyl]-2-methyl-phenyl]-3-(4-dimethylaminocyclohexyl)urea

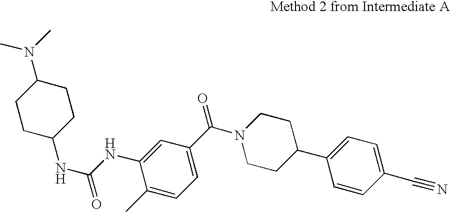

¹H NMR (300.072 MHz, CDCl₃) δ 0.98-1.20 (4H, m), 1.23-2.25 (10H, m), 2.30 (6H, s), 2.79-3.25 (4H, m), 3.46-3.53 (1H, m), 4.01 (1H, m), 4.85 (1H, m), 5.80 (0.5H, d), 6.06 (0.5H, d), 6.93-7.19 (3H, m), 7.32 (2H, d), 7.60 (2H, d), 7.67-7.69 (1H, m), m/z 488 (M+H)⁺.

### EXAMPLE 257

### 1-[5-[4-(4-cyanophenyl)piperidine-1-carbonyl]-2-methyl-phenyl]-3-[(1,5-dimethylpyrazol-3-yl)methyl]urea

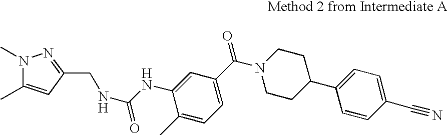

¹H NMR (300.072 MHz, CDCl₃) [symbol] 1.56-1.94 (m, 4H), 2.11 (s, 3H), 2.20 (s, 3H), 2.76-2.86 (m, 2H), 3.01-3.18 (m, 1H), 3.68 (s, 3H), 3.89-4.02 (m, 1H), 4.33 (d, 2H), 4.72-5.01 (m, 1H), 5.84-5.90 (m, 1H), 5.97 (s, 1H), 6.97-7.11 (m, 3H), 7.31 (d, 2H), 7.60 (d, 2H), 7.69 (d, 1H), m/z 471 (M+H)⁺.

### EXAMPLE 258

### 1-[5-[4-(4-cyanophenyl)piperidine-1-carbonyl]-2-methyl-phenyl]-3-[(1,3-dimethylpyrazol-4-yl)methyl]urea

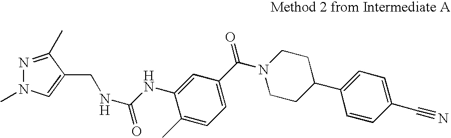

¹H NMR (300.072 MHz, CDCl₃) [symbol] 1.55-1.85 (m, 4H), 2.02 (s, 3H), 2.23 (s, 3H), 2.79-2.87 (m, 2H), 3.01-3.13 (m, 1H), 3.77 (s, 3H), 3.88-4.04 (m, 1H), 4.21 (d, 2H), 4.74-4.81 (m, 1H), 5.61 (t, 1H), 6.67 (s, 1H), 6.91-7.07 (m, 2H), 7.26-7.35 (m, 3H), 7.53 (s, 1H), 7.63 (d, 2H), m/z 471 (M+H)⁺.

### EXAMPLE 259

### (RS)-1-[5-[4-(4-cyanophenyl)piperidine-1-carbonyl]-2-ethyl-phenyl]-3-(1,4-dioxan-2-ylmethyl)urea

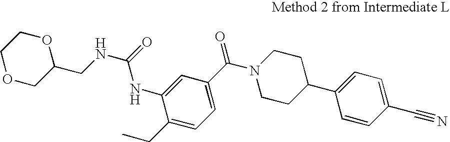

¹H NMR (300.073 MHz, d₆-DMSO, 30° C.) δ 1.15 (3H, tJ=7.7 Hz), 1.48-1.92 (4H, m), 2.45-2.64 (2H, m˜assumed to be 2H under the DMSO), 2.70-3.29 (6H, m), 3.37-3.98 (7H, m), 4.41-4.79 (1H, m), 6.76 (1H, tJ=6.0 Hz), 6.97 (1H, dJ=7.6 Hz), 7.18 (1H, dJ=10.1 Hz), 7.49 (2H, dJ=7.6 Hz), 7.76 (2H, dJ=9.3 Hz), 7.81 (1H, s), 7.92 (1H, s), m/z 477 (M+H)⁺.

### EXAMPLE 260

### 3-(1-amino-2-methyl-propan-2-yl)-1-[5-[4-(4-cyanophenyl)piperidine-1-carbonyl]-2-methyl-phenyl]urea

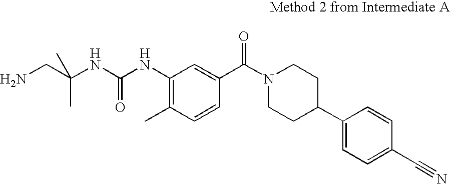

¹H NMR (300.072 MHz, CDCl₃) [symbol] 1.21 (s, 6H), 1.56-1.98 (m, 4H), 2.31 (s, 3H), 2.80-2.93 (m, 2H), 3.11-3.23 (m, 1H), 3.30-3.38 (m, 2H), 3.95-4.09 (m, 1H), 4.57-5.03 (m, 3H), 6.93 (d, 1H), 7.11 (d, 1H), 7.32 (d, 2H), 7.54 (s, 1H), 7.59 (d, 2H), 7.84 (s, 1H), 8.26 (s, 1H), m/z 434 (M+H)⁺.

### EXAMPLE 261

### N-[1-[[5-[4-(4-cyanophenyl)piperidine-1-carbonyl]-2-methyl-phenyl]carbamoylamino]-2-methyl-propan-2-yl]acetamide

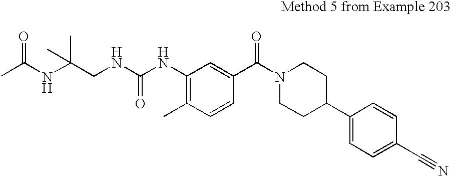

¹H NMR (300.072 MHz, CDCl₃) [symbol] 1.33 (s, 6H), 1.50-1.89 (m, 4H), 1.86 (s, 3H), 2.11 (s, 3H), 2.78-2.92 (m, 2H), 3.03-3.16 (m, 1H), 3.31 (d, 2H), 3.93-4.03 (m, 1H), 4.77-4.94 (m, 1H), 6.28 (t, 1H), 6.63 (s, 1H), 6.89 (s, 1H), 6.97-7.13 (m, 2H), 7.32 (d, 2H), 7.58-7.65 (m, 3H), m/z 476 (M+H)⁺.

### EXAMPLE 262

### N-[1-[[5-[4-(4-cyanophenyl)piperidine-1-carbonyl]-2-methyl-phenyl]carbamoylamino]-2-methyl-propan-2-yl]propanamide

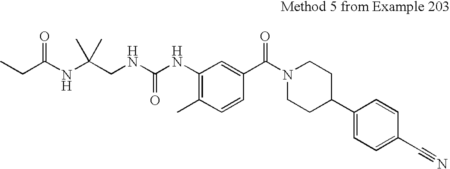

¹H NMR (300.072 MHz, CDCl₃) δ 1.07 (t, 3H), 1.34 (s, 6H), 1.60-2.01 (m, 4H), 2.11 (q, 2H), 2.14 (s, 3H), 2.80-2.89 (m, 2H), 3.02-3.19 (m, 1H), 3.32 (d, 2H), 3.92-4.04 (m, 1H), 4.67-5.01 (m, 1H), 6.22 (t, 1H), 6.46 (s, 1H), 6.76 (s, 1H), 6.99-7.15 (m, 2H), 7.32 (d, 2H), 7.58-7.64 (m, 3H), m/z 490 (M+H)⁺.

### EXAMPLE 263

### N-[1-[[5-[4-(4-cyanophenyl)piperidine-1-carbonyl]-2-methyl-phenyl]carbamoylamino]-2-methyl-propan-2-yl]-2-methyl-propanamide

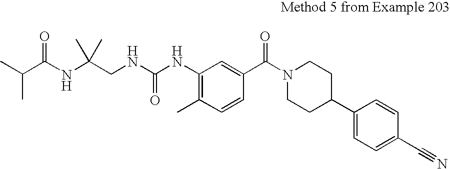

¹H NMR (300.072 MHz, CDCl₃) δ 1.06 (d, 6H), 1.33 (s, 6H), 1.55-1.79 (m, 4H), 2.14 (s, 3H), 2.25 (septet, 1H), 2.79-2.91 (m, 2H), 3.03-3.20 (m, 1H), 3.30 (d, 2H), 3.91-4.05 (m, 1H), 4.74-4.97 (m, 1H), 6.21 (t, 1H), 6.49 (s, 1H), 6.77 (s, 1H), 7.01-7.15 (m, 2H), 7.31 (d, 2H), 7.54-7.64 (m, 3H), m/z 504 (M+H)⁺.

### EXAMPLE 264

### 1-[5-[4-(4-cyanophenyl)piperidine-1-carbonyl]-2-methyl-phenyl]-3-[(4-methyl-1,3-thiazol-2-yl)methyl]urea

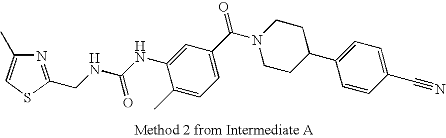

¹H NMR (300.072 MHz, CDCl₃) δ 1.50-2.04 (4H, m), 2.03-2.04 (3H, m), 2.40 (3H, d), 2.78-2.86 (2H, m), 3.11 (1H, m), 3.94 (1H, m), 4.65 (2H, d), 4.82 (1H, m), 6.45 (1H, t), 6.77 (1H, d), 6.91-6.95 (1H, m), 7.00-7.03 (1H, m), 7.32 (3H, m), 7.55-7.60 (3H, m), m/z 474 (M+H)⁺.

### EXAMPLE 265

### 1-[5-[4-(4-cyanophenyl)piperidine-1-carbonyl]-2-methyl-phenyl]-3-[(3R)-6-oxo-3-piperidyl]urea

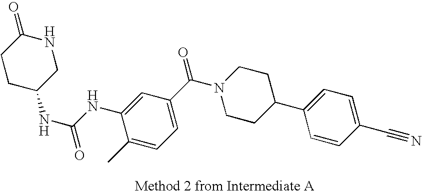

¹H NMR (300.072 MHz, CDCl₃) δ 1.50-2.05 (4H, m), 2.14 (3H, m), 2.40-2.60 (2H, m), 2.75-3.30 (6H, m), 3.62 (1H, m), 4.00 (1H, m), 4.20 (1H, m), 4.85 (1H, m), 6.45 (1H, s), 6.79 (1H, d), 6.90-6.93 (1H, m), 7.05-7.12 (1H, m), 7.33 (2H, d), 7.45 (1H, s), 7.59-7.62 (2H, m), 7.90 (1H, s), m/z 460 (M+H)⁺.

### EXAMPLE 266

### 1-[5-[4-(4-cyanophenyl)piperidine-1-carbonyl]-2-methyl-phenyl]-3-(pyrimidin-4-ylmethyl)urea

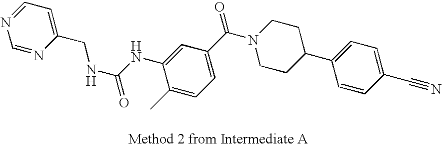

¹H NMR (300.072 MHz, CDCl₃) δ 1.50-2.05 (4H, m), 2.09 (3H, s), 2.79-2.87 (2H, m), 3.22-3.25 (1H, m), 3.99 (1H, m), 4.49 (2H, d), 4.85 (1H, m), 6.79 (1H, t), 6.92-6.96 (1H, m), 7.03-7.06 (1H, m), 7.28-7.35 (3H, m), 7.44 (1H, s), 7.58-7.61 (2H, m), 7.71 (1H, d), 8.62-8.64 (1H, m), 9.09 (1H, d), m/z 455 (M+H)⁺.

### EXAMPLE 267

### 1-[5-[4-(4-cyanophenyl)piperidine-1-carbonyl]-2-methyl-phenyl]-3-[(1-methylimidazol-4-yl)methyl]urea

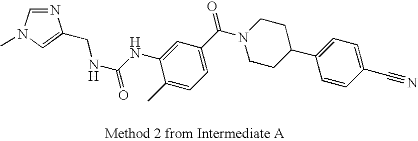

¹H NMR (300.072 MHz, CDCl₃) δ 1.50-2.05 (4H, s), 2.10 (3H, s), 2.78-3.25 (3H, m), 3.61 (3H, s), 4.05 (1H, m), 4.29 (2H, d), 4.85 (1H, m), 6.66 (1H, t), 6.84 (1H, d), 6.97-7.00 (1H, m), 7.07-7.10 (1H, m), 7.32 (2H, t), 7.34 (1H, s), 7.59-7.61 (2H, m), 7.67 (1H, s), 7.84 (1H, d), m/z 457 (M+H)⁺.

### EXAMPLE 268

### 1-[5-[4-(4-cyanophenyl)piperidine-1-carbonyl]-2-methyl-phenyl]-3-[(1-methylpyrrolidin-3-yl)methyl]urea

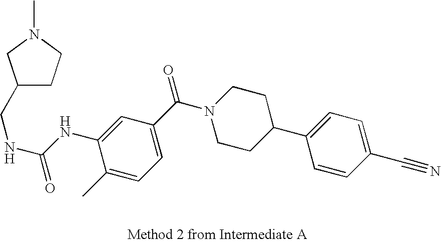

¹H NMR (300.072 MHz, CDCl₃) δ 1.45-2.05 (6H, m), 2.17 (3H, s), 2.32-2.34 (3H, m), 2.40 (1H, d), 2.46-2.67 (3H, m), 2.80-3.20 (3H, m), 3.20 (2H, t), 4.00 (1H, m), 4.85 (1H, m), 6.22 (1H, t), 6.95-6.98 (1H, m), 7.09-7.12 (1H, m), 7.17 (1H, s), 7.32 (2H, d), 7.60 (2H, d), 7.72 (1H, d), m/z 460 (M+H)⁺.

### EXAMPLE 269

### 1-[5-[4-(4-cyanophenyl)piperidine-1-carbonyl]-2-methyl-phenyl]-3-(1,3-oxazol-2-ylmethyl)urea

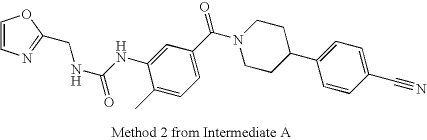

¹H NMR (300.072 MHz, CDCl₃) δ 1.50-2.05 (4H, m), 2.05 (3H, s), 2.78-3.20 (3H, m), 3.95 (1H, m), 4.52 (2H, d), 4.85 (1H, m), 6.53 (1H, t), 6.92-6.96 (1H, m), 7.03 (2H, d), 7.33 (3H, t), 7.59-7.61 (3H, m), 7.62 (1H, d), m/z 444 (M+H)⁺.

### EXAMPLE 270

### 3-[(8S)-1-azabicyclo[2.2.2]oct-8-yl]-1-[5-[4-(4-cyanophenyl)piperidine-1-carbonyl]-2-methyl-phenyl]urea

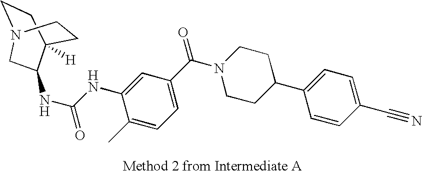

¹H NMR (300.072 MHz, CDCl₃) δ 1.50-2.05 (7H, m), 2.18 (2H, s), 2.38 (2H, s), 2.60 (1H, m), 2.75-3.25 (5H, m), 3.25-3.62 (3H, m), 4.00 (2H, m), 4.85 (1H, m), 5.10 (1H, m), 6.45 (1H, s), 6.95 (1H, m), 7.10-7.25 (2H, m), 7.33 (2H, m), 7.59-7.62 (2H, d), 7.98 (1H, m), m/z 472 (M+H)⁺.

### EXAMPLE 271

### N-[2-[[5-[4-(4-cyanophenyl)piperidine-1-carbonyl]-2-methyl-phenyl]carbamoylamino]ethyl]-N, 2-dimethyl-propanamide

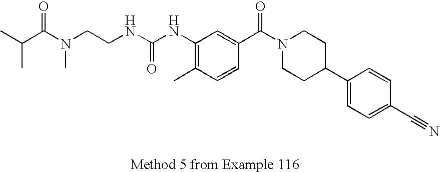

¹H NMR (300.072 MHz, CDCl₃) d 1.08 (d, 6H), 1.56-1.98 (m, 4H), 2.18 (s, 3H), 2.74-2.96 (m, 3H), 3.12 (s, 3H), 3.04-3.15 (m, 1H), 3.35-3.42 (m, 2H), 3.45-3.53 (m, 2H), 3.92-4.19 (m, 1H), 4.72-4.93 (m, 1H), 5.98 (t, 1H), 6.98-7.16 (m, 3H), 7.32 (d, 2H), 7.58-7.66 (m, 2H), 7.74 (s, 1H) Complicated due to rotamers, m/z 490 (M+H)⁺; HPLC tR=2.02 min.

### EXAMPLE 272

### 3-[5-[4-(4-cyanophenyl)piperidine-1-carbonyl]-2-methyl-phenyl]-1-[2-(ethylsulfonyl-methyl-amino)ethyl]urea

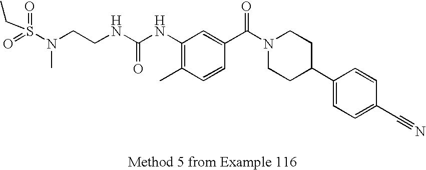

¹H NMR (300.072 MHz, CDCl₃) d 1.33 (t, 3H), 1.52-2.04 (m, 4H), 2.10 (s, 3H), 2.79-2.88 (m, 2H), 2.92 (s, 3H), 2.98 (q, 2H), 3.06-3.19 (m, 1H), 3.26-3.32 (m, 2H), 3.36-3.41 (m, 2H), 3.88-4.09 (m, 1H), 4.74-5.02 (m, 1H), 5.80-5.86 (m, 1H), 6.95 (s, 1H), 6.98-7.10 (m, 2H), 7.34 (d, 2H), 7.55 (d, 1H), 7.60 (d, 2H), m/z 512 (M+H)⁺; HPLC tR=2.07 min.

### EXAMPLE 273

### N-[3-[[5-[4-(4-cyanophenyl)piperidine-1-carbonyl]-2-methyl-phenyl]carbamoylamino]propyl]acetamide

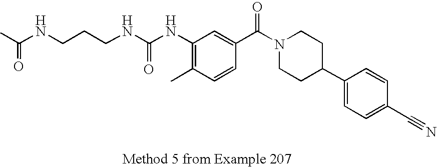

¹H NMR (300.072 MHz, CDCl₃) d 1.59-1.65 (m, 2H), 1.74-1.90 (m, 4H), 1.95 (s, 3H), 2.13 (s, 3H), 2.80-2.94 (m, 2H), 3.01-3.15 (m, 1H), 3.19-3.29 (m, 4H), 3.93-4.07 (m, 1H), 4.75-4.96 (m, 1H), 5.95 (t, 1H), 6.63 (t, 1H), 6.89 (s, 1H), 6.97-7.13 (m, 2H), 7.32 (d, 2H), 7.61 (d, 2H), 7.69 (s, 1H), m/z 462 (M+H)⁺; HPLC tR=1.79 min.

### EXAMPLE 274

### N-[3-[[5-[4-(4-cyanophenyl)piperidine-1-carbonyl]-2-methyl-phenyl]carbamoylamino]propyl]-2-methyl-propanamide

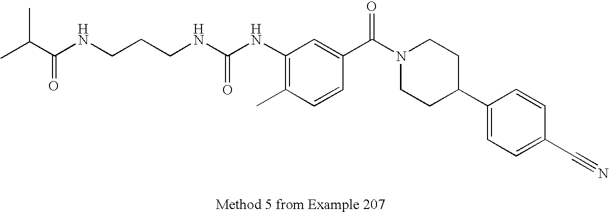

¹H NMR (300.072 MHz, CDCl₃) d 1.12 (d, 6H), 1.60 (quintet, 2H), 1.68-2.01 (m, 4H), 2.14 (s, 3H), 2.36 (septet, 1H), 2.78-2.91 (m, 2H), 3.08-3.30 (m, 5H), 3.91-4.16 (m, 1H), 4.75-4.96 (m, 1H), 5.99 (t, 1H), 6.54 (t, 1H), 6.91 (s, 1H), 6.96-7.01 (m, 1H), 7.12 (d, 1H), 7.32 (d, 2H), 7.61 (d, 2H), 7.68-7.71 (m, 1H), m/z 490 (M+H)⁺; HPLC tR=1.99 min.

### EXAMPLE 275

### 3-[(3R)-1-acetylpyrrolidin-3-yl]-1-[5-[4-(4-cyanophenyl)piperidine-1-carbonyl]-2-methyl-phenyl]urea

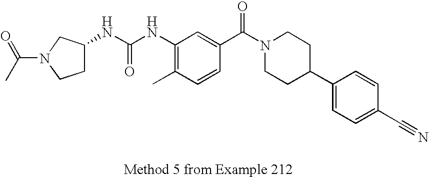

¹H NMR (300.072 MHz, CDCl₃) d 1.61-1.96 (m, 4H), 2.04 (s, 3H), 2.08 (s, 3H), 2.10-2.30 (m, 2H), 2.77-2.93 (m, 2H), 3.07-3.26 (m, 1H), 3.31-3.44 (m, 1H), 3.48-3.73 (m, 3H), 3.90-4.09 (m, 1H), 4.33-4.48 (m, 1H), 4.75-4.94 (m, 1H), 6.29-6.49 (m, 1H), 6.92-6.97 (m, 1H), 7.03-7.12 (m, 2H), 7.33 (d, 2H), 7.61 (d, 2H), 7.75 (d, 1H), m/z 474 (M+H)⁺; HPLC tR=1.82 min.

### EXAMPLE 276

### 3-[5-[4-(4-cyanophenyl)piperidine-1-carbonyl]-2-methyl-phenyl]-1-[(3R)-1-propanoylpyrrolidin-3-yl]urea

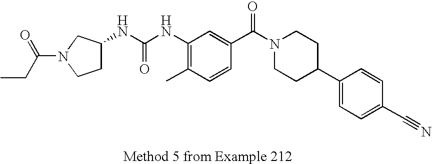

¹H NMR (300.072 MHz, CDCl₃) d 1.09-1.18 (m, 3H), 1.56-1.98 (m, 4H), 2.04-2.37 (m, 7H), 2.77-2.91 (m, 2H), 3.02-3.23 (m, 1H), 3.32-3.43 (m, 1H), 3.48-3.69 (m, 3H), 3.91-4.14 (m, 1H), 4.30-4.49 (m, 1H), 4.72-4.94 (m, 1H), 6.36-6.53 (m, 1H), 6.93-6.99 (m, 1H), 7.06-7.11 (m, 1H), 7.20 (d, 1H), 7.33 (d, 2H), 7.61 (d, 2H), 7.79 (d, 1H), m/z 488 (M+H)⁺; HPLC tR=1.92 min.

### EXAMPLE 277

### 3-[(3S)-1-acetylpyrrolidin-3-yl]-1-[5-[4-(4-cyanophenyl)piperidine-1-carbonyl]-2-methyl-phenyl]urea

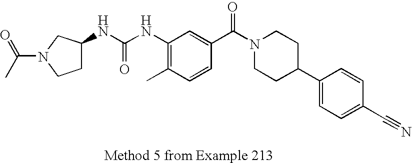

¹H NMR (300.072 MHz, CDCl₃) d 1.62-1.97 (m, 5H), 2.05 (s, 3H), 2.09 (d, 3H), 2.14-2.30 (m, 1H), 2.78-2.90 (m, 2H), 3.06-3.30 (m, 1H), 3.32-3.43 (m, 1H), 3.50-3.71 (m, 3H), 3.95-4.10 (m, 1H), 4.31-4.47 (m, 1H), 4.75-4.97 (m, 1H), 6.27-6.50 (m, 1H), 6.92-6.99 (m, 1H), 7.03-7.11 (m, 2H), 7.34 (d, 2H), 7.61 (d, 2H), 7.72-7.77 (m, 1H), m/z 474 (M+H)⁺; HPLC tR=1.81 min.

### EXAMPLE 278

### 1-[3-[4-(4-cyanophenyl)piperidine-1-carbonyl]-4-methyl-phenyl]-3-(3-methylsulfonylpropyl)urea

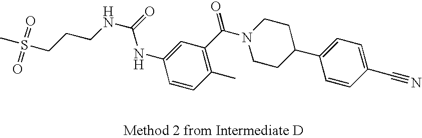

¹H NMR (300.073 MHz, d₆-DMSO, 30° C.) δ 1.32-1.96 (6H, m), 2.02-2.30 (3H, m), 2.68-3.01 (5H, m), 3.03-3.23 (5H, m), 3.43 (1H, dJ=12.5 Hz), 4.68 (1H, dJ=13.4 Hz), 6.25 (1H, tJ=5.7 Hz), 7.09 (1H, dJ=8.5 Hz), 7.15-7.34 (2H, m), 7.47 (2H, dJ=8.5 Hz), 7.76 (2H, dJ=8.5 Hz), 8.46 (1H, s), m/z 483 (M+H)⁺.

### EXAMPLE 279

### tert-butyl 3-[[5-[4-(4-cyanophenyl)piperidine-1-carbonyl]-2-methyl-phenyl]carbamoylamino]piperidine-1-carboxylate

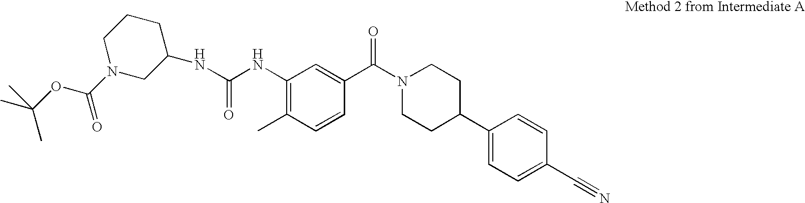

¹H NMR (300.072 MHz, CDCl₃) δ 1.44 (9H, s), 1.48-1.58 (2H, m), 1.65-1.75 (4H, m), 1.85-2.01 (2H, m), 2.11 (3H, s), 2.75-2.94 (2H, m), 3.04-3.27 (3H, m), 3.45-3.58 (1H, m), 3.68-3.86 (2H, m), 3.90-4.06 (1H, m), 4.73-4.95 (1H, m), 5.37-5.48 (1H, m), 6.54 (1H, s), 6.99-7.14 (2H, m), 7.32 (2H, d), 7.52 (1H, s), 7.61 (2H, d), m/z 546 (M+H)⁺; HPLC tR=2.52 min.

### EXAMPLE 280

### 1-benzyl-3-[5-[4-(4-cyanophenyl)piperidine-1-carbonyl]-2-(trifluoromethyl)phenyl]urea

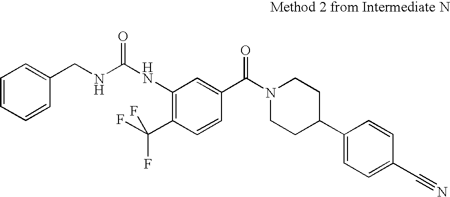

¹H NMR (300.072 MHz, CDCl₃, 30° C.) δ 1.48-2.00 (5H, m), 2.66-2.93 (2H, m), 3.03-3.24 (1H, m), 3.74-3.94 (1H, m), 4.67-4.90 (1H, m), 6.25 (1H, s), 7.01 (1H, dJ=8.8 Hz), 7.15-7.39 (8H, m), 7.46 (1H, dJ=8.1 Hz), 7.59 (2H, dJ=8.8 Hz), 7.87 (1H, s), m/z 507 (M+H)⁺.

### EXAMPLE 281

### 1-[5-[4-(4-cyanophenyl)piperidine-1-carbonyl]-2-methyl-phenyl]-3-(1-methylsulfonyl-4-piperidyl)urea

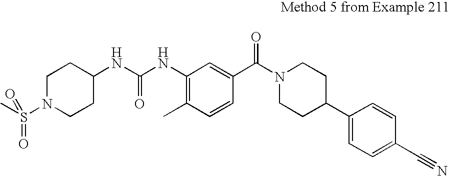

¹H NMR (300.073 MHz, d₆-DMSO) δ 1.31-1.97 (8H, m), 2.20 (3H, s), 2.75-3.21 (8H, m), 3.41-3.95 (4H, m), 4.34-4.88 (1H, m), 6.71 (1H, d), 6.92 (1H, d), 7.17 (1H, d), 7.49 (2H, d), 7.67 (1H, s), 7.76 (2H, d), 7.97 (1H, s), m/z 524 (M+H)⁺.

### EXAMPLE 282

### 3-(1-acetyl-4-piperidyl)-1-[5-[4-(4-cyanophenyl)piperidine-1-carbonyl]-2-methyl-phenyl]urea

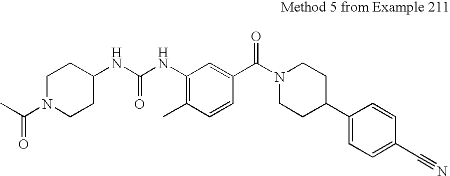

¹H NMR (300.073 MHz, d₆-DMSO) δ 1.23-1.93 (8H, m), 2.00 (3H, s), 2.19 (3H, s), 2.75-3.22 (5H, m), 3.55-3.96 (3H, m), 4.07-4.21 (1H, m), 4.38-4.79 (1H, m), 6.68 (1H, d), 6.91 (1H, d), 7.17 (1H, d), 7.49 (2H, d), 7.66 (1H, s), 7.76 (2H, d), 7.97 (1H, s), m/z 488 (M+H)⁺.

### EXAMPLE 283

### 1-[5-[4-(4-cyanophenyl)piperidine-1-carbonyl]-2-methyl-phenyl]-3-[1-(2-methylpropanoyl)-4-piperidyl]urea

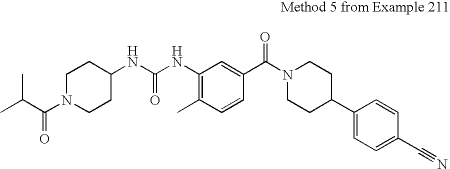

¹H NMR (300.073 MHz, d₆-DMSO) δ 0.99 (6H, d), 1.11-1.37 (2H, m), 1.45-1.95 (6H, m), 2.19 (3H, s), 2.32-2.45 (1H, m), 2.76-3.23 (5H, m), 3.59-3.94 (3H, m), 4.09-4.24 (1H, m), 4.35-4.80 (1H, m), 6.68 (1H, d), 6.87-6.96 (1H, m), 7.17 (1H, d), 7.49 (2H, d), 7.64 (1H, s), 7.76 (2H, d), 7.97 (1H, d), m/z 516 (M+H)⁺.

### EXAMPLE 284

### 4-[[5-[4-(4-cyanophenyl)piperidine-1-carbonyl]-2-methyl-phenyl]carbamoylamino]-N,N-dimethyl-piperidine-1-carboxamide

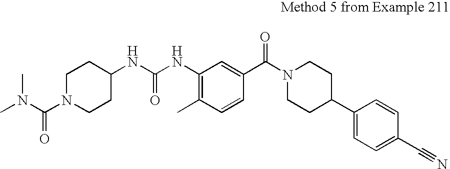

¹H NMR (300.073 MHz, d₆-DMSO) δ 1.25-1.93 (8H, m), 2.19 (3H, s), 2.72 (6H, s), 2.76-3.24 (5H, m), 3.36-3.51 (2H, m), 3.52-3.96 (2H, m), 4.37-4.82 (1H, m), 6.68 (1H, d), 6.91 (1H, d), 7.17 (1H, d), 7.49 (2H, d), 7.63 (1H, s), 7.76 (2H, d), 7.97 (1H, s), m/z 517 (M+H)⁺.

### EXAMPLE 285

### 1-[5-[4-(4-cyanophenyl)piperidine-1-carbonyl]-2-methyl-phenyl]-3-[1-(dimethylsulfamoyl)-4-piperidyl]urea

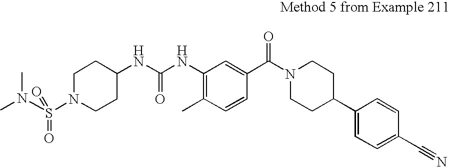

The reaction mixture was heated at 60° C. overnight. ¹H NMR (300.073 MHz, d₆-DMSO) δ 1.29-1.95 (8H, m), 2.20 (3H, s), 2.75 (6H, s), 2.83-3.23 (5H, m), 3.38-3.95 (4H, m), 4.37-4.84 (1H, m), 6.71 (1H, d), 6.91 (1H, d), 7.17 (1H, d), 7.49 (2H, d), 7.65 (1H, s), 7.76 (2H, d), 7.97 (1H, s), m/z 517 (M+H)⁺.

### EXAMPLE 286

### 1-[5-[4-(4-cyanophenyl)piperidine-1-carbonyl]-2-methyl-phenyl]-3-(3-piperidyl)urea

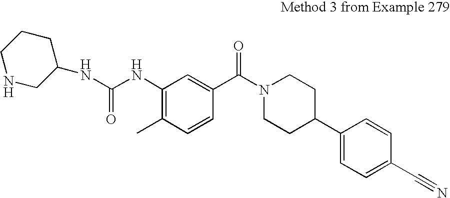

¹H NMR (300.072 MHz, CDCl₃) δ 1.46-1.98 (9H, m), 2.14 (3H, s), 2.53-2.72 (2H, m), 2.76-2.93 (3H, m), 2.99-3.22 (2H, m), 3.74-3.83 (1H, m), 3.91-4.16 (1H, m), 4.75-5.00 (1H, m), 5.71-5.85 (1H, m), 6.70 (1H, s), 6.96-7.14 (2H, m), 7.32 (2H, d), 7.56-7.66 (3H, m), m/z (EI+) (M+H)+=446; HPLC tR=1.27 min.

### EXAMPLE 287

### 1-[5-[4-(4-cyanophenyl)piperidine-1-carbonyl]-2-methyl-phenyl]-3-cyclobutyl-urea

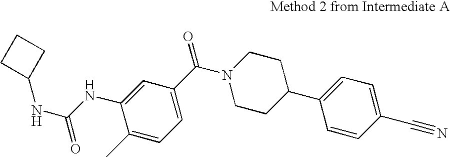

¹H NMR (300.072 MHz, CDCl₃) δ 1.50-2.10 (11H, m), 2.30 (1H, m), 2.85 (2H, m), 3.10 (1H, m), 3.95 (1H, m), 4.90 (1H, m), 6.02 (1H, d), 6.87 (1H, s), 6.95 (1H, m), 7.03 (1H, m), 7.35 (2H, d), 7.60 (3H, m), m/z 417 (M+H)⁺.

### EXAMPLE 288

### 1-[5-[4-(4-cyanophenyl)piperidine-1-carbonyl]-2-methyl-phenyl]-3-(cyclopropylmethyl)urea

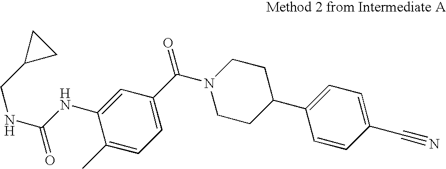

¹H NMR (300.072 MHz, CDCl₃) δ 0.19 (2H, m), 0.45-0.51 (2H, m), 1.50-2.10 (7H, m), 2.79-2.91 (2H, m), 3.00-3.15 (4H, m), 3.95 (1H, m), 4.90 (1H, m), 5.83 (1H, t), 6.90-6.95 (2H, m), 7.02-7.04 (1H, m), 7.32 (2H, d), 7.56-7.59 (2H, m), 7.62 (1H, s), m/z 417 (M+H)⁺.

### EXAMPLE 289

### 1-[5-[4-(4-cyanophenyl)piperidine-1-carbonyl]-2-methyl-phenyl]-3-(1H-pyrazol-3-ylmethyl)urea

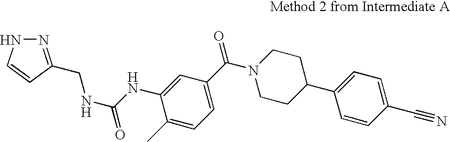

¹H NMR (300.072 MHz, CDCl₃) δ 1.40-2.05 (7H, m), 2.80-2.84 (1H, m), 2.76-2.88 (1H, m), 3.01-3.16 (1H, m), 3.85-3.95 (1H, m), 4.33 (2H, d), 4.79-4.84 (1H, m), 6.06 (1H, d), 6.67 (1H, t), 6.87-6.90 (1H, m), 7.00 (1H, d), 7.27-7.30 (2H, m), 7.34 (1H, s), 7.38 (1H, d), 7.57-7.59 (2H, m), 7.82 (1H, s), m/z 443 (M+H)⁺.

### EXAMPLE 290

### 1-[5-[4-(4-cyanophenyl)piperidine-1-carbonyl]-2-methyl-phenyl]-3-[(1-methylpyrazol-3-yl)methyl]urea

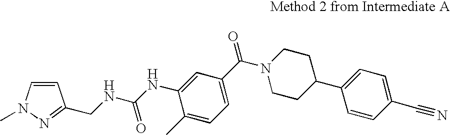

¹H NMR (300.072 MHz, CDCl₃) δ 1.50-2.10 (7H, m), 2.77-2.86 (2H, m), 3.10 (1H, d), 3.81 (3H, s), 3.85-4.04 (1H, m), 4.39 (2H, d), 4.90 (1H, m), 5.95 (1H, t), 6.18 (1H, d), 6.95-6.99 (1H, m), 7.05-7.09 (2H, m), 7.25 (1H, d), 7.31 (2H, d), 7.58-7.61 (2H, m), 7.64 (1H, d), m/z 457 (M+H)⁺.

### EXAMPLE 291

### 1-[5-[4-(4-cyanophenyl)piperidine-1-carbonyl]-2-methyl-phenyl]-3-(pyrimidin-2-ylmethyl)urea

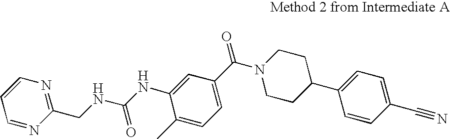

¹H NMR (300.072 MHz, CDCl₃) δ 1.50-2.00 (4H, m), 2.12 (3H, s), 2.76-2.85 (2H, m), 3.01-3.11 (1H, m), 3.95 (1H, m), 4.68 (2H, d), 4.85 (1H, m), 6.55 (1H, t), 6.96-7.00 (1H, m), 7.05 (1H, d), 7.14 (1H, t), 7.29 (2H, m), 7.43 (1H, s), 7.57-7.60 (2H, m), 7.69-7.72 (1H, m), 8.66-8.71 (2H, d), m/z 455 (M+H)⁺.

### EXAMPLE 292

### 1-[5-[4-(4-cyanophenyl)piperidine-1-carbonyl]-2-methyl-phenyl]-3-[(5-methyl-2H-pyrazol-3-yl)methyl]urea

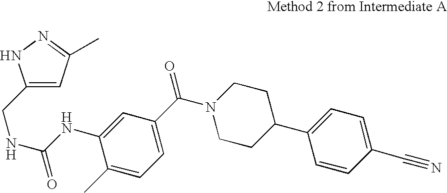

¹H NMR (300.072 MHz, CDCl₃) δ 1.50-2.05 (7H, m), 2.16 (3H, s), 2.76-2.84 (2H, m), 3.01-3.10 (1H, m), 3.95 (1H, m), 4.32 (2H, d), 4.80 (1H, m), 5.86 (1H, s), 6.59 (1H, t), 6.89-6.92 (1H, m), 7.00 (1H, d), 7.28 (2H, d), 7.39 (1H, s), 7.58 (3H, d), 7.79 (1H, d), m/z 457 (M+H)⁺.

### EXAMPLE 293

### 1-[5-[4-(4-cyanophenyl)piperidine-1-carbonyl]-2-methyl-phenyl]-3-(pyrazin-2-ylmethyl)urea

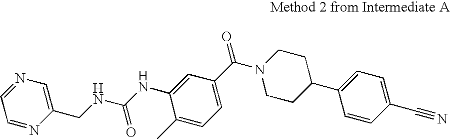

¹H NMR (300.072 MHz, CDCl₃) δ 1.50-2.00 (4H, m), 2.06 (3H, s), 2.83 (2H, m), 3.10 (1H, m), 3.95 (1H, m), 4.58 (2H, d), 4.85 (1H, m), 6.41 (1H, t), 6.93-6.96 (1H, m), 7.03-7.06 (1H, m), 7.14 (1H, s), 7.31 (2H, d), 7.54 (1H, d), 7.59 (2H, m), 8.53 (2H, m), 8.64 (1H, d), m/z 455 (M+H)⁺.

### EXAMPLE 294

### 1-[5-[4-(4-cyanophenyl)piperidine-1-carbonyl]-2,4-dimethyl-phenyl]-3-(3-methylsulfonylpropyl)urea

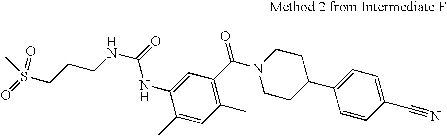

¹H NMR (300.072 MHz, CDCl₃, 30° C.) δ 1.38-1.88 (3H, m), 1.88-2.11 (6H, m), 2.12-2.35 (3H, m), 2.74-2.99 (5H, m), 3.01-3.22 (3H, m), 3.26-3.41 (2H, m), 3.70 (1H, dJ=13.6 Hz), 4.93 (1H, dJ=12.5 Hz), 5.90 (1H, s), 6.61-6.83 (1H, m), 6.87 (1H, s), 7.16-7.40 (3H, m), 7.60 (2H, dJ=8.3 Hz), m/z 497 (M+H)⁺.

### EXAMPLE 295

### 3-[5-[4-(4-cyanophenyl)piperidine-1-carbonyl]-2-methyl-phenyl]-1-(1-propanoyl-3-piperidyl)urea

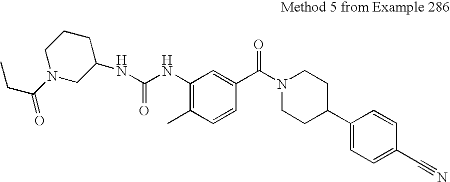

¹H NMR (400.132 MHz, CDCl₃) δ 1.15 (3H, t), 1.48-1.97 (8H, m), 2.16 (3H, d), 2.37 (2H, q), 2.79-2.89 (2H, m), 3.05-3.25 (1H, m), 3.30-3.41 (1H, m), 3.48-3.59 (2H, m), 3.63-3.71 (1H, m), 3.78-3.96 (1H, m), 4.00-4.07 (1H, m), 4.75-4.96 (1H, m), 5.66 (1H, d), 6.55-6.81 (1H, m), 7.01-7.17 (2H, m), 7.33 (2H, d), 7.57-7.64 (3H, m), m/z (ESI+) (M+H)+=502; HPLC tR=2.04 min.

### EXAMPLE 296

### 1-[5-[4-(4-cyanophenyl)piperidine-1-carbonyl]-2-methyl-phenyl]-3-(1-methylsulfonyl-3-piperidyl)urea

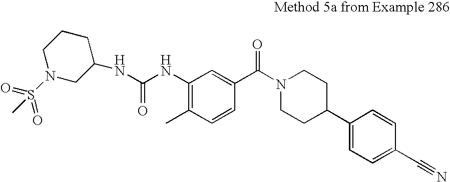

### Method 5a

The title compound was prepared by a procedure essentially analogous to that described in Method 5 using sodium carbonate as the base in place of pyridine, and performing the reaction in a 2-phase solvent system consisting of DCM—Water, ¹H NMR (400.132 MHz, CDCl₃) δ 1.63-1.86 (8H, m), 2.11 (3H, s), 2.78 (3H, s), 2.82-2.89 (2H, m), 2.97-3.08 (1H, m), 3.12-3.19 (1H, m), 3.24-3.28 (2H, m), 3.34-3.40 (1H, m), 3.92-4.07 (2H, m), 4.80-5.04 (1H, m), 5.67-5.76 (1H, m), 6.69 (1H, s), 6.98-7.12 (2H, m), 7.33 (2H, d), 7.52-7.56 (1H, m), 7.60 (2H, d), m/z (ESI+) (M+H)+=524; HPLC tR=2.12 min.

### EXAMPLE 297

### 3-[5-[4-(4-cyanophenyl)piperidine-1-carbonyl]-2-methyl-phenyl]-1-(1-ethylsulfonyl-3-piperidyl)urea

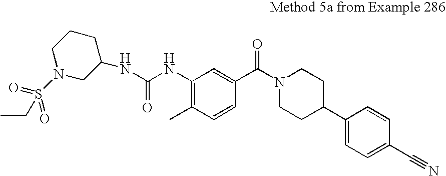

¹H NMR (400.132 MHz, CDCl₃) δ 1.34 (3H, t), 1.60-1.98 (8H, m), 2.18 (3H, s), 2.77-2.87 (2H, m), 2.95 (2H, q), 3.08-3.17 (2H, m), 3.25-3.47 (3H, m), 3.94-4.03 (2H, m), 4.77-4.97 (1H, m), 5.49 (1H, d), 6.46 (1H, s), 7.05-7.16 (2H, m), 7.33 (2H, d), 7.56-7.63 (3H, m), m/z (ESI+) (M+H)+=538; HPLC tR=2.19 min.

### EXAMPLE 298

### 1-[5-[4-(4-cyanophenyl)piperidine-1-carbonyl]-2-ethyl-phenyl]-3-(oxetan-3-yl)urea

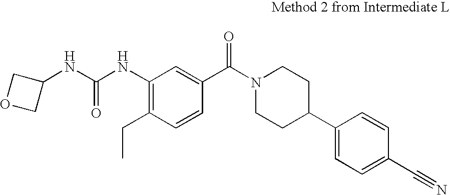

¹H NMR (300.073 MHz, d₆-DMSO) δ 1.16 (3H, t), 1.48-1.92 (4H, m), 2.58 (2H, q), 2.67-3.24 (3H, m), 3.60-3.91 (1H, m), 4.34-4.48 (2H, m), 4.49-4.68 (1H, m), 4.68-4.82 (3H, m), 7.00 (1H, d), 7.19 (1H, d), 7.34 (1H, d), 7.49 (2H, d), 7.71-7.81 (3H, m), 7.85 (1H, s), m/z (ESI+) (M+H)+=433.44; HPLC tR=1.95 min.

### EXAMPLE 299

### 1-[5-[4-(4-cyanophenyl)piperidine-1-carbonyl]-2-methyl-phenyl]-3-(oxetan-3-yl)urea

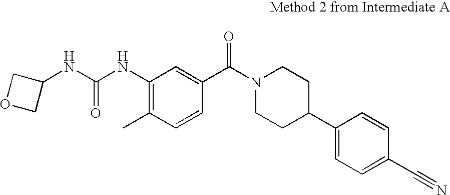

¹H NMR (300.073 MHz, d₆-DMSO) δ 1.49-1.91 (4H, m), 2.21 (3H, s), 2.67-3.22 (3H, m), 3.58-3.94 (1H, m), 4.35-4.44 (2H, m), 4.48-4.67 (1H, m), 4.69-4.81 (3H, m), 6.94 (1H, d), 7.19 (1H, d), 7.33 (1H, d), 7.49 (2H, d), 7.72-7.81 (3H, m), 7.88 (1H, s), m/z (ESI+) (M+H)+=419.42; HPLC tR=1.85 min.

### EXAMPLE 300

### 3-(azetidin-3-yl)-1-[5-[4-(4-cyanophenyl)piperidine-1-carbonyl]-2-methyl-phenyl]urea

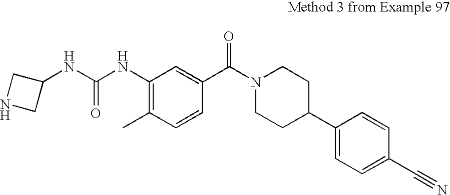

¹H NMR (300.072 MHz, CDCl₃) δ 1.58-2.05 (4H, m), 2.11 (3H, s), 2.78-2.93 (2H, m), 2.99-3.34 (1H, m), 3.44-3.59 (2H, m), 3.81-4.08 (3H, m), 4.23-4.44 (1H, m), 4.55-4.67 (1H, m), 4.79-4.91 (1H, m), 6.35 (1H, d), 6.97-7.13 (3H, m), 7.32 (2H, d), 7.57-7.64 (3H, m), m/z (ESI+) (M+H)+=418; HPLC tR=1.22 min.

### EXAMPLE 301

### 1-[5-[4-(4-cyanophenyl)piperidine-1-carbonyl]-2-methyl-phenyl]-3-[1-(2-methylpropanoyl)azetidin-3-yl]urea

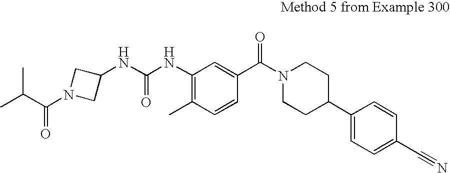

¹H NMR (300.072 MHz, CDCl₃) δ 1.10 (6H, d), 1.58-1.94 (4H, m), 2.04 (3H, s), 2.45 (1H, septet), 2.78-2.93 (2H, m), 3.05-3.26 (1H, m), 3.71-3.81 (1H, m), 3.92-4.06 (2H, m), 4.29 (1H, t), 4.45 (1H, t), 4.52-4.60 (1H, m), 4.75-4.94 (1H, m), 6.61 (1H, d), 6.91-7.00 (2H, m), 7.08 (1H, d), 7.32 (2H, d), 7.57-7.65 (3H, m), m/z (ESI+) (M+H)+=488; HPLC tR=1.96 min.

### EXAMPLE 302

### 1-[5-[4-(4-cyanophenyl)piperidine-1-carbonyl]-2-methyl-phenyl]-3-(1-methylsulfonylazetidin-3-yl)urea

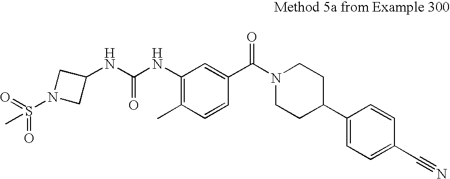

¹H NMR (300.072 MHz, CDCl₃) δ 1.52-1.90 (4H, m), 1.97 (3H, s), 2.80-2.89 (2H, m), 2.92 (3H, s), 3.06-3.24 (1H, m), 3.88-4.00 (3H, m), 4.06-4.14 (2H, m), 4.57 (1H, sextet), 4.82-4.99 (1H, m), 6.58 (1H, d), 6.73 (1H, s), 6.94-7.08 (2H, m), 7.34 (2H, d), 7.49-7.51 (1H, m), 7.62 (2H, d), m/z (ESI+) (M+H)+=496; HPLC tR=1.97 min.

### EXAMPLE 303

### 1-[5-[4-(4-cyanophenyl)piperidine-1-carbonyl]-2-methyl-phenyl]-3-[1-(dimethylsulfamoyl)azetidin-3-yl]urea

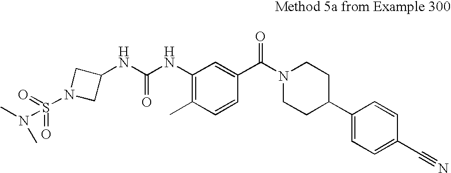

¹H NMR (300.072 MHz, CDCl₃) δ 1.70-1.92 (4H, m), 2.00 (3H, s), 2.81 (6H, s), 2.87-2.94 (2H, m), 3.09-3.28 (1H, m), 3.83 (2H, t), 3.91-3.99 (1H, m), 4.04 (2H, t), 4.62 (1H, sextet), 4.87-5.00 (1H, m), 6.43 (1H, d), 6.75 (1H, s), 6.95-7.09 (2H, m), 7.34 (2H, d), 7.44-7.47 (1H, m), 7.61 (2H, d), m/z (ESI+) (M+H)+=525; HPLC tR=2.16 min.

### EXAMPLE 304

### 3-[(cis)-2-aminocyclohexyl]-1-[5-[4-(4-cyanophenyl)piperidine-1-carbonyl]-2-methyl-phenyl]urea

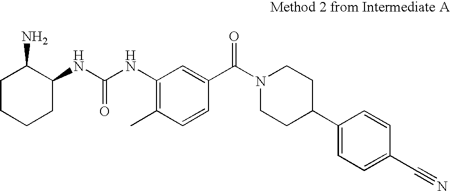

¹H NMR (300.072 MHz, CDCl₃) δ 2.02-0.90 (16H, m), 2.49 (3H, s), 3.07-2.87 (3H, m), 3.60-3.48 (2H, m), 4.04 (1H, d), 4.79 (1H, t), 6.89 (1H, s), 7.37-7.24 (4H, m), 7.71-7.54 (4H, m), 8.62 (1H, s), m/z (EI+) (M+H)+=460.37; HPLC tR=1.43 min; m/z (EI−) (M−H)−=458.13; HPLC tR=1.43 min.

### EXAMPLE 305

### 3-[(trans)-2-aminocyclohexyl]-1-[5-[4-(4-cyanophenyl)piperidine-1-carbonyl]-2-methyl-phenyl]urea

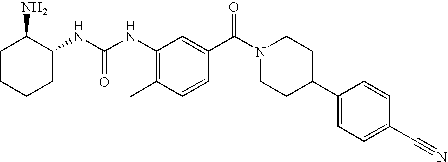

### Method 8

Hydrazine monohydrate (0.049 mL, 1.02 mmol) was added to a suspension of 1-(5-(4-(4-cyanophenyl)piperidine-1-carbonyl)-2-methylphenyl)-3-((1R,2R)-2-(1,3-dioxoisoindolin-2-yl)cyclohexyl)urea (Intermediate O) (601 mg, 1.02 mmol) in ethanol (3 mL) at ambient temperature, and the resulting solution was heated to reflux for 30 minutes. The mixture was then cooled to room temperature and the solid collected by filtration. The filtrate was concentrated under reduced pressure to give crude product as a yellow foam (428 mg). This was purified by flash silica chromatography, elution gradient 0 to 20% MeOH in DCM to give the desired product (35 mg, 7.5%) as a colourless dry film, ¹H NMR (300.072 MHz, CDCl₃) δ 1.95-0.86 (16H, m), 2.35 (3H, s), 3.34-2.87 (2H, m), 3.64-3.59 (1H, m), 4.06-4.00 (1H, m), 4.96-4.81 (1H, m), 6.94 (1H, d), 7.17 (1H, d), 7.33 (2H, d), 7.44 (1H, s), 7.60 (2H, d), 7.82 (1H, s), 7.82 (1H, s), m/z (EI+) (M+H)+=460.36; HPLC tR=1.38 min; m/z (EI−) (M−H)−=458.31; HPLC tR=1.38 min.

### EXAMPLE 306

### 3-amino-N-[5-[4-(4-cyanophenyl)piperidine-1-carbonyl]-2-methyl-phenyl]azetidine-1-carboxamide

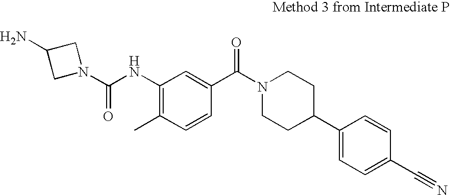

¹H NMR (300.072 MHz, CDCl₃) δ 1.56-1.93 (4H, m), 2.13 (2H, s), 2.24 (3H, s), 2.78-2.89 (2H, m), 3.00-3.23 (1H, m), 3.72-3.77 (2H, m), 3.90 (1H, quintet), 3.98-4.09 (1H, m), 4.26 (2H, t), 4.69-4.92 (1H, m), 6.06 (1H, s), 7.06-7.20 (2H, m), 7.32 (2H, d), 7.60 (2H, d), 7.80 (1H, s), m/z (ESI+) (M+H)+=418; HPLC tR=1.16 min.

### EXAMPLE 307

### 1-[5-[4-(4-cyanophenyl)piperidine-1-carbonyl]-2-methyl-phenyl]-3-[(cis)-2-methanesulfonamidocyclohexyl]urea

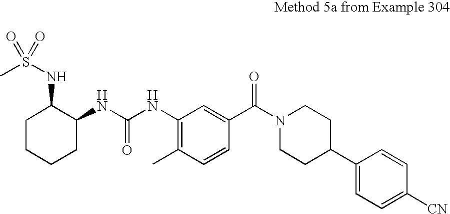

1H NMR (300.072 MHz, CDCl₃) δ 1.86-1.39 (11H, m), 2.00 (3H, s), 2.89-2.74 (2H, m), 2.97 (3H, s), 3.20-2.97 (3H, m), 3.61-3.51 (1H, m), 4.16-3.91 (2H, m), 5.00-4.71 (1H, m), 5.69 (1H, d), 6.18 (1H, d), 6.91 (1H, d), 7.01 (1H, s), 7.05 (1H, d), 7.33 (2H, d), 7.61 (2H, d), 7.71 (1H, s), m/z (EI+) (M+H)+=538.41; HPLC tR=2.17 min; m/z (EI−) (M−H)−=536.42; HPLC tR=2.17 min.

### EXAMPLE 308

### N-[(cis)-2-[[5-[4-(4-cyanophenyl)piperidine-1-carbonyl]-2-methyl-phenyl]carbamoylamino]cyclohexyl]acetamide

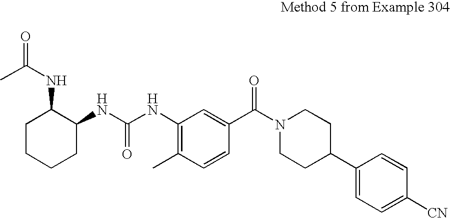

¹H NMR (300.072 MHz, CDCl₃) δ 1.90-1.37 (13H, m), 1.95 (3H, s), 2.01 (3H, s), 2.90-2.82 (2H, m), 3.27-3.06 (1H, m), 4.08-3.86 (2H, m), 5.00-4.61 (1H, m), 6.27 (1H, d), 6.92-6.87 (2H, m), 7.02 (1H, d), 7.22 (1H, s), 7.31 (2H, d), 7.61 (2H, d), 7.73 (1H, s), m/z (EI+) (M+H)+=502.48; HPLC tR=2.01 min; m/z (EI−) (M−H)−=500.45; HPLC tR=2.01 min.

### EXAMPLE 309

### 3-[5-[4-(4-cyanophenyl)piperidine-1-carbonyl]-2-methyl-phenyl]-1-[(cis)-2-(ethylsulfonylamino)cyclohexyl]urea

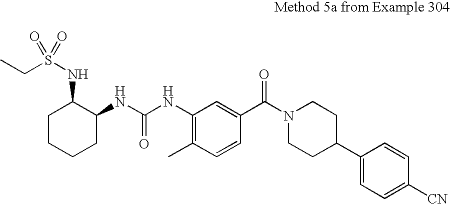

¹H NMR (300.072 MHz, CDCl₃) δ 1.33 (3H, t), 1.91-1.38 (12H, m), 2.03 (3H, s), 3.15-2.81 (5H, m), 3.57-3.52 (1H, m), 4.08-3.86 (2H, m), 5.00-4.69 (1H, m), 5.62 (1H, d), 6.14 (1H, d), 6.94 (1H, d), 7.05 (2H, d), 7.33 (2H, d), 7.61 (2H, d), 7.66 (1H, s), m/z (EI+) (M+H)+=552.45; HPLC tR=2.23 min; m/z (EI−) (M−H)−=550.43; HPLC tR=2.23 min m/z (EI+) (M+H)+=552.45; HPLC tR=2.23 min; m/z (EI−) (M−H)−=550.43; HPLC tR=2.23 min

### EXAMPLE 310

### 3-(1-acetylazetidin-3-yl)-1-[5-[4-(4-cyanophenyl)piperidine-1-carbonyl]-2-methyl-phenyl]urea

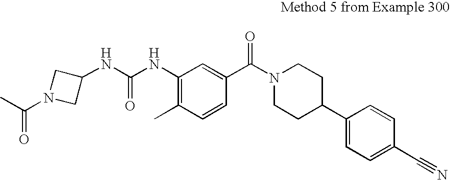

¹H NMR (300.072 MHz, CDCl₃) δ 1.59-1.85 (4H, m), 1.88 (3H, s), 2.06 (3H, s), 2.79-2.92 (2H, m), 3.07-3.25 (1H, m), 3.70-3.78 (1H, m), 3.91-4.05 (2H, m), 4.27 (1H, t), 4.39 (1H, t), 4.55 (1H, sextet), 4.78-4.91 (1H, m), 6.73 (1H, d), 6.95-7.10 (3H, m), 7.32 (2H, d), 7.59-7.66 (3H, m), m/z (ESI+) (M+H)+=460; HPLC tR=1.78 min.

### EXAMPLE 311

### 3-[5-[4-(4-cyanophenyl)piperidine-1-carbonyl]-2-methyl-phenyl]-1-(1-ethylsulfonylazetidin-3-yl)urea

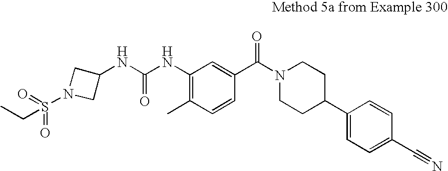

¹H NMR (300.072 MHz, CDCl₃) δ 1.36 (3H, t), 1.55-1.85 (4H, m), 1.98 (3H, s), 2.80-2.91 (2H, m), 3.00 (2H, q), 3.09-3.24 (1H, m), 3.87-3.98 (3H, m), 4.06 (2H, t), 4.61 (1H, sextet), 4.84-4.98 (1H, m), 6.57 (1H, d), 6.82 (1H, s), 6.96-7.08 (2H, m), 7.34 (2H, d), 7.45-7.46 (1H, m), 7.61 (2H, d), m/z (ESI+) (M+H)+=510; HPLC tR=2.08 min.

### EXAMPLE 312

### 3-acetamido-N-[5-[4-(4-cyanophenyl)piperidine-1-carbonyl]-2-methyl-phenyl]azetidine-1-carboxamide

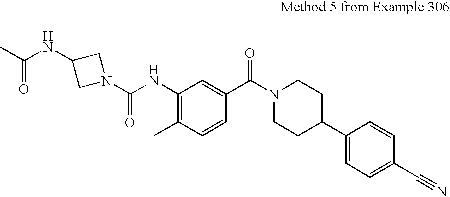

¹H NMR (300.072 MHz, CDCl₃) δ 1.46-1.93 (4H, m), 1.96 (3H, s), 2.22 (3H, s), 2.79-2.87 (2H, m), 2.99-3.23 (1H, m), 3.72-3.80 (2H, m), 3.88-3.96 (1H, m), 4.24 (2H, t), 4.56-4.67 (1H, m), 4.74-4.90 (1H, m), 6.46 (1H, s), 7.03-7.20 (2H, m), 7.31 (2H, d), 7.41 (1H, d), 7.58 (2H, d), 7.69-7.72 (1H, m), m/z (ESI+) (M+H)+=460; HPLC tR=1.73 min.

### EXAMPLE 313

### N-[5-[4-(4-cyanophenyl)piperidine-1-carbonyl]-2-methyl-phenyl]-3-methanesulfonamido-azetidine-1-carboxamide

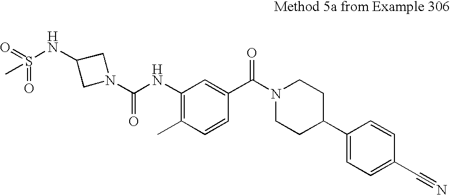

¹H NMR (300.072 MHz, CDCl₃) δ 1.47-2.01 (4H, m), 2.19 (3H, s), 2.75-2.84 (2H, m), 2.88 (3H, s), 3.01-3.21 (1H, m), 3.83-3.98 (3H, m), 4.18-4.28 (3H, m), 4.70-4.96 (1H, m), 6.52 (1H, s), 6.79 (1H, d), 7.05-7.20 (2H, m), 7.31 (2H, d), 7.58 (2H, d), 7.62-7.65 (1H, m), m/z (ESI+) (M+H)+=496; HPLC tR=1.89 min.

### EXAMPLE 314

### N-[5-[4-(4-cyanophenyl)piperidine-1-carbonyl]-2-methyl-phenyl]-3-(dimethylsulfamoylamino)azetidine-1-carboxamide

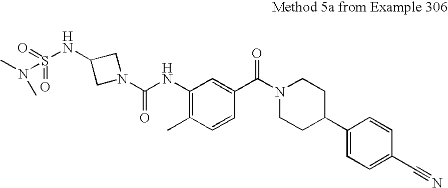

¹H NMR (300.072 MHz, CDCl₃) δ 1.53-1.93 (4H, m), 2.18 (3H, s), 2.75 (6H, s), 2.80-2.87 (2H, m), 2.97-3.19 (1H, m), 3.85-3.99 (3H, m), 4.10-4.25 (3H, m), 4.73-4.89 (1H, m), 6.41 (1H, d), 6.51 (1H, s), 7.03-7.19 (2H, m), 7.30 (2H, d), 7.57 (2H, d), 7.62-7.64 (1H, m), m/z (ESI+) (M+H)+=525; HPLC tR=2.04 min.

### EXAMPLE 315

### 1-[5-[4-(4-cyanophenyl)piperidine-1-carbonyl]-2-methyl-phenyl]-3-[(trans)-2-methanesulfonamidocyclohexyl]urea

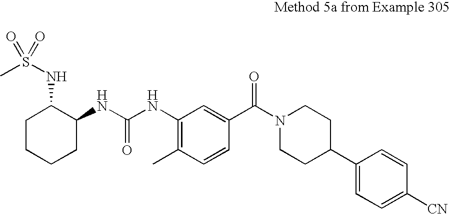

¹H NMR (300.072 MHz, CDCl₃) δ 2.04-1.25 (13H, m), 2.17 (3H, s), 2.92 (3H, s), 3.31-3.02 (3H, m), 3.54-3.48 (2H, m), 4.16-3.92 (1H, m), 4.95-4.67 (1H, m), 6.92 (1H, d), 7.10 (1H, d), 7.35 (3H, d), 7.41 (2H, s), 7.61 (3H, d), m/z (EI+) (M+H)+=538.42; HPLC tR=2.20 min; m/z (EI−) (M−H)−=536.40; HPLC tR=2.20 min.

### EXAMPLE 316

### tert-butyl N-[(1S,3S)-3-[[5-[4-(4-cyanophenyl)piperidine-1-carbonyl]-2-methyl-phenyl]carbamoylamino]cyclopentyl]carbamate

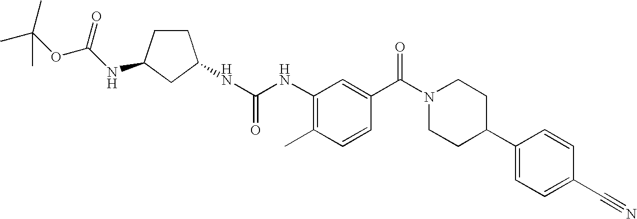

The title compound was prepared according to Method 2, starting from Intermediate A and tert-butyl (1S,3S)-3-aminocyclopentylcarbamate (preparation described in WO 2004/004726), ¹H NMR (300.073 MHz, d₆-DMSO) δ 1.38 (9H, s), 1.46-1.90 (8H, m), 2.19 (3H, s), 2.55-3.17 (5H, m), 3.54-4.11 (3H, m), 4.46-4.75 (1H, m), 6.70 (1H, d), 6.90 (2H, d), 7.16 (1H, d), 7.49 (2H, d), 7.56 (1H, s), 7.76 (2H, d), 7.98 (1H, s), m/z (ESI+) (M+H)+=546.48; HPLC tR=2.44 min.

### EXAMPLE 317

### 1-[5-[4-(4-cyanophenyl)piperidine-1-carbonyl]-2-methyl-phenyl]-3-[(cis)-2-(dimethylsulfamoylamino)cyclohexyl]urea

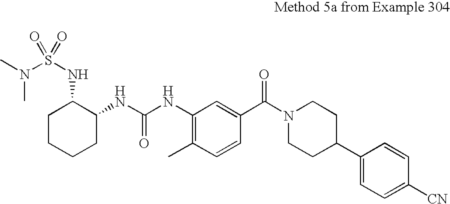

¹H NMR (300.072 MHz, CDCl₃) δ 1.94-1.36 (13H, m), 2.05 (3H, s), 2.78 (6H, s), 3.26-2.80 (2H, m), 3.50-3.45 (1H, m), 4.13-3.89 (2H, m), 5.00-4.67 (1H, m), 5.46 (1H, d), 5.94 (1H, d), 6.78 (1H, s), 6.98 (1H, dd), 7.07 (1H, d), 7.33 (2H, d), 7.62-7.58 (3H, m), m/z (EI+) (M+H)+=567.42; HPLC tR=2.32 min; m/z (EI−) (M−H)−=565.42; HPLC tR=2.32 min.

### EXAMPLE 318

### 3-[(1S,3S)-3-aminocyclopentyl]-1-[5-[4-(4-cyanophenyl)piperidine-1-carbonyl]-2-methyl-phenyl]urea

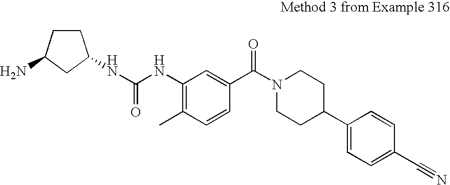

¹H NMR (300.073 MHz, d₆-DMSO) δ 1.36-2.15 (10H, m), 2.20 (3H, s), 2.56-3.40 (3H, m), 3.58-3.70 (1H, m), 3.72-3.88 (1H, m), 4.06-4.20 (1H, m), 4.27-4.90 (1H, m), 6.91 (1H, d), 7.16 (1H, d), 7.49 (2H, d), 7.76 (2H, d), 7.96 (1H, s), m/z (ESI+) (M+H)+=446.46; HPLC tR=1.32 min.

### EXAMPLE 319

### 1-[5-[4-(4-cyanophenyl)piperidine-1-carbonyl]-2-methyl-phenyl]-3-[(trans)-2-(dimethylsulfamoylamino)cyclohexyl]urea

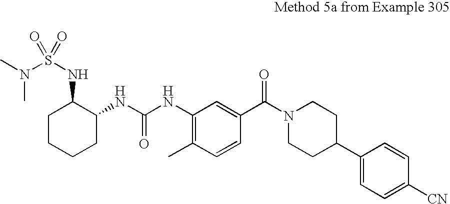

¹H NMR (300.072 MHz, CDCl₃) δ 1.38-1.13 (4H, m), 1.92-1.58 (6H, m), 2.10 (4H, s), 2.73 (6H, s), 3.11-2.79 (3H, m), 3.57-3.46 (1H, m), 4.17-3.88 (1H, m), 4.99-4.62 (1H, m), 5.79-5.71 (1H, m), 5.84 (1H, d), 7.00 (1H, d), 7.03 (1H, s), 7.09 (1H, d), 7.17 (1H, d), 7.24 (1H, d), 7.35 (2H, d), 7.60 (3H, d), m/z (EI+) (M+H)+=567.49; HPLC tR=2.36 min; m/z (EI−) (M−H)−=565.47; HPLC tR=2.36 min.

### EXAMPLE 320

### 1-[5-[4-(4-cyanophenyl)piperidine-1-carbonyl]-2-methyl-phenyl]-3-[(1S,3S)-3-methanesulfonamidocyclopentyl]urea

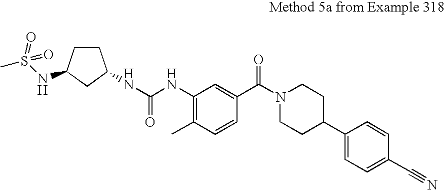

¹H NMR (300.073 MHz, d₆-DMSO) δ 1.20-2.12 (10H, m), 2.19 (3H, s), 2.69-2.99 (6H, m), 3.69-4.11 (3H, m), 4.45-4.74 (1H, m), 6.71 (1H, d), 6.90 (1H, d), 7.07-7.19 (2H, m), 7.49 (2H, d), 7.57 (1H, s), 7.76 (2H, d), 7.98 (1H, s), m/z (ESI+) (M+H)+=524.41; HPLC tR=2.03 min.

### EXAMPLE 321

### N-[(1S,3S)-3-[[5-[4-(4-cyanophenyl)piperidine-1-carbonyl]-2-methyl-phenyl]carbamoylamino]cyclopentyl]acetamide

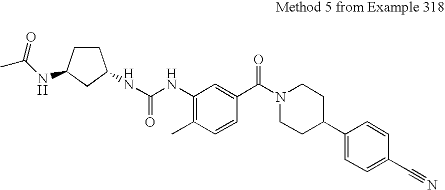

¹H NMR (300.073 MHz, d₆-DMSO) δ 1.10-2.14 (13H, m), 2.19 (3H, s), 2.61-3.20 (3H, m), 3.64-3.93 (1H, m), 3.98-4.23 (2H, m), 4.47-4.76 (1H, m), 6.74 (1H, d), 6.90 (1H, d), 7.16 (1H, d), 7.49 (2H, d), 7.57 (1H, s), 7.76 (2H, d), 7.87 (1H, d), 7.99 (1H, s), m/z (ESI+) (M+H)+=488.49; HPLC tR=1.89 min.

### EXAMPLE 322

### N-[5-[4-(4-cyanophenyl)piperidine-1-carbonyl]-2-methyl-phenyl]-4-methylsulfonyl-piperazine-1-carboxamide

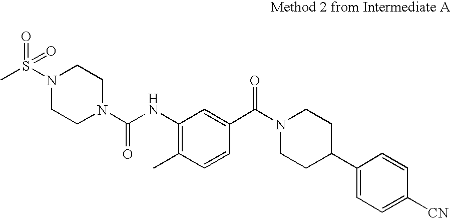

¹H NMR (300.073 MHz, d₆-DMSO) δ 1.49-1.90 (4H, m), 2.19 (3H, s), 2.83-2.97 (5H, m), 3.09-3.17 (5H, m), 3.50-3.59 (4H, m), 3.69-3.87 (1H, m), 4.34-4.87 (1H, m), 7.11 (1H, d), 7.20-7.29 (2H, m), 7.50 (2H, d), 7.76 (2H, d), 8.24 (1H, s), m/z (ESI+) (M+H)+=510.39; HPLC tR=2.02 min.

### EXAMPLE 323

### 1-[5-[4-(4-cyanophenyl)piperidine-1-carbonyl]-2-methyl-phenyl]-3-[(1S,3S)-3-(dimethylsulfamoylamino)cyclopentyl]urea

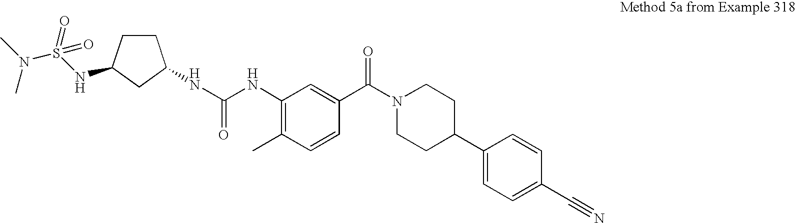

¹H NMR (300.073 MHz, d₆-DMSO) δ 1.21-2.08 (10H, m), 2.19 (3H, s), 2.64 (6H, s), 2.77-3.19 (3H, m), 3.60-3.85 (2H, m), 3.97-4.10 (1H, m), 4.46-4.71 (1H, m), 6.71 (1H, d), 6.90 (1H, d), 7.16 (1H, d), 7.23 (1H, d), 7.50 (2H, d), 7.56 (1H, s), 7.76 (2H, d), 7.98 (1H, s), m/z (ESI+) (M+H)+=553.44; HPLC tR=2.16 min.

### EXAMPLE 324

### 3-[5-[4-(4-cyanophenyl)piperidine-1-carbonyl]-2-methyl-phenyl]-1-[(1S,3S)-3-(ethylsulfonylamino)cyclopentyl]urea

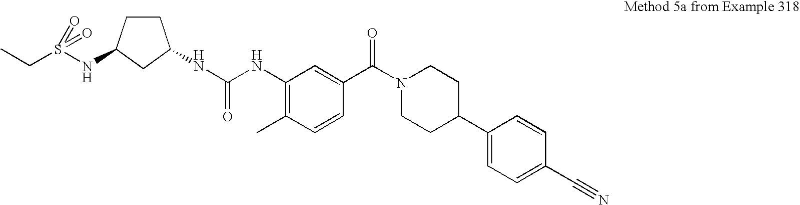

¹H NMR (300.073 MHz, d₆-DMSO) δ 1.19 (3H, t), 1.27-2.12 (10H, m), 2.18 (3H, s), 2.77-3.16 (5H, m), 3.60-3.90 (2H, m), 3.96-4.12 (1H, m), 4.40-4.75 (1H, m), 6.70 (1H, d), 6.90 (1H, d), 7.10-7.19 (2H, m), 7.49 (2H, d), 7.56 (1H, s), 7.76 (2H, d), 7.97 (1H, s), m/z (ESI+) (M+H)+=538.45; HPLC tR=2.10 min.

### EXAMPLE 325

### 1-[5-[4-(4-fluorophenyl)piperidine-1-carbonyl]-2-methyl-phenyl]-3-propan-2-yl-urea

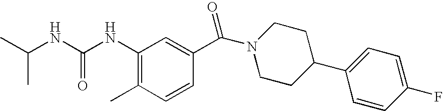

### Method 6b

The title compound was prepared by means of a standard amide coupling as described in Method 6 (EDAC, DMAP in DMF as solvent), starting from 4-methyl-3-(propan-2-ylcarbamoylamino) benzoic acid (Intermediate Q) and 4-(4-fluorophenyl)piperidine hydrochloride, ¹H NMR (300.073 MHz, d₆-DMSO) δ 1.10 (6H, d), 1.40-1.90 (4H, m), 2.19 (3H, s), 2.70-3.24 (3H, m), 3.60-3.87 (2H, m), 4.40-4.70 (1H, m), 6.52 (1H, d), 6.89 (1H, d), 7.04-7.19 (3H, m), 7.25-7.35 (2H, m), 7.57 (1H, s), 7.99 (1H, s), m/z 398 (M+H)⁺.

### EXAMPLE 326

### N-[2-[[2-methyl-5-[4-[4-(trifluoromethyl)phenyl]piperidine-1-carbonyl]phenyl]carbamoylamino]ethyl]acetamide

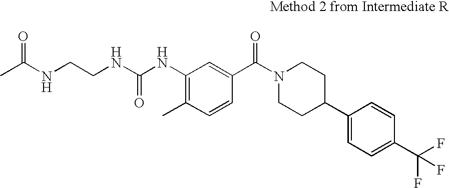

¹H NMR (300.073 MHz, d₆-DMSO) δ 1.51-1.90 (7H, m), 2.20 (3H, s), 2.77-3.07 (3H, m), 3.09-3.17 (4H, m), 3.62-3.96 (1H, m), 4.42-4.72 (1H, m), 6.58-6.69 (1H, m), 6.93 (1H, d), 7.17 (1H, d), 7.51 (2H, d), 7.65 (2H, d), 7.76 (1H, s), 7.84-7.94 (2H, m), m/z (ESI+) (M+H)+=491.43; HPLC tR=2.17 min.

### EXAMPLE 327

### 1-[5-[4-(3-cyanophenyl)piperidine-1-carbonyl]-2-methyl-phenyl]-3-propan-2-yl-urea

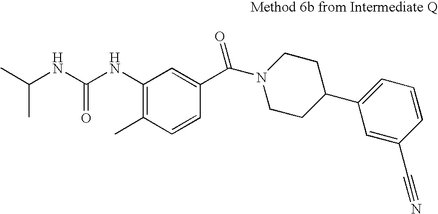

¹H NMR (300.073 MHz, d₆-DMSO) δ 1.10 (6H, d), 1.50-1.94 (4H, m), 2.19 (3H, s), 2.62-3.20 (3H, m), 3.60-3.89 (2H, m), 4.41-4.76 (1H, m), 6.52 (1H, d), 6.91 (1H, d), 7.16 (1H, d), 7.45-7.70 (4H, m), 7.77 (1H, s), 7.99 (1H, s), m/z 405 (M+H)⁺.

### EXAMPLE 328

### 1-[5-[4-(4-methoxyphenyl)piperidine-1-carbonyl]-2-methyl-phenyl]-3-propan-2-yl-urea

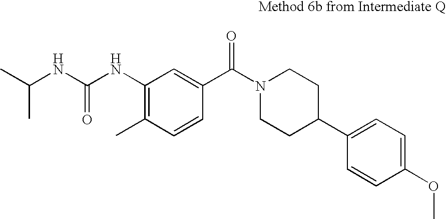

¹H NMR (300.073 MHz, d₆-DMSO) δ 1.10 (6H, d), 1.39-1.90 (4H, m), 2.19 (3H, s), 2.64-3.23 (3H, m), 3.60-3.90 (5H, m), 4.40-4.70 (1H, m), 6.52 (1H, d), 6.80-6.92 (3H, m), 7.11-7.21 (3H, m), 7.57 (1H, s), 7.99 (1H, s), m/z 410 (M+H)⁺.

### EXAMPLE 329

### N-methyl-4-[1-[4-methyl-3-(propan-2-ylcarbamoylamino)benzoyl]-4-piperidyl]benzamide

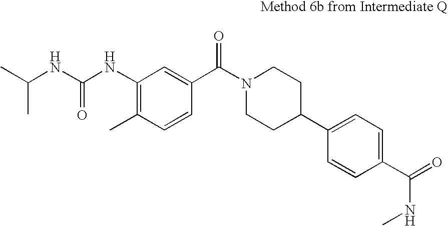

¹H NMR (300.073 MHz, d₆-DMSO) δ 1.10 (6H, d), 1.43-1.93 (4H, m), 2.19 (3H, s), 2.68-3.19 (6H, m), 3.60-3.90 (2H, m), 4.40-4.72 (1H, m), 6.52 (1H, d), 6.90 (1H, d), 7.16 (1H, d), 7.34 (2H, d), 7.58 (1H, s), 7.75 (2H, d), 8.00 (1H, s), 8.27-8.36 (1H, m), m/z 437 (M+H)⁺.

### EXAMPLE 330

### Ethyl 4-[[5-[4-(4-cyanophenyl)piperidine-1-carbonyl]-2-methyl-phenyl]carbamoyl]piperazine-1-carboxylate

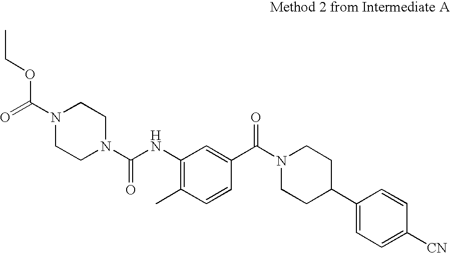

¹H NMR (400.132 MHz, d₆-DMSO) δ 1.21 (3H, t), 1.55-1.91 (4H, m), 2.21 (3H, s), 2.78-3.05 (3H, m), 3.39-3.50 (8H, m), 3.67-3.98 (1H, m), 4.08 (2H, q), 4.41-4.81 (1H, m), 7.12 (1H, d), 7.25 (1H, d), 7.29 (1H, s), 7.51 (2H, d), 7.77 (2H, d), 8.17 (1H, s), m/z (ESI+) (M+H)+=504.46; HPLC tR=2.15 min.

### EXAMPLE 331

### N-[5-[4-(4-cyanophenyl)piperidine-1-carbonyl]-2-methyl-phenyl]-4-(2-methylpropanoyl)piperazine-1-carboxamide

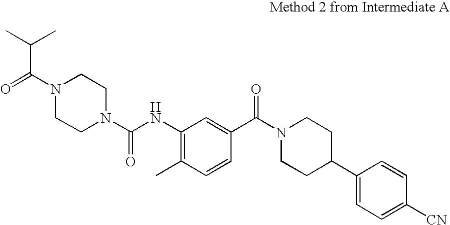

¹H NMR (400.132 MHz, d₆-DMSO) δ 1.03 (6H, d), 1.51-1.91 (4H, m), 2.21 (3H, s), 2.77-3.21 (4H, m), 3.40-3.60 (8H, m), 3.71-3.97 (1H, m), 4.43-4.77 (1H, m), 7.12 (1H, d), 7.25 (1H, d), 7.31 (1H, s), 7.51 (2H, d), 7.77 (2H, d), 8.17 (1H, s), m/z (ESI+) (M+H)+=502.36; HPLC tR=2.08 min.

### EXAMPLE 332

### N-[5-[4-(4-cyanophenyl)piperidine-1-carbonyl]-2-methyl-phenyl]-N′,N′-dimethyl-piperazine-1,4-dicarboxamide

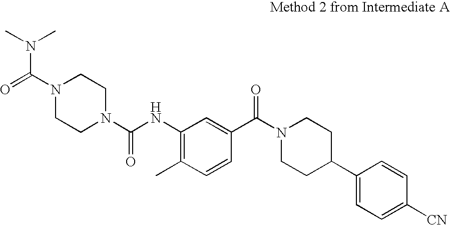

¹H NMR (300.072 MHz, CDCl₃) δ 1.61-2.00 (4H, m), 2.26 (3H, s), 2.61-3.09 (9H, m), 3.28-3.35 (4H, m), 3.50-3.57 (4H, m), 3.92-4.19 (1H, m), 4.71-5.00 (1H, m), 6.44 (1H, s), 7.08 (1H, d), 7.19 (1H, d), 7.32 (2H, d), 7.60 (2H, d), 7.67 (1H, s), m/z (ESI+) (M+H)+=503.47; HPLC tR=1.90 min.

### EXAMPLE 333

### tert-butyl 3-[[2-methyl-5-[4-[4-(trifluoromethyl)phenyl]piperidine-1-carbonyl]phenyl]carbamoylamino]piperidine-1-carboxylate

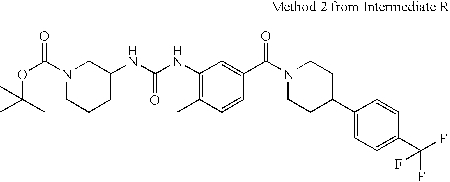

¹H NMR (400.132 MHz, d₆-DMSO) d1.32-1.42 (m, 9H), 1.42-1.55 (m, 3H), 1.57-1.75 (m, 3H), 1.76-1.93 (m, 3H), 2.22 (s, 3H), 2.62-3.25 (m, 5H), 3.39-3.99 (m, 3H), 4.47-4.82 (m, 1H), 6.74 (d, 1H), 6.93-7.00 (m, 1H), 7.23 (d, 1H), 7.56 (d, 2H), 7.70 (d, 2H), 7.74 (s, 1H), 8.04 (s, 1H), m/z (ESI+) (M+H)+=589.51; HPLC tR=2.93 min.

### EXAMPLE 334

### 3-(2-dimethylaminoethyl)-1-[2-methyl-5-[4-[4-(trifluoromethyl)phenyl]piperidine-1-carbonyl]phenyl]urea

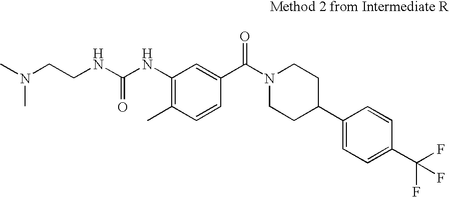

¹H NMR (300.073 MHz, d₆-DMSO) δ 1.48-1.91 (4H, m), 2.17-2.25 (9H, m), 2.38 (2H, t), 2.77-3.11 (3H, m), 3.15-3.24 (2H, m), 3.66-3.92 (1H, m), 4.44-4.74 (1H, m), 6.66 (1H, t), 6.91 (1H, d), 7.16 (1H, d), 7.51 (2H, d), 7.65 (2H, d), 7.89 (1H, s), 7.96 (1H, s), m/z (ESI+) (M+H)+=477.50; HPLC tR=1.53 min.

### EXAMPLE 335

### 1-[5-[4-(4-cyanophenyl)piperidine-1-carbonyl]-2-methyl-phenyl]-3-(3-methyl-1,1-dioxo-thiolan-3-yl)urea

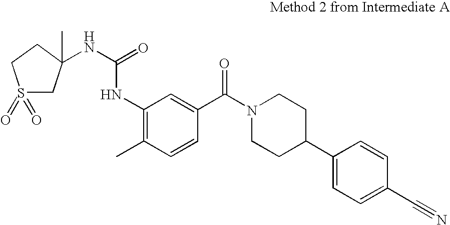

¹H NMR (300.072 MHz, CDCl₃) δ 1.65 (3H, s), 1.89-1.70 (3H, m), 1.89 (3H, s), 2.22-1.95 (2H, m), 2.97-2.79 (3H, m), 3.04 (1H, d), 3.23-3.15 (2H, m), 3.56-3.45 (1H, m), 3.74 (1H, d), 4.05-3.89 (1H, m), 4.92-4.79 (1H, m), 6.45 (1H, s), 6.66 (1H, s), 6.90 (1H, d), 7.00 (1H, d), 7.34 (2H, d), 7.53 (1H, s), 7.62 (2H, d), m/z (EI+) (M+H)+=495.43; HPLC tR=2.09 min.

### EXAMPLE 336

### tert-butyl N-[2-[[2-methyl-5-[4-[4-(trifluoromethyl)phenyl]piperidine-1-carbonyl]phenyl]carbamoylamino]ethyl]carbamate

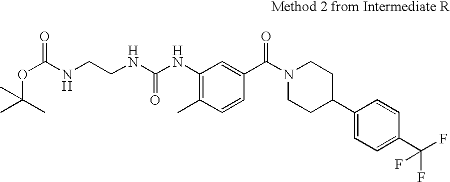

¹H NMR (300.073 MHz, d₆-DMSO) d1.30 (s, 9H), 1.51-1.70 (m, 2H), 1.70-1.93 (m, 2H), 2.15 (s, 3H), 2.82-2.96 (m, 1H), 2.96-3.06 (m, 3H), 3.06-3.18 (m, 3H), 3.58-3.98 (m, 1H), 4.28-4.80 (m, 1H), 6.59-6.70 (m, 1H), 6.75-6.86 (m, 1H), 6.92 (d, 1H), 7.17 (d, 1H), 7.51 (d, 2H), 7.65 (d, 2H), 7.73 (s, 1H), 7.89 (s, 1H), m/z (EI+) (M+H)+=549.51; HPLC tR=2.69 min.

### EXAMPLE 337

### 1-[2-methyl-5-[4-[4-(trifluoromethyl)phenyl]piperidine-1-carbonyl]phenyl]-3-(3-piperidyl)urea

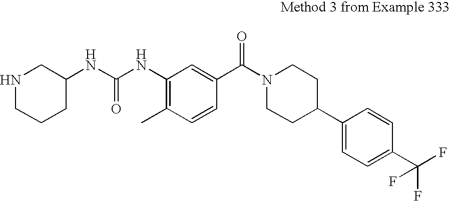

¹H NMR (300.073 MHz, d₆-DMSO) d1.39-1.50 (m, 1H), 1.54-1.73 (m, 3H), 1.74-1.94 (m, 4H), 2.20 (s, 3H), 2.62-2.77 (m, 1H), 2.82-3.00 (m, 2H), 3.03-3.20 (m, 2H), 3.23-3.34 (m, 1H), 3.74-3.98 (m, 2H), 4.40-4.81 (m, 1H), 6.90-6.98 (m, 1H), 7.12-7.25 (m, 2H), 7.50 (d, 2H), 7.66 (d, 2H), 7.92 (s, 1H), 7.97 (s, 1H), 8.72-9.07 (m, 2H), m/z (EI+) 489.50 (M+H)+=; HPLC tR=1.54 min.

### EXAMPLE 338

### N-[2-[[5-[4-(4-fluorophenyl)piperidine-1-carbonyl]-2-methyl-phenyl]carbamoylamino]ethyl]acetamide

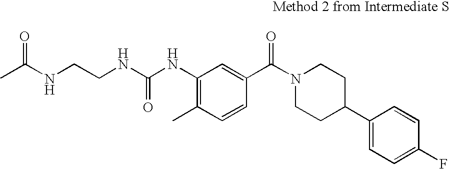

¹H NMR (500.133 MHz, d₆-DMSO) δ 1.53-1.64 (2H, m), 1.79-1.86 (5H, m), 2.23 (3H, s), 2.79-2.87 (1H, m), 2.96-3.04 (2H, m), 3.19 (4H, t), 4.12-4.28 (2H, m), 6.94 (1H, d), 7.04-7.09 (2H, m), 7.17 (1H, d), 7.26-7.33 (2H, m), 7.39-7.50 (1H, m), 7.40-7.50 (1H, m), 7.56 (1H, s), 7.82 (1H, s), m/z (ESI+) (M+H)+=441.46; HPLC tR=1.93 min.

### EXAMPLE 339

### tert-butyl 3-[[5-[4-(4-fluorophenyl)piperidine-1-carbonyl]-2-methyl-phenyl]carbamoylamino]azetidine-1-carboxylate

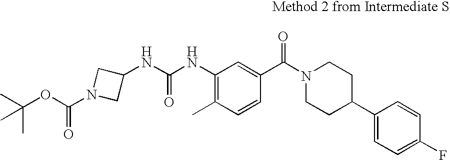

¹H NMR (300.073 MHz, d₆-DMSO) δ 0.98-1.08 (1H, m), 1.38 (9H, s), 1.49-1.88 (4H, m), 2.20 (3H, s), 2.69-3.00 (3H, m), 3.62-3.72 (2H, m), 4.03-4.12 (2H, m), 4.29-4.43 (1H, m), 4.47-4.74 (1H, m), 6.94 (1H, d), 7.06-7.15 (2H, m), 7.16-7.23 (2H, m), 7.26-7.34 (2H, m), 7.76 (1H, s), 7.86 (1H, s), m/z (ESI−) (M−H)−=509.57; HPLC tR=2.09 min.

### EXAMPLE 340

### 3-[2-(dimethylsulfamoylamino)ethyl]-1-[2-methyl-5-[4-[4-(trifluoromethyl)phenyl]piperidine-1-carbonyl]phenyl]urea

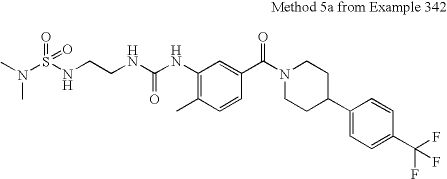

¹H NMR (300.073 MHz, d₆-DMSO) δ 1.48-1.91 (4H, m), 2.20 (3H, s), 2.65 (6H, s), 2.82-3.23 (7H, m), 3.66-3.92 (1H, m), 4.49-4.73 (1H, m), 6.71 (1H, t), 6.93 (1H, d), 7.15-7.25 (2H, m), 7.51 (2H, d), 7.65 (2H, d), 7.83 (1H, s), 7.91 (1H, s), m/z (ESI+) (M+H)+=556.51; HPLC tR=2.45 min.

### EXAMPLE 341

### 3-(2-methanesulfonamidoethyl)-1-[2-methyl-5-[4-[4-(trifluoromethyl)phenyl]piperidine-1-carbonyl]phenyl]urea

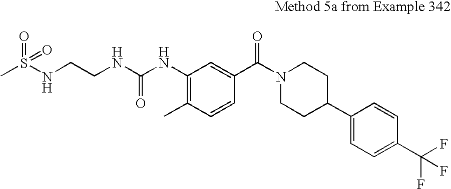

¹H NMR (300.073 MHz, d₆-DMSO) δ 1.47-1.92 (4H, m), 2.20 (3H, s), 2.83-3.24 (10H, m), 3.62-4.02 (1H, m), 4.48-4.71 (1H, m), 6.74 (1H, t), 6.93 (1H, d), 7.06 (1H, t), 7.18 (1H, d), 7.51 (2H, d), 7.65 (2H, d), 7.84 (1H, s), 7.92 (1H, s), m/z (ESI+) (M+H)+=527.47; HPLC tR=2.32 min.

### EXAMPLE 342

### 3-(2-aminoethyl)-1-[2-methyl-5-[4-[4-(trifluoromethyl)phenyl]piperidine-1-carbonyl]phenyl]urea

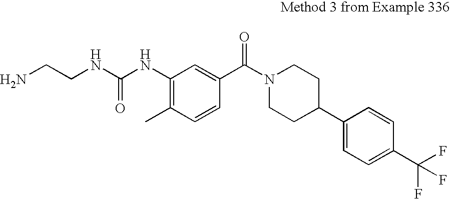

¹H NMR (300.072 MHz, CDCl₃) δ 1.58-2.00 (4H, m), 2.30 (3H, s), 2.75-3.21 (5H, m), 3.28-3.41 (2H, m), 3.85-4.02 (1H, m), 4.68-4.85 (1H, m), 6.92 (1H, d), 7.12 (1H, d), 7.30 (2H, d), 7.54 (2H, d), 7.85 (1H, s), 8.08-8.30 (4H, m), m/z (ESI+) (M+H)+=449.49; HPLC tR=1.42 min.

### EXAMPLE 343

### tert-butyl 3-[[5-[4-(4-fluorophenyl)piperidine-1-carbonyl]-2-methyl-phenyl]carbamoylamino]pyrrolidine-1-carboxylate

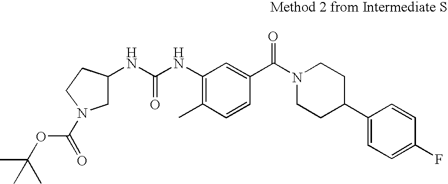

¹H NMR (400.132 MHz, d₆-DMSO) δ 1.41 (9H, s), 1.51-1.64 (2H, m), 1.71-1.85 (3H, m), 2.02-2.11 (1H, m), 2.21 (3H, s), 2.78-2.88 (2H, m), 3.06-3.14 (2H, m), 3.33 (2H, t), 3.42-3.55 (1H, m), 3.68-3.92 (1H, m), 4.11-4.20 (1H, m), 4.51-4.69 (1H, m), 6.90-6.95 (2H, m), 7.09-7.15 (2H, m), 7.19 (1H, d), 7.29-7.35 (2H, m), 7.66 (1H, s), 7.99 (1H, s), m/z (ESI+) (M+H)+=525.54; HPLC tR=2.58 min.

### EXAMPLE 344

### N-[5-[4-(4-cyanophenyl)piperidine-1-carbonyl]-2-methyl-phenyl]-3-methylsulfonyl-pyrrolidine-1-carboxamide

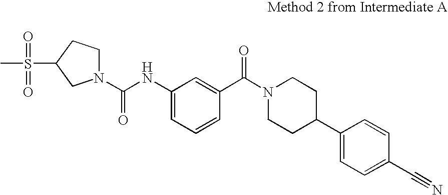

¹H NMR (300.073 MHz, d₆-DMSO) δ 1.50-1.90 (4H, m), 2.22 (3H, s), 2.25-2.37 (2H, m), 2.70-3.23 (3H, m), 3.04 (3H, s), 3.40-4.07 (6H, m), 4.40-4.80 (1H, m), 7.10 (1H, d), 7.24 (1H, d), 7.38 (1H, s), 7.50 (2H, d), 7.76 (2H, d), 7.81 (1H, s), m/z 495 (M+H)⁺.

### EXAMPLE 345

### N-[5-[4-(4-cyanophenyl)piperidine-1-carbonyl]-2-methyl-phenyl]-3-hydroxy-azetidine-1-carboxamide

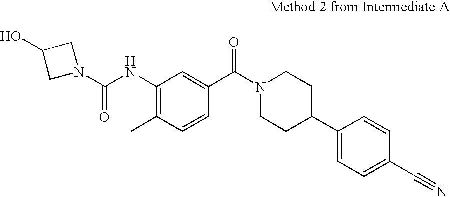

¹H NMR (300.073 MHz, d₆-DMSO) δ 1.50-1.91 (4H, m), 2.21 (3H, s), 2.66-3.22 (3H, m), 3.65-3.73 (2H, m), 3.74-4.00 (1H, m), 4.04-4.15 (2H, m), 4.37-4.49 (1H, m), 4.48-4.75 (1H, m), 5.60 (1H, d), 7.07 (1H, d), 7.22 (1H, d), 7.40 (1H, s), 7.50 (2H, d), 7.73-7.80 (3H, m), m/z 419 (M+H)⁺.

### EXAMPLE 346

### N-[5-[4-(4-cyanophenyl)piperidine-1-carbonyl]-2-methyl-phenyl]azetidine-1-carboxamide

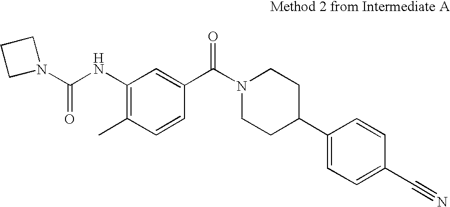

¹H NMR (300.073 MHz, d₆-DMSO) δ 1.50-1.95 (4H, m), 2.10-2.29 (5H, m), 2.70-3.22 (3H, m), 3.61-4.01 (5H, m), 4.39-4.70 (1H, m), 7.07 (1H, d), 7.22 (1H, d), 7.41 (1H, s), 7.50 (2H, d), 7.70-7.80 (3H, m), m/z 403 (M+H)⁺.

### EXAMPLE 347

### tert-butyl N-[2-[[5-[4-(4-fluorophenyl)piperidine-1-carbonyl]-2-methyl-phenyl]carbamoylamino]ethyl]-N-methyl-carbamate

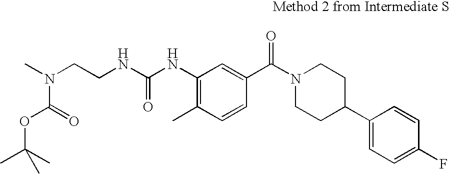

¹H NMR (300.072 MHz, CDCl₃) δ 1.43 (9H, s), 1.69-1.99 (4H, m), 2.10-2.22 (3H, m), 2.68-3.22 (6H, m), 3.36 (4H, s), 3.88-4.04 (1H, m), 4.74-4.95 (1H, m), 5.45-5.59 (1H, m), 6.53 (1H, s), 6.94-7.08 (3H, m), 7.10-7.21 (3H, m), 7.60 (1H, s), m/z (ESI+) (M+H)+=513.54; HPLC tR=2.55 min.

### EXAMPLE 348

### 3-(1-acetylpyrrolidin-3-yl)-1-[5-[4-(4-fluorophenyl)piperidine-1-carbonyl]-2-methyl-phenyl]urea

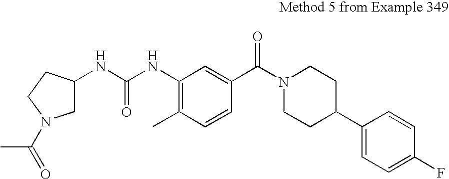

¹H NMR (300.073 MHz, d₆-DMSO) δ 1.49-1.66 (2H, m), 1.76-1.87 (2H, m), 1.94 (3H, s), 2.03-2.16 (1H, m), 2.22 (3H, s), 2.76-3.08 (5H, m), 3.18-3.31 (1H, m), 3.39-3.58 (1H, m), 3.64-3.75 (1H, m), 4.12-4.31 (3H, m), 6.61-6.73 (1H, m), 6.91-6.98 (1H, m), 7.03-7.12 (2H, m), 7.17 (1H, d), 7.25-7.33 (2H, m), 7.48-7.55 (1H, m), 7.90 (1H, s), m/z (ESI+) (M+H)+=467.50; HPLC tR=2.00 min.

### EXAMPLE 349

### 1-[5-[4-(4-fluorophenyl)piperidine-1-carbonyl]-2-methyl-phenyl]-3-pyrrolidin-3-yl-urea

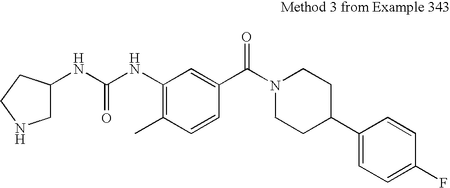

¹H NMR (400.132 MHz, d₆-DMSO) δ 0.84-0.92 (2H, m), 1.23-1.41 (4H, m), 1.46-1.86 (4H, m), 2.22 (3H, s), 2.73-3.07 (3H, m), 3.67-3.87 (1H, m), 4.12-4.18 (1H, m), 4.51-4.71 (1H, m), 6.90 (1H, d), 7.06-7.19 (4H, m), 7.28-7.35 (2H, m), 7.65-7.75 (1H, m), 7.83 (1H, s), 7.99 (1H, s), m/z (ESI+) (M+H)+=425.45; HPLC tR=1.43 min.

### EXAMPLE 350

### 1-[5-[4-(4-chlorophenyl)-4-hydroxy-piperidine-1-carbonyl]-2-methyl-phenyl]-3-propan-2-yl-urea

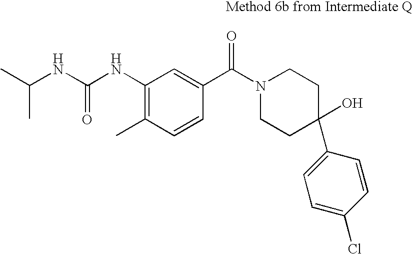

m/z (ESI+) (M+H)+=431; HPLC tR=2.10 min.

### EXAMPLE 351

### 1-[5-[4-(4-fluorophenyl)piperidine-1-carbonyl]-2-methyl-phenyl]-3-[2-(methyl-methylsulfonyl-amino)ethyl]urea

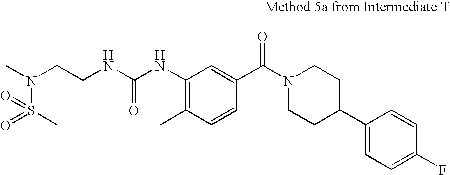

¹H NMR (300.073 MHz, d₆-DMSO) δ 1.46-1.86 (4H, m), 2.20 (3H, s), 2.70-2.92 (9H, m), 3.13 (4H, t), 3.69-3.85 (1H, m), 4.51-4.67 (1H, m), 6.69 (1H, t), 6.92 (1H, d), 7.06-7.20 (3H, m), 7.26-7.34 (2H, m), 7.88 (2H, d), m/z (ESI+) (M+H)+=491.43; HPLC tR=2.17 min.

### EXAMPLE 352

### N-[2-[[5-[4-(4-fluorophenyl)piperidine-1-carbonyl]-2-methyl-phenyl]carbamoylamino]ethyl]-N-methyl-acetamide

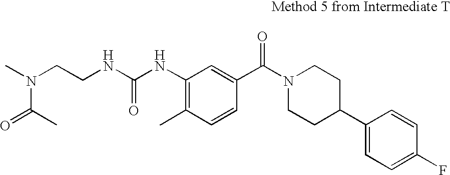

¹H NMR (300.073 MHz, d₆-DMSO) δ 1.43-1.87 (4H, m), 1.97 (3H, d), 2.19 (3H, s), 2.68-3.10 (8H, m), 3.32-3.40 (2H, m), 3.60-3.91 (1H, m), 4.40-4.71 (1H, m), 6.53-6.75 (1H, m), 6.89-6.97 (1H, m), 7.05-7.21 (3H, m), 7.26-7.35 (2H, m), 7.71-7.91 (2H, m), m/z (ESI+) (M+H)+=455.46; HPLC tR=1.99 min.

### EXAMPLE 353

### N-[2-[[5-[4-(4-cyanophenyl)piperidine-1-carbonyl]-2-methyl-phenyl]carbamoyl-methyl-amino]ethyl]propanamide

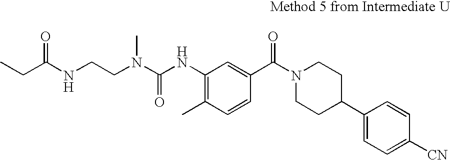

¹H NMR (300.073 MHz, d₆-DMSO) δ 0.96 (3H, t), 1.50-1.89 (4H, m), 2.05 (2H, q), 2.21 (3H, s), 2.74-3.04 (6H, m), 3.23 (2H, t), 3.34 (2H, t), 3.69-3.93 (1H, m), 4.45-4.72 (1H, m), 7.08 (1H, d), 7.22 (1H, d), 7.34 (1H, s), 7.50 (2H, d), 7.72-7.88 (4H, m), m/z (ESI+) (M+H)+=476.43; HPLC tR=1.83 min.

### EXAMPLE 354

### 1-[5-[4-(4-cyanophenyl)piperidine-1-carbonyl]-2-methyl-phenyl]-3-(1-pyridin-3-ylethyl)urea

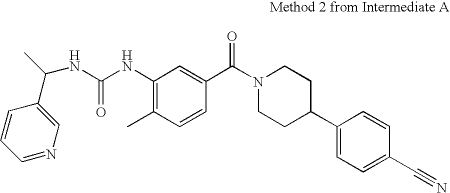

¹H NMR (400.132 MHz, d₆-DMSO) δ 1.43 (3H, d), 1.48-1.92 (4H, m), 2.21 (3H, s), 2.72-2.96 (2H, m), 2.98-3.21 (1H, m), 3.67-3.84 (1H, m), 4.80-4.90 (1H, m), 6.93 (1H, d), 7.18 (2H, d), 7.24 (1H, d), 7.35-7.40 (1H, m), 7.49 (2H, d), 7.72-7.80 (4H, m), 7.96 (1H, d), 8.46 (1H, d), 8.59 (1H, d), m/z (ESI+) (M+H)+=468.49; HPLC tR=1.53 min.

### EXAMPLE 355

### 1-[5-[4-(4-chlorophenyl)piperidine-1-carbonyl]-2-methyl-phenyl]-3-(pyridin-2-ylmethyl)urea

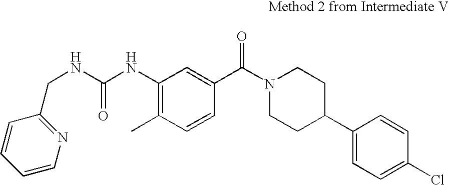

¹H NMR (400.132 MHz, d₆-DMSO) δ 1.41-1.95 (4H, m), 2.23 (3H, s), 2.71-2.92 (2H, m), 3.00-3.21 (1H, m), 3.68-3.87 (1H, m), 4.42 (2H, d), 4.50-4.69 (1H, m), 6.94 (1H, d), 7.20 (1H, d), 7.25-7.40 (7H, m), 7.75-7.82 (1H, m), 7.97 (1H, s), 8.05 (1H, s), 8.54 (1H, d), m/z (ESI+) (M+H)+=463.45; HPLC tR=1.79 min.

### EXAMPLE 356

### 1-[5-[4-(4-cyanophenyl)piperidine-1-carbonyl]-2-methyl-phenyl]-3-[(1-methylpyrazol-4-yl)methyl]urea

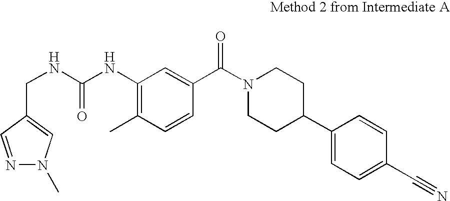

¹H NMR (400.132 MHz, d₆-DMSO) δ 1.50-1.94 (4H, m), 2.19 (3H, s), 2.74-2.99 (2H, m), 3.00-3.22 (1H, m), 3.68-3.87 (4H, m), 4.11 (2H, d), 4.53-4.70 (1H, m), 6.85 (1H, t), 6.93 (1H, d), 7.18 (1H, d), 7.37 (1H, s), 7.51 (2H, d), 7.62 (1H, s), 7.73 (1H, s), 7.76-7.81 (2H, m), 8.01 (1H, d), m/z (ESI+) (M+H)+=457.49; HPLC tR=1.94 min.

### EXAMPLE 357

### 1-[5-[4-(4-cyanophenyl)piperidine-1-carbonyl]-2-methyl-phenyl]-3-(1-pyridin-4-ylethyl)urea

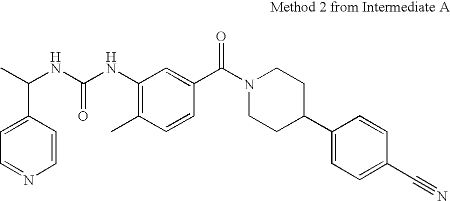

¹H NMR (400.132 MHz, d₆-DMSO) δ 1.39 (3H, d), 1.47-1.90 (4H, m), 2.23 (3H, s), 2.71-2.97 (2H, m), 3.00-3.21 (1H, m), 3.67-3.84 (1H, m), 4.52-4.68 (1H, m), 4.75-4.85 (1H, m), 6.91-6.95 (1H, m), 7.19 (1H, d), 7.26 (1H, d), 7.35 (2H, d), 7.49 (2H, d), 7.77 (2H, d), 7.83 (1H, s), 7.95 (1H, d), 8.50-8.55 (2H, m), m/z (ESI+) (M+H)+=468.5; HPLC tR=1.43 min.

### EXAMPLE 358

### 1-[5-[4-(4-cyanophenyl)piperidine-1-carbonyl]-2-methyl-phenyl]-3-[(6-morpholin-4-ylpyridin-2-yl)methyl]urea

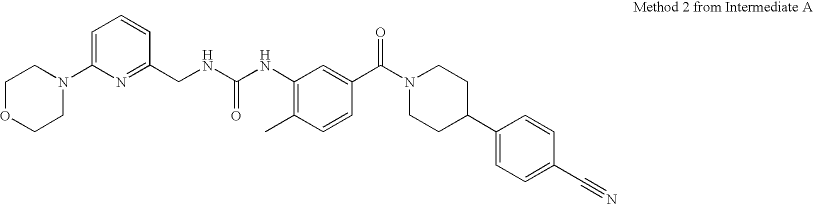

¹H NMR (400.132 MHz, d₆-DMSO) δ 1.46-1.95 (4H, m), 2.23 (3H, s), 2.70-3.00 (2H, m), 3.01-3.21 (1H, m), 3.40-3.49 (4H, m), 3.65-3.87 (5H, m), 4.25 (2H, d), 4.52-4.69 (1H, m), 6.65 (1H, d), 6.70 (1H, d), 6.95 (1H, d), 7.03-7.09 (1H, m), 7.20 (1H, d), 7.47-7.58 (3H, m), 7.77 (2H, d), 7.94 (1H, s), 7.99 (1H, s), m/z (ESI+) (M+H)+=539.56; HPLC tR=1.84

### EXAMPLE 359

### 1-[5-[4-(4-chlorophenyl)piperidine-1-carbonyl]-2-methyl-phenyl]-3-propan-2-yl-urea

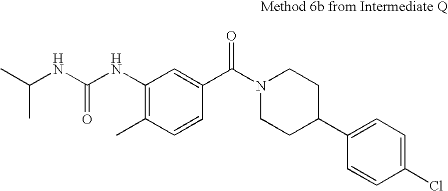

¹H NMR (400.132 MHz, d₆-DMSO) δ 1.04 (6H, d), 1.36-1.86 (4H, m), 2.13 (3H, s), 2.70-2.81 (2H, m), 2.95-3.14 (1H, m), 3.61-3.77 (2H, m), 4.45-4.63 (1H, m), 6.50 (1H, d), 6.83 (1H, d), 7.10 (1H, d), 7.24 (2H, d), 7.29 (2H, d), 7.54 (1H, s), 7.94 (1H, s), m/z (ESI+) (M+H)+=414.37; HPLC tR=2.53.

### EXAMPLE 360

### 1-[2-methyl-5-[4-(4-sulfamoylphenyl)piperidine-1-carbonyl]phenyl]-3-propan-2-yl-urea

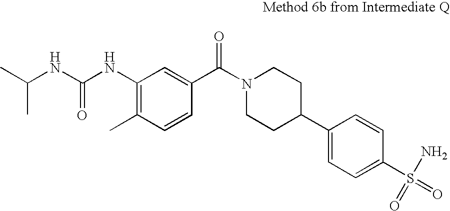

¹H NMR (400.132 MHz, d₆-DMSO) δ 1.11 (6H, d), 1.50-1.93 (4H, m), 2.20 (3H, s), 2.70-3.23 (3H, m), 3.65-3.88 (2H, m), 4.52-4.73 (1H, m), 6.57 (1H, d), 6.91 (1H, d), 7.18 (1H, d), 7.29 (2H, s), 7.48 (2H, d), 7.62 (1H, s), 7.76 (2H, d), 8.02 (1H, s), m/z (ESI+) (M+H)+=459.31; HPLC tR=1.77.

### EXAMPLE 361

### 1-[5-[4-(4-cyanophenyl)piperidine-1-carbonyl]-2-methyl-phenyl]-3-methyl-urea

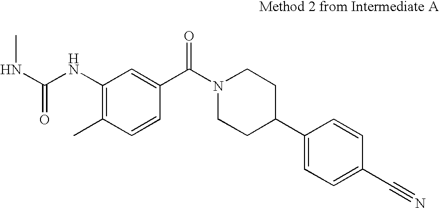

¹H NMR (400.132 MHz, d₆-DMSO) δ 1.51-1.95 (4H, m), 2.20 (3H, s), 2.66 (3H, d), 2.76-3.23 (3H, m), 3.64-3.94 (1H, m), 4.45-4.73 (1H, m), 6.43-6.50 (1H, m), 6.93 (1H, d), 7.18 (1H, d), 7.51 (2H, d), 7.72 (1H, s), 7.77 (2H, d), 7.93 (1H, s), m/z (ESI+) (M+H)+=377.43; HPLC tR=2.03 min.

### EXAMPLE 362

### 3-[5-[4-(4-cyanophenyl)piperidine-1-carbonyl]-2-methylsulfanylphenyl]-1-propan-2-ylurea

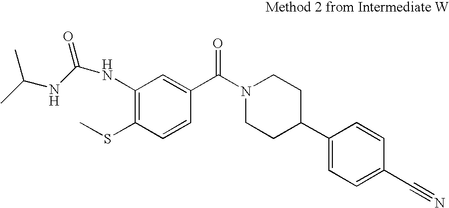

¹H NMR (300.072 MHz, CDCl₃) [symbol] 1.21 (d, 6H), 1.67-1.95 (m, 4H), 2.36 (s, 3H), 2.78-2.89 (m, 2H), 3.06-3.32 (m, 1H), 3.96 (septet, 1H), 4.04-4.09 (m, 1H), 4.78-4.96 (m, 2H), 7.05-7.08 (m, 1H), 7.22 (s, 1H), 7.32 (d, 2H), 7.40 (d, 1H), 7.60 (d, 2H), 8.08 (s, 1H), m/z (ESI+) (M+H)+=437; HPLC tR=2.32 min.

### EXAMPLE 363

### 3-benzyl-1-[5-[4-(4-cyanophenyl)piperidine-1-carbonyl]-2-methylsulfanylphenyl]urea

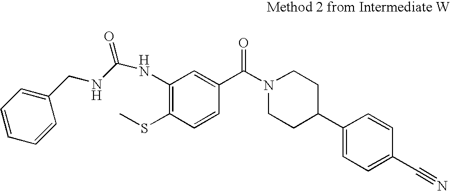

¹H NMR (300.072 MHz, CDCl₃) [symbol] 1.63-1.97 (m, 4H), 2.32 (s, 3H), 2.75-2.88 (m, 2H), 3.05-3.23 (m, 1H), 3.82-4.13 (m, 1H), 4.46 (d, 2H), 4.74-4.96 (m, 1H), 5.37 (t, 1H), 7.03-7.08 (m, 1H), 7.27-7.40 (m, 9H), 7.60 (d, 2H), 8.07-8.10 (m, 1H), m/z (ESI+) (M+H)+=485; HPLC tR=2.52 min.

### EXAMPLE 364

### 3-[5-[4-(4-cyanophenyl)piperidine-1-carbonyl]-2-methylsulfonylphenyl]-1-propan-2-ylurea

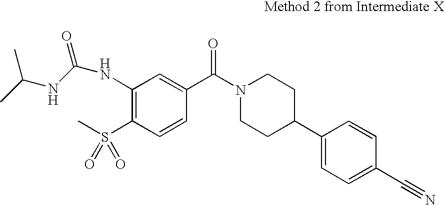

¹H NMR (300.072 MHz, CDCl₃) [symbol] 1.22 (d, 6H), 1.64-1.99 (m, 4H), 2.80-2.94 (m, 2H), 3.08 (s, 3H), 3.16-3.27 (m, 1H), 3.79-4.01 (m, 2H), 4.81-4.94 (m, 1H), 5.14 (d, 1H), 7.16-7.21 (m, 1H), 7.34 (d, 2H), 7.61 (d, 2H), 7.90 (d, 1H), 8.32-8.35 (m, 1H), 8.51 (s, 1H), m/z (ESI⁻) (M−H)⁻=467; HPLC tR=2.19 min.

### EXAMPLE 365

### 3-[5-[4-(4-cyanophenyl)piperidine-1-carbonyl]-2-(methoxymethyl)phenyl]-1-propan-2-ylurea

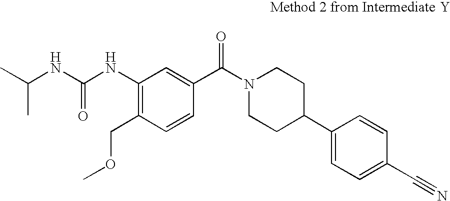

¹H NMR (300.073 MHz, d₆-DMSO) δ 1.10 (6H, d), 1.45-2.00 (4H, m), 2.68-3.23 (3H, m), 3.30 (3H, s), 3.64-3.83 (2H, m), 4.40 (2H, s), 4.49-4.75 (1H, m), 6.80 (1H, d), 6.97 (1H, d), 7.29 (1H, d), 7.50 (2H, d), 7.64 (1H, s), 7.76 (2H, d), 8.01 (1H, s), m/z (ESI+) (M+H)+=435.35; HPLC tR=2.21 min.

### EXAMPLE 366

### [5-[4-(4-cyanophenyl)piperidine-1-carbonyl]-2-methylphenyl]urea

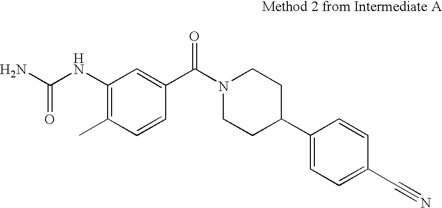

1H NMR (400.132 MHz, d₆-DMSO) δ 1.54-1.69 (2H, m), 1.71-1.92 (2H, m), 2.22 (3H, s), 2.63-3.22 (3H, m), 3.66-3.97 (1H, m), 4.44-4.78 (1H, m), 6.09 (2H, s), 6.94 (1H, d), 7.18 (1H, d), 7.50 (2H, d), 7.74-7.81 (3H, m), 7.97 (1H, s), m/z (ESI+) (M+H)+=363.33; HPLC tR=1.85 min.

### EXAMPLE 367

### 1-[5-[4-(4-cyanophenyl)piperidine-1-carbonyl]-2-methylphenyl]-3-[(3,5-difluoropyridin-2-yl)methyl]urea

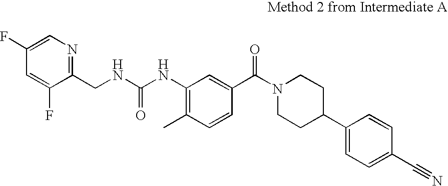

¹H NMR (400.132 MHz, d₆-DMSO) δ 1.47-1.94 (4H, m), 2.21 (3H, d), 2.71-3.23 (3H, m), 3.65-3.88 (1H, m), 4.49 (2H, d), 4.53-4.72 (1H, m), 6.94 (1H, dd), 7.19 (1H, d), 7.28 (1H, t), 7.50 (2H, d), 7.77 (2H, dd), 7.91-8.00 (2H, m), 8.07 (1H, s), 8.51 (1H, d), m/z (ESI+) (M+H)+=490.48; HPLC tR=2.33 min.

### EXAMPLE 368

### 1-[5-[4-(4-cyanophenyl)piperidine-1-carbonyl]-2-methylphenyl]-3-(1-pyridin-2-ylethyl)urea

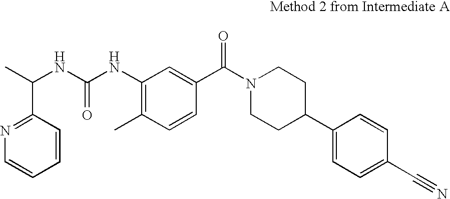

¹H NMR (400.132 MHz, d₆-DMSO) δ 1.39 (3H, d), 1.47-1.92 (4H, m), 2.22 (3H, s), 2.64-3.23 (3H, m), 3.63-3.87 (1H, m), 4.49-4.70 (1H, m), 4.91 (1H, quintet), 6.92 (1H, dd), 7.18 (1H, d), 7.24-7.36 (2H, m), 7.39 (1H, d), 7.49 (2H, d), 7.73-7.81 (3H, m), 7.96-8.01 (2H, m), 8.56 (1H, ddd), m/z (ESI+) (M+H)+=468.54; HPLC tR=1.76 min.

### EXAMPLE 369

### 2-[[5-[4-(4-cyanophenyl)piperidine-1-carbonyl]-2-methylphenyl]carbamoylamino]-3-(4-fluorophenyl)propanoic acid

### EXAMPLE 370

### (2R)-3-[[5-[4-(4-cyanophenyl)piperidine-1-carbonyl]-2-methylphenyl]carbamoylamino]-2-[(2-methylpropan-2-yl)oxycarbonylamino]propanoic acid

### EXAMPLE 371

### 2-[[5-[4-(4-cyanophenyl)piperidine-1-carbonyl]-2-methylphenyl]carbamoylamino]-3-methylbutanoic acid

### EXAMPLE 372

### 4-[[5-[4-(4-cyanophenyl)piperidine-1-carbonyl]-2-methylphenyl]carbamoylamino]butanoic acid

### EXAMPLE 373

### 2-[[5-[4-(4-cyanophenyl)piperidine-1-carbonyl]-2-methylphenyl]carbamoylamino]acetic acid

### EXAMPLE 374

### 1-[2-methyl-5-[4-(4-methylsulfonylphenyl)piperidine-1-carbonyl]phenyl]-3-propan-2-ylurea

### EXAMPLE 375

### 1-[3-[4-(4-cyanophenyl)piperidine-1-carbonyl]-4-methylphenyl]-3-propan-2-ylurea

### EXAMPLE 376

### 3-tert-butyl-1-[3-[4-(4-cyanophenyl)piperidine-1-carbonyl]-4-methylphenyl]urea

### EXAMPLE 377

### 3-[3-[4-(4-cyanophenyl)piperidine-1-carbonyl]-4-fluorophenyl]-1-propan-2-ylurea

### EXAMPLE 378

### 3-[(4-cyanophenyl)methyl]-1-[3-[4-(4-cyanophenyl)piperidine-1-carbonyl]-4-methylphenyl]urea

### EXAMPLE 379

### 1-[3-[4-(4-cyanophenyl)piperidine-1-carbonyl]-4-methylphenyl]-3-(pyridin-4-ylmethyl)urea

### EXAMPLE 380

### 3-[3-[4-(4-cyanophenyl)piperidine-1-carbonyl]-4-methylphenyl]-1,1-dimethylurea

### EXAMPLE 381

### 1-benzyl-3-[5-[4-(4-cyanophenyl)piperidine-1-carbonyl]-2-methylphenyl]-1-methylurea

### EXAMPLE 383

### 1-[5-[4-(4-cyanophenyl)piperidine-1-carbonyl]-2-methylphenyl]-3-(3-oxo-1,2-oxazolidin-4-yl)urea

### EXAMPLE 384

### 1-[5-[4-(4-cyanophenyl)piperidine-1-carbonyl]-2-methylphenyl]-3-(2-methylbut-3-yn-2-yl)urea

### EXAMPLE 385

### Ethyl 2-[[5-[4-(4-cyanophenyl)piperidine-1-carbonyl]-2-methylphenyl]carbamoylamino]-propanoate

### EXAMPLE 386

### 3-[5-[4-(4-cyanophenyl)piperidine-1-carbonyl]-2-methylsulfinylphenyl]-1-propan-2-ylurea

### EXAMPLE 387

### 3-benzyl-1-[5-[4-(4-cyanophenyl)piperidine-1-carbonyl]-2-methylsulfinylphenyl]urea

### EXAMPLE 388

### 2-[[5-[4-(4-cyanophenyl)piperidine-1-carbonyl]-2-methylphenyl]carbamoylamino]-cyclopentane-1-carboxylic acid

### EXAMPLE 389

### 1-[5-[4-(4-cyanophenyl)piperidine-1-carbonyl]-2-methylphenyl]-3-(1-methylcyclopropyl)-urea

### EXAMPLE 390

### 3-(1-acetylpiperidin-3-yl)-1-[5-[4-(4-cyanophenyl)piperidine-1-carbonyl]-2-methylphenyl]urea

### EXAMPLE 391

### 1-[2-methyl-5-(4-phenylpiperidine-1-carbonyl)phenyl]-3-propan-2-ylurea

### EXAMPLE 392

### 3-[5-[4-(4-cyanophenyl)piperidine-1-carbonyl]-2-methylphenyl]-1-(2-hydroxyethyl)-1-methylurea

### EXAMPLE 393

### 3-[5-[4-(4-cyanophenyl)piperidine-1-carbonyl]-2-methylphenyl]-1-methyl-1-(1-methylpiperidin-4-yl)urea

### EXAMPLE 394

### 3-[5-[4-(4-cyanophenyl)piperidine-1-carbonyl]-2-methylphenyl]-1-(2-dimethylaminoethyl)-1-methylurea

### EXAMPLE 395

### 3-[5-[4-(4-cyanophenyl)piperidine-1-carbonyl]-2-methylphenyl]-1-(2-hydroxyethyl)-1-propan-2-ylurea

### EXAMPLE 396

### 3-[5-[4-(4-cyanophenyl)piperidine-1-carbonyl]-2-methylphenyl]-1-methyl-1-(oxan-4-yl)urea

### EXAMPLE 397

### 3-[5-[4-(4-cyanophenyl)piperidine-1-carbonyl]-2-methylphenyl]-1-(1,1-dioxothiolan-3-yl)-1-propylurea

### EXAMPLE 398

### 2-[[5-[4-(4-cyanophenyl)piperidine-1-carbonyl]-2-methylphenyl]carbamoyl-methylamino]-N-propan-2-ylacetamide

### EXAMPLE 399

### N-[5-[4-(4-cyanophenyl)piperidine-1-carbonyl]-2-methylphenyl]-2-(ethoxymethyl)pyrrolidine-1-carboxamide

### EXAMPLE 400

### 1-[5-[4-(4-fluorophenyl)piperidine-1-carbonyl]-2-methylphenyl]-3-(2-methylaminoethyl)urea

### EXAMPLE 401

### N-[2-[[5-[4-(4-cyanophenyl)piperidine-1-carbonyl]-2-methylphenyl]carbamoyl-methylamino]ethyl]acetamide

### EXAMPLE 402

### 3-[5-[4-(4-cyanophenyl)piperidine-1-carbonyl]-2-methylphenyl]-1-[2-(ethylsulfonylamino)ethyl]-1-methylurea

### EXAMPLE 403

### 3-[5-[4-(4-cyanophenyl)piperidine-1-carbonyl]-2-(methylsulfonylmethyl)phenyl]-1-propan-2-ylurea

### EXAMPLE 404

### 1-[3-[4-(4-cyanophenyl)piperidine-1-carbonyl]-4-methylphenyl]-3-propan-2-ylurea

### EXAMPLE 405

### 3-[3-[4-(4-cyanophenyl)piperidine-1-carbonyl]-4-methylsulfanylphenyl]-1-propan-2-ylurea

### EXAMPLE 406

### 4-[[5-[4-(4-cyanophenyl)piperidine-1-carbonyl]-2-methylphenyl]carbamoylamino]cyclohexane-1-carboxylic acid

### EXAMPLE 407

### 4-[[5-[4-(4-cyanophenyl)piperidine-1-carbonyl]-2-methylphenyl]carbamoylamino]butanoic acid

### EXAMPLE 408

### 2-[[5-[4-(4-cyanophenyl)piperidine-1-carbonyl]-2-methylphenyl]carbamoylamino]acetic acid

### EXAMPLE 409

### 1-[5-[4-(4-cyanophenyl)piperidine-1-carbonyl]-2,4-dimethylphenyl]-3-(pyridin-2-ylmethyl)urea

### EXAMPLE 410

### 3-[5-[4-(4-cyanophenyl)piperidine-1-carbonyl]-2,4-dimethylphenyl]-1-ethylurea

### EXAMPLE 411

### 1-[5-[4-(4-cyanophenyl)piperidine-1-carbonyl]-2,4-dimethylphenyl]-3-cyclopropylurea

### EXAMPLE 412

### 1-[5-[4-(4-cyanophenyl)piperidine-1-carbonyl]-2,4-dimethylphenyl]-3-(2-methoxyethyl)urea

### EXAMPLE 413

### 1-[5-[4-(4-cyanophenyl)piperidine-1-carbonyl]-2,4-dimethylphenyl]-3-prop-2-ynylurea

### EXAMPLE 414

### 1-[3-[4-(4-cyanophenyl)piperidine-1-carbonyl]-4-methylphenyl]-3-(pyridin-2-ylmethyl)urea

### EXAMPLE 415

### 1-[3-[4-(4-cyanophenyl)piperidine-1-carbonyl]-4-methylphenyl]-3-cyclopropylurea

### EXAMPLE 416

### 3-[3-[4-(4-cyanophenyl)piperidine-1-carbonyl]-4-methylphenyl]-1-(3-ethoxypropyl)urea

### EXAMPLE 417

### 1-[3-[4-(4-cyanophenyl)piperidine-1-carbonyl]-4-methylphenyl]-3-(2-methoxyethyl)urea

### EXAMPLE 418

### 1-[3-[4-(4-cyanophenyl)piperidine-1-carbonyl]-4-methylphenyl]-3-prop-2-ynylurea

### Preparation of Intermediates

### Intermediate A

### 4-[1-(3-amino-4-methyl-benzoyl)-4-piperidyl]benzonitrile

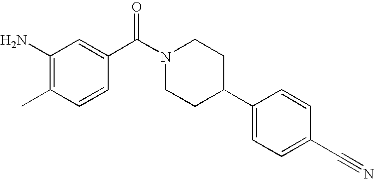

A solution of 3-amino-4-methyl benzoic acid (4.05 g, 26.792 mmol), 4-(4′-cyanophenyl)piperidine (5 g, 26.79 mmol), N-(3-Dimethylaminopropyl)-N′-ethylcarbodiimide hydrochloride [EDAC] (5.64 g, 29.47 mmol, 1.1 eq) and DMAP (328 mg, 2.68 mmol, 0.1 eq) in DMF (60 mL) was stirred at ambient temperature for 2 hrs. Ethyl acetate (200 mL) was added and the resulting solution was washed sequentially with KHSO₄ solution (100 mL of 2M), and brine (100 ml); a precipitate formed and was filtered off to give the title compound as a colourless solid (5.25 g), ¹H NMR (300.073 MHz, d₆-DMSO, 30° C.) δ 1.47-1.67 (2H, m), 1.68-1.89 (2H, m), 2.05 (3H, s), 2.68-3.15 (3H, m), 3.59-4.13 (1H, m), 4.22-4.76 (1H, m), 4.97 (2H, s), 6.45-6.53 (1H, m), 6.63 (1H, s), 6.90-6.99 (1H, m), 7.44-7.54 (2H, m), 7.71-7.81 (2H, m), m/z 320 (M+H)⁺.

### Intermediate B

### 4-[1-[3-amino-4-(trifluoromethoxy)benzoyl]-4-piperidyl]benzonitrile

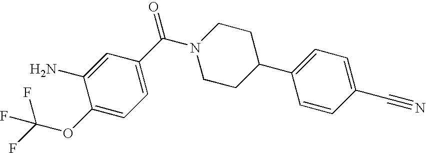

### Step 1: 3-nitro-4-(trifluoromethoxy)benzoic acid

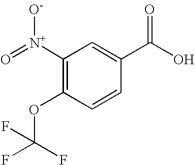

4-(trifluoromethoxy)benzoic acid (4 g, 26.3 mmol) was added slowly to a stirred mixture of concentrated sulfuric acid ((6.6 mL, 65.7 mmol) and concentrated nitric acid (4 mL, 44.7 mmol) at 40° C. When the addition was complete, the reaction mixture was heated to 50° C. and became a pale yellow slurry. After 1 h the reaction appeared to have gone to completion and so was poured onto ice and water. A white precipitate formed which was isolated by filtration and washed with water to give the title compound as a colourless crystalline solid (4.2 g), ¹H NMR (300.073 MHz, DMSO-d₆) δ7.83-7.87 (1H, m), 8.32-8.36 (1H, m), 8.56 (1H, d), m/z 198 (M+H)⁺.

### Step 2: 3-amino-4-(trifluoromethoxy)benzoic acid

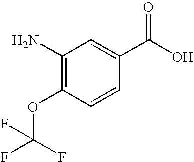

A solution of 3-nitro-4-(trifluoromethoxy)benzoic acid (Step 1) (3.51 g, 14 mmol) in MeOH (150 mL) was hydrogenated at ambient temperature and pressure in the presence of 10% palladium on charcoal catalyst (500 mg). The catalyst was removed by filtration and washed through with more MeOH; the filtrate and washings were combined and evaporated to give the title compound as a pale cream solid (2.9 g), ¹H NMR (300.073 MHz, d₆-DMSO) δ 5.60 (br s, 2H), 7.07-7.23 (m, 2H), 7.39-7.46 (m, 1H), 12.02-13.40 (br s, 1H), m/z 220 (M−H)⁻.

### Step 3: 4-[1-[3-amino-4-(trifluoromethoxy)benzoyl]-4-piperidyl]benzonitrile

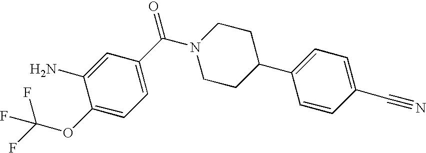

A mixture of 3-amino-4-trifluoromethoxy benzoic acid (Step 2) (2.8 g, 12.66 mmol), 4-(4′-cyanophenyl)piperidine (2.36 g, 12.66 mmol, 1 eq), N-(3-Dimethylaminopropyl)-N′-ethylcarbodiimide hydrochloride (EDAC) (2.67 g, 13.93 mmol, 1.1 eq) and DMAP (155 mg, 1.27 mmol, 0.1 eq) in DMF (30 mL) was stirred at room temperature for 2 hrs. EtOAc (200 mL) was added and the resulting mixture washed sequentially with aqueous potassium bisulfate solution (100 mL of 2M KHSO₄), brine (100 mL), dried (MgSO₄), filtered and reduced in vacuo to give a brown oil. Ethyl acetate was added and the resulting colourless solid isolated by filtration. A precipitate also appeared during the extraction process; this was isolated and the solids combined to give the title compound (3.16 g), ¹H NMR (300.072 MHz, CDCl₃) δ 1.43-2.02 (4H, m), 2.77-3.22 (3H, m), 3.80-4.21 (3H, m), 4.63-5.03 (1H, m), 6.72-6.77 (1H, m), 6.87 (1H, d), 7.13-7.18 (1H, m), 7.32 (2H, d), 7.61 (2H, d), m/z 390 (M+H)⁺.

### Intermediate C

### 4-[1-(3-amino-4-methoxy-benzoyl)-4-piperidyl]benzonitrile

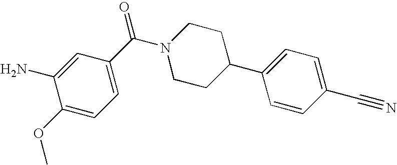

A stirred mixture of 4-(4′-cyanophenyl)piperidine (3 g, 16 mmol); 3-amino-4-methoxybenzoic acid (2.675 g, 16 mmol, 1 eq) and DIPEA (4.2 ml, 24 mmol, 1.5 eq) in DCM (100 mL) was blanketed with nitrogen and treated with N-(3-Dimethylaminopropyl)-N′-ethylcarbodiimide hydrochloride (EDAC) (3.4 g, 17.6 mmol, 1.1 eq). The reaction mixture was stirred for three days. Addition of water to the reaction mixture resulted in an emulsion and a colourless precipitate. The solid was isolated by filtration and washed with EtOAc (2×75 mL portions) to give a colourless solid (2.5 g). The ethyl acetate washings were combined, washed with water, dried (MgSO₄) and evaporated to give a further 2 g; the solids thus prepared were identical and combined to give the title compound as (4.5 g, 83%), ¹H NMR (300.073 MHz, d₆-DMSO, 30° C.) δ 1.48-1.67 (2H, m), 1.71-1.87 (2H, m), 2.80-3.08 (3H, m), 3.78 (3H, s), 3.88-4.63 (2H, m), 4.84 (2H, s), 6.56-6.64 (1H, m), 6.67-6.73 (1H, m), 6.80 (1H, dJ=8.1 Hz), 7.49 (2H, dJ=8.1 Hz), 7.76 (2H, dJ=9.4 Hz), m/z 336 (M+H)⁺.

### Intermediate D

### 4-[1-(5-amino-2-methyl-benzoyl)-4-piperidyl]benzonitrile

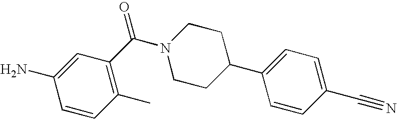

### Step 1:

### 4-[1-(2-methyl-5-nitro-benzoyl)-4-piperidyl]benzonitrile

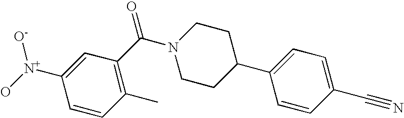

The title compound was prepared in a manner similar to that described for Intermediate A, starting from 2-methyl-5-nitrobenzoic acid and 4-(4′-cyanophenyl)piperidine, ¹H NMR (300.072 MHz, CDCl₃) δ 1.64-2.11 (4H, m), 2.42-2.53 (3H, m), 2.79-3.00 (2H, m), 3.09-3.23 (1H, m), 3.55 (1H, d), 4.98 (1H, d), 7.29-7.36 (2H, m), 7.42 (1H, d), 7.63 (2H, d), 8.04-8.18 (2H, m), m/z 348 (M−H)⁻.

### Step 2:

### 4-[1-(5-amino-2-methyl-benzoyl)-4-piperidyl]benzonitrile

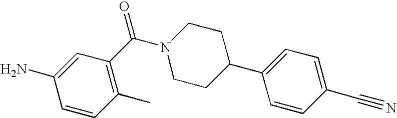

10% Palladium on carbon (0.6 g, 15% by weight) was added to a solution of 4-[1-(2-methyl-5-nitro-benzoyl)-4-piperidyl]benzonitrile (Step 1) (4 g, 11.45 mmol) in ethanol (100 mL) and THF (100 mL). After purging the reaction flask with nitrogen, hydrogen was introduced and the resulting reaction mixture was stirred for 4 hrs. The catalyst was removed by filtration through celite and the filtrate was evaporated in vacuo to give a yellow foam. This still contained unreacted starting material so the hydrogenation procedure was repeated for a further 1 hr. The catalyst was removed by filtration through celite and the solvent removed in vacuo to give a yellow foam. This was dissolved in EtOAc and the solution was then washed sequentially with water and brine, dried (MgSO₄) and the solvent removed in vacuo to give the title compound as a yellow solid (3.3 g), ¹H NMR (400.132 MHz, d₆-DMSO) 61.39-1.62 (2H, m), 1.69-1.78 (1H, m), 1.88 (1H, d), 1.99-2.12 (3H, m), 2.80 (1H, t), 2.90-2.99 (1H, m), 3.08 (1H, t), 3.45-3.51 (1H, m), 4.63-4.72 (1H, m), 5.01 (2H, s), 6.37 (1H, d), 6.48-6.52 (1H, m), 6.89 (1H, d), 7.46-7.52 (2H, m), 7.78 (2H, d), m/z 320 (M+H)⁺.

### Intermediate E

### 4-[1-(3-amino-4-fluoro-benzoyl)-4-piperidyl]benzonitrile

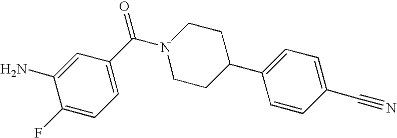

The title compound was prepared in a manner similar to that described for Intermediate A, starting from 3-amino-4-fluoro-benzoic acid and 4-(4′-cyanophenyl)piperidine. After work-up of the reaction, the crude product was purified by trituration with ethanol to give a colourless solid, ¹H NMR (300.073 MHz, d₆-DMSO) δ 1.43-2.00 (m, 4H), 2.68-3.23 (m, 3H), 3.59-4.05 (m, 1H), 4.21-4.88 (m, 1H), 5.29 (s, 2H), 6.49-6.61 (m, 1H), 6.80 (d, 1H), 6.95-7.08 (m, 1H), 7.49 (d, 2H), 7.76 (d, 2H), m/z 324 (M+H)⁺.

### Intermediate F

### 4-[1-(5-amino-2,4-dimethyl-benzoyl)-4-piperidyl]benzonitrile

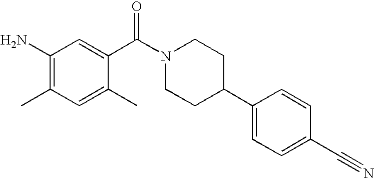

### Step 1: 4-[1-(2,4-dimethyl-5-nitro-benzoyl)-4-piperidyl]benzonitrile

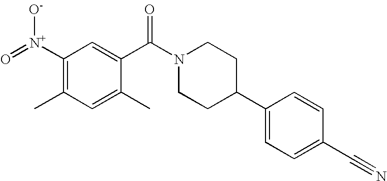

The title compound was prepared in a manner similar to that described for Intermediate A, starting from 2,4-dimethyl-5-nitro-benzoic acid and 4-(4′-cyanophenyl)piperidine; ¹H NMR (300.072 MHz, CDCl₃) δ 1.44-1.64 (m, 1H), 1.66-1.91 (m, 2H), 1.95-2.09 (m, 1H), 2.30-2.49 (br s, 3H), 2.61 (s, 3H), 2.79-2.95 (m, 2H), 3.05-3.26 (m, 1H), 3.59 (d, 1H), 4.95 (d, 1H), 7.22 (s, 1H), 7.32 (d, 2H), 7.61 (d, 2H), 7.81 (br s, 1H), m/z 405 (M+MeCN+H)⁺.

### Step 2: 4-[1-(5-amino-2,4-dimethyl-benzoyl)-4-piperidyl]benzonitrile

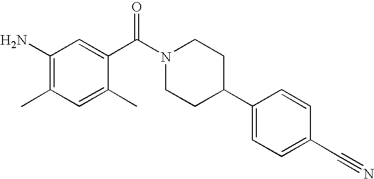

The title compound was prepared in a manner similar to that described for Intermediate D, Step 2, starting from 4-[1-(2,4-dimethyl-5-nitro-benzoyl)-4-piperidyl]benzonitrile (Step 1), and using a methanol/THF mixture (1:1) as solvent; ¹H NMR (300.073 MHz, d₆-DMSO) δ 1.36-1.63 (m, 2H), 1.64-1.79 (m, 1H), 1.80-1.95 (m, 1H), 1.96-2.08 (br s, 3H), 2.02 (s, 3H), 2.69-2.85 (m, 1H), 2.85-2.97 (m, 1H), 2.98-3.12 (m, 1H), 3.40-3.56 (m, 1H), 4.59-4.70 (m, 1H), 4.73 (br s, 2H), 6.30-6.55 (br m, 1H), 6.78 (s, 1H), 7.47 (d, 2H), 7.77 (d, 2H); peak broadening is observed due to conformations of amide group, m/z 334 (M+H)⁺.

### Intermediate G

### 2-amino-4-[4-(4-cyanophenyl)piperidine-1-carbonyl]benzonitrile

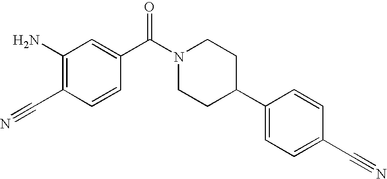

### Step 1: Ethyl 4-cyano-3-nitro-benzoate

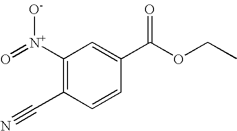

Water (0.01 mL) was added to a solution of 4-iodo-3-nitro benzoic acid ethyl ester (0.4 g, 1.25 mmol) and zinc cyanide (79 mg, 0.67 mmol) in NMP (5 mL) and nitrogen was bubbled through the mixture for 5 mins. Bis(dibenzylideneacetone)palladium(0) (29 mg, 0.05 mmol) and 1,1′-Bis(diphenylphosphino)ferrocene (83 mg, 0.15 mmol) were added and the vessel sealed and filled with nitrogen. The reaction was heated in the microwave oven at 150° C. for 5 mins. EtOAc (50 ml) was added and the resulting mixture was filtered through celite and then washed sequentially with dilute aqueous hydrochloric acid (50 mL of 1M), saturated aqueous sodium bicarbonate solution (50 mL), water (50 mL) and brine (50 mL), dried (MgSO₄), filtered and reduced in vacuo to give a brown oil which was chromatographed (40 g silica column, Companion, eluting with a gradient consisting of isohexane containing 0-20% EtOAc to give the title compound as a yellow solid (200 mg), ¹H NMR (300.072 MHz, CDCl₃) [symbol] 1.45 (t, 3H), 4.49 (q, 2H), 8.01 (d, 1H), 8.42-8.47 (m, 1H), 8.92 (d, 1H), m/z 220 (M^{.+}).

### Step 2: Ethyl 3-amino-4-cyano-benzoate

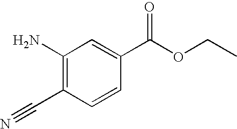

This was prepared by hydrogenation of ethyl 4-cyano-3-nitro-benzoate (Step 1) using a procedure similar to that described in Intermediate B, Step 2, to give the title compound as a yellow solid, ¹H NMR (300.072 MHz, CDCl₃) δ 1.39 (3H, t), 4.38 (2H, q), 4.57 (2H, s), 7.34-7.47 (3H, m), m/z 190 (M^{.+}).

### Step 3: 3-amino-4-cyano-benzoic acid

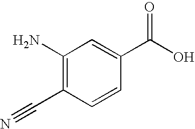

A solution of ethyl 3-amino-4-cyano-benzoate (Step 2) (140 mg, 0.74 mmol) in THF (6 mL) was treated with a solution of lithium hydroxide monohydrate (47 mg, 1.10 mmol) in water (3 ml), and the mixture stirred at ambient temperature for 2 hrs. The THF was removed in vacuo and the aqueous residue washed with EtOAc (30 mL) to remove any unreacted starting material. The aqueous was then adjusted to pH3 with citric acid solution (1M), and extracted with EtOAc (20 mL). The organic extracts were washed with brine (20 mL), dried (MgSO₄), filtered and reduced in vacuo to give the title compound as a yellow solid (60 mg), ¹H NMR (300.073 MHz, d₆-DMSO) [symbol] 6.27 (s, 2H), 7.04-7.08 (m, 1H), 7.38-7.40 (m, 1H), 7.47 (d, 1H), 13.03 (s, 1H), m/z 161 (M−H)⁻.

### Step 4: 2-amino-4-[4-(4-cyanophenyl)piperidine-1-carbonyl]benzonitrile

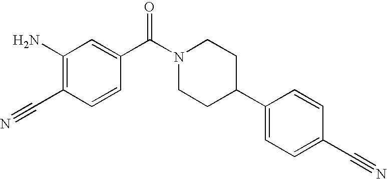

The title compound was prepared in a manner similar to that described for Intermediate A, starting from 3-amino-4-cyano-benzoic acid (Step 3) and 4-(4′-cyanophenyl)piperidine, ¹H NMR (300.072 MHz, CDCl₃) [symbol] 1.53-2.06 (m, 4H), 2.78-2.92 (m, 2H), 3.05-3.22 (m, 1H), 3.71-3.96 (m, 1H), 4.61 (s, 2H), 4.79-4.97 (m, 1H), 6.71-6.75 (m, 1H), 6.78-6.81 (m, 1H), 7.32 (d, 2H), 7.42 (d, 1H), 7.62 (d, 2H), m/z 331 (M+H)⁺.

### Intermediate H

### (3-amino-4-methyl-phenyl)-[4-(4-bromophenyl)-4-hydroxy-1-piperidyl]methanone

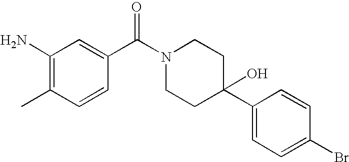

### Step 1: [4-(4-bromophenyl)-4-hydroxy-1-piperidyl]-(4-methyl-3-nitro-phenyl)methanone

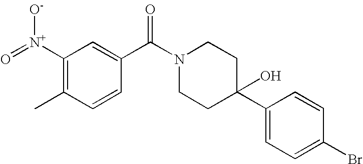

DIPEA (2.04 mL, 14.05 mmol) was added to a stirred solution of 4-(4-bromophenyl)piperidin-4-ol (2.5 g, 9.76 mmol) and 4-methyl-3-nitrobenzoyl chloride (1.42 mL, 9.76 mmol) in DCM (30 mL), and the reaction mixture stirred at room temperature for 20 hrs. It was then washed sequentially with 1M citric acid (40 mL), saturated sodium bicarbonate solution (40 mL), brine (40 mL), dried (MgSO₄), filtered and reduced in vacuo to give a yellow oil. DCM was added and the resulting colourless solid isolated by filtration (2.1 g). The filtrate was chromatographed (120 g silica column, eluting with a gradient consisting of 20-70% EtOAc in isohexane) to give a colourless solid (0.97 g). This was combined with the product isolated previously to give the title compound as a colourless solid (3.07 g, 75%), ¹H NMR (300.072 MHz, CDCl₃) [symbol] 1.67 (s, 1H), 1.73-2.20 (m, 4H), 2.65 (s, 3H), 3.25-3.74 (m, 3H), 4.51-4.81 (m, 1H), 7.34-7.39 (m, 2H), 7.42 (d, 1H), 7.49-7.54 (m, 2H), 7.57-7.61 (m, 1H), 8.06 (d, 1H).

### Step 2: (3-amino-4-methyl-phenyl)-[4-(4-bromophenyl)-4-hydroxy-1-piperidyl]methanone

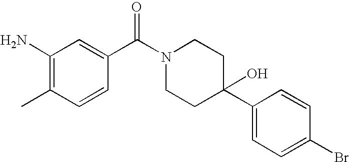

A mixture of [4-(4-bromophenyl)-4-hydroxy-1-piperidyl]-(4-methyl-3-nitro-phenyl)methanone (Step 1, 0.5 g, 1.19 mmol), iron (III) chloride hexahydrate (968 mg, 3.58 mmol) and zinc dust (778 mg, 11.9 mmol) in DMF (10 mL) and water (5 mL) was heated at 100° C. for 4 hrs. The reaction mixture was filtered through celite and reduced in vacuo. EtOAc (30 mL) was added and the solution washed sequentially with water (2×30 mL) and brine (30 mL), dried (MgSO₄), filtered and reduced in vacuo to give the title compound as a colourless solid (0.43 g, 93%), which was used without further purification, ¹H NMR (300.072 MHz, CDCl₃) [symbol] 1.61-2.06 (m, 5H), 2.16 (s, 3H), 3.19-3.55 (m, 3H), 3.65-3.81 (m, 2H), 4.52-4.78 (m, 1H), 6.68-6.74 (m, 2H), 7.05 (d, 1H), 7.34 (d, 2H), 7.48 (d, 2H), m/z 389, 391 (M+H)⁺ [B].

### Intermediate I

### 4-[1-(3-aminobenzoyl)-4-piperidyl]benzonitrile

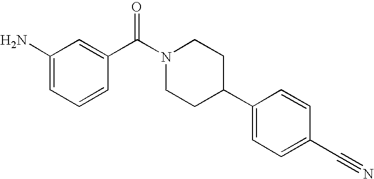

The title compound was prepared in a manner similar to that described for Intermediate A, starting from 3-amino benzoic acid and 4-(4′-cyanophenyl)piperidine. After work-up of the reaction, the crude product was triturated with ether and recrystallised from EtOAc to give a pink solid, ¹H NMR (300.072 MHz, CDCl₃) δ1.50-2.04 (4H, m), 2.79-2.89-3.20 (3H, m), 3.76-3.97 (2H, s), 4.00 (1H, s), 4.90 (1H, s), 6.70-6.78 (3H, m), 7.15-7.20 (1H, m), 7.32 (2H, d), 7.60-7.63 (2H, m), m/z 306 (M+H)⁺.

### Intermediate J

### 4-[1-(3-amino-4-methyl-benzoyl)-4-hydroxy-4-piperidyl]benzonitrile

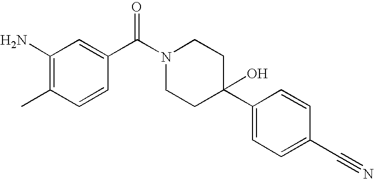

### Step 1: [4-(4-bromophenyl)-4-hydroxy-1-piperidyl]-(4-methyl-3-nitro-phenyl)methanone

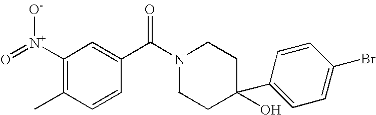

A solution of 4-(4-bromophenyl)-4-piperidinol (2.5 g, 9.76 mmol) and 4-methyl-3-nitrobenzoyl chloride (1.42 mL, 9.76 mmol) in DCM (30 mL) was treated with DIPEA (2.04 mL, 14.05 mmol) and the reaction mixture stirred at ambient temperature for 20 hrs. It was then washed sequentially with aqueous citric acid (40 mL of 1M), saturated sodium bicarbonate solution (40 mL) and brine (40 mL), dried (MgSO₄), and evaporated in vacuo to give a yellow oil. DCM was added and a colourless solid filtered off (2.1 g). The filtrate was purified by chromatography (120 g silica column, gradient eluting with 20-70% EtOAc in isohexane) to give a colourless solid (0.97 g). This was combined with the product isolated previously to give the title compound (3.07 g), ¹H NMR (300.072 MHz, CDCl₃) [symbol] 1.67 (s, 1H), 1.73-2.20 (m, 4H), 2.65 (s, 3H), 3.25-3.74 (m, 3H), 4.51-4.81 (m, 1H), 7.34-7.39 (m, 2H), 7.42 (d, 1H), 7.49-7.54 (m, 2H), 7.57-7.61 (m, 1H), 8.06 (d, 1H).

### Step 2: 4-[4-hydroxy-1-(4-methyl-3-nitro-benzoyl)-4-piperidyl]benzonitrile

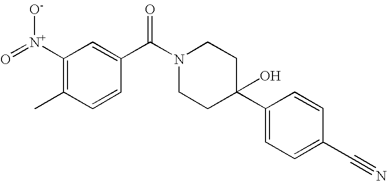

A mixture of [4-(4-bromophenyl)-4-hydroxy-1-piperidyl]-(4-methyl-3-nitro-phenyl)methanone (1.57 g, 3.74 mmol) and copper (I) cyanide (504 mg, 5.62 mmol) in NMP (20 mL) was stirred in the microwave at 190° C. for 12 hrs. EtOAc (30 mL) was added and the resulting mixture was washed sequentially with water (30 mL) and brine (30 mL), dried (MgSO₄) and evaporated in vacuo to give a brown oil which was purified by chromatograph (12 g silica column, eluting with 20-70% EtOAc in isohexane to give the title compound as a colourless solid (0.2 g), ¹H NMR (300.072 MHz, CDCl₃) [symbol] 1.49-2.30 (m, 5H), 2.66 (s, 3H), 3.22-3.85 (m, 3H), 4.53-4.89 (m, 1H), 7.43 (d, 1H), 7.58-7.64 (m, 3H), 7.67-7.71 (m, 2H), 8.07 (d, 1H), m/z 366 (M+H)⁺.

### Step 3: 4-[1-(3-amino-4-methyl-benzoyl)-4-hydroxy-4-piperidyl]benzonitrile

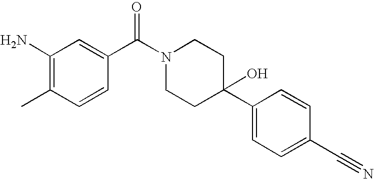

A mixture of 4-[4-hydroxy-1-(4-methyl-3-nitro-benzoyl)-4-piperidyl]benzonitrile (Step 2) (0.2 g, 0.55 mmol), iron (III) chloride hexahydrate (444 mg, 1.64 mmol) and zinc dust (360 mg, 5.5 mmol) in DMF (6 mL) and water (3 mL) was heated at 100° C. for 4 hrs. The reaction mixture was filtered through celite and the filtrate evaporated in vacuo. EtOAc (30 ml) was added to the residue and the resulting solution was washed sequential with water (2×30 mL) and brine (30 mL), dried (MgSO₄) and evaporated in vacuo to give the title compound as a colourless solid (0.17 g), ¹H NMR (300.072 MHz, CDCl₃) [symbol] 1.59-2.10 (m, 5H), 2.18 (s, 3H), 3.10-3.48 (m, 3H), 3.60-4.02 (m, 2H), 4.47-4.73 (m, 1H), 6.69-6.75 (m, 2H), 7.06 (d, 1H), 7.57-7.68 (m, 4H), m/z 336 (M+H)⁺.

### Intermediate K

### 4-[1-(3-amino-4-methyl-benzoyl)-4-piperidyl]-N,N-dimethyl-benzamide

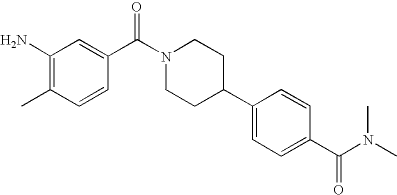

### Step 1: Methyl 4-[1-(4-methyl-3-nitro-benzoyl)-4-piperidyl]benzoate

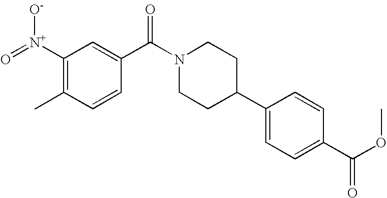

A solution of 4-methyl-3-nitrobenzoyl chloride (3.9 g, 19.55 mmol) in DCM (50 mL) was added dropwise to a solution of methyl 4-(4-piperidyl)benzoate (5 g, 19.55 mmol) and DIPEA in DCM (100 mL). The reaction mixture was stirred at ambient temperature for 72 hrs. The reaction mixture was then washed sequentially with saturated aqueous sodium hydrogen carbonate solution, 1M aqueous citric acid and water. The solvent was dried (phase separating cartridge) and evaporated to give the title compound as a brown waxy solid (6.85 g, 92%), ¹H NMR (300.073 MHz, DMSO-d₆) δ1.56-1.95 (4H, m, 2.56 (3H, s), 2.94 (2H, t), 3.60-3.67 (1H, m), 3.86 (3H, s), 4.64 (1H, s), 7.46 (2H, d), 7.60 (1H, d), 7.71-7.74 (1H, m), 7.92 (2H, d), 8.05 (1H, d), m/z 383 (M+H)⁺.

### Step 2: 4-[1-(4-methyl-3-nitro-benzoyl)-4-piperidyl]benzoic acid

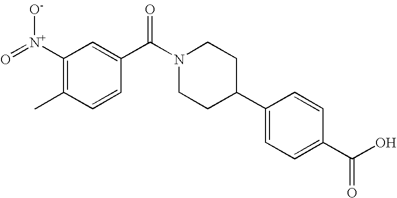

A suspension of methyl 4-[1-(4-methyl-3-nitro-benzoyl)-4-piperidyl]benzoate (Step 1) (5.68 g, 14.9 mmol) in MeOH (57 mL) was treated with aqueous sodium hydroxide solution (19 mL of 2M, 37.1 mmol, 2 eq.) and the reaction mixture stirred at 50° C. for two hrs. More MeOH (25 mL) was added and stirring for continued for one hr. The reaction mixture was cooled and treated with 2M aqueous hydrochloric acid to <pH5, diluted with EtOAc, and the organic layer separated. The aqueous portion was shaken with more EtOAc and the organic layer again separated. The organic extracts were combined, washed with brine, dried over MgSO₄, filtered and evaporated to give the title compound as a yellow solid (4.92 g), ¹H NMR (300.073 MHz, d₆-DMSO) δ 1.56-1.77 (m, 4H), 2.54 (s, 3H), 2.68-3.00 (m, 3H), 3.51-3.73 (m, 1H), 4.52-4.71 (m, 1H), 7.41 (d, J=8.3 Hz, 2H), 7.58 (d, J=7.9 Hz, 1H), 7.70 (d, J=9.4 Hz, 1H), 7.87 (d, J=8.2 Hz, 2H), 8.02 (s, 1H), 12.69-12.99 (m, 1H), m/z 367 (M−H)⁻.

### Step 3: N,N-dimethyl-4-[1-(4-methyl-3-nitro-benzoyl)-4-piperidyl]benzamide

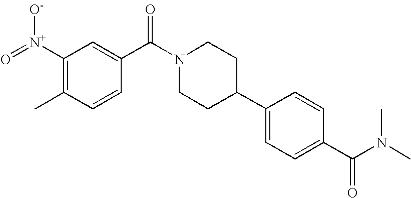

A solution of 4-[1-(4-methyl-3-nitro-benzoyl)-4-piperidyl]benzoic acid (Step 2) (2.89 g, 7.84 mmol), dimethylamine (5.89 mL of a 2M solution in THF, 11.8 mmol, 1.5 eq.), DIPEA (2.7 mL, 15.7 mmol, 2 eq.), and DMAP (2.1 g, 17.3.mmol, 2.2 eq.) in DCM (58 mL) was treated with EDAC (1.8 g, 9.4 mmol, 1.2 eq.), and the reaction mixture stirred for 16 hrs at ambient temperature. Further reagents were added and the reaction mixture stirred for a further 16 hrs. by which time reaction was essentially complete. The reaction mixture was washed with water and the phases separated; the organic portion was concentrated to a brown solid. The crude product was re-dissolved in DCM and the solution washed sequentially with water, citric acid solution (1M in water) and saturated sodium bicarbonate solution. The organic phase was dried (MgSO₄), filtered and concentrated to a brown solid, which was purified by chromatography (120 g silica column, gradient eluting with 10-50% EtOAc in iso-hexane) to give the title compound as a yellow gum, (1.74 g), ¹H NMR (300.073 MHz, d₆-DMSO) δ 1.57-1.92 (m, 4H), 2.54 (s, 3H), 2.76-3.03 (m, 9H), 3.57-3.71 (m, 1H), 4.51-4.70 (m, 1H), 7.34 (s, 4H), 7.58 (d, J=7.9 Hz, 1H), 7.70 (d, J=6.2 Hz, 1H), 8.02 (s, 1H), m/z 396 (M+H)⁺.

### Step 4: 4-[1-(3-amino-4-methyl-benzoyl)-4-piperidyl]-N,N-dimethyl-benzamide

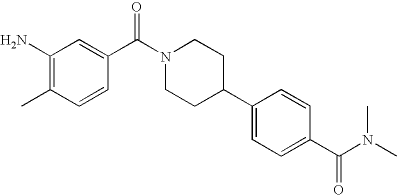

A solution of N,N-dimethyl-4-[1-(4-methyl-3-nitro-benzoyl)-4-piperidyl]benzamide (Step 3) (1.74 g) in MeOH (35 mL) was treated with palladium-on-charcoal catalyst (122 mg of 10% Pd/C). The reaction mixture was stirred in an atmosphere of hydrogen at ambient temperature and pressure for 2 hours. The catalyst was removed by filtration and the filtrate concentrated to give the title compound as a colourless solid (1.51 g), ¹H NMR (300.073 MHz, d₆-DMSO) δ 1.46-1.68 (m, 2H), 1.69-1.88 (m, 2H), 2.06 (s, 3H), 2.74-3.07 (m, 9H), 3.64-3.97 (m, 1H), 4.43-4.70 (m, 1H), 4.97 (s, 2H), 6.49 (d, J=7.4 Hz, 1H), 6.64 (s, 1H), 6.95 (d, J=7.5 Hz, 1H), 7.32 (s, 4H), m/z 366 (M+H)⁺.

### Intermediate L

### 4-[1-(3-amino-4-ethyl-benzoyl)-4-piperidyl]benzonitrile

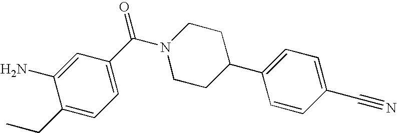

### Step 1: 4-ethyl-3-nitro-benzoic acid

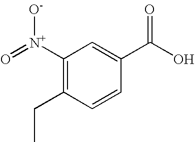

Concentrated nitric acid (80 mL)was cooled to approximately 0-5° C. in an ice bath and 4-ethyl benzoic acid (10 g, 66.59 mmol) was added portionwise. The resultant mixture was allowed to warm up to ambient temperature and the reaction mixture was stirred for approx. 72 hrs. It was then warmed to 60° C. at which it was maintained overnight at this temperature. The reaction mixture was quenched into ice/water (200 mL) and the resulting precipitate isolated by filtration and washed with water to give the title compounds as a colourless solid (9.52 g, 73%), ¹H NMR (300.073 MHz, d₆-DMSO, 30° C.) δ 1.22 (3H, t J=8.3 Hz), 2.87 (2H, q J=7.8 Hz), 7.65 (1H, d J=8.3 Hz), 8.13 (1H, d J=9.0 Hz), 8.34 (1H, s), 13.00-13.80 (1H, m), m/z 194 (M−H)⁻.

### Step 2: 4-[1-(4-ethyl-3-nitro-benzoyl)-4-piperidyl]benzonitrile

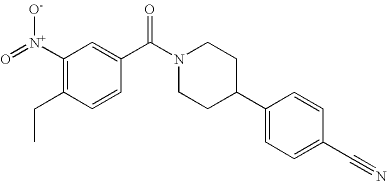

The title compound was prepared by an amide coupling reaction starting from 4-ethyl-3-nitro-benzoic acid (Step 1) and 4-(4-piperidyl)benzonitrile as described for Intermediate A,

¹H NMR (300.073 MHz, d₆-DMSO, 30° C.) δ 1.22 (3H, tJ=7.4 Hz), 1.56-1.96 (4H, m), 2.77-3.01 (4H, m—contains q from ethyl), 3.04-3.25 (1H, m), 3.63 (1H, br s), 4.61 (1H, br s), 7.50 (2H, dJ=9.1 Hz), 7.59 (1H, dJ=7.4 Hz), 7.67-7.81 (3H, m), 7.94-7.98 (1H, m).

### Step 3: 4-[1-(3-amino-4-ethyl-benzoyl)-4-piperidyl]benzonitrile

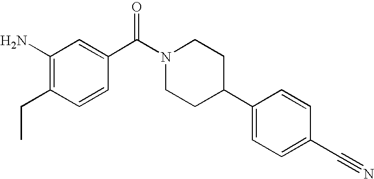

The title compound was prepared by hydrogenation of 4-[1-(4-ethyl-3-nitro-benzoyl)-4-piperidyl]benzonitrile (Step 2) as described for Intermediate I, Step 3, ¹H NMR (300.073 MHz, d₆-DMSO, 30° C.) δ 1.13 (3H, tJ=6.8 Hz), 1.46-1.67 (2H, m), 1.67-1.91 (2H, m), 2.38-2.48 (2H, m), 2.64-3.21 (3H, m), 3.59-4.05 (1H, m), 4.33-4.72 (1H, m), 4.98 (2H, s), 6.53 (1H, dJ=7.3 Hz), 6.64 (1H, s), 6.95 (1H, dJ=6.1 Hz), 7.49 (2H, dJ=7.2 Hz), 7.76 (2H, dJ=9.5 Hz), m/z 334 (M+H)⁺.

### Intermediate M

### 4-[1-(5-amino-2-fluoro-benzoyl)-4-piperidyl]benzonitrile

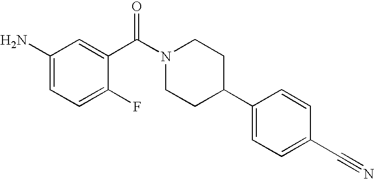

The title compound was prepared by the method described for Intermediate A, starting from 5-amino-2-fluoro-benzoic acid and 4-(4′-cyanophenyl)piperidine, ¹H NMR (300.073 MHz, DMSO-d₆) δ1.55 (2H, d), 1.74-1.79 (1H, m), 1.88 (1H, d), 2.88 (2H, d), 3.13 (1H, d), 3.57 (1H, d), 4.64 (1H, d), 5.10 (2H, s), 6.50 (1H, s), 6.56-6.60 (1H, m), 6.89 (1H, d), 7.46 (2H, d), 7.76 (2H, d), m/z 324 (M+H)⁺

### Intermediate N

### 4-[1-[3-amino-4-(trifluoromethyl)benzoyl]-4-piperidyl]benzonitrile

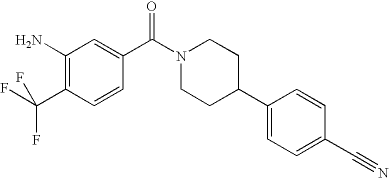

### Step 1:

### 3-amino-4-(trifluoromethyl)benzoic acid

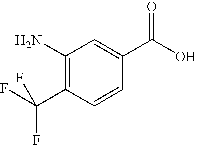

A mixture of 3-nitro-4-(trifluoromethyl)benzoic acid (4.56 g, 19.39 mmol) and palladium-on-charcoal catalyst (500 mg, 10% Pd) in methanol (100 ml) was stirred under an atmosphere of hydrogen until uptake of hydrogen was complete. The solvent was removed under reduced pressure to give the title compound as a cream solid (3.7 g), ¹H NMR (300.073 MHz, DMSO-d₆) δ5.81 (2H, s), 7.12 (1H, d), 7.40-7.45 (2H, m).

### Step 2:

### 4-[1-[3-amino-4-(trifluoromethyl)benzoyl]-4-piperidyl]benzonitrile

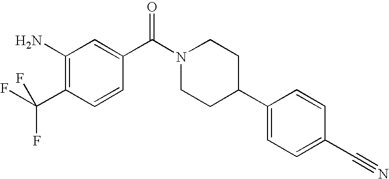

A stirred solution of 17029/82/1 (3.7 g, 18 mmol), 4-(4′-cyanophenyl)piperidine (3.36 g, 18 mmol) and DIPEA (9.4 ml, 54.1 mmol) in DCM (100 ml) was treated with N-(3-dimethylaminopropyl)-N′-ethylcarbodiimide hydrochloride (EDAC) (3.8 g, 19.8 mmol), and the reaction mixture stirred for 24 hrs and then allowed to stand for a further 24 hrs at ambient temperature. The bulk of the DCM was removed in vacuo and the residue partioned between water and EtOAc was attempted. The organic phase was separated and dried (MgSO₄); evaporation of the solvent gave a gum (5.8 g) which was recrystallised from EtOH (100 mL) to give a solid (2.2 g) which was purified by column chromatography (120 g silica cartridge, eluting with a gradient of 0-50% MeOH in DCM to give the title compound as a pale yellow solid (1.6 g 24%), ¹H NMR (300.073 MHz, d₆-DMSO, 30° C.) δ 1.49-1.95 (4H, m), 2.70-3.01 (2H, m), 3.02-3.23 (1H, m), 3.54-3.78 (1H, m), 4.49-4.71 (1H, m), 5.75 (2H, s), 6.63 (1H, dJ=7.3 Hz), 6.83 (1H, s), 7.37 (1H, dJ=8.5 Hz), 7.49 (2H, dJ=7.3 Hz), 7.77 (2H, dJ=7.9 Hz).

### Intermediate O

### 1-(5-(4-(4-cyanophenyl)piperidine-1-carbonyl)-2-methylphenyl)-3-((trans)-2-(1,3-dioxoisoindolin-2-yl)cyclohexyl)urea

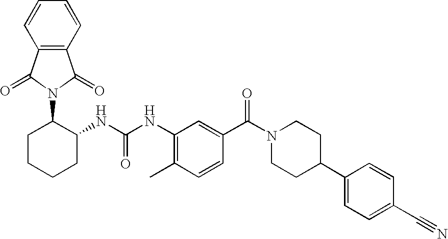

### Step 1:

### 2-[(trans)-2-aminocyclohexyl]isoindole-1,3-dione

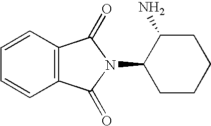

The title compound was prepared as described in Tetrahedron Asymmetry 14 1559-1563 (2003).

### Step B:

### 1-(5-(4-(4-cyanophenyl)piperidine-1-carbonyl)-2-methylphenyl)-3-((trans)-2-(1,3-dioxoisoindolin-2-yl)cyclohexyl)urea

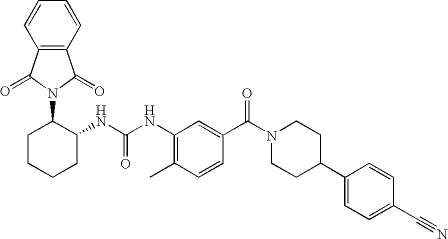

The title compound was prepared by the procedure described in Method 2, starting from 4-[1-(3-amino-4-methyl-benzoyl)-4-piperidyl]benzonitrile (Intermediate A) and 2-[(trans)-2-aminocyclohexyl]isoindole-1,3-dione (Step 1), ¹H NMR (300.072 MHz, CDCl₃) δ 1.85-1.21 (11H, m), 1.89 (3H, s), 2.58-2.47 (1H, m), 3.11-2.80 (2H, m), 3.70-3.63 (1H, m), 3.94-3.86 (1H, m), 4.44-4.33 (1H, m), 5.03-4.75 (2H, m), 6.16 (1H, s), 7.00 (1H, s), 7.19-7.02 (2H, m), 7.37-7.31 (3H, m), 7.66-7.60 (2H, m), 7.75 (2H, s), m/z (EI+) (M+H)+=590.52; HPLC tR=2.57 min. m/z (EI−) (M−H)−=588.45; HPLC tR=2.57 min.

### Intermediate P

### tert-butyl N-[1-[[5-[4-(4-cyanophenyl)piperidine-1-carbonyl]-2-methyl-phenyl]carbamoyl]azetidin-3-yl]carbamate

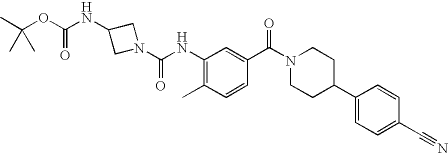

The title compound was prepared by the procedure described in Method 2, starting from 4-[1-(3-amino-4-methyl-benzoyl)-4-piperidyl]benzonitrile (Intermediate A) and 3-N-Boc-amino-azetidine, 1H NMR (300.072 MHz, CDCl₃) δ 1.45 (9H, s), 1.66-1.95 (4H, m), 2.20 (3H, s), 2.76-2.89 (2H, m), 2.97-3.05 (1H, m), 3.84-3.90 (2H, m), 3.97-4.03 (1H, m), 4.30 (2H, t), 4.42-4.54 (1H, m), 4.77-4.93 (1H, m), 5.11-5.24 (1H, m), 6.12 (1H, s), 7.05-7.19 (2H, m), 7.32 (2H, d), 7.60 (2H, d), 7.76 (1H, s), m/z (ESI+) (M+H)+=518; HPLC tR=2.27 min.

### Intermediate Q

### 4-methyl-3-(propan-2-ylcarbamoylamino)benzoic acid

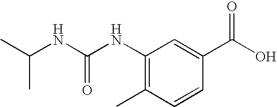

### Step 1:

### Methyl 4-methyl-3-(propan-2-ylcarbamoylamino)benzoate

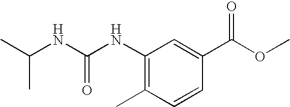

Isopropyl isocyanate (5.07 mL, 51.76 mmol) was added to methyl 3-amino-4-methyl benzoate (5.7 g, 34.51 mmol) and DMAP (4.22 g, 34.51 mmol) in MeCN (125 mL) and the reaction mixture warmed to 55° C. over a period of 5 minutes under nitrogen. The resulting solution was stirred at 55° C. for 6 hours. The precipitate which formed was collected by filtration, washed with MeCN (100 mL) and dried under vacuum to afford methyl 3-(3-isopropylureido)-4-methylbenzoate (3.38 g, 39.1%) as a colourless solid, which was used without further purification, ¹H NMR (300.073 MHz, d₆-DMSO) δ 1.11 (6H, d), 2.22 (3H, s), 3.68-3.86 (4H, m), 6.50-6.59 (1H, m), 7.23 (1H, d), 7.44 (1H, d), 7.62 (1H, s), 8.58 (1H, s), m/z (ESI+) (M+H)+=251.33; HPLC tR=1.80 min.

### Step 2:

### 4-methyl-3-(propan-2-ylcarbamoylamino)benzoic acid

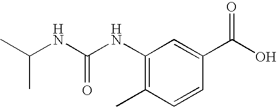

The material from Step 1 was hydrolysed using aqueous sodium hydroxide in a similar procedure to that described in Method 7 to give the title compound as a colourless solid, ¹H NMR (300.073 MHz, d₆-DMSO) δ 1.11 (6H, d), 2.21 (3H, s), 3.66-3.84 (1H, m), 6.50 (1H, d), 7.20 (1H, d), 7.42 (1H, d), 7.59 (1H, s), 8.52 (1H, s), 12.56 (1H, s), m/z (ESI+) (M+H)+=237.26; HPLC tR=1.45 min.

### Intermediate R

### (3-amino-4-methyl-phenyl)-[4-[4-(trifluoromethyl)phenyl]-1-piperidyl]methanone

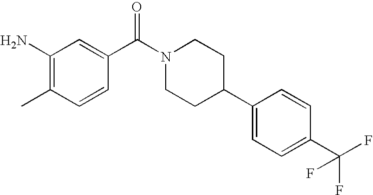

The title compound was prepared by the method described for Intermediate A, starting from 3-amino-4-methyl-benzoic acid and 4-[4-(trifluoromethyl)phenyl]piperidine hydrochloride,

¹H NMR (300.073 MHz, d₆-DMSO) d1.48-1.68 (m, 2H), 1.69-1.94 (m, 2H), 2.06 (s, 3H), 2.84-3.02 (m, 3H), 3.49-4.20 (m, 1H), 4.23-4.82 (m, 1H), 4.91-5.03 (m, 2H), 6.49 (d, 1H), 6.62 (s, 1H), 6.95 (d, 1H), 7.51 (d, 2H), 7.65 (d, 2H), m/z (ESI+) (M+H)+=363.37; HPLC tR=2.53 min.

### Intermediate S

### (3-amino-4-methyl-phenyl)-[4-(4-fluorophenyl)-1-piperidyl]methanone

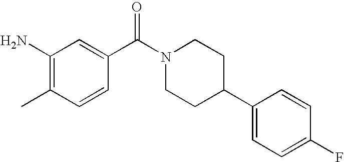

The title compound was prepared by the method described for Intermediate A, starting from 3-amino-4-methyl-benzoic acid and 4-(4-fluorophenyl)piperidine hydrochloride, ¹H NMR (300.073 MHz, d₆-DMSO) d1.43-1.62 (m, 2H), 1.65-1.87 (m, 2H), 2.01 (s, 3H), 2.73-2.87 (m, 2H), 2.89-3.13 (m, 1H), 3.54-4.14 (m, 1H), 4.20-4.76 (m, 1H), 4.88 (s, 2H), 6.45-6.51 (m, 1H), 6.61-6.66 (m, 1H), 6.94 (d, 1H), 7.05-7.15 (m, 2H), 7.24-7.35 (m, 2H), m/z (ESI+) (M+H)+=313.33; HPLC tR=2.22 min.

### Intermediate T

### 1-[5-[4-(4-fluorophenyl)piperidine-1-carbonyl]-2-methyl-phenyl]-3-(2-methylaminoethyl)urea

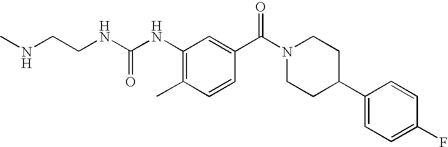

The title compound was prepared by the method described in Method 3, starting from tert-butyl N-[2-[[5-[4-(4-fluorophenyl)piperidine-1-carbonyl]-2-methyl-phenyl]carbamoylamino]ethyl]-N-methyl-carbamate (Example 347), ¹H NMR (300.073 MHz, d₆-DMSO) δ 1.44-1.86 (4H, m), 1.90 (3H, s), 2.24 (3H, s), 2.55 (2H, t), 2.71-3.06 (3H, m), 3.30-3.43 (2H, m), 3.70-3.87 (1H, m), 4.51-4.67 (1H, m), 6.94 (1H, d), 7.04-7.35 (6H, m), 7.92 (1H, s), 8.16 (1H, s), 8.63-8.76 (2H, m), m/z (ESI+) (M+H)+=413.54; HPLC tR=1.33 min.

### Intermediate U

### 1-(2-aminoethyl)-3-[5-[4-(4-cyanophenyl)piperidine-1-carbonyl]-2-methyl-phenyl]-1-methyl-urea

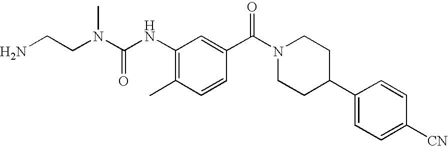

### Step 1:

### tert-butyl N-[2-[[5-[4-(4-cyanophenyl)piperidine-1-carbonyl]-2-methylphenyl]carbamoyl-methylamino]ethyl]carbamate

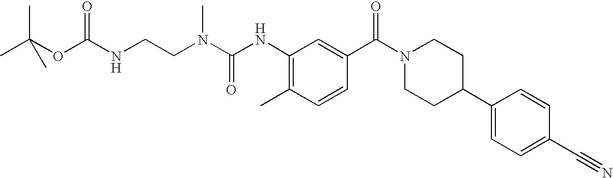

The title compound was prepared by the procedure described in Method 2, starting from 4-[1-(3-amino-4-methyl-benzoyl)-4-piperidyl]benzonitrile (Intermediate A) and tert-butyl N-(2-methylaminoethyl)carbamate, ¹H NMR (300.073 MHz, d₆-DMSO) δ 1.35 (9H, s), 1.49-1.91 (4H, m), 2.21 (3H, s), 2.75-3.16 (8H, m), 3.30-3.38 (2H, m), 3.57-4.05 (1H, m), 4.30-4.80 (1H, m), 6.75-6.85 (1H, m), 7.08 (1H, d), 7.22 (1H, d), 7.35 (1H, s), 7.49 (2H, d), 7.68-7.81 (3H, m), m/z (ESI+) (M+H)+=420.39; HPLC tR=2.32 min.

### Step 2:

### 1-(2-aminoethyl)-3-[5-[4-(4-cyanophenyl)piperidine-1-carbonyl]-2-methyl-phenyl]-1-methyl-urea

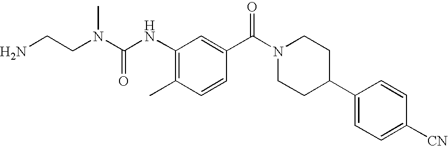

The title compound was prepared by the process described in Method 3, starting from tert-butyl N-[2-[[5-[4-(4-cyanophenyl)piperidine-1-carbonyl]-2-methylphenyl]carbamoyl-methylamino]ethyl]carbamate (Step 1) to give the title compound as the hydrochloride salt, 1H NMR (300.073 MHz, d₆-DMSO) δ 1.51-1.87 (4H, m), 2.23 (3H, s), 2.80-3.22 (8H, m), 3.49-3.59 (2H, m), 3.64-3.97 (1H, m), 4.09-4.77 (1H, m), 7.10 (1H, d), 7.23 (1H, d), 7.37 (1H, s), 7.50 (2H, d), 7.77 (2H, d), 7.93-8.18 (4H, m), m/z (ESI+) (M+H)+=420.47; HPLC tR=1.19 min.

### Intermediate V

### (3-amino-4-methylphenyl)-[4-(4-chlorophenyl)piperidin-1-yl]methanone

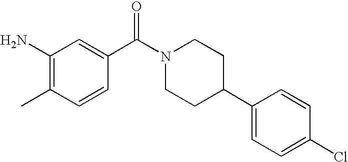

The title compound was prepared by the process described for Intermediate A, staring from 3-amino-4-methylbenzoic acid and 4-(4-chlorophenyl)piperidine hydrochloride, ¹H NMR (300.073 MHz, d₆-DMSO) δ 1.40-1.63 (2H, m), 1.63-1.89 (2H, m), 2.06 (3H, s), 2.65-3.17 (3H, m), 3.61-4.05 (1H, m), 4.32-4.73 (1H, m), 4.97 (2H, s), 6.48 (1H, d), 6.63 (1H, s), 6.95 (1H, d), 7.29 (2H, d), 7.35 (2H, d), m/z (ESI+) (M+H)+=329.36; HPLC tR=2.44 min.

### Intermediate W

### 4-[1-(3-amino-4-methylsulfanylbenzoyl)piperidin-4-yl]benzonitrile

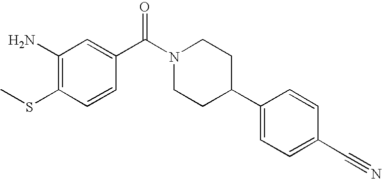

### Step 1:

### 4-[1-(4-methylsulfanyl-3-nitrobenzoyl)piperidin-4-yl]benzonitrile

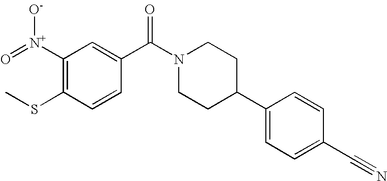

Oxalyl chloride (2.5 mL, 28 mmol) was added to a stirred suspension of 4-methylsulfanyl-3-nitro-benzoic acid (5 g, 23.45 mmol) in dichloromethane (50 mL), followed by the addition of DMF (2 drops), and the reaction mixture was stirred at ambient temperature for 2 hrs. The volatiles were removed in vacuo and the residue redissolved in dichloromethane (25 mL). This solution was added to a stirred solution of 4-(4′-cyanophenyl)piperidine (4.36 g, 23.45 mmol) and DIPEA (8.99 mL, 51.59 mmol) in DCM (25 mL) and the reaction mixture stirred for 20 hrs. It was then diluted with DCM and the resulting solution was washed sequentially with 0.5M HCl solution, saturated NaHCO₃ solution, and brine. The organic phase was dried and concentrated in vacuo to give a yellow solid which was purified by chromatography on silica (120 g column, eluting with 10-100% EtOAc in isohexane to give the title compound as a yellow solid (2.6 g), ¹H NMR (300.072 MHz, CDCl₃) [symbol] 1.59-2.04 (m, 4H), 2.54 (s, 3H), 2.80-2.93 (m, 2H), 3.01-3.18 (m, 1H), 3.77-4.20 (m, 1H), 4.44-5.03 (m, 1H), 7.33 (d, 2H), 7.44 (d, 1H), 7.63 (d, 2H), 7.68-7.72 (m, 1H), 8.34 (d, 1H), m/z (ESI+) (M+H)+=382; HPLC tR=2.54 min.

### Step 2:

### 4-[1-(3-amino-4-methylsulfanylbenzoyl)piperidin-4-yl]benzonitrile

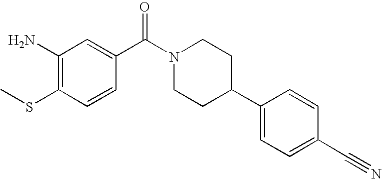

A mixture of 4-[1-(4-methylsulfanyl-3-nitrobenzoyl)piperidin-4-yl]benzonitrile (Step 1) (2.6 g, 6.82 mmol), iron (III) chloride hexahydrate (5.53 g, 20.45 mmol) and zinc dust (4.46 g, 68.2 mmol) in DMF (70 ml) and water (35 ml) was heated at 100° C. for 4 hrs. The reaction mixture was filtered through celite and evaporated in vacuo. Ethyl acetate (30 ml) was added to the filtrate and the resulting mixture was washed sequentially with water (2×30 mL) and saturated brine (30 mL). A beige solid impurity was removed by filtration and the organic filtrate was dried (MgSO₄) and evaporated in vacuo to give a yellow foam which was purified by chromatography on silica (40 g column, eluting with 20-80% EtOAc in isohexane) to give the title compound as a colourless solid (0.83 g), ¹H NMR (300.072 MHz, CDCl₃) δ 1.52-1.98 (m, 4H), 2.36 (s, 3H), 2.79-2.90 (m, 2H), 2.94-3.05 (m, 1H), 3.86-4.11 (m, 1H), 4.30 (s, 2H), 4.67-5.06 (m, 1H), 6.71-6.78 (m, 2H), 7.29-7.36 (m, 3H), 7.61 (d, 2H), m/z (ESI+) (M+H)+=352; HPLC tR=2.27 min.

### Intermediate X

### 4-[1-(3-amino-4-methylsulfonylbenzoyl)piperidin-4-yl]benzonitrile

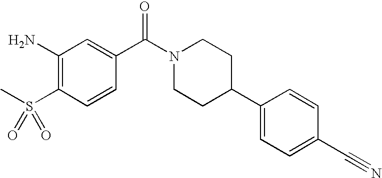

### Step 1:

### 4-[1-(4-methylsulfonyl-3-nitrobenzoyl)piperidin-4-yl]benzonitrile

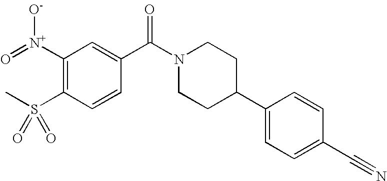

The title compound was prepared by the method described for Intermediate W, Step 1, starting from 4-methylsulfonyl-3-nitrobenzoic acid and 4-(4′-cyanophenyl)piperidine, ¹H NMR (300.072 MHz, CDCl₃) δ 1.62-2.12 (m, 4H), 2.84-2.97 (m, 2H), 3.11-3.34 (m, 1H), 3.45 (s, 3H), 3.61-3.82 (m, 1H), 4.72-5.08 (m, 1H), 7.33 (d, 2H), 7.63 (d, 2H), 7.79-7.82 (m, 1H), 7.89 (d, 1H), 8.27 (d, 1H), m/z (ESI+) (M+H+MeCN)+=455; HPLC tR=2.28 min.

### Step 2:

### 4-[1-(3-amino-4-methylsulfonylbenzoyl)piperidin-4-yl]benzonitrile

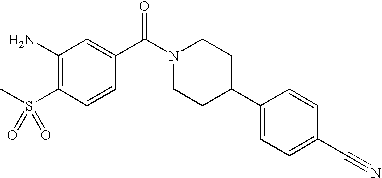

The title compound was prepared by the method described for Intermediate W, Step 2, starting from 4-[1-(4-methylsulfonyl-3-nitrobenzoyl)piperidin-4-yl]benzonitrile (Step 1), ¹H NMR (300.072 MHz, CDCl₃) [symbol] 1.51-2.08 (m, 4H), 2.79-2.93 (m, 2H), 3.06 (s, 3H), 3.11-3.24 (m, 1H), 3.74-3.91 (m, 1H), 4.74-4.92 (m, 1H), 5.16 (s, 2H), 6.79-6.83 (m, 2H), 7.32 (d, 2H), 7.62 (d, 2H), 7.78 (d, 1H), m/z (ESI−) (M−H)⁻=382; HPLC tR=2.03 min.

### Intermediate Y

### 4-[1-[3-amino-4-(methoxymethyl)benzoyl]piperidin-4-yl]benzonitrile

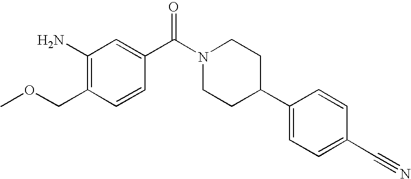

### Step 1:

### 4-(methoxymethyl)-3-nitrobenzoic acid

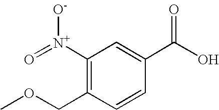

A solution of sodium methoxide in methanol (0.5 M, 115 mL, 57.68 mmol) was added dropwise to a stirred mixture of 4-(bromomethyl)-3-nitrobenzoic acid (5 g, 19.23 mmol) in methanol (100 mL) over a period of 5 minutes. The resulting mixture was stirred at 62° C. for 1 hour and was then quenched with water (100 mL) and the bulk of the methanol removed under reduced pressure. The reaction mixture was acidified with 2M HCl. The resulting precipitate was collected by filtration, washed with water (150 mL) and dried in the vacuum oven to give the title compound as a pale orange solid (2.96 g, 72.9%), which was used without further purification, ¹H NMR (300.073 MHz, d₆-DMSO) δ 3.39 (3H, s), 4.82 (2H, s), 7.86 (1H, d), 8.22-8.28 (1H, m), 8.47 (1H, d), 13.58 (1H, s), m/z (ESI−) (M−H)−=210.25; HPLC tR=1.69 min.

### Step 2:

### 4-[1-[4-(methoxymethyl)-3-nitrobenzoyl]piperidin-4-yl]benzonitrile

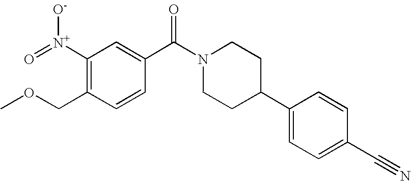

The title compound was prepared by the method described for Intermediate W, Step 1, starting from 4-(methoxymethyl)-3-nitrobenzoic acid and 4-(4′-cyanophenyl)piperidine (Step 1), ¹H NMR (300.073 MHz, d₆-DMSO) δ 1.59-1.97 (4H, m), 2.74-3.02 (2H, m), 3.06-3.24 (1H, m), 3.37 (3H, s), 3.50-3.80 (1H, m), 4.50-4.72 (1H, m), 4.77 (2H, s), 7.51 (2H, d), 7.73-7.85 (4H, m), 8.09 (1H, s), m/z—no mass ion observed; HPLC tR=2.45 min.

### Step 3:

### 4-[1-[3-amino-4-(methoxymethyl)benzoyl]piperidin-4-yl]benzonitrile

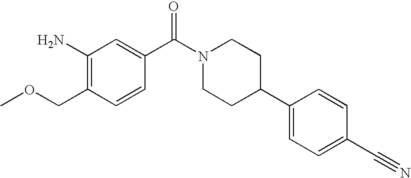

The title compound was prepared by the method described for Intermediate D, Step 2, starting from 4-[1-[4-(methoxymethyl)-3-nitrobenzoyl]piperidin-4-yl]benzonitrile (Step 2), and using a MeOH and THF mixture (1:1.5 by volume) as solvent, ¹H NMR (300.073 MHz, d₆-DMSO) δ 1.48-1.67 (2H, m), 1.68-1.92 (2H, m), 2.65-3.22 (3H, m), 3.27 (3H, s), 3.58-3.96 (1H, m), 4.32 (2H, s), 4.40-4.75 (1H, m), 5.09 (2H, s), 6.55 (1H, d), 6.67 (1H, s), 7.07 (1H, d), 7.49 (2H, d), 7.76 (2H, d), m/z (ESI+) (M+H)+=350.28; HPLC tR=2.01 min.

1) A compound of formula I 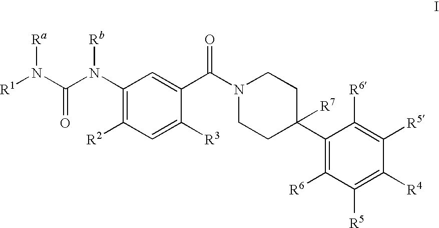
or a pharmaceutically acceptable salt thereof, in which
R¹ represents 1) a C₁₋₆alkyl group optionally substituted by one or two groups selected from A-Y below and/or by one to five groups selected from X below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) a group CONR^{e}R^{f} in which R^{e} and R^{f} are as defined below; vii) C₁₋₆alkanoyl; viii) benzoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulfamoyl; xvi) N—C₁₋₆alkylsulfamoyl; xvii) N,N-diC₁₋₆alkylsulfamoyl; xviii) benzyl or benzyloxy; xix) nitro; xx) heteroaryl; xxi) heteroaryloxy; xxii) phenyl xxiii) phenoxy xxiv) phenylsulfamoyl; xxv) heteroarylsulfamoyl; xxvi) a carbon linked saturated or partially unsaturated 4 to 10 membered heterocyclic group as defined in c) below; xxvii) phenylsulfonyl; xxviii) heteroarylsulfonyl;
xxix) a group of formula NR^{c}R^{d} in which R^{c} and R^{d} independently represent:
a) H;
b) C₁₋₆alkanoyl optionally substituted by carboxy or a C₁₋₆alkoxycarbonyl group;
c) a carbon linked saturated or partially unsaturated 4 to 10 membered heterocyclic group containing one or more N, S or O, wherein the S may be in its oxidised form of SO or SO₂, which is optionally fused to a benz ring and any ring is optionally substituted by one or more of the following: hydroxy, halo, oxo, carboxy, a C₁₋₆ alkoxycarbonyl group, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 10 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; halo; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl or nitro;
k) heteroaryl optionally substituted by one or more carboxy; fluoro; hydroxy; a C₁₋₆alkyl group (which is optionally substituted by one or more of the following: a C₁₋₆alkoxy group, hydroxy or a group of formula NR^{e}R^{f} in which R^{e} and R^{f} are as defined above); a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group optionally substituted by phenyl;
n) heteroarylcarbonyl;
o) sulfamoyl optionally substituted by one or two independently selected C₁₋₆alkyl groups or the terminal nitrogen is included in a 5 or 6 membered saturated or partially unsaturated heterocyclic ring optionally containing an additional N, S or O, wherein the S may be in its oxidised form of SO or SO₂;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 10 membered heterocyclic group containing one or more N, S or O, wherein the S may be in its oxidised form of SO or SO₂, which is optionally fused to a benz ring or a heteroaryl ring and is optionally substituted on any ring by one or more of the following: hydroxy; halo; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; a C₁₋₆alkyl optionally substituted by one or more hydroxy or C₁₋₆alkoxy; or a C₁₋₆ alkoxycarbonyl group;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) C₁₋₆alkylthio;
R) ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
S) phenoxy;
T) hydroxy;
U) oxo
V) carboxy;
W) cyano;
X) sulfamoyl optionally substituted by one or two independently selected C₁₋₆alkyl groups or the nitrogen is included in a 4 or 7 membered saturated or partially unsaturated heterocyclic ring optionally containing an additional N, S or O, wherein the S may be in its oxidised form of SO or SO₂;
Y) sulfamoylamino optionally substituted by one or two independently selected C₁₋₆alkyl groups or the terminal nitrogen is included in a 4 or 7 membered saturated or partially unsaturated heterocyclic ring optionally containing an additional N, S or O, wherein the S may be in its oxidised form of SO or SO₂;
Z) fluoro or chloro;
or R¹ represents
2) a C₃₋₁₀cycloalkyl group optionally substituted by one or two groups selected from A to Y above and/or by one to five groups selected from Z above;
3) a C₂₋₆alkynyl group optionally substituted by one or two groups selected from A to Y above and/or by one to five groups selected from Z above;
4) a carbon linked saturated or partially unsaturated 4 to 10 membered heterocyclic group containing one or more N, S or O, wherein the S may be in its oxidised form of SO or SO₂, which is optionally fused to a benz ring or a heteroaryl ring and any ring is optionally substituted by one or two groups A to Y as defined above and/or by one to five groups selected from Z above;
5) a C₂₋₆alkenyl group optionally substituted by one or two groups selected from A to Y above and/or by one to five groups selected from Z above;
wherein any alkyl chain mentioned in any of the definitions from A to Y above or in any of the definitions i to xxix above is optionally substituted by 1) one or two groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; a C₁₋₄alkanoyloxy group or a C₁₋₄alkyl optionally substituted by one or more hydroxy, C₁₋₃alkoxy or a group —NR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 10 membered heterocyclic group in the list of optional substituents from A to Y above or in any of the definitions i to xxix above, for which specific substitution has not been previously mentioned, is optionally substituted by one, two or three groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R^{a} represents H; or a C₁₋₄alkyl group, a C₃₋₆cycloalkyl group or a C₃₋₆cycloalkylC₁₋₄alkyl group each of which groups is optionally substituted by one or more carboxy; fluoro; hydroxy; a C₁₋₃alkoxy group optionally substituted on C2 or C3 by carboxy; a group of formula NR^{e}R^{f} in which R^{e} and R^{f} are as defined above; or a group CONR^{e}R^{f} in which R^{e} and R^{f} are as defined above;
or R¹ and R^{a} together with the nitrogen atom to which they are attached represent a saturated or partially unsaturated 4 to 10 membered heterocyclic ring optionally containing an additional sulphur including oxidised as SO or SO₂, oxygen or nitrogen which is optionally fused to a benz ring and wherein any ring is optionally substituted by one or two of the groups A to Y above and/or by from 1 to 5 groups Z;
R^{b} represents H;
R² represents H, halo, cyano, a C₁₋₃alkyl group which is optionally substituted by one or more of the following: halo; a C₁₋₃alkoxy group; or by a group C₁₋₃alkylS(O)ᵤ— which is optionally substituted by one or more fluoro and u is 0, 1 or 2; or R² represents a C₁₋₃alkoxy group optionally substituted by one or more halo or R² represents a C₁₋₆alkylS(O)ₐ(O)_{b}— group wherein the C₁₋₆alkyl is optionally substituted by one or more fluoro and a is 0, 1 or 2 and b is 0 except when a is 2 then b may also be 1;
R³ represents H, halo, cyano, a C₁₋₃alkyl group which is optionally substituted by one or more of the following: halo; C₁₋₃alkoxy group; or by a group C₁₋₃alkylS(O)ₜ— which is optionally substituted by one or more fluoro and t is 0, 1 or 2; or R² represents a C₁₋₃alkoxy group optionally substituted by one or more halo or R² represents a C₁₋₆alkylS(O)_{c}(O)_{d}— group wherein the C₁₋₆alkyl is optionally substituted by one or more fluoro and c is 0, 1 or 2 and d is 0 except when c is 2 then d may also be 1;
R⁴ represents i) H
ii) a C₁₋₃alkyl group optionally substituted by cyano, hydroxy, a C₁₋₃alkoxy group or optionally substituted by one or more halo
iii) a C₁₋₃alkoxy group optionally substituted by one or more halo or optionally substituted by cyano, hydroxy, a C₁₋₃alkoxy group, an amino group of formula NR^{u}R^{v} in which R^{u} and R^{v} independently represent H, a C₁₋₃alkylsulphonyl group, an aminoC₁₋₃alkylsulphonyl group in which the amino is optionally substituted by one or more C₁₋₃alkyl groups, a C₁₋₃alkanoyl group, a C₁₋₃alkoxycarbonyl group or a C₁₋₃alkyl group optionally substituted by hydroxy or R^{u} and R^{v} together with the nitrogen atom to which they are attached represent azetidinyl, pyrrolidinyl, piperidinyl, piperazinyl or morpholinyl each of which is optionally substituted by one or more of the following: oxo, C₁₋₃alkyl or hydroxy;
iv) halo v) nitro vi) cyano
vii) a C₁₋₆alkylS(O)_{y}(O)_{z}— optionally substituted by one or more fluoro wherein y is 0, 1 or 2 and z is 0 except when y is 2 when z is 0 or 1
viii) a group -L-R^{g} in which L represents a bond, a C₃₋₆cycloalkylene group, a C₃₋₆cycloalkylidene group, a C₁₋₆alkylene group or a C₁₋₆alkoxyC₁₋₆alkylene group wherein each group is optionally substituted by one or more of the following: carboxy, hydroxy, a C₁₋₃alkyl group optionally substituted by hydroxy; and R^{g} represents carboxy or a group NR^{u}R^{v} in which R^{u} and R^{v} are as defined above and additionally R^{v} represents cyano or R^{g} represents a group CO₂R^{w} in which R^{w} is a C₁₋₃alkyl group; or R^{g} represents a group CONR^{x}R^{y} in which R^{x} and R^{y} independently represent H, a C₁₋₃alkylsulphonyl group, a C₁₋₃alkyl group or a C₃₋₆cycloalkyl group wherein the alkyl and cycloalkyl groups are optionally substituted by one or more hydroxy, carboxy or NR^{u}R^{v} in which R^{u} and R^{v} are as previously defined, or R^{x} and R^{y} together with the nitrogen atom to which they are attached represent azetidinyl; pyrrolidinyl, piperidinyl or morpholinyl; or R^{g} represents tetrazolyl, thiazolidin-2,4-dion-5-yl or R^{g} represents ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
ix) a group -L₁-N(R^{h})SO₂-L₂-Rⁱ in which L₁ and L₂ independently represent a bond or a C₁₋₆alkylene optionally substituted by one or more C₁₋₃alkyl groups, R^{h} is H or C₁₋₃alkyl and Rⁱ represents cyano or a group NR^{u}R^{v} in which R^{u} and R^{v} are as previously defined, or Rⁱ represents a group CO—R^{j} in which R^{j} represents hydroxy, C₁₋₃alkoxy or a group NR^{u}R^{v} in which R^{u} and R^{v} are as previously defined;
x) phenyl(O)_{f}— wherein f is 0 or 1 optionally substituted by one or more halo, C₁₋₃alkyl optionally substituted by one or more halo or C₁₋₃alkoxy optionally substituted by one or more halo;
xi) phenylthio optionally substituted by one or more halo, C₁₋₃alkyl optionally substituted by one or more halo or C₁₋₃alkoxy optionally substituted by one or more halo;
xii) monocyclic heteroaryl(O)_{g}— wherein g is 0 or 1 optionally substituted by one or more halo, C₁₋₃alkyl optionally substituted by one or more halo or C₁₋₃alkoxy optionally substituted by one or more halo;
xiii) a nitrogen containing 5 or 6 membered heteroarylCO— wherein the heteroaryl is linked through nitrogen to the carbonyl group optionally substituted by one or more halo, C₁₋₃alkyl optionally substituted by one or more halo or C₁₋₃alkoxy optionally substituted by one or more halo;
xiv) a C₂₋₆alkynyl group optionally substituted by one or more C₁₋₃alkyl, hydroxy, C₁₋₃alkoxy, C₁₋₃alkoxyC₁₋₃alkoxy, or a group —NR^{u}R^{v} as defined above;
xv) a group -L₃-S(O)ₑC₁₋₆alkyl in which L₃ is a C₁₋₆alkylene optionally substituted by one or more of the following: hydroxy or a C₁₋₃alkyl group, and e is 0, 1 or 2;
xvi) a group SO₂NR^{o}R^{p} in which R^{o} and R^{p} independently represent H; a C₁₋₆alkyl group optionally substituted by one or more of the following: hydroxy, C₁₋₆alkoxy or a group —NR^{u}R^{v} in which R^{k} and R^{l} are as defined above, or R^{o} and R^{p} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₃alkoxy group; carboxy; a C₁₋₃alkylsulfonyl group; C₁₋₃alkanoyl; benzoyl; hydroxy; oxo; carboxy; or by a C₁₋₃alkyl group optionally substituted by one or more of the following: hydroxy, C₁₋₃alkoxy or carboxy; or
xvii) —C(NH₂)═N—OH
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and
R⁷ is H or OH; with the proviso that the compound is not one of the following:
3-[5-[4-(4-cyanophenyl)piperidine-1-carbonyl]-2-methylphenyl]-1,1-dimethylurea
3-[3-[4-(4-cyanophenyl)piperidine-1-carbonyl]-4-methoxyphenyl]-1-propan-2-ylurea
3-benzyl-1-[3-[4-(4-cyanophenyl)piperidine-1-carbonyl]-4-methoxyphenyl]urea
Methyl (2S)-2-[[5-[4-(4-cyanophenyl)piperidine-1-carbonyl]-2-methylphenyl]carbamoylamino]-3-methylbutanoate
Methyl (2S)-2-[[5-[4-(4-cyanophenyl)piperidine-1-carbonyl]-2-methylphenyl]carbamoylamino]-2-phenylacetate
Methyl 2-[[5-[4-(4-cyanophenyl)piperidine-1-carbonyl]-2-methylphenyl]carbamoylamino]-3-(4-fluorophenyl)propanoate
Methyl 2-[[5-[4-(4-cyanophenyl)piperidine-1-carbonyl]-2-methylphenyl]carbamoylamino]-4-(tetrazol-1-yl)butanoate
Methyl (2S)-2-[[5-[4-(4-cyanophenyl)piperidine-1-carbonyl]-2-methylphenyl]carbamoylamino]-4-(1H-tetrazol-5-yl)butanoate
Methyl (2S)-2-[[5-[4-(4-cyanophenyl)piperidine-1-carbonyl]-2-methylphenyl]carbamoylamino]-3-(1-methylimidazol-4-yl)propanoate
Methyl 2-[[5-[4-(4-cyanophenyl)piperidine-1-carbonyl]-2-methylphenyl]carbamoylamino]-2-methylpropanoate
Ethyl (2S)-2-[[5-[4-(4-cyanophenyl)piperidine-1-carbonyl]-2-methylphenyl]carbamoylamino]-3-hydroxypropanoate
3-benzyl-1-[5-[4-(4-cyanophenyl)piperidine-1-carbonyl]-2-methylsulfonylphenyl]urea
N-[(trans)-2-[[5-[4-(4-cyanophenyl)piperidine-1-carbonyl]-2-methylphenyl]carbamoylamino]cyclohexyl]acetamide
1-[5-(4-hydroxy-4-phenylpiperidine-1-carbonyl)-2-methylphenyl]-3-propan-2-ylurea
1-[5-[4-(2-methoxyphenyl)piperidine-1-carbonyl]-2-methylphenyl]-3-propan-2-ylurea
1-[5-[4-hydroxy-4-[3-(trifluoromethyl)phenyl]piperidine-1-carbonyl]-2-methylphenyl]-3-propan-2-ylurea
1-[5-[4-(2-fluorophenyl)-4-hydroxypiperidine-1-carbonyl]-2-methylphenyl]-3-propan-2-ylurea
3-[5-[4-(4-cyanophenyl)piperidine-1-carbonyl]-2-methylphenyl]-1-methyl-1-(oxolan-2-ylmethyl)urea
3-[5-[4-(4-cyanophenyl)piperidine-1-carbonyl]-2-methylphenyl]-1-(2-methoxyethyl)-1-methylurea
3-[5-[4-(4-cyanophenyl)piperidine-1-carbonyl]-2-methylphenyl]-1-(2-cyanopropan-2-yl)-1-methylurea
3-[5-[4-(4-cyanophenyl)piperidine-1-carbonyl]-2-methylphenyl]-1-cyclopropyl-1-(1,1-dioxothiolan-3-yl)urea
N-[2-[[5-[4-(4-cyanophenyl)piperidine-1-carbonyl]-2-methylphenyl]carbamoyl-methylamino]ethyl]-2-methylpropanamide
N-[5-[4-(4-cyanophenyl)piperidine-1-carbonyl]-2-methylphenyl]-1,1-dioxo-1,4-thiazinane-4-carboxamide
1-[5-[4-(3-fluorophenyl)piperidine-1-carbonyl]-2-methylphenyl]-3-propan-2-ylurea
1-[5-[4-(3-chlorophenyl)piperidine-1-carbonyl]-2-methylphenyl]-3-propan-2-ylurea
1-[5-[4-(3-methoxyphenyl)piperidine-1-carbonyl]-2-methylphenyl]-3-propan-2-ylurea
1-[5-[4-(2-cyanophenyl)piperidine-1-carbonyl]-2-methylphenyl]-3-propan-2-ylurea
3-[3-[4-(4-cyanophenyl)piperidine-1-carbonyl]-4-methylsulfinylphenyl]-1-propan-2-ylurea or
3-[3-[4-(4-cyanophenyl)piperidine-1-carbonyl]-4-methylsulfonylphenyl]-1-propan-2-ylurea.

2) A compound according to claim 1 as represented by formula II 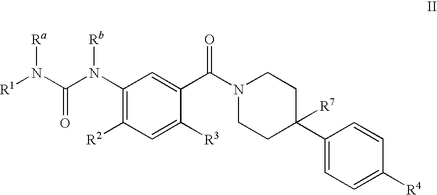
or a pharmaceutically acceptable salt thereof, in which
R¹ represents 1) a C₁₋₆alkyl group optionally substituted by one or two groups selected from A-S below and/or by one to five groups selected from T below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) carbamoyl; vii) N—C₁₋₆alkylcarbamoyl; viii) N,N-diC₁₋₆alkylcarbamoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl xix) benzyloxy; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl;
xxix) a group of formula NR^{c}R^{d} in which R^{c} and R^{d} independently represent:
a) H;
b) C₁₋₆alkanoyl;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl;
k) heteroaryl optionally substituted by one or more carboxy; fluoro; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; or a C₁₋₆alkyl optionally substituted by one or more hydroxy or C₁₋₆alkoxy;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) hydroxy;
R) oxo;
S) carboxy;
T) fluoro
or R¹ represents
2) a C₃₋₇cycloalkyl group optionally substituted by one or two groups selected from A to T above;
3) a C₂₋₆alkynyl group optionally substituted by one or two groups selected from A to T above;
4) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by a group A to T as defined above;
5) a C₂₋₆alkenyl group optionally substituted by one or two groups selected from A to T above;
wherein any alkyl chain mentioned in any of the definitions from A to P above or in any of the definitions i to xxix above is optionally substituted by 1) one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 8 membered heterocyclic group in the list of optional substituents from A to P above or in any of the definitions i to xxix above, for which specific substitution has not been previously mentioned, is optionally substituted by one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R^{a} represents H; or a C₁₋₄alkyl group, a C₃₋₆cycloalkyl group or a C₃₋₆cycloalkylC₁₋₄alkyl group each of which groups is optionally substituted by one or more carboxy; fluoro; hydroxy; a C₁₋₃alkoxy group optionally substituted on C2 or C3 by carboxy; a group of formula NR^{e}R^{f} in which R^{e} and R^{f} are as defined above; or a group CONR^{e}R^{f} in which R^{e} and R^{f} are as defined above; or R¹ and R^{a} together with the nitrogen atom to which they are attached represent a saturated or partially unsaturated 4 to 8 membered heterocyclic ring optionally containing an additional sulphur including oxidised as SO or SO₂, oxygen or nitrogen which is optionally fused to a benz ring and wherein any ring is optionally substituted by one or two of the groups A to S above and/or by from 1 to 5 groups T;
R^{b} represents H,
R² represents H, halo, cyano, a C₁₋₃alkyl group optionally substituted by one or more halo, or a C₁₋₃alkoxy group optionally substituted by one or more halo;
R³ represents H, halo, cyano, a C₁₋₃alkyl group optionally substituted by one or more halo, or a C₁₋₃alkoxy group optionally substituted by one or more halo;
R⁴ represents cyano, halo or a C₁₋₄alkylsulphonyl group: and
R⁷ represents H or hydroxy.

3) A compound according to claim 1 as represented by formula IIA 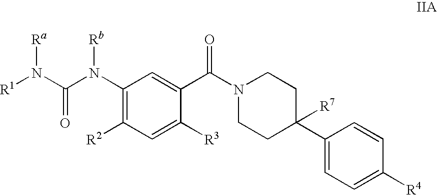
or a pharmaceutically acceptable salt thereof, in which
R¹ represents 1) a C₁₋₆alkyl group optionally substituted by one or more of the following:
a) phenyl optionally substituted by one or more of the following: halo; a C₁₋₄alkoxy group or cyano; b) pyridyl c) a carbon linked saturated 5 or 6 membered heterocyclic group containing one N or O; d) a C₁₋₆alkoxycarbonyl group or e) a C₂₋₄alkynyl group or 2) a C₃₋₇cycloalkyl group
R^{a} represents H; or a C₁₋₄alkyl group;
or R¹ and R^{a} together with the nitrogen atom to which they are attached represent morpholinyl, pyrrolidinyl or piperidinyl;
R^{b} represents H,
R² represents H, halo, trifluoromethoxy, a C₁₋₃alkyl group; a C₁₋₃alkoxy group; cyano; or when R¹ is other than phenyl then R^{b} together with the nitrogen to which is attached plus the carbon on the phenyl ring to which the nitrogen is attached and R² together with the carbon to which it is attached together represent a pyrrolidine ring fused to phenyl;
R³ represents H, halo, trifluoromethoxy, a C₁₋₃alkyl group; a C₁₋₃alkoxy group; cyano;
R⁴ represents bromo, cyano or a C₁₋₂alkylsulphonyl group: and
R⁷ represents H or hydroxy.

4) A compound according to any previous claim in which R⁷ represents H.

5) A compound according to any previous claim in which R⁷ represents H, R¹ represents 1) a C₁₋₆alkyl group optionally substituted by one or more of the following: a) phenyl optionally substituted by one or more of the following: halo; a C₁₋₄alkoxy group or cyano; b) pyridyl c) oxan-4-yl d) a C₁₋₄alkoxycarbonyl group or e) a C₂₋₄alkynyl group 2) a C₃₋₇cycloalkyl group and R^{a} represents H or R¹ and R^{a} together with the nitrogen atom to which they are attached represent morpholino or pyrrolidino, and R², R^{b}, R³, R⁴ are as described above provided that one of R² and R³ is other than H.

6) A compound according to any previous claim in which R² is methyl and R³ is H.

7) A compound according to any one of claims 1 to 5 in which R² and R³ are both methyl.

8) A compound according to any previous claim in which R⁴ is cyano or methylsulphonyl.

9) A compound according to any previous claim in which R^{a} is H.

10) A compound according to any one of claims 1 to 5 or claim 8 or claim 9 in which R³ is methyl and R² is H.

11) A compound selected from one or more of the compounds described in List 1 in the specification or a pharmaceutically acceptable salt thereof.

12) A compound selected from one or more of the compounds described in List 2 in the specification or a pharmaceutically acceptable salt thereof.

13) A method of treating obesity or being overweight, eating disorders, dyslipidaemia and type 2 diabetes mellitus comprising administering a pharmacologically effective amount of a compound of formula I as defined in any one of claims 1 to 12 to a patient in need thereof.

14) A method of treating cancer comprising administering a pharmacologically effective amount of a compound of formula I as defined in any one of claims 1 to 12 to a patient in need thereof.

15) A method of treating infection comprising administering a pharmacologically effective amount of a compound of formula I as defined in any one of claims 1 to 12 to a patient in need thereof.

16) A pharmaceutical formulation comprising a compound of formula I as defined in any one of claims 1 to 12, or pharmaceutically acceptable salt thereof, in admixture with pharmaceutically acceptable adjuvants, diluents and/or carriers.

17) A process for preparing a compound of formula I as claimed in claim 1 wherein R¹, R^{a}, R²R^{b}, R³, R⁴, R⁵, R^{5′}, R⁶, R^{6′} and R⁷ are as claimed in claim 1 unless otherwise specified which comprises
(a) reacting a compound of formula VI 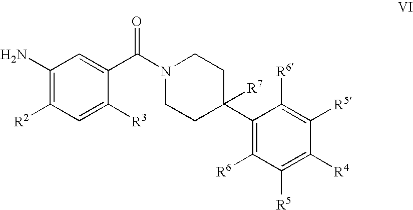
with an isocyanate of formula VII
R¹—N═C═O VII
to give compounds of formula I in which R^{a} is H or
b) reacting a compound of formula VI 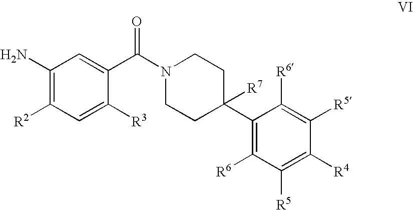
with phosgene or an equivalent thereof and then further reacting the intermediate obtained with an amine of formula VIII 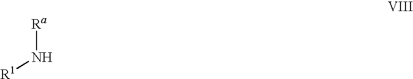
or c) reacting a compound of formula IX 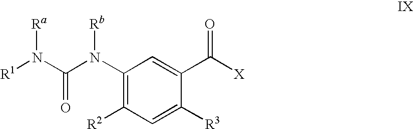
in which X represents a leaving group with a compound of formula X 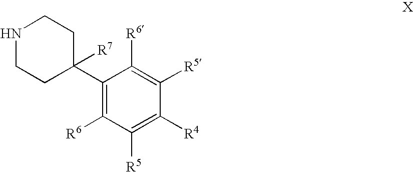
in the presence of a diluent and optionally in the presence of a base at a temperature in the range of 0-150° C.
or d) reacting a compound of formula XI 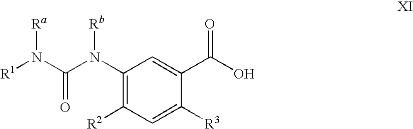
with a compound of formula X optionally in the presence of a coupling agent and optionally in the presence of a diluent at a temperature in the range of 0-150° C.
e) reacting a compound of formula XII 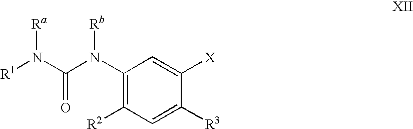
in which X represents a replaceable group, with a compound of formula X in the presence of carbon monoxide and in the presence of a metal catalyst in a solvent in the temperature range 0-150° C. or
f) reacting a compound of formula XIII 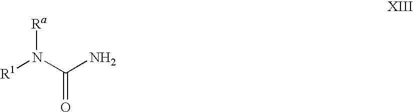
with a compound of formula XIV 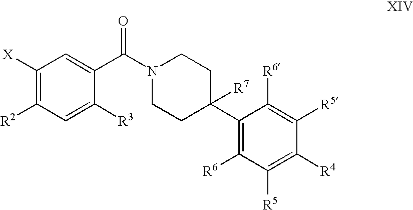
in which X represents a replaceable group in the presence of a metal catalyst in an organic diluent at a temperature in the range 0-150° C.

18) A compound of formula VI as described in the previous claim.
